SEQUENCE LISTING (108 sequences)

SEQ ID NO: 1 - DNA, 864 bp, Synthetic construct
  source 1..864: mol_type = other DNA; note = Synthetic ORF for target gene Rpl7a, Insert Myc/Myc/mRpl7a
atggaacaaaaactcatctcagaagaggatctggagcagaagctgatcagcgaggaggacctgatgcccaaagggaagaaggccaaggggaagaaggtggccccggcccccgccgtcgtcaagaagcaggaggccaagaaggtggtcaaccctctgttcgagaagaggcccaagaacttcggcattggacaggacatccagcccaaaagagatttaacgcgcttcgtcaaatggccccgctacatcagactgcagcggcaaagagctatcctctacaagcggctcaaagtccctcctgccattaaccagttcacccaggccctggacaggcagacagctactcagctgcttaagcttgcccacaagtacaggccagagacaaagcaagagaagaagcaaaggctactggcccgtgctgagaagaaagctgctggcaaaggcgacgtcccaactaagagaccacctgtcctccgagcaggagtcaatacagtcaccaccttggtggagaacaagaaggctcagctggtggtgattgcccatgacgtagaccccattgagctggtggttttcctacctgctctgtgtcgaaagatgggggtgccctactgcatcatcaagggaaaggccaggctggggcacctggtccacaggaagacatgcaccaccgttgccttcacacaggttaactcggaagacaagggtgctctggctaagctggtggaagctattaggaccaattataatgacagatatgacgagatccgtcgccactggggaggcaacgtcctgggtcctaagtctgtggctcgaattgccaagctggaaaaagcaaaggctaaagaactcgccactaaattgggttaa

SEQ ID NO: 2 - DNA, 876 bp, Synthetic construct
  source 1..876: mol_type = other DNA; note = Synthetic ORF for target gene Rpl7, Insert Myc/Myc/mRpl7
atggaacaaaaactcatctcagaagaggatctggagcagaagctgatcagcgaggaggacctgatggaggctgttccagagaagaaaaagaaggttgccactgtgccaggaacccttaagaaaaaggttcctgctgggccaaaaactctcaagaaaaaggttcccgctgtgccagaaaccctcaagaaaaagcgaaggaatttcgcagagttgaaggtgaagcgcctgaggaagaagtttgccctgaagacacttcgaaaggcaaggaggaagctcatctatgagaaggcaaagcactatcacaaggagtacaggcagatgtaccgcactgagattcggatggccaggatggcaaggaaagctggcaacttctatgtgcccgcagaaccaaagctggcctttgtcatcagaattcgaggtatcaatggagtaagcccaaaggttcgtaaggttctgcagctccttcgtcttcgacagatcttcaatggcacctttgttaagctcaacaaggcttcaattaacatgctgcggattgtggagccatacattgcatgggggtaccccaacctgaagtcagtaaacgagctcatctacaagcgaggctacggcaaaatcaacaagaagcggattgccttgacagataattccttgattgctcggtctcttggtaagtttggcatcatctgcatggaggatctaattcatgagatctatacagtcgggaaacgcttcaaggaagcaaataacttcctgtggcccttcaagttatcttccccacgaggtgggatgaagaaaaagacaactcactttgtagaaggtggagatgctggcaacagggaagaccagataaacaggcttattagacggatgaactaa

SEQ ID NO: 3 - DNA, 420 bp, Synthetic construct
  source 1..420: mol_type = other DNA; note = Synthetic ORF for target gene Tceb2 Myc/Myc/mTceb2
atggaacaaaaactcatctcagaagaggatctggagcagaagctgatcagcgaggaggacctgatggacgtgtttctcatgatccggcgccacaagaccaccatctttacggacgccaaggagtcgagcaccgtgttcgaactgaagcgcatcgtcgagggcatcctcaagcggccgccagaggagcagcggctttacaaggatgaccagctccttgatgatggcaaaactctgggcgagtgtggcttcactagccagacagcacggccacaggccccagccacagtgggcctggccttccgagcagatgacaccttcgaagctctccgcatcgagcccttttccagccctccagagcttccagatgtgatgaagccacaggattctggaggcagtgccaatgaacaagctgtgcagtga

SEQ ID NO: 4 - DNA, 1770 bp, Synthetic construct
  source 1..1770: mol_type = other DNA; note = ORF for target gene Dypsl3 Myc/Myc/mDpysl3
atggaacaaaaactcatctcagaagaggatctggagcagaagctgatcagcgaggaggacctgatgtattcaccatcaagaaacaaaatccggcagagtgaccgtcttctaatcaagggagggagaatcgtcaatgatgatcagtccttttatgctgatatttacatggaggatggcttaataaagcaaattggagacaatctgattgtccccggaggtgtgaagaccatcgaggccaacgggaagatggtgatccctggaggcattgacgtccatacccacttccagatgccttacaagggaatgaccacagtggacgatttcttccaagggacaaaggctgccttagcgggaggcaccaccatgatcattgaccacgtggtacctgaacctgagtctagcctgacagaggcctatgaaaagtggcgtgagtgggccgatgggaagagctgctgtgactatgctttgcatgtggacatcacccactggaatgacagcgtcaagcaggaggtacagagcctcagcaaggaaaaaggcgttaactccttcatggtttacatggcctacaaggatttatatcaagtgtccaacacagagctctatgagatcttcacctgcctgggagagctgggggccattgctcaagttcatgccgagaatggagatatcattgcccaggagcaggcacggatgctggaaatggggataaccggcccagaaggccacgttctgagcagaccggaagagctggaagctgaggctgtgttccgtgccatcaccgtcgccagccagaccaactgccccctctatgtcaccaaggtcatgagcaagagcgcggctgatctcatctcacaagccaggaagaaaggaaatgtggtctttggcgagcccatcactgccagcctgggaatagatggaactcattactggagtaagaactgggccaaagcagctgcgtttgtgacatccccacctctgagccctgaccctaccacacctgactacatcaactccttgctggccagcggggatctgcagctctctggaagtgcccactgtacctttagcactgcccagaaagccattgggaaggacaacttcacagccatccctgaaggcaccaatggcgtggaggagcgtatgtctgtcatctgggacaaggctgtggccacggggaagatggatgaaaaccagtttgtggccgtgacaagcaccaatgctgccaagatattcaacctgtaccctcgaaaggggagaatagctgtgggctctgacagcgaccttgtcatctgggatccagatgccttgaagattgtctctgccaagaaccaccagtcggttgccgaatacaacatctttgaagggatggagctgcgtggtgcacctctggtggttatctgccagggcaagatcatgctggaagatggcaacctgcacgtgacccagggggctggccgcttcattccctgcagcccattctctgactatgtctataagcgcattaaagcaaggaggaagatggcagacctgcatgcagtcccaagaggcatgtatgatggaccagtgtttgacttgaccaccacccccaaggggggcaccccagctggctctactcggggctctcccactcggccaaacccgccagtgaggaacctccatcagtcaggatttagcctgtcaggcacccaagtggatgagggtgtccgctcagctagcaaacgcattgtggcaccccctggaggccgttctaacatcacatccctgagttaa

SEQ ID NO: 5 - DNA, 1758 bp, Synthetic construct
  source 1..1758: mol_type = other DNA; note = Synthetic ORF for target gene Dpysl5 Myc/Myc/mDpysl5
atggaacaaaaactcatctcagaagaggatctggagcagaagctgatcagcgaggaggacctgatgcttgccaattcagccagcgtgcggattctcatcaagggaggcaaggtggtgaacgatgactgtacccacgaggcagacgtctacatcgagagcggcatcatccagcaggtgggccgtgagctcatgatccccggtggggccaaggtgattgacgccaccgggaaactggtgatcccgggagggattgacaccagcacccacttccaccagaccttcatgaacgccacctgtgtggacgacttctaccatgggaccaaggcggccctcgttggaggcaccaccatgatcatcggtcacgtcctgccagacaaggagacctccctcgtggaagcctatgagaagtgcagggctctagctgaccccaaggtctgctgtgactatgccctccatgtggggatcacctggtgggcacccaaggtgaaagcagaaatggagacactggtgcgggagaagggggtcaactccttccagatgttcatgacctacaaggacttgtacatgcttcgagacagtgagctgtaccaagtgtttcatgcctgcagggacatcggggcgatcccccgggtccacgctgaaaatggagagctcgtggctgagggtgctaaggaggctctagacttgggtatcacaggcccagaaggaatcgagatcagccatccagaggagctggaagcagaagccactcaccgtgtcatcaccattgcaaaccggactcactgtcccatctacctggtcaatgtgtctagtatctcagctggtgatgtcattgcagctgctaagatgcaagggaaggtggtgctagccgagaccaccaacgcacacgccacgctcacgggcttgcactactaccatcaggactggtcgcatgcagccgcctatgtcacggtgcctcctctgagactggacaccaacacttccacctacctcatgagcctgctggccaatgacactctgaacattgtggcatccgatcaccggccattcaccacaaagcagaaagccatgggcaaggaagacttcactaagatcccccacggtgtgagcggtgtacaggatcgaatgagcgtcgtctgggagaggggcgtggttggaggaaagatggatgagaaccgtttcgtggccgtcaccagttctaacgcagccaagatcctcaacctgtatcctcgcaagggccgtatcatccccggagccgacgctgatgtcgtggtgtgggacccggaagccacaaagaccatctcagccagcacccaggtgcagggtggagacttcaatctgtatgagaacatgcgctgccacggtgtaccgctggtcaccatcagccggggacgagtcgtgtatgagaacggcgtcttcatgtgtgcagagggcactggcaagttctgtcccctgcggtctttcccagacattgtctataagaagctggtacagagagagaagaccttaaaagtgaggggagtggaccgcactccctacctaggggatgtcgccattgttgtccaccctgggaaaaaagagatgggaacaccattggcagacacgcccacacggccagtcacccggcacgggggcatgcgggaccttcatgaatccagtttcagcctctctggttctcagatcgatgaccacgttccaaagcgggcctcagctcggatccttgcgccccctggaggcaggtcgagcggtatttggtaa

SEQ ID NO: 6 - DNA, 1191 bp, Synthetic construct
  source 1..1191: mol_type = other DNA; note = Synthetic ORF for target gene Fblim1 Myc/Myc/mFblim1
atggaacaaaaactcatctcagaagaggatctggagcagaagctgatcagcgaggaggacctgatggcctccaagcctgagaaaagggtggcctcctctgtcttcatcaccctggcaccgccacgccgagatgtagccgtgagtgaggaagtgggccaggcagcttgtgaagccagacgcgctcggccctgggagatgcttcccacaaagacacctggggccgcagtgggcaggagccccaagacctggacgccctctggcaagaccaatgcctcactctctggagtcacacctcagctctccaatggaggatgctctctcccacctccttccctgaatgaggaggacctagacctcccgcctcctccaccgcccccttctgcctacctgcctctcccagaagaggagcctcctgtcttaccagggaagtcgctcatttccgacttggagcaactgcacctgcccccacccccgcctccacccccgccacaggctccatcaaagggatcgtctgtccaccctccgcctggtcacgccataccctcggaagaggagcttcctcctcctccagaagagcctgtcacacttccagagagagaagtgtccacggatgtctgtggtttctgtcacaagcctgtgtctcctcgagagctggctgttgaggccatgaagaggcagtaccacgcccagtgcttcacctgtcgtacctgccgccgccagttggctggacagagattctaccagaaggatgggcgccccctgtgcgaaccctgctaccaggatactctggagaagtgtggcaagtgcggagaggtggtccaagagcacgtgatccgggccctgggcaaggccttccacccaccctgcttcacctgcgtgacctgtgcccgctgcatcagcgacgagagctttgcgctggacagccagaaccaggtgtactgtgtggctgatttctacaggaaatttgcccccgtgtgcagcatctgcgagaatcccatcatcccccgagacgggaaggacgccttcaaaatcgagtgcatgggaaggaatttccacgagaactgttaccgctgtgaggactgcagcgtgctcctgtctgtggaacctaccgaccaaggctgctacccactgaatgaccacctcttctgcaagccctgtcacctgaagcggagtgctgctggttgctgctga

SEQ ID NO: 7 - DNA, 2046 bp, Synthetic construct
  source 1..2046: mol_type = other DNA; note = Synthetic ORF for target gene Nfe213 Myc/Myc/mNfe2l3
atggaacaaaaactcatctcagaagaggatctggagcagaagctgatcagcgaggaggacctgatgaagctcccgaagccttggtgggcgggcggcggcctcctgcaactcacgattctgttgagcttggtagggctccgtgtggacctggacctcttcctgccgccgccggccgccgcactttgggaagagctgctcccactctgccccacccgccccgcctcggcttctaaccccttctcggcttcggaaggctgggaacgcacccccctgctacctgccaaaggccggctgctgcacgaagtgcgcgcgctcggggtccccttcattccccgcacccgcgtggacgcgtggctagtgcacagcgtggcgactgggaacgcagacggggcccacgggctgctcggcaccgccgcctcgtcggccgtgggagacggcggccagtcagcatccgcgggcggcggggacccccgagcggctcatagcagccctctggccgctgaggaagaggaggagaaggcggccgaaccgacggcgcaggtgccagatgcaggcggatgcgggagccaggagaacgggatgctaagagaaaagagcgaagctgtggatcacagttcccagcaggaggaaaacgaggaaggggtgtcagcccaggcaaaatcgcgacagcagagtaagacggaggaacacaaaatggcgtgtgcctctgccagggaagccgagaagatcaccgaggctaggaacgagagccatctacattggtcagatacatctttctctttggaagatttattccaactactttcatcacagcctgaacattctctggagggcatctcagtggaagacattccaccttttctgagcagtgtctgtgaaagtgttaattcctcagcacagaacataaatttgagccaagctataagccatgatgtcaatcttcatgaagccatgctgttgtaccccaacaacacatttagaagagatccatcagcaaggtcttcacaggcacaagaaccatttctgcagttaaatcctcatacaaatcctgagcaagccactcctgcaatgtctcttccaccgtttgacaatcaaatgaggaacctaacaagccaagatctcctgtatgacctggattcaaatatatttgatggaataaacctaatgtcattggccacaggtttcagtccattggaagtttctcaactttttgaagaaccagattccgggctctcattaaattcaagttacaacagcacctctctcacaaattcttaccgtatttatgatgggactgtaggttacaatagcgacctacagtctctttgtcatgatttaggtgctgtaggaggctgctacccagaaccccataaacattgtcatatggaccataggactatagctggctttcatgtgtcccttgaatgtcagcaagtatttcatgaccacacttaccatttacagtcaggtgcatcagagcccacatctgaatcttttgcatggtctgagaagtcgcagaaaaccagtggatgccttgataacccagatagaaacttgagccgtgatgaacagcgtgctaaagctctgcacattcccttttctgtagatgaaattgtccgtatgcctgttgactctttcaacagcatgttaagcagatactacctgacagacttacaagtgtctctcatccgtgatatcaggcgaagaggaaaaaataaagttgctgctcagaactgccgtaaacgtaaactagacatcattttgaatctagaggatgatatatgtaacttgcaagcaaagaaagaagcccttaaaaatgagcaaactcaatgtagcaaagctattgacataatgaggcagaagctacatggccttcaccaggatgtttttaatagattaagagatgaccaaggtaggccagtcaatccaagccaatatgcacttcagtacagccatgatggaactgttttgattgtacccaaagaactcatctcatcaggccacaaaaaggaagccccaaagggaaagagagagagaagaaactga

SEQ ID NO: 8 - DNA, 59 bp, Synthetic construct
  source 1..59: mol_type = other DNA; note = shRNA sequence for target gene Pdnp
tattatccagaaagatgcagctttttggcctctgactgaagctgcccttctggataata

SEQ ID NO: 9 - DNA, 59 bp, Synthetic construct
  source 1..59: mol_type = other DNA; note = shRNA sequence for target gene Prdm13
aaataattgcaacttgaccaatttttggcctctgactgaattggtgcatgcaattattt

SEQ ID NO: 10 - DNA, 59 bp, Synthetic construct
  source 1..59: mol_type = other DNA; note = shRNA sequence for target gene Crx
ttaagagcaacctcctcacgtgttttggcctctgactgacacgtgagtgttgctcttaa

SEQ ID NO: 11 - DNA, 59 bp, Synthetic construct
  source 1..59: mol_type = other DNA; note = shRNA sequence for target gene Rax
agtttgtccctctgacagcgagttttggcctctgactgactcgctcctagggacaaact

SEQ ID NO: 12 - DNA, 2772 bp, Synthetic construct
  source 1..2772: mol_type = other DNA; note = Synthetic ORF for target gene Lars2 2xMyc/mLars2
atggaacaaaaactcatctcagaagaggatctggaacaaaaactcatctcagaagaggatctgatggcatccacttgtcagaggctgagtttctatgtctctcctctgaaaaggcagctagttagcaggccacctgtcatcctgtgggaaagactaattccagggtgttccagaagtatctacagtgccaccgggaagtggacaaaggagtacacactgcagacaaggaaggatgtggagaagtggtggcaccaacagataaaggagcaagcgtccagagtctcagaggaagataaattaaagcccaaattctacttgctctccatgttcccatacccttctggcaagctgcacatgggccatgtccgtgtctacaccctcagcgacaccattgcacggttccagaaaatgagaggcatgcaggttatcaaccccatggggtgggatgcatttggattgcctgctgaaaatgctgccatagagaggaatttgcaccctgaaagctggacccaaagcaatattaaacacatgaggaagcagctcgaccgcctcggcctgtgcttcagctgggacagggaaatcactacatgtttgccagattactacaaatggactcaatacctgtttatcaaactctatgaagcaggactggcctatcaaaaggaggccttggttaactgggatccagtggatcaaacagtgctagccaatgagcaggtgaatgaatatggctgttcatggcgttctggagccaaggtagagaagaaatacctccgacagtggttcattaagacaactgcttatgcaaaggccatgcaggatgcgttggcagacctcccagagtggtacggaatcaaaggcatgcaggcccactggattggggactgtgtaggctgtcacctggacttcacattaaaggttgatggagaagacacaggagagaaattgacagcatacacagccacccctgaggccatctatggcatctcccacgtggccatctctcctagccacgggcttctacatggctgcagctctgtgaagaaagccttgcagaaggcactagtccctggcagagattgcctcacaccagtgatggctgtgagcatgctcaccctgcaggaggtccccatcgttatcatggctaaccctgacttggaaggctctctagattcaaaaataggaatccccagcaccagctcagaggacaccagactggcccaagctctgggcctaccctactctgaggtcattgaagcttcacccgatggcacagagagactgagtggatctgctgagtttacaggtatgaccaggcaggatgcctttgtagccctgactcggaaagcccgtgggatgagagtgggtggacacgtgacaagtaacaagctgaaggactggctgatctcacggcagcgctactggggcacaccgatccccattgtccactgcccagcctgtggccctgtgcctgtgccactgcaagacttgcctgtgattttgcccagcattgcatctttaacgggcagaggaggctccccgctggccacggctttggagtgggtgaactgctcctgcccaaggtgcaagggatcagcaaagagagagacggacaccatggacacctttgtggattctgcgtggtactacttcagatacactgaccctcataatactcaaagtccttttggctcggcactggcagacttctggatgcccgtggatctgtacatcggaggaaaggaacatgctgtcatgcacttgttctatgcacgattcctcagtcatttttgccacgaccaaaagatggtgaagcacagggaacctttccataaactactggcccaaggcctcattaagggacagacattccgccttccatctggacagtgtctgaagaaggaggatatagatttcacaggtcctgctcctgtttgtgcaaaaacaaaagagaagttagaggtgacgtgggagaagatgagcaagtccaaacacaatggggtggaccctgaggagattgtggcccaatatggaattgacacaatccggctctacatcctctttgcagcccctcctgagaaggacatcttgtgggatgtaaagactgacgcactccctggagtgctaagatggcaacagcggttgtggtccctgacaacaagattcatcgaggcaaggacttccgggacagtccctcagcctcagctgctgaacagcaaggagaaaaccaaggcccaaaacctctgggagtacaaaaatgcagtcatagctcaggtgaccactcatttcacagaggactttgccctgaattctgtagtttctcagctgatgggactcagcagtgccctctcgcaagcctctcagagagtggttctccacagccctgagtttgaagatgctctgtgtgccctgctggtgatggctgctcccctggcccctcatgtaacctcagaactttgggcaggcctgacgctggtaccaagcaagctctgtgaccattatgcctgggattctggtgtgatgcttcaggcctggcccactgtggactcacagttccttcagaaacctgacatggtgcagatggctgttctgatcaacaacaaggcctgtggcaagatccctgtgccccagcatgttgcccaggaccaggacaaagtccatgaacttgttctccagagcgagctaggcatgaagctgctgcaggggagaagcatcaagaaggccttcctctcccccaggactgccctcatcaacttcttggtgcaggagtga

SEQ ID NO: 13 - DNA, 2853 bp, Synthetic construct
  source 1..2853: mol_type = other DNA; note = Synthetic ORF for target gene Mrtfa 2xMyc/mMrtfa
atggaacaaaaactcatctcagaagaggatctggaacaaaaactcatctcagaagaggatctgatgccgcctttgaaaagccccgctgcatttcatgagcagagaagaagcctggagcgggccaggaccgaggactatttgaaacggaagatccgttcccggcccgagagagcagagctggtcaggatgcacattctggaagagacctcggctgagccttcgctccaggccaagcagctgaagctgaagagagccaggctggctgatgacctcaatgaaaagattgcacagaggcctggccccatggagctggtggagaagaatatcctgcctgtggagtccagcctgaaggaggctatcattgtgggccaggtaaattacccaaaggtagcagacagttcctccttcgacgaggacagcagcgatgccctgtctcctgagcagcctgccagccatgagtcccagggttcagtgccatcacccttggagtcccgagtcagtgatccactgcccagtgccacctccatatcacccactcaggttctttctcaactcccaatggctccggatcctggagagacgctttttctggcagagcagcctcctctgcctcccgcacctctgctgcccccaagcctagccaatggaagcatcgtccccactgccaagcctgctcccacactcatcaagcaaagccaacccaagtctgccagcgagaaatcacagcgcagcaagaaggccaaggagctgaagccaaaggtgaagaagctcaagtaccaccagtacatccccccggaccagaagcaggacaagggggcgcccgccatggactcctcctatgccaagatcctgcagcagcagcagctcttcctgcagctgcagatcctcaaccagcagcagcagcagcagcagcaacagcactacaactaccaggccatcctgcctgcccctcccaagccctcggctgagactcctggaagcagtgcccctaccccatcacgcagcctctccaccagtagcagccccagctcaggcaccccagggcccagcgggctggcacgccagagcagcaccgcactagctgccaaaccaggagccctgccagccaacctggatgacatgaaggtggcagagctgaagcaagaactgaagttgcggtcccttcccgtctcaggcaccaagacagagctgatagaacgcctgcgtgcctaccaagaccaagtcagcccagctccaggagcccccaaggcccctgccaccacctctgtgctgtccaaggctggtgaggtagtggtcgccttccctgcggccctgctaagcacagggtcagctcttgtaacagcaggccttgcaccagctgagatggtggtggccacagtaaccagcaatggcatggtgaagtttggcagcacaggctccacaccccccgtgtctcccaccccttcagagcgctcactgctcagcacgggtgatgagaattccacacctggggatgcctttggtgaaatggtgacatcgccgctgacacagctcaccctgcaggcctccccactgcagatcgtgaaggaggagggtgcccgtgctgcgtcctgctgtctaagccctggtgctcgggctgagctggagggactggacaaggaccagatgctgcaggagaaggacaagcagattgaggagctgacccgaatgctccaacagaagcagcagctggttgagctgctgcggctacagctggagcagcagaagcgggcccagcagccagccccagccagcagccctgtgaagagggaaagtggtttctccagttgccagctgagctgccagccccagggctctgcccatgcttttggctctggcctagtggttcccactaccaaccatggagacactcaggccccagcgccagagtccccacctgtggtggtgaagcaggaagctgggccacctgagccagatctggcccccagctcccagctgctcttgggctcccagggcaccagcttcctcaagagggtcagccctcctaccctggtcactgactctacagggactcacctcatcctcactgtgaccaataagagtgctgatggccctggcttgcctgcagggagcccccagcagcccttgtcccagcctggttctccagcccctggtccacctgcccagatggacctggagcacccacctcagcctccgtttgcaacccccacatctctgctgaagaaggagccccctggttatgaagagactgtgacccagcagcctaagcagcaggaaaatggctcctccagtcagcacatggatgatctgtttgatattcttattcagagtggagagatttcagcagatttcaaagagccaccatccctaccaggcaaggaaaagtcacctccagcagcagcagcgtatgggcctccattgacaccacaaccctcgcctttgagtgaactcccccaagctgctcctccaccaggttcccccaccctcccagggcgccttgaagacttcctggagagcagcacagggctgcccctgctgacaagtgggcacgagggaccagaacccctttccctcattgatgacctccacagccagatgctgagcagctccgccatcctggaccaccccccatcacccatggacacctctgaattgcactttgctcctgagcccagcagtggtatgggcctggacctggctgttggccacctggacagcatggactggctggagctgtcgtctggtggccctgtgctcagcctggctcccctcagcactgcagcccccagcctcttctcgatggacttcctggatggacacgacttgcagctccactgggattcctgcttgtag

SEQ ID NO: 14 - DNA, 59 bp, Synthetic construct
  source 1..59: mol_type = other DNA; note = shRNA sequence for target gene Mmp9
tttgagtttccatagtaagtggttttggcctctgactgaccacttctgggaaactcaaa

SEQ ID NO: 15 - DNA, 405 bp, Synthetic construct
  source 1..405: mol_type = other DNA; note = Synthetic ORF for target gene Dynlt1a 2xMyc/mDynlt1a
atggaacaaaaactcatctcagaagaggatctggaacaaaaactcatctcagaagaggatctgatggaagacttccaggcctccgaggagactgcatttgttgttgatgaagtgagcagcattgtaaaggaggctatagaaagcgccatcggtggtaatgcctaccagcacagcaaagtcaaccagtggaccactaatgtcctagaacagactttgagccaactcaccaaactggggagaccatttaaatacattgtgacctgtgtgatcatgcagaagaacggtgctgggttacactccgcaagttcctgcttctgggacagctccacagacggaagctgcacagtccgatgggagaacaagaccatgtactgcatcgtcagtaccttcggactgtccatctga

SEQ ID NO: 16 - DNA, 531 bp, Synthetic construct
  source 1..531: mol_type = other DNA; note = Synthetic ORF for target gene Atp6v0c 2xMyc/mAtp6v0c
atggaacaaaaactcatctcagaagaggatctggaacaaaaactcatctcagaagaggatctgatggctgacatcaagaacaaccccgaatattcttcgtttttcggtgtcatgggcgcctcgtccgccatggtcttcagcgccatgggagccgcctatggcacagccaagagtggcactggcatcgcagccatgtcagtcatgaggccagagctgatcatgaagtccatcatcccagtggttatggctgggatcatcgccatctacggcctggtggtggcagtacttatcgctaactccctgactgatggcatcaccctctacaggagttttcttcaactgggtgctggcctgagtgtggggctgagtggcctggctgctggctttgccattggcatcgtcggagatgctggtgttcggggcactgcccagcagcctcgactgttcgtgggcatgatcctgatcctcatctttgcggaggtgcttggcctctacggtctcatcgtggccctaatcctctccacaaagtag

SEQ ID NO: 17 - DNA, 1263 bp, Synthetic construct
  source 1..1263: mol_type = other DNA; note = Synthetic ORF for target gene Lancl1, 2xMyc/mLancl1
atggaacaaaaactcatctcagaagaggatctggaacaaaaactcatctcagaagaggatctgatggctcagagggcctttccgaatccttatgccgattataacaagtcgctggccgaaaactactttgattctactgggaggctgactcctgagttctcacatcgcttgaccaataagatccgagagctccttcagcaaatggagagaggcctaaagtctgcagaccctcgggatggcactgggtacactggctgggcaggcatcgctgtgctttacctccacctccacaatgtgtttggggacccagcctatctacagatggcgcacagctatgtaaagcaaagcctcaactgtctgagcaggcgttccatcaccttcctgtgtggcgatgcgggccccctggctgtggctgctgtgctgtaccacaagatgaacagtgagaagcaggccgaggagtgcatcacacggctcattcacctaaataagattgatccccacgtaccaaatgaaatgctctatggccgcataggctacatctttgctctgctttttgtcaataagaactttggagaggagaagattcctcagagccatattcagcagatttgtgaaaacatcttaacctctggggaaaacctatctagaaagagaaacttggcggcaaagtctccactgatgtatgagtggtaccaggaatattacgtgggggctgctcatggcttggcaggcatttattactacctgatgcagcccagccttcaggtgaaccaaggaaagttgcatagtttggtgaagcccagtgtagactttgtctgccggctgaagtttccttccggcaattaccctccatgtttggatgataccagagacctgcttgtccattggtgccacggtgctcctggggtcatctatatgctcatccaagcatacaaggtgttcaaagaagagcgttacctgtgtgatgcccagcagtgtgctgacgtgatctggcagtacgggctactgaagaagggctacgggctgtgccatggtgctgcagggaatgcctacgctttcctggcactctacaacctcacacaggatctgaagtatctgtacagggcctgcaagtttgctgagtggtgcttagactacggggagcacggatgcaggacagcggacacccccttctccctctttgaagggatggctggaacaatatatttcttggctgacctgttagtccccacaaaggccaagttccctgcatttgaactctga

SEQ ID NO: 18 - DNA, 59 bp, Synthetic construct
  source 1..59: mol_type = other DNA; note = Insert sequence for miR-29a-3p shRNA
taaccgatttcagatggtgctattttggcctctgactgatagcacattgaaatcggtta

SEQ ID NO: 19 - DNA, 59 bp, Synthetic construct
  source 1..59: mol_type = other DNA; note = Insert sequence for miR-1290 shRNA
tccctgatccaaaaatccaaaattttggcctctgactgattttggttttggatcaggga

SEQ ID NO: 20 - DNA, 61 bp, Synthetic construct
  source 1..61: mol_type = other DNA; note = Insert sequence for miR-132-3p shRNA
tcgaccatggctgtagactgttattttggcctctgactgataacagttctgccatggtcga

SEQ ID NO: 21 - DNA, 61 bp, Synthetic construct
  source 1..61: mol_type = other DNA; note = Insert sequence for miR-210-3p shRNA
tctgtgcgtgtgacagcggctgattttggcctctgactgatcagccgtatacacgcacaga

SEQ ID NO: 22 - DNA, 59 bp, Synthetic construct
  source 1..59: mol_type = other DNA; note = Insert sequence for miR-135b-3p shRNA
atgtagggctaaaagccatgggttttggcctctgactgacccatgctttagccctacat

SEQ ID NO: 23 - DNA, 59 bp, Synthetic construct
  source 1..59: mol_type = other DNA; note = Insert sequence for miR-129-5p shRNA
tgcaagcccagaccgcaaaaagttttggcctctgactgactttttcagctgggcttgca

SEQ ID NO: 24 - DNA, 81 bp, Synthetic construct
  source 1..81: mol_type = other DNA; note = Insert sequence for miR-129-1-3p shRNA
atactttttggggtaagggcttttttggcctctgactgaaagccctctccaaaaagtatatactttttggggtaagggctt

SEQ ID NO: 25 - DNA, 59 bp, Synthetic construct
  source 1..59: mol_type = other DNA; note = Insert sequence for miR-6090 for shRNA
tgccccgcccctcgctcccccattttggcctctgactgatggggggcgggggcggggca

SEQ ID NO: 26 - DNA, 59 bp, Synthetic construct
  source 1..59: mol_type = other DNA; note = Insert sequence for miR-1247-5p shRNA
acccgtcccgttcgtccccggattttggcctctgactgatccgggacgacgggacgggt

SEQ ID NO: 27 - DNA, 59 bp, Synthetic construct
  source 1..59: mol_type = other DNA; note = Insert sequence for miR-3914-1 shRNA
aaggaaccagaaaatgagaagtttttggcctctgactgaacttctatttctggttcctt

SEQ ID NO: 28 - DNA, 59 bp, Synthetic construct
  source 1..59: mol_type = other DNA; note = Insert sequence for miR-5109 shRNA
tgttgcggaccaggggaatccgattttggcctctgactgatcggattcctggtccgcaa

SEQ ID NO: 29 - DNA, 71 bp, Synthetic construct
  source 1..71: mol_type = other DNA; note = Insert sequence for piR16295 shRNA
acaccgtccacgggctgggcctcgatcattttggcctctgactgatgatcgagcctgcccgtggacggtgt

SEQ ID NO: 30 - DNA, 64 bp, Synthetic construct
  source 1..64: mol_type = other DNA; note = Insert sequence for Ift20 shRNA
aacactcggagcttgttcagttcatcaaattttggcctctgactgatttgatgaataacaagct

SEQ ID NO: 31 - DNA, 73 bp, Synthetic construct
  source 1..73: mol_type = other DNA; note = Insert sequence for sncRNA VB200810-1013mnb shRNA for overexpression
tgagcaccaggctgttgaaaaatgacccattttggcctctgactgatgggtcatttttacagcctggtgctca

SEQ ID NO: 32 - DNA, 71 bp, Synthetic construct
  source 1..71: mol_type = other DNA; note = Insert sequence for sncRNA VB200810-1256arq shRNA for overexpression
aacatccccaccaactcttagcactggattttggcctctgactgatccagtgcacggttggtggggatgtt

SEQ ID NO: 33 - DNA, 77 bp, Synthetic construct
  source 1..77: mol_type = other DNA; note = Insert sequence for sncRNA VB200810-1300pah shRNA for overexpression
tgctgtgaaggtgaatctggatttccttgaattttggcctctgactgattcaaggaaaccgattcaccttcacagca

SEQ ID NO: 34 - DNA, 73 bp, Synthetic construct
  source 1..73: mol_type = other DNA; note = Insert sequence for sncRNA sequence VB200810-1670qan shRNA for overexpression
tgctctctgaacgagtgaggcatctcccattttggcctctgactgatgggagatgcttctcgttcagagagca

SEQ ID NO: 35 - DNA, 73 bp, Synthetic construct; identical to SEQ ID NO: 34
  source 1..73: mol_type = other DNA; note = Insert sequence for sncRNA VB200810-1670qan shRNA for overexpression

SEQ ID NO: 36 - DNA, 77 bp, Synthetic construct
  source 1..77: mol_type = other DNA; note = Oligo dT for reverse transcription
  misc_feature 38..45: note = NNNNNNNN, Unique Molecular Identifier
  misc_feature 76: note = V is A, C, G
  misc_feature 77: note = N is A, C, G, T
aagcagtggtatcaacgcagagtacaacaagtcgctgnnnnnnnnttttttttttttttttttttttttttttttvn

SEQ ID NO: 37 - DNA, 32 bp, Synthetic construct
  source 1..32: mol_type = other DNA; note = template switching oligo
  misc_feature 28..31: note = rGrG is riboguanosines
aagcagtggtatcaacgcagagtgaatrgrgg

SEQ ID NO: 38 - DNA, 23 bp, Synthetic construct
  source 1..23: mol_type = other DNA; note = template switching oligo for in situ PCT
aagcagtggtatcaacgcagagt

SEQ ID NO: 39 - DNA, 66 bp, Synthetic construct
  source 1..66: mol_type = other DNA; note = primer
  misc_feature 65..66: note = A and C are phosphorothioate base modified
aatgatacggcgaccaccgagatctacacgcctgtccgcggaagcagtggtatcaacgcagagtac

SEQ ID NO: 40 - DNA, 28 bp, Synthetic construct
  source 1..28: mol_type = other DNA; note = primer
cggaagcagtggtatcaacgcagagtac

SEQ ID NO: 41 - protein, 170 aa, Homo sapiens
DSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSP

SEQ ID NO: 42 - protein, 19 aa, Synthetic construct
  source 1..19: note = signal peptide
MGWSCIILFLVATATGVHS

SEQ ID NO: 43 - protein, 26 aa, Synthetic construct
  source 1..26: note = calmodulin binding peptide
KRRWKKNFIAVSAANRFKKISSSGAL

SEQ ID NO: 44 - protein, 13 aa, Synthetic construct
  source 1..13: note = E-tag
GAPVPYPDPLEPR

SEQ ID NO: 45 - protein, 8 aa, Synthetic construct
  source 1..8: note = FLAG tag
DYKDDDDK

SEQ ID NO: 46 - protein, 8 aa, Synthetic construct
  source 1..8: note = HA-tag
YPYDVPDY

SEQ ID NO: 47 - protein, 7 aa, Synthetic construct
  source 1..7: note = Gly-His tag
GHHHHHH

SEQ ID NO: 48 - protein, 9 aa, Synthetic construct
  source 1..9: note = Gly-His tag
GSSHHHHHH

SEQ ID NO: 49 - protein, 10 aa, Synthetic construct
  source 1..10: note = Myc-tag
EQKLISEEDL

SEQ ID NO: 50 - protein, 18 aa, Synthetic construct
  source 1..18: note = NE-tag
TKENPRSNQEESYDDNES

SEQ ID NO: 51 - protein, 38 aa, Synthetic construct
  source 1..38: note = Streptavidin-binding peptide-tag
MDEKTTGWRGGHVVEGLAGELEQLRARLEHHPQGQREP

SEQ ID NO: 52 - protein, 8 aa, Synthetic construct
  source 1..8: note = Step II tag
WSHPQFEK

SEQ ID NO: 53 - protein, 15 aa, Synthetic construct
  source 1..15: note = S-tag
KETAAAKFERQHMDS

SEQ ID NO: 54 - protein, 11 aa, Synthetic construct
  source 1..11: note = T7-tag
MASMTGGQQMG

SEQ ID NO: 55 - protein, 14 aa, Synthetic construct
  source 1..14: note = V5-tag
GKPIPNPLLGLDST

SEQ ID NO: 56 - protein, 195 aa, Synthetic construct
  source 1..195: note = fibronectin-based peptide variant
MGWSCIILFLVATATGVHSDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPHHHHHH

SEQ ID NO: 57 - DNA, 59 bp, Synthetic construct
  source 1..59: mol_type = other DNA; note = Insert sequence for shRNA overexpression of target miR0135a-3p
tatagggattggagccgtggcgttttggcctctgactgacgccacgctcaatccctata

SEQ ID NO: 58 - DNA, 59 bp, Synthetic construct
  source 1..59: mol_type = other DNA; note = shRNA sequence for target gene Pdnp
tcattaagccctccagtagcacttttggcctctgactgagtgctagtggggcttaatga

SEQ ID NO: 59 - DNA, 59 bp, Synthetic construct
  source 1..59: mol_type = other DNA; note = shRNA sequence for target gene Pdnp
atctttcttatctgttgtctgcttttggcctctgactgagcagacaacataagaaagat

SEQ ID NO: 60 - DNA, 59 bp, Synthetic construct
  source 1..59: mol_type = other DNA; note = shRNA sequence for target gene Pdnp
tttgacagtggttgtactctcgttttggcctctgactgacgagagaacccactgtcaaa

SEQ ID NO: 61 - DNA, 59 bp, Synthetic construct
  source 1..59: mol_type = other DNA; note = shRNA sequence for target gene Prdm13
ggattaaccctatccactccagttttggcctctgactgactggagcgcagggttaatcc

SEQ ID NO: 62 - DNA, 59 bp, Synthetic construct
  source 1..59: mol_type = other DNA; note = shRNA sequence for target gene Prdm13
tggtaagtctgcaatagcttccttttggcctctgactgaggaagccgtcagacttacca

SEQ ID NO: 63 - DNA, 59 bp, Synthetic construct
  source 1..59: mol_type = other DNA; note = shRNA sequence for target gene Prdm13
tttctcatcatgcgttggagtcttttggcctctgactgagactccgctatgatgagaaa

SEQ ID NO: 64 - DNA, 59 bp, Synthetic construct
  source 1..59: mol_type = other DNA; note = shRNA sequence for target gene Crx
tgaatttggaggtctcactttgttttggcctctgactgacaaagttgcctccaaattca

SEQ ID NO: 65 - DNA, 59 bp, Synthetic construct
  source 1..59: mol_type = other DNA; note = shRNA sequence for target gene Crx
tctttgtagtccagagggtccattttggcctctgactgatggaccttggactacaaaga

SEQ ID NO: 66 - DNA, 59 bp, Synthetic construct
  source 1..59: mol_type = other DNA; note = shRNA sequence for target gene Crx
atttcgccctacgattcttgaattttggcctctgactgattcaagctctagggcgaaat

SEQ ID NO: 67 - DNA, 59 bp, Synthetic construct
  source 1..59: mol_type = other DNA; note = shRNA sequence for target gene Rax
ttcgatgctgtgcaaacgcgacttttggcctctgactgagtcgcgtgtacagcatcgaa

SEQ ID NO: 68 - DNA, 59 bp, Synthetic construct
  source 1..59: mol_type = other DNA; note = shRNA sequence for target gene Rax
atggacgacacttccagtttctttttggcctctgactgaagaaacagtgtgtcgtccat

SEQ ID NO: 69 - DNA, 59 bp, Synthetic construct
  source 1..59: mol_type = other DNA; note = shRNA sequence for target gene Rax
atgccgtcttccttggtaaagcttttggcctctgactgagctttagacgaagacggcat

SEQ ID NO: 70 - DNA, 59 bp, Synthetic construct
  source 1..59: mol_type = other DNA; note = shRNA sequence for target gene Mmp9
tatgtcgtctttattcagagggttttggcctctgactgaccctctactaagacgacata

SEQ ID NO: 71 - DNA, 59 bp, Synthetic construct
  source 1..59: mol_type = other DNA; note = shRNA sequence for target gene Mmp9
ttatgatggtcccacttgaggcttttggcctctgactgagcctcagtggaccatcataa

SEQ ID NO: 72 - DNA, 59 bp, Synthetic construct
  source 1..59: mol_type = other DNA; note = shRNA sequence for target gene Mmp9
tttatcctggtcatagttggctttttggcctctgactgaagccaatctaccaggataaa

SEQ ID NO: 73 - DNA, 6604 bp, Synthetic construct
  source 1..6604: mol_type = other DNA; note = pAAV[miR155]-UBC>4x mMmp9[shRNA]
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgttatgatggtcccacttgaggcttttggcctctgactgagcctcagtggaccatcataacaggacacaaggcctgttactagcactcacatggaacaaatggccaccgtgggaggatgctgattgcgtaaccggtaaataggcttgctgaaggctgtatgctgtttatcctggtcatagttggctttttggcctctgactgaagccaatctaccaggataaacaggacacaaggcctgttactagcactcacatggaacaaatggccaccgtgggaggatggagatcttcttggtcacaagaaggcttgctgaaggctgtatgctgtttgagtttccatagtaagtggttttggcctctgactgaccacttctgggaaactcaaacaggacacaaggcctgttactagcactcacatggaacaaatggccaccgtgggaggatgagcaatgttctgttagaccgaaggcttgctgaaggctgtatgctgtatgtcgtctttattcagagggttttggcctctgactgaccctctactaagacgacatacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 74 - DNA, 6604 bp, Synthetic construct
  source 1..6604: mol_type = other DNA; note = pAAV[miR155]-UBC>mRax[shRNA#1]:mRax[shRNA#2]:mRax[s hRNA#3]:mRax[shRNA#4]:Myc/mCherry
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgagtttgtccctctgacagcgagttttggcctctgactgactcgctcctagggacaaactcaggacacaaggcctgttactagcactcacatggaacaaatggccaccgtgggaggatgctgattgcgtaaccggtaaataggcttgctgaaggctgtatgctgttcgatgctgtgcaaacgcgacttttggcctctgactgagtcgcgtgtacagcatcgaacaggacacaaggcctgttactagcactcacatggaacaaatggccaccgtgggaggatggagatcttcttggtcacaagaaggcttgctgaaggctgtatgctgatggacgacacttccagtttctttttggcctctgactgaagaaacagtgtgtcgtccatcaggacacaaggcctgttactagcactcacatggaacaaatggccaccgtgggaggatgagcaatgttctgttagaccgaaggcttgctgaaggctgtatgctgatgccgtcttccttggtaaagcttttggcctctgactgagctttagacgaagacggcatcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 75 - DNA, 6604 bp, Synthetic construct
  source 1..6604: mol_type = other DNA; note = pAAV[miR155]-UBC>mCrx[shRNA#1]:mCrx[shRNA#2]:mCrx[s hRNA#3]:mCrx[shRNA#4]:Myc/mCherry
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgttaagagcaacctcctcacgtgttttggcctctgactgacacgtgagtgttgctcttaacaggacacaaggcctgttactagcactcacatggaacaaatggccaccgtgggaggatgctgattgcgtaaccggtaaataggcttgctgaaggctgtatgctgtgaatttggaggtctcactttgttttggcctctgactgacaaagttgcctccaaattcacaggacacaaggcctgttactagcactcacatggaacaaatggccaccgtgggaggatggagatcttcttggtcacaagaaggcttgctgaaggctgtatgctgtctttgtagtccagagggtccattttggcctctgactgatggaccttggactacaaagacaggacacaaggcctgttactagcactcacatggaacaaatggccaccgtgggaggatgagcaatgttctgttagaccgaaggcttgctgaaggctgtatgctgatttcgccctacgattcttgaattttggcctctgactgattcaagctctagggcgaaatcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 76 - DNA, 6604 bp, Synthetic construct
  source 1..6604: mol_type = other DNA; note = pAAV[miR155]-UBC>mPdpn[shRNA#1]:mPdpn[shRNA#2]:mPdp n[shRNA#3]:mPdpn[shRNA#4]:Myc/mCherry
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtattatccagaaagatgcagctttttggcctctgactgaagctgcccttctggataatacaggacacaaggcctgttactagcactcacatggaacaaatggccaccgtgggaggatgctgattgcgtaaccggtaaataggcttgctgaaggctgtatgctgtcattaagccctccagtagcacttttggcctctgactgagtgctagtggggcttaatgacaggacacaaggcctgttactagcactcacatggaacaaatggccaccgtgggaggatggagatcttcttggtcacaagaaggcttgctgaaggctgtatgctgatctttcttatctgttgtctgcttttggcctctgactgagcagacaacataagaaagatcaggacacaaggcctgttactagcactcacatggaacaaatggccaccgtgggaggatgagcaatgttctgttagaccgaaggcttgctgaaggctgtatgctgtttgacagtggttgtactctcgttttggcctctgactgacgagagaacccactgtcaaacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 77 - DNA, 6604 bp, Synthetic construct
  source 1..6604: mol_type = other DNA; note = pAAV[miR155]-UBC>mPrdm13[shRNA#1]:mPrdm13[shRNA#2]: mPrdm13[shRNA#3]:mPrdm13[shRNA#4]:Myc/
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgaaataattgcaacttgaccaatttttggcctctgactgaattggtgcatgcaattatttcaggacacaaggcctgttactagcactcacatggaacaaatggccaccgtgggaggatgctgattgcgtaaccggtaaataggcttgctgaaggctgtatgctgggattaaccctatccactccagttttggcctctgactgactggagcgcagggttaatcccaggacacaaggcctgttactagcactcacatggaacaaatggccaccgtgggaggatggagatcttcttggtcacaagaaggcttgctgaaggctgtatgctgtggtaagtctgcaatagcttccttttggcctctgactgaggaagccgtcagacttaccacaggacacaaggcctgttactagcactcacatggaacaaatggccaccgtgggaggatgagcaatgttctgttagaccgaaggcttgctgaaggctgtatgctgtttctcatcatgcgttggagtcttttggcctctgactgagactccgctatgatgagaaacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 78 - DNA, 6660 bp, Synthetic construct
  source 1..6660: mol_type = other DNA; note = pAAV[miR155]-UBC>4x Anti-ift20 shRNA VB200810-1671efc
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgaacactcggagcttgttcagttcatcaaattttggcctctgactgatttgatgaataacaagctccgagtgttcaggacacaaggcctgttactagcactcacatggaacaaatggccaccgtgggaggatgctgattgcgtaaccggtaaataggcttgctgaaggctgtatgctgaacactcggagcttgttcagttcatcaaattttggcctctgactgatttgatgaataacaagctccgagtgttcaggacacaaggcctgttactagcactcacatggaacaaatggccaccgtgggaggatggagatcttcttggtcacaagaaggcttgctgaaggctgtatgctgaacactcggagcttgttcagttcatcaaattttggcctctgactgatttgatgaataacaagctccgagtgttcaggacacaaggcctgttactagcactcacatggaacaaatggccaccgtgggaggatgagcaatgttctgttagaccgaaggcttgctgaaggctgtatgctgaacactcggagcttgttcagttcatcaaattttggcctctgactgatttgatgaataacaagctccgagtgttcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 79 - DNA, 6652 bp, Synthetic construct
  source 1..6652: mol_type = other DNA; note = pAAV[miR155]-UBC>4x anti(piR-16295)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgacaccgtccacgggctgggcctcgatcattttggcctctgactgatgatcgagcctgcccgtggacggtgtcaggacacaaggcctgttactagcactcacatggaacaaatggccaccgtgggaggatgctgattgcgtaaccggtaaataggcttgctgaaggctgtatgctgacaccgtccacgggctgggcctcgatcattttggcctctgactgatgatcgagcctgcccgtggacggtgtcaggacacaaggcctgttactagcactcacatggaacaaatggccaccgtgggaggatggagatcttcttggtcacaagaaggcttgctgaaggctgtatgctgacaccgtccacgggctgggcctcgatcattttggcctctgactgatgatcgagcctgcccgtggacggtgtcaggacacaaggcctgttactagcactcacatggaacaaatggccaccgtgggaggatgagcaatgttctgttagaccgaaggcttgctgaaggctgtatgctgacaccgtccacgggctgggcctcgatcattttggcctctgactgatgatcgagcctgcccgtggacggtgtcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 80 - DNA, 6660 bp, Synthetic construct
  source 1..6660: mol_type = other DNA; note = pAAV[miR155]-UBC>4x shRNA VB200810-1013mnb
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtgagcaccaggctgttgaaaaatgacccattttggcctctgactgatgggtcatttttacagcctggtgctcacaggacacaaggcctgttactagcactcacatggaacaaatggccaccgtgggaggatgctgattgcgtaaccggtaaataggcttgctgaaggctgtatgctgtgagcaccaggctgttgaaaaatgacccattttggcctctgactgatgggtcatttttacagcctggtgctcacaggacacaaggcctgttactagcactcacatggaacaaatggccaccgtgggaggatggagatcttcttggtcacaagaaggcttgctgaaggctgtatgctgtgagcaccaggctgttgaaaaatgacccattttggcctctgactgatgggtcatttttacagcctggtgctcacaggacacaaggcctgttactagcactcacatggaacaaatggccaccgtgggaggatgagcaatgttctgttagaccgaaggcttgctgaaggctgtatgctgtgagcaccaggctgttgaaaaatgacccattttggcctctgactgatgggtcatttttacagcctggtgctcacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 81 - DNA, 6676 bp, Synthetic construct
  source 1..6676: mol_type = other DNA; note = pAAV[miR155]-UBC>4x shRNA VB200810-1300pah
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtgctgtgaaggtgaatctggatttccttgaattttggcctctgactgattcaaggaaaccgattcaccttcacagcacaggacacaaggcctgttactagcactcacatggaacaaatggccaccgtgggaggatgctgattgcgtaaccggtaaataggcttgctgaaggctgtatgctgtgctgtgaaggtgaatctggatttccttgaattttggcctctgactgattcaaggaaaccgattcaccttcacagcacaggacacaaggcctgttactagcactcacatggaacaaatggccaccgtgggaggatggagatcttcttggtcacaagaaggcttgctgaaggctgtatgctgtgctgtgaaggtgaatctggatttccttgaattttggcctctgactgattcaaggaaaccgattcaccttcacagcacaggacacaaggcctgttactagcactcacatggaacaaatggccaccgtgggaggatgagcaatgttctgttagaccgaaggcttgctgaaggctgtatgctgtgctgtgaaggtgaatctggatttccttgaattttggcctctgactgattcaaggaaaccgattcaccttcacagcacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 82 - DNA, 6652 bp, Synthetic construct
  source 1..6652: mol_type = other DNA; note = pAAV[miR155]-UBC>4x shRNA VB200810-1256arq
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgaacatccccaccaactcttagcactggattttggcctctgactgatccagtgcacggttggtggggatgttcaggacacaaggcctgttactagcactcacatggaacaaatggccaccgtgggaggatgctgattgcgtaaccggtaaataggcttgctgaaggctgtatgctgaacatccccaccaactcttagcactggattttggcctctgactgatccagtgcacggttggtggggatgttcaggacacaaggcctgttactagcactcacatggaacaaatggccaccgtgggaggatggagatcttcttggtcacaagaaggcttgctgaaggctgtatgctgaacatccccaccaactcttagcactggattttggcctctgactgatccagtgcacggttggtggggatgttcaggacacaaggcctgttactagcactcacatggaacaaatggccaccgtgggaggatgagcaatgttctgttagaccgaaggcttgctgaaggctgtatgctgaacatccccaccaactcttagcactggattttggcctctgactgatccagtgcacggttggtggggatgttcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 83 - DNA, 6660 bp, Synthetic construct
  source 1..6660: mol_type = other DNA; note = pAAV[miR155]-UBC>4x shRNA VB200810-1672kgn
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgactcacttgttggggcgcagtcttaagcattttggcctctgactgatgcttaagatatccccaacaagtgagtcaggacacaaggcctgttactagcactcacatggaacaaatggccaccgtgggaggatgctgattgcgtaaccggtaaataggcttgctgaaggctgtatgctgactcacttgttggggcgcagtcttaagcattttggcctctgactgatgcttaagatatccccaacaagtgagtcaggacacaaggcctgttactagcactcacatggaacaaatggccaccgtgggaggatggagatcttcttggtcacaagaaggcttgctgaaggctgtatgctgactcacttgttggggcgcagtcttaagcattttggcctctgactgatgcttaagatatccccaacaagtgagtcaggacacaaggcctgttactagcactcacatggaacaaatggccaccgtgggaggatgagcaatgttctgttagaccgaaggcttgctgaaggctgtatgctgactcacttgttggggcgcagtcttaagcattttggcctctgactgatgcttaagatatccccaacaagtgagtcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 84 - DNA, 6660 bp, Synthetic construct
  source 1..6660: mol_type = other DNA; note = pAAV[miR155]-UBC>4x shRNA VB200810-1670qan
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtgctctctgaacgagtgaggcatctcccattttggcctctgactgatgggagatgcttctcgttcagagagcacaggacacaaggcctgttactagcactcacatggaacaaatggccaccgtgggaggatgctgattgcgtaaccggtaaataggcttgctgaaggctgtatgctgtgctctctgaacgagtgaggcatctcccattttggcctctgactgatgggagatgcttctcgttcagagagcacaggacacaaggcctgttactagcactcacatggaacaaatggccaccgtgggaggatggagatcttcttggtcacaagaaggcttgctgaaggctgtatgctgtgctctctgaacgagtgaggcatctcccattttggcctctgactgatgggagatgcttctcgttcagagagcacaggacacaaggcctgttactagcactcacatggaacaaatggccaccgtgggaggatgagcaatgttctgttagaccgaaggcttgctgaaggctgtatgctgtgctctctgaacgagtgaggcatctcccattttggcctctgactgatgggagatgcttctcgttcagagagcacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 85 - DNA, 6604 bp, Synthetic construct
  source 1..6604: mol_type = other DNA; note = pAAV[miR155]-UBC>4x anti(mmu-miR-129-1-3p)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgatactttttggggtaagggcttttttggcctctgactgaaagccctctccaaaaagtatcaggacacaaggcctgttactagcactcacatggaacaaatggccaccgtgggaggatgctgattgcgtaaccggtaaataggcttgctgaaggctgtatgctgatactttttggggtaagggcttttttggcctctgactgaaagccctctccaaaaagtatcaggacacaaggcctgttactagcactcacatggaacaaatggccaccgtgggaggatggagatcttcttggtcacaagaaggcttgctgaaggctgtatgctgatactttttggggtaagggcttttttggcctctgactgaaagccctctccaaaaagtatcaggacacaaggcctgttactagcactcacatggaacaaatggccaccgtgggaggatgagcaatgttctgttagaccgaaggcttgctgaaggctgtatgctgatactttttggggtaagggcttttttggcctctgactgaaagccctctccaaaaagtatcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 6604 bp, Synthetic construct
  source 1..6604: mol_type = other DNA; note = pAAV[miR155]-UBC>4x anti(mmu-miR-129-5p)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtgcaagcccagaccgcaaaaagttttggcctctgactgactttttcagctgggcttgcacaggacacaaggcctgttactagcactcacatggaacaaatggccaccgtgggaggatgctgattgcgtaaccggtaaataggcttgctgaaggctgtatgctgtgcaagcccagaccgcaaaaagttttggcctctgactgactttttcagctgggcttgcacaggacacaaggcctgttactagcactcacatggaacaaatggccaccgtgggaggatggagatcttcttggtcacaagaaggcttgctgaaggctgtatgctgtgcaagcccagaccgcaaaaagttttggcctctgactgactttttcagctgggcttgcacaggacacaaggcctgttactagcactcacatggaacaaatggccaccgtgggaggatgagcaatgttctgttagaccgaaggcttgctgaaggctgtatgctgtgcaagcccagaccgcaaaaagttttggcctctgactgactttttcagctgggcttgcacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 87 - DNA, 6604 bp, Synthetic construct
  source 1..6604: mol_type = other DNA; note = pAAV[miR155]-UBC>4x anti(hsa-mir-1290)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtccctgatccaaaaatccaaaattttggcctctgactgattttggttttggatcagggacaggacacaaggcctgttactagcactcacatggaacaaatggccaccgtgggaggatgctgattgcgtaaccggtaaataggcttgctgaaggctgtatgctgtccctgatccaaaaatccaaaattttggcctctgactgattttggttttggatcagggacaggacacaaggcctgttactagcactcacatggaacaaatggccaccgtgggaggatggagatcttcttggtcacaagaaggcttgctgaaggctgtatgctgtccctgatccaaaaatccaaaattttggcctctgactgattttggttttggatcagggacaggacacaaggcctgttactagcactcacatggaacaaatggccaccgtgggaggatgagcaatgttctgttagaccgaaggcttgctgaaggctgtatgctgtccctgatccaaaaatccaaaattttggcctctgactgattttggttttggatcagggacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 88 - DNA, 6604 bp, Synthetic construct
  source 1..6604: mol_type = other DNA; note = pAAV[miR155]-UBC>4x anti(hsa-mir-6090)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtgccccgcccctcgctcccccattttggcctctgactgatggggggcgggggcggggcacaggacacaaggcctgttactagcactcacatggaacaaatggccaccgtgggaggatgctgattgcgtaaccggtaaataggcttgctgaaggctgtatgctgtgccccgcccctcgctcccccattttggcctctgactgatggggggcgggggcggggcacaggacacaaggcctgttactagcactcacatggaacaaatggccaccgtgggaggatggagatcttcttggtcacaagaaggcttgctgaaggctgtatgctgtgccccgcccctcgctcccccattttggcctctgactgatggggggcgggggcggggcacaggacacaaggcctgttactagcactcacatggaacaaatggccaccgtgggaggatgagcaatgttctgttagaccgaaggcttgctgaaggctgtatgctgtgccccgcccctcgctcccccattttggcctctgactgatggggggcgggggcggggcacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 89 - DNA, 6612 bp, Synthetic construct
  source 1..6612: mol_type = other DNA; note = pAAV[miR155]-UBC>4x anti(mmu-miR-132-3p)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtcgaccatggctgtagactgttattttggcctctgactgataacagttctgccatggtcgacaggacacaaggcctgttactagcactcacatggaacaaatggccaccgtgggaggatgctgattgcgtaaccggtaaataggcttgctgaaggctgtatgctgtcgaccatggctgtagactgttattttggcctctgactgataacagttctgccatggtcgacaggacacaaggcctgttactagcactcacatggaacaaatggccaccgtgggaggatggagatcttcttggtcacaagaaggcttgctgaaggctgtatgctgtcgaccatggctgtagactgttattttggcctctgactgataacagttctgccatggtcgacaggacacaaggcctgttactagcactcacatggaacaaatggccaccgtgggaggatgagcaatgttctgttagaccgaaggcttgctgaaggctgtatgctgtcgaccatggctgtagactgttattttggcctctgactgataacagttctgccatggtcgacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 90 - DNA, 6604 bp, Synthetic construct
  source 1..6604: mol_type = other DNA; note = pAAV[miR155]-UBC>4x anti(mmu-miR-29a-3p)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtaaccgatttcagatggtgctattttggcctctgactgatagcacattgaaatcggttacaggacacaaggcctgttactagcactcacatggaacaaatggccaccgtgggaggatgctgattgcgtaaccggtaaataggcttgctgaaggctgtatgctgtaaccgatttcagatggtgctattttggcctctgactgatagcacattgaaatcggttacaggacacaaggcctgttactagcactcacatggaacaaatggccaccgtgggaggatggagatcttcttggtcacaagaaggcttgctgaaggctgtatgctgtaaccgatttcagatggtgctattttggcctctgactgatagcacattgaaatcggttacaggacacaaggcctgttactagcactcacatggaacaaatggccaccgtgggaggatgagcaatgttctgttagaccgaaggcttgctgaaggctgtatgctgtaaccgatttcagatggtgctattttggcctctgactgatagcacattgaaatcggttacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 91 - DNA, 5804 bp, Synthetic construct
  source 1..5804: mol_type = other DNA; note = pAAV[Exp]-CMV>Myc/Myc/mRpl7a[NM_013721.3]:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggagcagaagctgatcagcgaggaggacctgatgcccaaagggaagaaggccaaggggaagaaggtggccccggcccccgccgtcgtcaagaagcaggaggccaagaaggtggtcaaccctctgttcgagaagaggcccaagaacttcggcattggacaggacatccagcccaaaagagatttaacgcgcttcgtcaaatggccccgctacatcagactgcagcggcaaagagctatcctctacaagcggctcaaagtccctcctgccattaaccagttcacccaggccctggacaggcagacagctactcagctgcttaagcttgcccacaagtacaggccagagacaaagcaagagaagaagcaaaggctactggcccgtgctgagaagaaagctgctggcaaaggcgacgtcccaactaagagaccacctgtcctccgagcaggagtcaatacagtcaccaccttggtggagaacaagaaggctcagctggtggtgattgcccatgacgtagaccccattgagctggtggttttcctacctgctctgtgtcgaaagatgggggtgccctactgcatcatcaagggaaaggccaggctggggcacctggtccacaggaagacatgcaccaccgttgccttcacacaggttaactcggaagacaagggtgctctggctaagctggtggaagctattaggaccaattataatgacagatatgacgagatccgtcgccactggggaggcaacgtcctgggtcctaagtctgtggctcgaattgccaagctggaaaaagcaaaggctaaagaactcgccactaaattgggttaaacccagctttcttgtacaaagtggcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggcaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtgtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaa

SEQ ID NO: 92 - DNA, 5816 bp, Synthetic construct
  source 1..5816: mol_type = other DNA; note = pAAV[Exp]-CMV>Myc/Myc/mRpl7[NM_011291.5]:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggagcagaagctgatcagcgaggaggacctgatggaggctgttccagagaagaaaaagaaggttgccactgtgccaggaacccttaagaaaaaggttcctgctgggccaaaaactctcaagaaaaaggttcccgctgtgccagaaaccctcaagaaaaagcgaaggaatttcgcagagttgaaggtgaagcgcctgaggaagaagtttgccctgaagacacttcgaaaggcaaggaggaagctcatctatgagaaggcaaagcactatcacaaggagtacaggcagatgtaccgcactgagattcggatggccaggatggcaaggaaagctggcaacttctatgtgcccgcagaaccaaagctggcctttgtcatcagaattcgaggtatcaatggagtaagcccaaaggttcgtaaggttctgcagctccttcgtcttcgacagatcttcaatggcacctttgttaagctcaacaaggcttcaattaacatgctgcggattgtggagccatacattgcatgggggtaccccaacctgaagtcagtaaacgagctcatctacaagcgaggctacggcaaaatcaacaagaagcggattgccttgacagataattccttgattgctcggtctcttggtaagtttggcatcatctgcatggaggatctaattcatgagatctatacagtcgggaaacgcttcaaggaagcaaataacttcctgtggcccttcaagttatcttccccacgaggtgggatgaagaaaaagacaactcactttgtagaaggtggagatgctggcaacagggaagaccagataaacaggcttattagacggatgaactaaacccagctttcttgtacaaagtggcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggcaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattg

SEQ ID NO: 93 - DNA, 5350 bp, Synthetic construct
  source 1..5350: mol_type = other DNA; note = pAAV[Exp]-CMV>Myc/Myc/mTceb2[NM_026305.2]:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaacagaggatctggagcagaagctgatcagcgaggaggacctgatggacgtgtttctcatgatccggcgccacaagaccaccatctttacggacgccaaggagtcgagcaccgtgttcgaactgaagcgcatcgtcgagggcatcctcaagcggccgccagaggagcagcggctttacaaggatgaccagctccttgatgatggcaaaactctgggcgagtgtggcttcactagccagacagcacggccacaggccccagccacagtgggcctggccttccgagcagatgacaccttcgaagctctccgcatcgagcccttttccagccctccagagcttccagatgtgatgaagccacaggattctggaggcagtgccaatgaacaagctgtgcagtgaacccagctttcttgtacaaagtggcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggcaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattg

SEQ ID NO: 94 - DNA, 5661 bp, Synthetic construct
  source 1..5661: mol_type = other DNA; note = pAAV[Exp]-CMV>{2xMyc/mLancl1[NM_001190984.1]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggaacaaaaactcatctcagaagaggatctgatggctcagagggcctttccgaatccttatgccgattataacaagtcgctggccgaaaactactttgattctactgggaggctgactcctgagttctcacatcgcttgaccaataagatccgagagctccttcagcaaatggagagaggcctaaagtctgcagaccctcgggatggcactgggtacactggctgggcaggcatcgctgtgctttacctccacctccacaatgtgtttggggacccagcctatctacagatggcgcacagctatgtaaagcaaagcctcaactgtctgagcaggcgttccatcaccttcctgtgtggcgatgcgggccccctggctgtggctgctgtgctgtaccacaagatgaacagtgagaagcaggccgaggagtgcatcacacggctcattcacctaaataagattgatccccacgtaccaaatgaaatgctctatggccgcataggctacatctttgctctgctttttgtcaataagaactttggagaggagaagattcctcagagccatattcagcagatttgtgaaaacatcttaacctctggggaaaacctatctagaaagagaaacttggcggcaaagtctccactgatgtatgagtggtaccaggaatattacgtgggggctgctcatggcttggcaggcatttattactacctgatgcagcccagccttcaggtgaaccaaggaaagttgcatagtttggtgaagcccagtgtagactttgtctgccggctgaagtttccttccggcaattaccctccatgtttggatgataccagagacctgcttgtccattggtgccacggtgctcctggggtcatctatatgctcatccaagcatacaaggtgttcaaagaagagcgttacctgtgtgatgcccagcagtgtgctgacgtgatctggcagtacgggctactgaagaagggctacgggctgtgccatggtgctgcagggaatgcctacgctttcctggcactctacaacctcacacaggatctgaagtatctgtacagggcctgcaagtttgctgagtggtgcttagactacggggagcacggatgcaggacagcggacacccccttctccctctttgaagggatggctggaacaatatatttcttggctgacctgttagtccccacaaaggccaagttccctgcatttgaactctgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 95 - DNA, 4929 bp, Synthetic construct
  source 1..4929: mol_type = other DNA; note = pAAV[Exp]-CMV>{2xMyc/mAtp6v0c[NM_009729.4]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggaacaaaaactcatctcagaagaggatctgatggctgacatcaagaacaaccccgaatattcttcgtttttcggtgtcatgggcgcctcgtccgccatggtcttcagcgccatgggagccgcctatggcacagccaagagtggcactggcatcgcagccatgtcagtcatgaggccagagctgatcatgaagtccatcatcccagtggttatggctgggatcatcgccatctacggcctggtggtggcagtacttatcgctaactccctgactgatggcatcaccctctacaggagttttcttcaactgggtgctggcctgagtgtggggctgagtggcctggctgctggctttgccattggcatcgtcggagatgctggtgttcggggcactgcccagcagcctcgactgttcgtgggcatgatcctgatcctcatctttgcggaggtgcttggcctctacggtctcatcgtggccctaatcctctccacaaagtagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 96 - DNA, 4803 bp, Synthetic construct
  source 1..4803: mol_type = other DNA; note = pAAV[Exp]-CMV>{2xMyc/mDynlt1a[NM_001166629.2]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggaacaaaaactcatctcagaagaggatctgatggaagacttccaggcctccgaggagactgcatttgttgttgatgaagtgagcagcattgtaaaggaggctatagaaagcgccatcggtggtaatgcctaccagcacagcaaagtcaaccagtggaccactaatgtcctagaacagactttgagccaactcaccaaactggggagaccatttaaatacattgtgacctgtgtgatcatgcagaagaacggtgctgggttacactccgcaagttcctgcttctgggacagctccacagacggaagctgcacagtccgatgggagaacaagaccatgtactgcatcgtcagtaccttcggactgtccatctgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 97 - DNA, 7251 bp, Synthetic construct
  source 1..7251: mol_type = other DNA; note = pAAV[Exp]-CMV>{2xMyc/mMrtfa[NM_153049.3]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggaacaaaaactcatctcagaagaggatctgatgccgcctttgaaaagccccgctgcatttcatgagcagagaagaagcctggagcgggccaggaccgaggactatttgaaacggaagatccgttcccggcccgagagagcagagctggtcaggatgcacattctggaagagacctcggctgagccttcgctccaggccaagcagctgaagctgaagagagccaggctggctgatgacctcaatgaaaagattgcacagaggcctggccccatggagctggtggagaagaatatcctgcctgtggagtccagcctgaaggaggctatcattgtgggccaggtaaattacccaaaggtagcagacagttcctccttcgacgaggacagcagcgatgccctgtctcctgagcagcctgccagccatgagtcccagggttcagtgccatcacccttggagtcccgagtcagtgatccactgcccagtgccacctccatatcacccactcaggttctttctcaactcccaatggctccggatcctggagagacgctttttctggcagagcagcctcctctgcctcccgcacctctgctgcccccaagcctagccaatggaagcatcgtccccactgccaagcctgctcccacactcatcaagcaaagccaacccaagtctgccagcgagaaatcacagcgcagcaagaaggccaaggagctgaagccaaaggtgaagaagctcaagtaccaccagtacatccccccggaccagaagcaggacaagggggcgcccgccatggactcctcctatgccaagatcctgcagcagcagcagctcttcctgcagctgcagatcctcaaccagcagcagcagcagcagcagcaacagcactacaactaccaggccatcctgcctgcccctcccaagccctcggctgagactcctggaagcagtgcccctaccccatcacgcagcctctccaccagtagcagccccagctcaggcaccccagggcccagcgggctggcacgccagagcagcaccgcactagctgccaaaccaggagccctgccagccaacctggatgacatgaaggtggcagagctgaagcaagaactgaagttgcggtcccttcccgtctcaggcaccaagacagagctgatagaacgcctgcgtgcctaccaagaccaagtcagcccagctccaggagcccccaaggcccctgccaccacctctgtgctgtccaaggctggtgaggtagtggtcgccttccctgcggccctgctaagcacagggtcagctcttgtaacagcaggccttgcaccagctgagatggtggtggccacagtaaccagcaatggcatggtgaagtttggcagcacaggctccacaccccccgtgtctcccaccccttcagagcgctcactgctcagcacgggtgatgagaattccacacctggggatgcctttggtgaaatggtgacatcgccgctgacacagctcaccctgcaggcctccccactgcagatcgtgaaggaggagggtgcccgtgctgcgtcctgctgtctaagccctggtgctcgggctgagctggagggactggacaaggaccagatgctgcaggagaaggacaagcagattgaggagctgacccgaatgctccaacagaagcagcagctggttgagctgctgcggctacagctggagcagcagaagcgggcccagcagccagccccagccagcagccctgtgaagagggaaagtggtttctccagttgccagctgagctgccagccccagggctctgcccatgcttttggctctggcctagtggttcccactaccaaccatggagacactcaggccccagcgccagagtccccacctgtggtggtgaagcaggaagctgggccacctgagccagatctggcccccagctcccagctgctcttgggctcccagggcaccagcttcctcaagagggtcagccctcctaccctggtcactgactctacagggactcacctcatcctcactgtgaccaataagagtgctgatggccctggcttgcctgcagggagcccccagcagcccttgtcccagcctggttctccagcccctggtccacctgcccagatggacctggagcacccacctcagcctccgtttgcaacccccacatctctgctgaagaaggagccccctggttatgaagagactgtgacccagcagcctaagcagcaggaaaatggctcctccagtcagcacatggatgatctgtttgatattcttattcagagtggagagatttcagcagatttcaaagagccaccatccctaccaggcaaggaaaagtcacctccagcagcagcagcgtatgggcctccattgacaccacaaccctcgcctttgagtgaactcccccaagctgctcctccaccaggttcccccaccctcccagggcgccttgaagacttcctggagagcagcacagggctgcccctgctgacaagtgggcacgagggaccagaacccctttccctcattgatgacctccacagccagatgctgagcagctccgccatcctggaccaccccccatcacccatggacacctctgaattgcactttgctcctgagcccagcagtggtatgggcctggacctggctgttggccacctggacagcatggactggctggagctgtcgtctggtggccctgtgctcagcctggctcccctcagcactgcagcccccagcctcttctcgatggacttcctggatggacacgacttgcagctccactgggattcctgcttgtagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 98 - DNA, 7170 bp, Synthetic construct
  source 1..7170: mol_type = other DNA; note = pAAV[Exp]-CMV>{2xMyc/mLars2[NM_001348167.1]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggaacaaaaactcatctcagaagaggatctgatggcatccacttgtcagaggctgagtttctatgtctctcctctgaaaaggcagctagttagcaggccacctgtcatcctgtgggaaagactaattccagggtgttccagaagtatctacagtgccaccgggaagtggacaaaggagtacacactgcagacaaggaaggatgtggagaagtggtggcaccaacagataaaggagcaagcgtccagagtctcagaggaagataaattaaagcccaaattctacttgctctccatgttcccatacccttctggcaagctgcacatgggccatgtccgtgtctacaccctcagcgacaccattgcacggttccagaaaatgagaggcatgcaggttatcaaccccatggggtgggatgcatttggattgcctgctgaaaatgctgccatagagaggaatttgcaccctgaaagctggacccaaagcaatattaaacacatgaggaagcagctcgaccgcctcggcctgtgcttcagctgggacagggaaatcactacatgtttgccagattactacaaatggactcaatacctgtttatcaaactctatgaagcaggactggcctatcaaaaggaggccttggttaactgggatccagtggatcaaacagtgctagccaatgagcaggtgaatgaatatggctgttcatggcgttctggagccaaggtagagaagaaatacctccgacagtggttcattaagacaactgcttatgcaaaggccatgcaggatgcgttggcagacctcccagagtggtacggaatcaaaggcatgcaggcccactggattggggactgtgtaggctgtcacctggacttcacattaaaggttgatggagaagacacaggagagaaattgacagcatacacagccacccctgaggccatctatggcatctcccacgtggccatctctcctagccacgggcttctacatggctgcagctctgtgaagaaagccttgcagaaggcactagtccctggcagagattgcctcacaccagtgatggctgtgagcatgctcaccctgcaggaggtccccatcgttatcatggctaaccctgacttggaaggctctctagattcaaaaataggaatccccagcaccagctcagaggacaccagactggcccaagctctgggcctaccctactctgaggtcattgaagcttcacccgatggcacagagagactgagtggatctgctgagtttacaggtatgaccaggcaggatgcctttgtagccctgactcggaaagcccgtgggatgagagtgggtggacacgtgacaagtaacaagctgaaggactggctgatctcacggcagcgctactggggcacaccgatccccattgtccactgcccagcctgtggccctgtgcctgtgccactgcaagacttgcctgtgattttgcccagcattgcatctttaacgggcagaggaggctccccgctggccacggctttggagtgggtgaactgctcctgcccaaggtgcaagggatcagcaaagagagagacggacaccatggacacctttgtggattctgcgtggtactacttcagatacactgaccctcataatactcaaagtccttttggctcggcactggcagacttctggatgcccgtggatctgtacatcggaggaaaggaacatgctgtcatgcacttgttctatgcacgattcctcagtcatttttgccacgaccaaaagatggtgaagcacagggaacctttccataaactactggcccaaggcctcattaagggacagacattccgccttccatctggacagtgtctgaagaaggaggatatagatttcacaggtcctgctcctgtttgtgcaaaaacaaaagagaagttagaggtgacgtgggagaagatgagcaagtccaaacacaatggggtggaccctgaggagattgtggcccaatatggaattgacacaatccggctctacatcctctttgcagcccctcctgagaaggacatcttgtgggatgtaaagactgacgcactccctggagtgctaagatggcaacagcggttgtggtccctgacaacaagattcatcgaggcaaggacttccgggacagtccctcagcctcagctgctgaacagcaaggagaaaaccaaggcccaaaacctctgggagtacaaaaatgcagtcatagctcaggtgaccactcatttcacagaggactttgccctgaattctgtagtttctcagctgatgggactcagcagtgccctctcgcaagcctctcagagagtggttctccacagccctgagtttgaagatgctctgtgtgccctgctggtgatggctgctcccctggcccctcatgtaacctcagaactttgggcaggcctgacgctggtaccaagcaagctctgtgaccattatgcctgggattctggtgtgatgcttcaggcctggcccactgtggactcacagttccttcagaaacctgacatggtgcagatggctgttctgatcaacaacaaggcctgtggcaagatccctgtgccccagcatgttgcccaggaccaggacaaagtccatgaacttgttctccagagcgagctaggcatgaagctgctgcaggggagaagcatcaagaaggccttcctctcccccaggactgccctcatcaacttcttggtgcaggagtgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 99 - DNA, 6156 bp, Synthetic construct
  source 1..6156: mol_type = other DNA; note = pAAV[Exp]-CMV>{Myc/Myc/mDpysl5[NM_023047.3]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggagcagaagctgatcagcgaggaggacctgatgcttgccaattcagccagcgtgcggattctcatcaagggaggcaaggtggtgaacgatgactgtacccacgaggcagacgtctacatcgagagcggcatcatccagcaggtgggccgtgagctcatgatccccggtggggccaaggtgattgacgccaccgggaaactggtgatcccgggagggattgacaccagcacccacttccaccagaccttcatgaacgccacctgtgtggacgacttctaccatgggaccaaggcggccctcgttggaggcaccaccatgatcatcggtcacgtcctgccagacaaggagacctccctcgtggaagcctatgagaagtgcagggctctagctgaccccaaggtctgctgtgactatgccctccatgtggggatcacctggtgggcacccaaggtgaaagcagaaatggagacactggtgcgggagaagggggtcaactccttccagatgttcatgacctacaaggacttgtacatgcttcgagacagtgagctgtaccaagtgtttcatgcctgcagggacatcggggcgatcccccgggtccacgctgaaaatggagagctcgtggctgagggtgctaaggaggctctagacttgggtatcacaggcccagaaggaatcgagatcagccatccagaggagctggaagcagaagccactcaccgtgtcatcaccattgcaaaccggactcactgtcccatctacctggtcaatgtgtctagtatctcagctggtgatgtcattgcagctgctaagatgcaagggaaggtggtgctagccgagaccaccaacgcacacgccacgctcacgggcttgcactactaccatcaggactggtcgcatgcagccgcctatgtcacggtgcctcctctgagactggacaccaacacttccacctacctcatgagcctgctggccaatgacactctgaacattgtggcatccgatcaccggccattcaccacaaagcagaaagccatgggcaaggaagacttcactaagatcccccacggtgtgagcggtgtacaggatcgaatgagcgtcgtctgggagaggggcgtggttggaggaaagatggatgagaaccgtttcgtggccgtcaccagttctaacgcagccaagatcctcaacctgtatcctcgcaagggccgtatcatccccggagccgacgctgatgtcgtggtgtgggacccggaagccacaaagaccatctcagccagcacccaggtgcagggtggagacttcaatctgtatgagaacatgcgctgccacggtgtaccgctggtcaccatcagccggggacgagtcgtgtatgagaacggcgtcttcatgtgtgcagagggcactggcaagttctgtcccctgcggtctttcccagacattgtctataagaagctggtacagagagagaagaccttaaaagtgaggggagtggaccgcactccctacctaggggatgtcgccattgttgtccaccctgggaaaaaagagatgggaacaccattggcagacacgcccacacggccagtcacccggcacgggggcatgcgggaccttcatgaatccagtttcagcctctctggttctcagatcgatgaccacgttccaaagcgggcctcagctcggatccttgcgccccctggaggcaggtcgagcggtatttggtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 100 - DNA, 5589 bp, Synthetic construct
  source 1..5589: mol_type = other DNA; note = pAAV[Exp]-CMV>{Myc/Myc/mFblim1[NM_133754.5]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggagcagaagctgatcagcgaggaggacctgatggcctccaagcctgagaaaagggtggcctcctctgtcttcatcaccctggcaccgccacgccgagatgtagccgtgagtgaggaagtgggccaggcagcttgtgaagccagacgcgctcggccctgggagatgcttcccacaaagacacctggggccgcagtgggcaggagccccaagacctggacgccctctggcaagaccaatgcctcactctctggagtcacacctcagctctccaatggaggatgctctctcccacctccttccctgaatgaggaggacctagacctcccgcctcctccaccgcccccttctgcctacctgcctctcccagaagaggagcctcctgtcttaccagggaagtcgctcatttccgacttggagcaactgcacctgcccccacccccgcctccacccccgccacaggctccatcaaagggatcgtctgtccaccctccgcctggtcacgccataccctcggaagaggagcttcctcctcctccagaagagcctgtcacacttccagagagagaagtgtccacggatgtctgtggtttctgtcacaagcctgtgtctcctcgagagctggctgttgaggccatgaagaggcagtaccacgcccagtgcttcacctgtcgtacctgccgccgccagttggctggacagagattctaccagaaggatgggcgccccctgtgcgaaccctgctaccaggatactctggagaagtgtggcaagtgcggagaggtggtccaagagcacgtgatccgggccctgggcaaggccttccacccaccctgcttcacctgcgtgacctgtgcccgctgcatcagcgacgagagctttgcgctggacagccagaaccaggtgtactgtgtggctgatttctacaggaaatttgcccccgtgtgcagcatctgcgagaatcccatcatcccccgagacgggaaggacgccttcaaaatcgagtgcatgggaaggaatttccacgagaactgttaccgctgtgaggactgcagcgtgctcctgtctgtggaacctaccgaccaaggctgctacccactgaatgaccacctcttctgcaagccctgtcacctgaagcggagtgctgctggttgctgctgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 101 - DNA, 6167 bp, Synthetic construct
  source 1..6167: mol_type = other DNA; note = pAAV[Exp]-CMV>{Myc/Myc/mDpysl3[NM_001136086]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggagcagaagctgatcagcgaggaggacctgatgtattcaccatcaagaaacaaaatccggcagagtgaccgtcttctaatcaagggagggagaatcgtcaatgatgatcagtccttttatgctgatatttacatggaggatggcttaataaagcaaattggagacaatctgattgtccccggaggtgtgaagaccatcgaggccaacgggaagatggtgatccctggaggcattgacgtccatacccacttccagatgccttacaagggaatgaccacagtggacgatttcttccaagggacaaaggctgccttagcgggaggcaccaccatgatcattgaccacgtggtacctgaacctgagtctagcctgacagaggcctatgaaaagtggcgtgagtgggccgatgggaagagctgctgtgactatgctttgcatgtggacatcacccactggaatgacagcgtcaagcaggaggtacagagcctcagcaaggaaaaaggcgttaactccttcatggtttacatggcctacaaggatttatatcaagtgtccaacacagagctctatgagatcttcacctgcctgggagagctgggggccattgctcaagttcatgccgagaatggagatatcattgcccaggagcaggcacggatgctggaaatggggataaccggcccagaaggccacgttctggcagaccggaagagctggaagctgaggctgtgttccgtgccatcaccgtcgccagccagaccaactgccccctctatgtcaccaaggtcatgagcaagagcgcggctgatctcatctcacaagccaggaagaaaggaaatgtggtctttggcgagcccatcactgccagcctgggaatagatggaactcattactggagtaagaactgggccaaagcagctgcgtttgtgacatccccacctctgagccctgaccctaccacacctgactacatcaactccttgctggccagcggggatctgcagctctctggaagtgcccactgtacctttagcactgcccagaaagccattgggaaggacaacttcacagccatccctgaaggcaccaatggcgtggaggagcgtatgtctgtcatctgggacaaggctgtggccacggggaagatggatgaaaaccagtttgtggccgtgacaagcaccaatgctgccaagatattcaacctgtaccctcgaaaggggagaatagctgtgggctctgacagcgaccttgtcatctgggatccagatgccttgaagattgtctctgccaagaaccaccagtcggttgccgaatacaacatctttgaagggatggagctgcgtggtgcacctctggtggttatctgccagggcaagatcatgctggaagatggcaacctgcacgtgacccagggggctggccgcttcattccctgcagcccattctctgactatgtctataagcgcattaaagcaaggaggaagatggcagacctgcatgcagtcccaagaggcatgtatgatggaccagtgtttgacttgaccaccacccccaaggggggcaccccagctggctctactcggggctctcccactcggccaaacccgccagtgaggaacctccatcagtcaggatttagcctgtcaggcacccaagtggatgagggtgtccgctcagctagcaaacgcattgtggcaccccctggaggccgttctaacatcacatccctgagttaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 102 - DNA, 6444 bp, Synthetic construct
  source 1..6444: mol_type = other DNA; note = pAAV[Exp]-CMV>{Myc/Myc/mNfe2l3[NM_010903.2]}:WPRE
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggaacaaaaactcatctcagaagaggatctggagcagaagctgatcagcgaggaggacctgatgaagctcccgaagccttggtgggcgggcggcggcctcctgcaactcacgattctgttgagcttggtagggctccgtgtggacctggacctcttcctgccgccgccggccgccgcactttgggaagagctgctcccactctgccccacccgccccgcctcggcttctaaccccttctcggcttcggaaggctgggaacgcacccccctgctacctgccaaaggccggctgctgcacgaagtgcgcgcgctcggggtccccttcattccccgcacccgcgtggacgcgtggctagtgcacagcgtggcgactgggaacgcagacggggcccacgggctgctcggcaccgccgcctcgtcggccgtgggagacggcggccagtcagcatccgcgggcggcggggacccccgagcggctcatagcagccctctggccgctgaggaagaggaggagaaggcggccgaaccgacggcgcaggtgccagatgcaggcggatgcgggagccaggagaacgggatgctaagagaaaagagcgaagctgtggatcacagttcccagcaggaggaaaacgaggaaggggtgtcagcccaggcaaaatcgcgacagcagagtaagacggaggaacacaaaatggcgtgtgcctctgccagggaagccgagaagatcaccgaggctaggaacgagagccatctacattggtcagatacatctttctctttggaagatttattccaactactttcatcacagcctgaacattctctggagggcatctcagtggaagacattccaccttttctgagcagtgtctgtgaaagtgttaattcctcagcacagaacataaatttgagccaagctataagccatgatgtcaatcttcatgaagccatgctgttgtaccccaacaacacatttagaagagatccatcagcaaggtcttcacaggcacaagaaccatttctgcagttaaatcctcatacaaatcctgagcaagccactcctgcaatgtctcttccaccgtttgacaatcaaatgaggaacctaacaagccaagatctcctgtatgacctggattcaaatatatttgatggaataaacctaatgtcattggccacaggtttcagtccattggaagtttctcaactttttgaagaaccagattccgggctctcattaaattcaagttacaacagcacctctctcacaaattcttaccgtatttatgatgggactgtaggttacaatagcgacctacagtctctttgtcatgatttaggtgctgtaggaggctgctacccagaaccccataaacattgtcatatggaccataggactatagctggctttcatgtgtcccttgaatgtcagcaagtatttcatgaccacacttaccatttacagtcaggtgcatcagagcccacatctgaatcttttgcatggtctgagaagtcgcagaaaaccagtggatgccttgataacccagatagaaacttgagccgtgatgaacagcgtgctaaagctctgcacattcccttttctgtagatgaaattgtccgtatgcctgttgactctttcaacagcatgttaagcagatactacctgacagacttacaagtgtctctcatccgtgatatcaggcgaagaggaaaaaataaagttgctgctcagaactgccgtaaacgtaaactagacatcattttgaatctagaggatgatatatgtaacttgcaagcaaagaaagaagcccttaaaaatgagcaaactcaatgtagcaaagctattgacataatgaggcagaagctacatggccttcaccaggatgtttttaatagattaagagatgaccaaggtaggccagtcaatccaagccaatatgcacttcagtacagccatgatggaactgttttgattgtacccaaagaactcatctcatcaggccacaaaaaggaagccccaaagggaaagagagagagaagaaactgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 103 - DNA, 6612 bp, Synthetic construct
  source 1..6612: mol_type = other DNA; note = pAAV[miR155]-UBC>4x mmu-miR-5109
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtgttgcggaccaggggaatccgattttggcctctgactgatcggattcctggtccgcaacacaggacacaaggcctgttactagcactcacatggaacaaatggccaccgtgggaggatgctgattgcgtaaccggtaaataggcttgctgaaggctgtatgctgtgttgcggaccaggggaatccgattttggcctctgactgatcggattcctggtccgcaacacaggacacaaggcctgttactagcactcacatggaacaaatggccaccgtgggaggatggagatcttcttggtcacaagaaggcttgctgaaggctgtatgctgtgttgcggaccaggggaatccgattttggcctctgactgatcggattcctggtccgcaacacaggacacaaggcctgttactagcactcacatggaacaaatggccaccgtgggaggatgagcaatgttctgttagaccgaaggcttgctgaaggctgtatgctgtgttgcggaccaggggaatccgattttggcctctgactgatcggattcctggtccgcaacacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 104 - DNA, 6604 bp, Synthetic construct
  source 1..6604: mol_type = other DNA; note = pAAV[miR155]-UBC>4x mmu-miR-1247-5p
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgacccgtcccgttcgtccccggattttggcctctgactgatccgggacgacgggacgggtcaggacacaaggcctgttactagcactcacatggaacaaatggccaccgtgggaggatgctgattgcgtaaccggtaaataggcttgctgaaggctgtatgctgacccgtcccgttcgtccccggattttggcctctgactgatccgggacgacgggacgggtcaggacacaaggcctgttactagcactcacatggaacaaatggccaccgtgggaggatggagatcttcttggtcacaagaaggcttgctgaaggctgtatgctgacccgtcccgttcgtccccggattttggcctctgactgatccgggacgacgggacgggtcaggacacaaggcctgttactagcactcacatggaacaaatggccaccgtgggaggatgagcaatgttctgttagaccgaaggcttgctgaaggctgtatgctgacccgtcccgttcgtccccggattttggcctctgactgatccgggacgacgggacgggtcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 105 - DNA, 6612 bp, Synthetic construct
  source 1..6612: mol_type = other DNA; note = pAAV[miR155]-UBC>4x mmu-miR-210-3p
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtctgtgcgtgtgacagcggctgattttggcctctgactgatcagccgtatacacgcacagacaggacacaaggcctgttactagcactcacatggaacaaatggccaccgtgggaggatgctgattgcgtaaccggtaaataggcttgctgaaggctgtatgctgtctgtgcgtgtgacagcggctgattttggcctctgactgatcagccgtatacacgcacagacaggacacaaggcctgttactagcactcacatggaacaaatggccaccgtgggaggatggagatcttcttggtcacaagaaggcttgctgaaggctgtatgctgtctgtgcgtgtgacagcggctgattttggcctctgactgatcagccgtatacacgcacagacaggacacaaggcctgttactagcactcacatggaacaaatggccaccgtgggaggatgagcaatgttctgttagaccgaaggcttgctgaaggctgtatgctgtctgtgcgtgtgacagcggctgattttggcctctgactgatcagccgtatacacgcacagacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 106 - DNA, 6604 bp, Synthetic construct
  source 1..6604: mol_type = other DNA; note = pAAV[miR155]-UBC>4x hsa-mir-3914-1
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgaaggaaccagaaaatgagaagtttttggcctctgactgaacttctatttctggttccttcaggacacaaggcctgttactagcactcacatggaacaaatggccaccgtgggaggatgctgattgcgtaaccggtaaataggcttgctgaaggctgtatgctgaaggaaccagaaaatgagaagtttttggcctctgactgaacttctatttctggttccttcaggacacaaggcctgttactagcactcacatggaacaaatggccaccgtgggaggatggagatcttcttggtcacaagaaggcttgctgaaggctgtatgctgaaggaaccagaaaatgagaagtttttggcctctgactgaacttctatttctggttccttcaggacacaaggcctgttactagcactcacatggaacaaatggccaccgtgggaggatgagcaatgttctgttagaccgaaggcttgctgaaggctgtatgctgaaggaaccagaaaatgagaagtttttggcctctgactgaacttctatttctggttccttcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 107 - DNA, 6604 bp, Synthetic construct
  source 1..6604: mol_type = other DNA; note = pAAV[miR155]-UBC>4x mmu-miR-135b-3p
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgatgtagggctaaaagccatgggttttggcctctgactgacccatgctttagccctacatcaggacacaaggcctgttactagcactcacatggaacaaatggccaccgtgggaggatgctgattgcgtaaccggtaaataggcttgctgaaggctgtatgctgatgtagggctaaaagccatgggttttggcctctgactgacccatgctttagccctacatcaggacacaaggcctgttactagcactcacatggaacaaatggccaccgtgggaggatggagatcttcttggtcacaagaaggcttgctgaaggctgtatgctgatgtagggctaaaagccatgggttttggcctctgactgacccatgctttagccctacatcaggacacaaggcctgttactagcactcacatggaacaaatggccaccgtgggaggatgagcaatgttctgttagaccgaaggcttgctgaaggctgtatgctgatgtagggctaaaagccatgggttttggcctctgactgacccatgctttagccctacatcaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 108 - DNA, 6604 bp, Synthetic construct
  source 1..6604: mol_type = other DNA; note = pAAV[miR155]-UBC>4x mmu-miR-135a-1-3p
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaaactggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatagaaccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaaggcgcgcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagatcccatcgattcgaattcaaagtcgactggaggcttgctgaaggctgtatgctgtatagggattggagccgtggcgttttggcctctgactgacgccacgctcaatccctatacaggacacaaggcctgttactagcactcacatggaacaaatggccaccgtgggaggatgctgattgcgtaaccggtaaataggcttgctgaaggctgtatgctgtatagggattggagccgtggcgttttggcctctgactgacgccacgctcaatccctatacaggacacaaggcctgttactagcactcacatggaacaaatggccaccgtgggaggatggagatcttcttggtcacaagaaggcttgctgaaggctgtatgctgtatagggattggagccgtggcgttttggcctctgactgacgccacgctcaatccctatacaggacacaaggcctgttactagcactcacatggaacaaatggccaccgtgggaggatgagcaatgttctgttagaccgaaggcttgctgaaggctgtatgctgtatagggattggagccgtggcgttttggcctctgactgacgccacgctcaatccctatacaggacacaaggcctgttactagcactcacatggaacaaatggccaccgtgggaggatgacaactcgagcctctagacaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcaagcttaaggatccaccatggagcagaagctgatctccgaggaggacctgggatccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2024/0368252 A1
(43) Pub. Date: Nov. 7, 2024

(54) NOVEL COMPOSITIONS FOR NEUROPROTECTION AND AXON REGENERATION

(71) Applicant: University of Connecticut, Farmington, CT (US)

(72) Inventors: Feliks Ephraim Trakhtenberg, West Hartford, CT (US); Agnieszka Lukomska, Hartford, CT (US); Bruce Rheaume, Farmington, CT (US); Jian Xing, Hartford, CT (US)

(21) Appl. No.: 18/626,022

(22) Filed: Apr. 3, 2024

Related U.S. Application Data
(60) Provisional application No. 63/493,890, filed on Apr. 3, 2023.
(60) Provisional application No. 63/499,359, filed on May 1, 2023.

(51) Int. Cl. C07K 14/78; A61K 38/00; A61K 48/00; A61P 25/00; C12N 15/113; C12N 15/86

(52) U.S. Cl. CPC C07K 14/78; A61K 48/0058; A61P 25/00; C12N 15/113; C12N 15/86; A61K 38/00; C07K 2319/21; C12N 2310/531; C12N 2750/14143

(57) ABSTRACT

Disclosed are fibronectin-based peptides, and novel nucleic acids and compositions comprising the same, for promoting neuroprotection and/or axon regeneration in neurons. Disclosed herein are methods to treat or prevent neuronal injury and disease and disorders characterized by neuronal injury.

### CROSS REFERENCE TO RELATED APPLICATIONS

This application claims priority to U.S. Provisional Application 63/499,359, filed on May 1, 2023, and U.S. Provisional Application 63/493,890 filed on Apr. 3, 2023, both of which are incorporated by reference in their entirety herein.

### GOVERNMENTAL SUPPORT

This invention was made with government support under EY029739 awarded by the National Institutes of Health. The government has certain rights in the invention.

### INCORPORATION OF ELECTRONICALLY FILED MATERIAL

The Instant Application contains a Sequence Listing which has been submitted electronically in XML format and is hereby incorporated by reference in its entirety. Said XML copy, created on Apr. 3, 2024 is named “UCT0306US2” and is 367,769 bytes in size.

### BACKGROUND

Mammalian central nervous system (CNS) projection neurons fail to spontaneously regenerate axons damaged by injury or neurodegenerative disease. Currently, there is no efficient neuroprotective or neuroregenerative treatments for neural connections, such as efficient neuroprotective or neuroregenerative eye treatments for optic neuropathies, as the existing clinical interventions and products, relying on early detection, focus on innervations and treatments which delay and reduce the extent of damage. However, if the damage has already occurred, there is no efficient neuroprotective or neuroregenerative treatments for neural connections, such as efficient neuroprotective or neuroregenerative eye treatments for optic neuropathies.

Genes which are developmentally regulated in the maturing central nervous system (CNS) projection neurons can be involved in the control of developmental axonal growth and the failure of axonal regeneration after injury. CNS projection neurons have been shown to lose their capacity for regenerating axons in vivo as they mature. Furthermore, mature CNS projection neurons, such as the retinal ganglion cells (RGCs), also do not grow axons in culture on laminin-coated surface. However, there is robust axon growth when immature RGCs, and other CNS neurons, are grown on laminin.

A number of developmentally regulated genes have been found to underlie the developmental decline in intrinsic capacity of retinal ganglion cells (RGCs) (and other CNS projection neurons) to grow axons. Several approaches succeeded in promoting various extents of axonal regeneration in the CNS, for example, after traumatic optic neuropathy modeled by optic nerve crush (ONC) in animal models. Nevertheless, even in the approaches targeting potent pro-growth tumorigenic factors (which are clinically concerning), only a rare subset of the axons regenerates the full-length. The tumor suppressor gene, Pten is one of the most potent gene-regulators of axon regeneration discovered to date. Pten suppresses axon regeneration through inhibition of the mammalian target of rapamycin (mTOR) pathway, and phosphatase and tensin homolog (Pten) knockout was shown to promote various extents of axon regeneration from the RGCs that included a small subset of RGCs. Although experimental gene therapy knockdown (KD) of Pten expression in adult RGCs promotes long-distance axon regeneration in a small subset of RGCs, it is concerning for clinical use, and safer downstream effectors of Pten KD and mTOR pathway-regulation are needed.

There is a need for efficient clinical alternatives for promoting neuroprotection and delaying degeneration of axons as a preventive treatment in case of early diagnosis, as well as compounds for axon regeneration due to injury or damage.

### SUMMARY

The inventors of the present disclosure surprisingly and unexpectedly discovered that a fibronectin-based/derived peptide (also referenced herein as “an agent,” “the recombinant peptide,” or “therapeutic peptide”, RP-RGD (i.e. recombinant peptide containing the RGD domain of fibronectin protein)) can be utilized with various signal peptides, with or without a purification tag, as a peptide therapeutic, such as a peptide therapeutic to repair neural connections or prevent damage to neural connection (e.g., a preventive treatment or co-treatment and a post-damage treatment or co-treatment). The surprising use of vitronectin, fibronectin, and fibronectin-based peptides as a preventive treatment or co-treatment and a post-damage treatment or co-treatment was unexpectedly discovered by the present inventors.

An aspect of the present disclosure relates to a fibronectin-based peptide (also referenced herein as “an agent,” “the recombinant peptide,” or “therapeutic peptide”) comprising, or consisting of, a fibronectin peptide that is derived from (e.g., a fragment of), or has, the amino acid sequence

(SEQ ID NO: 41)
DSPTGIDFSDITANSFTVHWIAPRATITGYRIRHHPEHFSGRPREDRVPH
SRNSITLTNLTPGTEYVVSIVALNGREESPLLIGQQSTVSDVPRDLEVVA
ATPTSLLISWDAPAVTVRYYRITYGETGGNSPVQEFTVPGSKSTATISGL
KPGVDYTITVYAVTGRGDSP.

A further aspect of the present disclosure relates to a fibronectin-based peptide (also referenced herein as “an agent,” “the recombinant peptide,” or “therapeutic peptide”) comprising, or consisting of, a fibronectin peptide that has the amino acid sequence

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin peptide has an amino acid sequence that is at least 90% or at least 95% (e.g., at least 90%, 91%, 92%, 93%, 94%, 95%, 96%, 97%, 98%, 99%, or 99.5%) identity or homology with (e.g., a fragment of) the amino acid sequence:

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin peptide has the amino acid sequence:

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin-based peptide further comprises a signal peptide (e.g., a signal peptide on the N-terminus of the fibronectin peptide or C-terminus of the fibronectin peptide).

In any aspect or embodiment described herein, the signal peptide comprises or consists of the amino acid sequence MGWSCIILFLVATATGVHS (SEQ ID NO:42).

In any aspect or embodiment described herein, the fibronectin-based peptide further comprises an affinity tag (e.g., an affinity tag on the N-terminus of the fibronectin peptide or C-terminus of the fibronectin peptide).

In any aspect or embodiment described herein, the affinity tag comprises or is: biotin (e.g., binds streptavidin, avidin, or neutravidin), calmodulin-binding peptide (CBP) tag (KRRWKKNFIAVSAANRFKKISSSGAL; SEQ ID NO:43) (e.g., binds calmodulin), chitin-binding domain (CBD) tag (e.g., binds chitin), E-tag (GAPVPYPDPLEPR; SEQ ID NO:44) (e.g., binds anti-E-tag antibody), Fc-tag (e.g., binds Protein-A Sepharose), FLAG tag (DYKDDDDK; SEQ ID NO:45) (e.g., binds anti-FLAG monoclonal antibody, such as M1, M2, and M5, or a derivative thereof), glutathione S-transferase (GST) tag (e.g., binds glutathione), HA-tag (YPYDVPDYA; SEQ ID NO:46) (e.g., binds anti-hemagglutinin antibody), HaloTag (binds a reactive chloroalkane linker), Histidine tag (5-10 histidines) (e.g., binds metal ions, such as Ni²⁺, Co²⁺, Cu²⁺, Zn²⁺, Fe³⁺), Gly-His-tags (e.g. GHHHH, or GHHHHHH (SEQ ID NO:47), or GSSHHHHHH (SEQ ID NO:48)) (e.g., bind immobilized metal cations), maltose-binding protein (MBP) (binds amylose), Myc-tag (EQKLISEEDL; SEQ ID NO:49) (e.g., binds an anti-Myc antibody), NE-tag (TKENPRSNQEESYDDNES; SEQ ID NO:50) (e.g., binds anti-NE antibody), polyarginine tag (binds cation-exchange resin), polyglutamate tag (binds anion-exchange resin), SNAP-Tag® (binds, e.g., guanine or chloropyrimidine with a benzyl linker to the label)), Streptavidin-Binding Peptide (SBP)-Tag (MDEKTTGWRGGHVVEGLAGELEQLRARLEHHPQGQREP; SEQ ID NO:51) (e.g., binds streptavidin), Strep II Tag (WSHPQFEK; SEQ ID NO:52) (e.g., binds streptavidin or Strep-Tactin), S-tag (KETAAAKFERQHNIDS; SEQ ID NO:53) (e.g., binds S-protein of RNase A), T7-tag (MASMTGGQQMG; SEQ ID NO:54) (e.g., binds anti-T7 antibodies), V5-tag (GKPIPNPLLGLDST; SEQ ID NO:55) (e.g., binds anti-V5 polyclonal antibody, monoclonal antibody, or nanobody), or a combination thereof.

In any aspect or embodiment described herein, the affinity tag is a histidine tag (e.g., a 6×His tag).

An additional aspect of the present disclosure relates to a composition (e.g., a composition, such as a pharmaceutical composition or therapeutic composition, for promoting neuroprotection or axon regeneration of an injured axon), comprising the fibronectin-based peptide (also referenced herein as “an agent,” “the recombinant peptide,” or “therapeutic peptide”) of the present disclosure, and optionally a pharmaceutically acceptable carrier, vehicle, additive, excipient, diluent, adjuvant, or stabilizer (e.g., the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus).

In any aspect or embodiment described herein, the second composition further comprises a vehicle, wherein the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus.

In any aspect or embodiment described herein, the composition comprising the fibronectin-based peptide further comprises (i) the nucleic acid molecule, (ii) the expression cassette, (iii) the protein product, or (iv) a combination thereof.

Another aspect of the present disclosure relates to a method for promoting axon regeneration (e.g., long-distance axon regeneration) or neuroprotection of axons (e.g., long-distance axons) in a subject, the method comprising administering to the subject vitronectin, fibronectin, and/or the fibronectin-based peptide (also referenced herein as “an agent,” “the recombinant peptide,” or “therapeutic peptide”) of the present disclosure, or the composition of the present disclosure comprising the fibronectin-based peptide (also referenced herein as “an agent,” “the recombinant peptide,” or “therapeutic peptide”) of the present disclosure.

In any aspect or embodiment described herein, the method prevents damage to neural connection or repairs neural connections (e.g., neural connections in white matter of the central version system (e.g., brain, spinal cord, visual system, etc.). In any aspect or embodiment described herein, the method further comprising administering (e.g., simultaneously or co-administering with the vitronectin, fibronectin, fibronectin-based peptide, or a composition comprising one or more of the same): (i) a nucleic acid molecule comprising one or more synthetic small hairpin RNA (shRNA) molecules with one or more shRNA stem-loop regions, wherein the shRNA comprises one or more regions complementary to a portion of the target RNA, wherein hybridization of the complementary region of the shRNA to the target RNA of a target gene blocks target RNA function and promotes axon neuroprotection and/or regeneration; (ii) an expression cassette, the expression cassette comprising a synthetic nucleic acid open reading frame (ORF) molecule of a gene involved in axon regeneration, wherein the gene comprises Rpl7, Rpl7a, Tceb2, Lancl1, Atp6v0c, Dynlt1a, Mrtfa, Lars2, Dpysl5, Fblim1, Dypsl3, or Nfe213, wherein expression of the ORF promotes axon neuroprotection and/or regeneration; (iii) a protein product expressed from one or more of the open reading frames in (ii), wherein the protein product promotes axon neuroprotection and/or regeneration; a nucleic acid molecule comprising an miRNA sequence or an miRNA mimic of said miRNA involved in axon regeneration, wherein the miRNA is miR-5109, miR-1247-5p, miR-210-3p, miR-3914-1, miR-135b-3p, miR-135a-3p, wherein expression of the miRNA promotes axon neuroprotection and/or regeneration; or (iv) a combination thereof.

In any aspect or embodiment described herein, a second composition comprises (i) the nucleic acid molecule, (ii) the expression cassette, (iii) the protein product, or (iv) the combination thereof, wherein the second composition is administered.

In any aspect or embodiment described herein, the target RNA in is an mRNA or a small noncoding RNA (sncRNA).

In any aspect or embodiment described herein, the target RNA in is an mRNA of target gene Mmp9, Rax, Crx, Pdnp, Prdm13, or ift20 mRNA and its lncRNA isoform ENSMUST00000128788.

In any aspect or embodiment described herein, the nucleic acid comprises at least 2 shRNA stem-loop regions, or at least 3 shRNA stem-loop regions, or at least 4 shRNA stem-loop regions.

In any aspect or embodiment described herein, the synthetic ORF for target gene Rpl7 comprises the Rpl7 sequence identified in SEQ ID NO: 2, the synthetic ORF for target gene Rpl7a comprises the Rpl7a sequence identified in SEQ ID NO: 1, ORF for target gene Tceb2 comprises the Tceb2 sequence identified in SEQ ID NO: 3, the synthetic ORF for target gene Lancl1 comprises the Lancl1 sequence identified in SEQ ID NO:17, the synthetic ORF for target gene Atp6v0c comprises the Atp6v0c sequence identified in SEQ ID NO: 16, the synthetic ORF for target gene Dynlt1a comprises the Dynlt1a sequence identified in SEQ ID NO: 15, the synthetic ORF for target gene Mrtfa comprises the Mrtfa sequence identified in SEQ ID NO:13, the synthetic ORF for target gene Lars2 comprises the Lars2 sequence identified in SEQ ID NO: 12, the synthetic ORF of target gene Dpysl5 comprises the Dpys15 sequence identified in SEQ ID NO: 5, the synthetic ORF for target gene Fblim1 comprises the Fblim1 sequence identified in SEQ ID NO: 6, ORF for target gene Dypsl3 comprises the Dypsl3 sequence identified in SEQ ID NO: 4, ORF of target gene Nfe213 comprises the Nfe213 sequence identified in SEQ ID NO:7.

In any aspect or embodiment described herein, the target gene is Mmp9 and the shRNA sequence is identified in SEQ ID NO: 14, the target gene is Rax and shRNA sequence is identified in SEQ ID NO: 11, the target gene is Crx and the shRNA sequence is identified in SEQ ID NO:10, the target gene is Pdnp and the shRNA sequence is identified in SEQ ID NO: 8, or the target gene is Prdm13 and the shRNA sequence is identified in SEQ ID NO: 9.

In any aspect or embodiment described herein, the sncRNA is Piwi-interacting RNA (piRNA). For example, in any aspect or embodiment described herein, the piRNA is piR-16295. By way of further example, in any aspect or embodiment described herein, the piRNA is piR-16295 and the shRNA sequence is SEQ ID NO: 29.

In any aspect or embodiment described herein, a composition for promoting neuroprotection or axon regeneration of an injured axon comprises a nucleic acid molecule comprising an miRNA sequence or an miRNA mimic of said miRNA involved in axon regeneration, wherein the miRNA is miR-5109, miR-1247-5p, miR-210-3p, miR-3914-1, miR-135b-3p, miR-135a-3p, wherein expression of the miRNA promotes axon neuroprotection and/or regeneration.

In any aspect or embodiment described herein, the shRNA molecule is expressed from a viral vector selected from the group consisting of retrovirus, lentivirus, adenovirus, herpesvirus, poxvirus, alpha virus, vaccinia virus, and adeno-associated (AAV) virus (e.g., the ORF is expressed from a viral vector selected from the group consisting of retrovirus, lentivirus, adenovirus, herpesvirus, poxvirus, alpha virus, vaccinia virus, and adeno-associated (AAV) virus).

In any aspect or embodiment described herein, the second composition further comprises a vehicle, wherein the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus.

In any aspect or embodiment described herein, the administration is prior to injury or post injury.

In any aspect or embodiment described herein, the administration is intravitreally, intravenously, intracortically, intracerebrally, intrathecally, intranasally, ocularly, or locally at the injured neuron.

In any aspect or embodiment described herein, the composition, the second composition, or a combination thereof is delivered with other drugs or agents used for neuroprotection or axon regeneration.

In any aspect or embodiment described herein, the subject has a disease or disorder comprising a neurodegenerative diseases, optic neuropathy, traumatic optic neuropathy, stroke or optic nerve damage from a stroke, spinal cord injury or traumatic spinal cord injury, glaucoma, head injury or head trauma, or a combination thereof.

In any aspect or embodiment described herein, the subject has a neurodegenerative disease selected from the group consisting of sporadic Parkinson's disease, autosomal recessive early-onset Parkinson's disease, Alzheimer's disease, Friedreich ataxia, Lewy body disease, Spinal muscular atrophy, stroke, amyotrophic lateral sclerosis, Binswanger's disease, Huntington's Disease, Huntington's chorea, multiple sclerosis, myasthenia gravis, and Pick's disease, Alpers' disease, Batten disease, cerebro-oculo-facio-skeletal syndrome, corticobasal degeneration, Gerstmann-Straussler-Scheinker disease, kuru, Leigh Syndrome, Monomelic amyotrophy, Multiple system atrophy, neurodegeneration with brain iron accumulation, opsoclonus myoclonus, striatonigral degeneration, transmissible spongiform encephalopathies, neuromyelitis optica, glaucoma, and optic nerve diseases.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure.

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, and 1G show regeneration of injured RGCs long-distance axons in response to Pten KD isolated for scRNA-seq. (FIG. 1A): Adult mice were pre-treated with intravitreally injected anti-Pten or scrambled shRNA (control) AAV2 viruses co-expressing mCherry reporter. Two weeks later, an ONC injury was performed. In another 2 weeks, Alexa Fluor 488 conjugated CTB was injected into the optic nerve 3 mms distally from the ONC site (FIG. 1B), and retrogradely transported via the regenerated axons into the RGC soma located in the retina (FIG. 1C). Twelve hours later, animals were sacrificed (FIG. 1A) either for histological analysis, or live RGCs were isolated by Thy1 immunopanning and FACS'ed for mCherry/CTB double-positive cells, which were immediately processed by droplet-based scRNA-seq. (FIGS. 1D and 1E) Representative confocal images of the retinal flatmount sections of the ganglion cell layer (GCL) at 2 weeks after ONC, with CTB-488 injected into the optic nerve 12 hours prior to sacrifice, from the animals pre-treated with AAV2 anti-Pten shRNA (FIG. 1D) or AAV2 scrambled shRNA control (FIG. 1E). Retinal flatmount sections were immunostained for DAPI (to label nuclei). Only a small percent of anti-Pten shRNA AAV2-transduced RGCs expressing an mCherry reporter were also CTB+, and examples of CTB+ and CTB− RGCs expressing an mCherry are shown (FIG. 1D). Examples of CTB− (FIG. 1E) RGCs expressing an mCherry reporter of transduction with scrambled shRNA control are also shown, but no CTB+ RGCs were found in the control condition. Scale bars, 20 μm (main panels), 10 μm (insets). (FIG. 1F) Gene expression violin plots of canonical pan-RGC marker genes in single cells, as marked, show that these markers are expressed in the Pten KD long-distance axon-regenerating RGCs in a similar range as in uninjured or injured (non-treated) adult RGCs (NE=normalized expression). (FIG. 1G) Pten gene expression is substantially upregulated developmentally (from 0.18 NE in embryonic to 0.54 NE in adult RGCs; p<0.001) but only modestly downregulated in RGCs after ONC (to 0.45 NE two weeks after injury; p<0.001). Whereas in the injured RGCs transduced with AAV2 anti-Pten shRNA (which regenerated long-distance axons after ONC), Pten gene expression is substantially knocked down (to 0.12 NE; p<0.001), to even below embryonic level (although not significantly different from embryonic; p=3.1). Data analyzed using ANOVA, overall F=238.9, p<0.001, with p-values of pairwise comparisons determined by posthoc LSD. Significant differences (p<0.001) indicated by an asterisk *; error bars=SEM (NS=not significant; NE=normalized expression; see Methods and next Figure for more details on scRNA-seq analysis pipeline).

FIGS. 2A, 2B, 2C, 2D, 2E, 2F, 2G, 2H, 2I, and 2J show adult RGC subtypes that are more similar to embryonic RGCs regenerate long-distance axons in response to treatment through dedifferentiating towards an embryonic state. (FIG. 2A) UMAP of Pten KD long-distance axon-regenerating RGCs shows segregation into 2 clusters (A and B, color-coded; see Methods for clustering method details). (FIGS. 2B and 2C) UMAP of embryonic (gray color; sub-clustered below in inset F upper panel) and adult RGC clusters (FIG. 2B), color-coded by cluster assignment per original publications, with a color-coded pseudo-timeline (FIG. 2C) indicating the relative transcriptomic changes progressing from immature embryonic into a mature adult RGC state, and showing that within the adult RGC subtypes some remain more similar to embryonic state whereas others change more during developmental maturation (i.e., localized further away from embryonic along the pseudo-timeline). Pseudo-timeline color-coded scale bar, in arbitrary units (AU), of relative distance along the pseudo-timeline, is shown below FIG. 2C. (FIG. 2D) Gene expression violin plots of embryonic RGC marker genes in single cells, as marked, show that expression of these markers was retained in embryonic-like adult RGC subtypes C33 and C40, as well as in the Pten KD long-distance axon-regenerating RGCs, but was downregulated in other adult RGC subtypes during maturation (NE=normalized expression). (FIGS. 2E and 2F) Pten KD long-distance axon-regenerating RGCs assignment to the UMAP of embryonic and adult atlas RGCs (from FIGS. 2B and 2C; implementation of integration algorithm is detailed in the Methods), shows that almost all Pten KD long-distance axon-regenerating RGCs are similar to embryonic RGCs (i.e., bioinformatically mapped to embryonic RGCs and to ipRGC clusters C33/C40, which are the closest to embryonic) (FIG. 2E). Zoomed-in inset below (lower panel) shows that, long-distance regenerating RGCs cluster A (shown in FIG. 2A) mapped to the ipRGC cluster C40 and embryonic RGCs, whereas long-distance regenerating RGCs cluster B (shown in FIG. 2A) mapped to the ipRGC cluster C33 (FIG. 2F). Assignments of Pten KD long-distance axon-regenerating RGCs by the integration algorithm only to the adult atlas RGC UMAP (without embryonic RGCs) results in almost all (96%) mapping only to the ipRGC clusters C33 and C40 (see FIG. 2H below). (FIG. 2G) Averages of pseudo-timeline scores (from 2C and 2E) for individual RGCs comprising the adult atlas, injured, and Pten KD long-distance axon-regenerating groups show that, adult RGC transcriptomes partially revert towards an embryonic state after axonal injury, and that the long-distance regenerating RGC transcriptomes are significantly closer to the embryonic state than untreated injured RGC transcriptomes overall. Mean±SEM shown; overall F=728.1, p<0.0001 by ANOVA, with pairwise comparisons by posthoc LSD showing significant differences at p<0.001 (indicated by asterisk *). (FIG. 2H) Average pseudo-timeline scores (from FIG. 2C, per scale bar there) of the adult atlas RGC clusters (green), ranked lower-to-higher on the embryonic-adult RGC state pseudo-timeline (x-axis in FIG. 2H; the lower the score the closer to embryonic state the cluster is). Of the survived RGC clusters, 19 shifted closer to the embryonic state by 2 weeks after injury (indicated by black asterisk *) based on their average pseudo-timeline scores (red), but still not as close as uninjured ipRGC clusters C33 and C40 (which regenerated long-distance axons in response to Pten KD treatment, see FIG. 2I below). All RGC atlas Opn4+ clusters, which include all known ipRGC amongst other clusters, are the closest to the embryonic state on the pseudo-timeline, as indicated on the y-axis (and see FIG. 2J below). Mean±SEM shown for clusters; overall F=190.8, p<0.0001 by ANOVA, with pairwise comparisons by posthoc LSD at p<0.05 showing cluster means which shifted significantly closer to the embryonic state (indicated by black asterisk *). Clusters C24, C38, and C39 did not survive by 2 weeks after injury (indicated by brown asterisk * in FIG. 2H). (FIG. 2I) Compared to cluster proportion in the adult RGC atlas, the proportion of Pten KD long-distance axon-regenerating RGCs (which bioinformatically mapped to adult atlas RGC clusters) is significantly enriched in the adult RGC clusters that are the closest to the embryonic RGC state (on the embryonic-adult RGC state pseudo-timeline, x-axis in 1H; p<0.05 by EdgeR indicated by asterisk *, see Methods). (FIG. 2J) Opn4 expression is enriched in RGC atlas clusters C7, C8, C43, C22, C31, C40, and C33 (of which the last four are ipRGC clusters) consistent with previously published data. These Opn4+ clusters are the closest to the embryonic state on the pseudo-timeline, as indicated in FIG. 2H above. Error bars=SEM; NE=normalized expression.

FIGS. 3A, 3B, 3C, 3D, 3E, 3F, 3G, and 3H show developmentally regulated and non-regulated DEGs in the Pten KD long-distance axon-regenerating RGCs. (FIGS. 3A and 3B) Gene expression violin plots of clusters C33 and C40 marker genes in single cells, as marked, show that expression of these markers was upregulated during RGC maturation only in these, but not in other, adult RGC subtypes, and that these markers are expressed in the Pten KD long-distance axon-regenerating RGCs (FIG. 3A). Some of these developmentally-upregulated C33 and C40 marker genes were downregulated by injury in (non-treated) adult RGCs, as marked, but injury-induced downregulation of their expression was rescued in the Pten KD long-distance axon-regenerating RGCs (FIG. 3B). NE=normalized expression. (FIG. 3C) Heatmap of genes which are differentially expressed between clusters C33 and C40 of ONC RGCs and Pten KD long-distance axon-regenerating RGCs (upregulated: log 2 fold change (FC)≥1.5 and Pten KD RGC expression ≥0.5 NE; downregulated: log 2 FC≤−1.5 and ONC RGC expression ≥0.5 NE). The genes displayed in the topmost and bottom-most sections of the heatmap also demonstrate a developmentally-regulated pattern. Developmentally upregulated genes are those which also have a log 2 FC≥0.5 between Embryonic and Adult clusters C33 and C40 and ≥0.01 NE in the adult C33 and C40; downregulated: log 2 FC≤−1.5 and ≥0.01 NE in the Embryonic timepoint. All gene FCs displayed in heatmap are significantly differentially expressed (p<0.05; Mann-Whitney U test). (FIG. 3D) Dynlt1a gene expression is substantially downregulated developmentally (from 0.4 NE in embryonic to 0.02 NE in adult RGCs; p<0.001) and does not change after ONC. Whereas in the Pten KD long-distance axon-regenerating RGCs, Dynlt1a gene expression is substantially upregulated (to 0.55 NE; p<0.001), to even above embryonic level. Data analyzed using ANOVA, overall F=1756.3, p<0.0001, with p-values of pairwise comparisons determined by posthoc LSD. Significant differences (p<0.001) indicated by an asterisk *; error bars=SEM. (FIG. 3E) Lars2 gene is expressed at a relatively low basal level and is not regulated developmentally or after ONC (ranging from 0.04-0.05 NE through development and after injury). Whereas in the Pten KD long-distance axon-regenerating RGCs, Lars2 gene expression is substantially upregulated (to 2.24 NE; p<0.001). Data analyzed using ANOVA, overall F=2334.9, p<0.0001, with p-values of pairwise comparisons determined by posthoc LSD. Significant differences (p<0.001) indicated by an asterisk *; error bars=SEM. (FIG. 3F) Heatmap of mt-tRNA-specific aaRSs genes, which are differentially expressed between clusters C33 and C40 of ONC RGCs and Pten KD long-distance axon-regenerating RGCs (downregulated and upregulated, with Lars2 being the most highly upregulated, as marked). (FIGS. 3G and 3H) Average percent of amino acids (AAs) in all mtDNA-encoded proteins, ranked from the highest (left) to lowest (right), showing that Lars2-aminoacylated L (outlined with dashed line) is significantly more enriched than any other AA in all mtDNA-encoded proteins; error bars=SEM (FIG. 3G). Data analyzed using ANOVA, overall F=21.4, p<0.001, with p-values for pairwise comparisons determined by posthoc LSD, showing that L is the only AA that is significantly (p<0.001) overrepresented in mitochondrial DNA-encoded proteins compared every other AA (and not just compared to some AAs) (FIG. 3H).

FIGS. 4A, 4B, 4C, 4D, and 4E show Dynlt1a and Lars2 promote long-distance axon regeneration and RGC survival after optic nerve injury. (FIG. 4A) Experimental timeline: 8 weeks old mice were pre-treated with AAV2 vectors expressing Dynlt1a, Lars2, or mCherry control. ONC injury was performed 2 weeks later. Animals were sacrificed for histological analysis 2 weeks after ONC. Axonal tracer CTB was injected intravitreally prior to sacrifice. (FIG. 4B) Representative images of the optic nerve longitudinal sections with CTB-labeled axons at 2 weeks after ONC from the animals pre-treated with AAV2 expressing Dynlt1a, Lars2, or mCherry control, as marked. The edges of the tissue were optically trimmed (i.e., cropped-out) due to artefactual autofluorescence that is common at tissue edges. Insets: Representative images of the optic nerve regions proximal and distal to the injury site are magnified for better visualization of the axons or their absence. The longest regenerating (by 2 weeks after ONC) axons in the Lars2-treated condition are indicated by arrows. Scale bars, 500 μm (main panels), 50 μm (insets). (FIG. 4C) Quantitation of CTB− labeled regenerating axons at 2 weeks after ONC, at increasing distances from the injury site, after pre-treatment with AAV2 expressing Dynlt1a, Lars2, or mCherry control, as marked (mean±SEM shown; n=4 cases per group). Data analyzed using repeated measures ANOVA, sphericity assumed, overall F=13.4, p<0.001, with p-values of pairwise comparisons determined by posthoc LSD shown in the inset table, and significant differences (p<0.03) indicated by an asterisk *. (FIGS. 4D and 4E) Quantitation of RGC survival in retinal flatmounts immunostained for an RGC marker αIII-Tubulin (Tuj1 antibody) at 2 weeks after ONC, pre-treated with AAV2 expressing Dynlt1a, Lars2, or mCherry control (mean±SEM shown, n=4 cases per group). Data analyzed using ANOVA, overall F=5.6, p<0.04, with p-values of pairwise comparisons determined by posthoc LSD. Significant differences (p<0.03) indicated by an asterisk * (FIG. 4D), and corresponding representative images are shown (scale bar, 50 μm; FIG. 4E).

FIGS. 5A, 5B, 5C, and 5D show expression profiles of the genes which promoted axon regeneration in vivo. (FIGS. 5A and 5B) Bulk mRNA-seq showing developmental upregulation (FIG. 5A) and downregulation (FIG. 5B) of gene expression in the RGCs from E18 to adult age. The RGCs were purified by immunopanning for Thy1, a well-established method, and then RNA was extracted for processing by Illumina RNA-seq kit. (FIGS. 5C and 5D) Singe cell RNA-seq (scRNA-seq) showing upregulation of high-level expression (FIG. 5C) and low-level expression (FIG. 5D) genes in the adult RGCs, which regenerated long-distance (at least 3 mm) axons in response to treatment with anti-Pten shRNAs, as compared to untreated uninjured adult RGCs, which do not regenerate axons without treatment.

FIGS. 6A and 6B show Representations of axon regeneration promoted by treatments with experimental ORFs and shRNAs. (FIG. 6A) Experimental timeline: 8 weeks old mice were pre-treated with AAV2 vectors expressing ORFs for overexpression (OE) or shRNAs for knockdown (KD), as marked, or control scrambled shRNA with an mCherry reporter. Optic nerve crush (ONC) injury was performed 2 weeks later. Animals were sacrificed for histological analysis 2 weeks after ONC. Axonal tracer CTB was injected intravitreally 1 day prior to sacrifice. (FIG. 6B) Representative images of the optic nerve longitudinal sections with axonal tracer CTB-labeled axons at 2 weeks after ONC from the animals pre-treated with experimental ORFs and shRNAs, or a control AAV2 vectors, as marked. No axons regenerating past the injury site are present in the control, but axons regenerating substantial lengths are present in the experimental conditions. Scale bar, 500 μm.

FIG. 7 shows quantifications of axon regeneration promoted by treatments with experimental ORFs and shRNAs. Quantitation of CTB-labeled regenerating axons at 2 weeks after ONC (shown in FIG. 1) at increasing distances from the injury site, as marked (mean±S.E.M shown; n=3-4 cases per group). Data analyzed using ANOVA with repeated measures, sphericity assumed, overall F=8.78, p<0.05 by posthoc LSD pairwise comparisons for the main effects of each treatment condition compared to control.

FIGS. 8A, 8B, and 8C show axon regeneration and RGC survival after treatment with recombinant experimental shRNAs targeting or expressing miRNAs or piRNAs. (FIG. 8A) Experimental timeline: 8 weeks old mice were pre-treated with AAV2 vectors expressing experimental shRNAs (as indicated) or scrambled shRNA control. Optic nerve crush (ONC) injury was performed 2 weeks later. Animals were sacrificed for histological analysis 2 weeks after ONC. Axonal tracer CTB was injected intravitreally 1 day prior to sacrifice. (FIG. 8B) Representative images of the optic nerve longitudinal sections with CTB-labeled axons at 2 weeks after ONC from the animals pre-treated with scrambled shRNA control or experimental shRNAs, as marked. The edges of the tissue were optically trimmed (i.e., cropped-out) due to artefactual autofluorescence that is common at tissue edges. Scale bar, 500 μm. (FIG. 8C) Representative images of retinal flatmounts immunostained for an RGC marker βIII-Tubulin (Tuj1) at 2 weeks after ONC, scale bar, 50 μm.

FIG. 9 shows quantification of axon regeneration promoted by treatments with recombinant experimental shRNAs targeting or expressing miRNAs or piRNAs. Quantitation of CTB-labeled regenerating axons at 2 weeks after ONC (shown in FIG. 8), at increasing distances from the injury site, as marked (mean±S.E.M shown; n=4 cases per group). Data analyzed using ANOVA with repeated measures, sphericity assumed, overall F=14.2, p<0.001, and p<0.05 by posthoc LSD pairwise comparisons for each of the treatment conditions compared to control main effects.

FIGS. 10A, 10B, 10C, and 10D show axon regeneration and RGC survival after treatment with recombinant experimental shRNAs targeting. (FIG. 10A) Experimental timeline: 8 weeks old mice were pre-treated with AAV2 vectors expressing experimental shRNAs (as indicated) or scrambled shRNA control. Optic nerve crush (ONC) injury was performed 2 weeks later. Animals were sacrificed for histological analysis 2 weeks after ONC. Axonal tracer CTB was injected intravitreally 1 day prior to sacrifice. (FIG. 10B) Representative images of the optic nerve longitudinal sections with CTB-labeled axons at 2 weeks after ONC from the animals pre-treated with scrambled shRNA control or experimental shRNAs, as marked. The edges of the tissue were optically trimmed (i.e., cropped-out) due to artefactual autofluorescence that is common at tissue edges. Scale bar, 500 μm. (FIG. 10C) Representative images of retinal flatmounts immunostained for an RGC marker βIII-Tubulin (Tuj1 antibody) at 2 weeks after ONC, scale bar, 50 μm. (FIG. 10D) Quantitation of CTB-labeled regenerating axons at 2 weeks after ONC, at increasing distances from the injury site, as marked (mean±S.E.M shown; n=4 cases per group). Data analyzed using ANOVA with repeated measures, sphericity assumed, overall F=11.9, p<0.002, and p<0.02 by posthoc LSD pairwise comparisons for each of the treatment conditions compared to control main effects.

FIGS. 11A, 11B, and 11C show axon regeneration and RGC survival over long-term after treatment with recombinant experimental shRNAs targeting representative miRNA or piRNA. (FIG. 11A) Experimental timeline: 8 weeks old mice were pre-treated with AAV2 vectors expressing experimental shRNAs (as indicated) or scrambled shRNA control. Optic nerve crush (ONC) injury was performed 2 weeks later. Animals were sacrificed for histological analysis 6 weeks after ONC. Axonal tracer CTB was injected intravitreally 1 day prior to sacrifice. (FIG. 11B) Representative images of the optic nerve longitudinal sections with CTB-labeled axons at 6 weeks after ONC from the animals pre-treated with scrambled shRNA control or experimental shRNAs, as marked. The edges of the tissue were optically trimmed (i.e., cropped-out) due to artefactual autofluorescence that is common at tissue edges. Scale bar, 500 μm. (FIG. 11C) Representative images of retinal flatmounts immunostained for an RGC marker βIII-Tubulin (Tuj1) at 6 weeks after ONC, scale bar, 50 μm.

FIGS. 12A, 12B, 12C, and 12D. Adult retinal ganglion cells survive and regenerate axons when cultured on fibronectin. (FIG. 12A) Representative images of 10-week-old adult RGCs cultured on either laminin, vitronectin, or fibronectin for 4 days in vitro (DIV) or 8 days DIV. (FIGS. 12B and 12C) Quantifications showed that the fibronectin condition had the longest regenerating axons (FIG. 12B) and the most regenerating axons (FIG. 12C), specifically at 8 DIV. (FIG. 12D) Quantifications of the RGC survival showed that by 4 DIV and 8 DIV both fibronectin and vitronectin conditions/treatment had similarly significantly higher RGC survival as compared to laminin condition. N=3; n≥45; * p<0.05 by ANOVA; Error bars, SEM. Scale bars, main panels=50 μm; insets=10 μm. Lm: Laminin; Vn: Vitronectin; Fn: Fibronectin.

FIGS. 13A, 13B, 13C, 13D and 13E. Axon regeneration promoted, or enhanced in a combination with another treatment, by experimental treatment with the fibronectin-derived peptide. (FIG. 13A) Experimental timeline for the short-term study: 8 weeks old mice were pre-treated with experimental AAV2 vector VB200810-1300pah expressing shRNA. Optic nerve crush (ONC) injury was performed 2 weeks later, followed by treatment with the (1) experimental emulsion containing the recombinant peptide in PBS or (2) control emulsion containing PBS without the peptide. Animals were sacrificed for histological analysis 2 weeks later. Axonal tracer CTB was injected intravitreally 1 day prior to sacrifice. (FIG. 13B) Representative images of the optic nerve longitudinal sections with axonal tracer Cholera toxin subunit B (CTB)-labeled axons through the optic nerve at 2 weeks after ONC, from the animals treated with experimental emulsion containing the recombinant peptide in PBS, control emulsion containing the PBS without the peptide, or pre-treated with AAV2 shRNA VB200810-1300pah, are shown as marked. No axons regenerating past the injury site were present in the control, but axons regenerating substantial lengths were present in the experimental conditions. Insets: Optic nerve regions, which are proximal (˜1 mm), and distal (˜2.0 mm) to the injury site, were magnified for better visualization of the regenerating axons or their absence in control condition. Scale bars: 500 μm (main panel), 50 μm (insets). (FIG. 13C) Experimental timeline for the long-term study: 8 weeks old mice were pre-treated with experimental AAV2 vector VB200810-1300pah expressing shRNA. ONC injury was performed 2 weeks later, followed by treatment with experimental emulsion containing the recombinant peptide in PBS or control emulsion containing the PBS without the peptide. Animals were sacrificed for histological analysis 6 weeks later. Axonal tracer CTB was injected intravitreally 1 day prior to sacrifice. (FIG. 13D) Representative images of the optic nerve longitudinal sections with axonal tracer CTB-labeled axons through the optic nerve at 6 weeks after ONC, from the animals treated with experimental emulsion containing the recombinant peptide in PBS, control emulsion containing the PBS without the peptide, AAV2 shRNA VB200810-1300pah, or co-treated with emulsion containing the recombinant peptide and AAV2 shRNA VB200810-1300pah, are shown as marked. No axons regenerating past the injury site were present in the control, but axons regenerating substantial lengths were present in the experimental conditions, with the longest (through the end of the optic nerve) regenerating axons detected in the co-treatment condition. FIG. 13E—Insets: Optic nerve regions, which are proximal (˜1 mm), middle (˜2 mm), distal (˜3 mm), and at the end of the optic nerve (˜4 mm), to the injury side were magnified for better visualization of the regenerating axons or their absence in control conditions. Scale bars: 500 μm (main panel), 50 μm (insets).

FIG. 14. Quantifications of axon regeneration promoted by experimental treatments. Quantitation of CTB-labeled regenerating axons at 2 weeks after ONC (shown in FIG. 2) at increasing distances from the injury site, as marked (mean±S.E.M shown; n=3 cases per group). Data analyzed using ANOVA with repeated measures, sphericity assumed, overall F=10.4, p<0.05 by posthoc LSD pairwise comparisons for the main effect of the treatment condition compared to control.

FIG. 15. Axon regeneration over 6 weeks after ONC in the optic chiasm and tract in response to co-treatment with emulsion containing the recombinant peptide and AAV2 shRNA VB200810-1300pah. Top left is a representative image of the optic chiasm horizontal section, which includes partial optic nerves and tracts sections, with axonal tracer CTB-labeled RGC axons at 6 weeks after ONC, from the animal that received co-treatment (emulsion containing the recombinant peptide and AAV2 shRNA VB200810-1300pah) in the right eye, and control treatment into the left eye (indicated by arrows). Insets: (1, 2) axons regenerating through the optic tracks, (3-7) axons regenerating through the middle of optic chiasm, (8-10) regenerating axons from the right optic nerve entering the optic chiasm. Scale bars: 500 μm (main image, top left), 50 μm (insets).

FIGS. 16A-16F. Axon regeneration and RGC survival over long-term after treatment with recombinant experimental shRNAs targeting Itf20. (FIG. 16A) Experimental timeline: 8 weeks old mice were pre-treated with AAV2 vectors expressing experimental anti-ift20 shRNAs or scrambled shRNA control. Optic nerve crush (ONC) injury was performed 2 weeks later. Animals were sacrificed for histological analysis 4 weeks after ONC. Axonal tracer CTB was injected intravitreally 1 day prior to sacrifice. (FIG. 16B) Representative images of the optic nerve longitudinal sections with CTB-labeled axons at 4 weeks after ONC from the animals pre-treated with scrambled shRNA control or experimental anti-ift20 shRNAs, as marked. The edges of the tissue were optically trimmed (i.e., cropped-out) due to artefactual autofluorescence that is common at tissue edges. Scale bar, 500 μm. (FIG. 16C) Insets: Representative images of the optic nerve regions proximal and distal to the injury site are magnified for better visualization of the axons or their absence. Scale bar, 50 μm. (FIG. 16D) Representative images of retinal flatmounts immunostained for an RGC marker βIII-Tubulin (Tuj1) at 4 weeks after ONC, scale bar, 20 μm. (FIGS. 16E-16F) Representative images of the optic chiasm horizontal sections with CTB-labeled axons at 4 weeks after ONC from two different mice pre-treated with experimental anti-ift20 shRNAs. Insets: Representative images of the optic chiasm regions are magnified for better visualization of the axons or their absence in the distal optic nerve regions entering the optic chiasm, medial optic chiasm, and the optic tracts. Scale bars, 500 μm (main panels), 50 μm (insets).

FIGS. 17A-17C. Quantifications of axon regeneration and RGC survival over long-term promoted by treatment with recombinant experimental shRNAs targeting Itf20. (FIG. 17A) Experimental timeline: 8 weeks old mice were pre-treated with AAV2 vectors expressing scrambled shRNA control or experimental anti-itf20 shRNAs. Optic nerve crush (ONC) injury was performed 2 weeks later. Animals were sacrificed for histological analysis 4 weeks after ONC. Axonal tracer CTB was injected intravitreally 1 day prior to sacrifice. (FIG. 17B) Quantitation of CTB-labeled regenerating axons at 4 weeks after ONC (shown in FIG. x1), at increasing distances from the injury site, as marked. Data analyzed using ANOVA with repeated measures, sphericity assumed, overall F=139.49, p<0.001, and p<0.001 by posthoc LSD for the main effect. Mean±S.E.M shown; n=4 cases per group. (FIG. 17C) Quantitation of RGC survival in retinal flatmounts immunostained for an RGC marker βIII-Tubulin (Tuj1 antibody) at 4 weeks after ONC, pre-treated with AAV2 expressing scrambled shRNA control or anti-itf20 shRNAs. Data analyzed using t-test 2-tailed, and significant difference (p<0.001) is indicated by an asterisk *. Mean±SEM shown, n=4 cases per group.

FIGS. 18A-18F. Axon regeneration and RGC survival over long-term after treatment with recombinant experimental shRNAs targeting piR-16295. (FIG. 18A) Experimental timeline: 8 weeks old mice were pre-treated with AAV2 vectors expressing experimental anti-piR16295 shRNA or scrambled shRNA control. Optic nerve crush (ONC) injury was performed 2 weeks later. Animals were sacrificed for histological analysis 6 weeks after ONC. Axonal tracer CTB was injected intravitreally 1 day prior to sacrifice. (FIG. 18B) Representative images of the optic nerve longitudinal sections with CTB-labeled axons at 6 weeks after ONC from the animals pre-treated with scrambled shRNA control or experimental anti-piR16295 shRNAs, as marked. The edges of the tissue were optically trimmed (i.e., cropped-out) due to artefactual autofluorescence that is common at tissue edges. Scale bar, 500 μm. (FIG. 18C) Insets: Representative images of the optic nerve regions proximal and distal to the injury site are magnified for better visualization of the axons or their absence. Scale bar, 50 μm. (FIG. 18D) Representative images of retinal flatmounts immunostained for an RGC marker βIII-Tubulin (Tuj1) at 6 weeks after ONC, scale bar, 20 μm. (FIGS. 18E-18F) Representative images of the optic chiasm horizontal sections with CTB-labeled axons at 6 weeks after ONC from two different mice pre-treated with experimental anti-piR16295 shRNAs. Insets: Representative images of the optic chiasm regions are magnified for better visualization of the axons or their absence in the distal optic nerve regions entering the optic chiasm, medial optic chiasm, and the optic tracts. Scale bars, 500 μm (main panels), 50 μm (insets).

FIGS. 19A-19C. Quantifications of axon regeneration and RGC survival over long-term promoted by treatment with recombinant experimental shRNAs targeting piR-16295. (FIG. 19A) Experimental timeline: 8 weeks old mice were pre-treated with AAV2 vectors expressing scrambled shRNA control or experimental anti-piR16295 shRNAs. Optic nerve crush (ONC) injury was performed 2 weeks later. Animals were sacrificed for histological analysis 6 weeks after ONC. Axonal tracer CTB was injected intravitreally 1 day prior to sacrifice. (FIG. 19B) Quantitation of CTB-labeled regenerating axons at 6 weeks after ONC (shown in FIG. 18), at increasing distances from the injury site, as marked. Data analyzed using ANOVA with repeated measures, sphericity assumed, overall F=18.74, p<0.049, and p<0.049 by posthoc LSD for the main effect. Mean±S.E.M shown; n=4 cases per group. (FIG. 19C) Quantitation of RGC survival in retinal flatmounts immunostained for an RGC marker βIII-Tubulin (Tuj1 antibody) at 6 weeks after ONC, pre-treated with AAV2 expressing scrambled shRNA control or anti-piR16295 shRNAs. Data analyzed using t-test 2-tailed, and significant difference (p<0.001) is indicated by an asterisk *. Mean±SEM shown, n=4 cases per group.

FIGS. 20A-20E. Axon regeneration over long-term promoted by treatment with recombinant experimental fibronectin-derived peptide or Rpl7a ORF and the enhancement of the effect by co-treatment. (FIG. 20A) Experimental timeline: 8 weeks old mice were injected intravitreally with AAV2 vectors expressing mCherry (control) or Rpl7a ORF. Optic nerve crush (ONC) was performed 2 weeks later, and immediately after ONC mice were intravitreally injected with the fibronectin-derived peptide (in emulsion) or vehicle (control in emulsion, PBS buffer in which the fibronectin-derived peptide was dissolved and stored at −80C). Animals were sacrificed for histological analysis 6 weeks after ONC. Axonal tracer CTB was injected intravitreally 1 day prior to sacrifice. (FIGS. 20B-20C) Representative images of CTB-labeled axons in the optic nerve longitudinal sections at 6 weeks after ONC from the animals pre-treated with mCherry control or Rpl7a AAV2 or peptide or both, as marked. The edges of the tissues were optically trimmed (i.e., cropped-out) due to artefactual autofluorescence that is common at tissue edges. Injured area of the co-treatment condition (bottom panel) is also shown below with exposure reduced, in order to indicate the crush site, which is otherwise masked by densely regenerated axons when exposure is increased for visualization of sparse axons that regenerated distally from the injury site (FIG. 20B). Insets: Representative images of the optic nerve regions at various distances from the injury site are magnified for better visualization of the axons or their absence. Majority of the injured axons that remained connected to RGC soma after ONC have degenerated retrogradely 6 weeks later, as indicated by the absence of CTB signal in most pre-crush areas (outlined by the dashed-line) of the control condition, compared to the pre-crush area of the experimental conditions with only sparse indications of retrograde degeneration (FIG. 20C). Scale bars, 500 μm (main), 50 μm (insets). (FIGS. 20D-20E) Representative image of the optic chiasm horizontal section at 6 weeks after ONC from animal co-treated with Rpl7a AAV2 and peptide (FIG. 20D), with insets of outlined regions (marked 1-4), where CTB-labeled axons were found, magnified for visualization of axons (FIG. 20E). Scale bars, 250 μm (main), 15 μm (insets).

FIGS. 21A-21B. Quantifications of axon regeneration over long-term promoted by treatment with recombinant experimental fibronectin-derived peptide or Rpl7a ORF and the enhancement of the effect by co-treatment. (FIG. 21A) Experimental timeline: 8 weeks old mice were injected intravitreally with AAV2 vectors expressing mCherry (control) or Rpl7a ORF. Optic nerve crush (ONC) was performed 2 weeks later, and immediately after ONC mice were intravitreally injected with the fibronectin-derived peptide (in emulsion) or vehicle (emulsion). Animals were sacrificed for histological analysis 6 weeks after ONC. Axonal tracer CTB was injected intravitreally 1 day prior to sacrifice. (FIG. 21B) Quantitation of CTB-labeled regenerating axons at 6 weeks after ONC (shown in FIG. 20), at increasing distances from the injury site, that received various treatments (detailed in FIG. 21A), as marked. Data analyzed using repeated measures ANOVA, sphericity assumed (overall F=27.6, p<0.001), with p-values of pairwise comparisons determined by postdoc LSD indicating that the differences between all the conditions (except between peptide and Rpl7a individually) were significant (p<0.05). Mean±SEM shown; n=4-6 cases per group.

FIGS. 22A-22C. Axon regeneration over long-term promoted by treatment with recombinant experimental fibronectin-derived peptide or Lancl1 ORF and the enhancement of the effect by co-treatment. (FIG. 22A) Experimental timeline: 8 weeks old mice were injected intravitreally with AAV2 vectors expressing mCherry (control) or Lancl1 ORF. Optic nerve crush (ONC) was performed 2 weeks later, and immediately after ONC mice were intravitreally injected with the fibronectin-derived peptide (in emulsion) or vehicle (emulsion). Animals were sacrificed for histological analysis 6 weeks after ONC. Axonal tracer CTB was injected intravitreally 1 day prior to sacrifice. (FIG. 22B) Representative images of CTB-labeled axons in the optic nerve longitudinal sections at 6 weeks after ONC from the animals pre-treated with mCherry control or Lancl1 AAV2 or peptide or both, as marked. The edges of the tissues were optically trimmed (i.e., cropped-out) due to artefactual autofluorescence that is common at tissue edges. Scale bar, 500 μm. (FIG. 22C) Insets: Representative images of the optic nerve regions at various distances from the injury site are magnified for better visualization of the axons or their absence. Majority of the injured axons that remained connected to RGC soma after ONC have degenerated retrogradely 6 weeks later, as indicated by the absence of CTB signal in most pre-crush areas (outlined by the dashed-line) of the control condition, compared to the pre-crush area of the experimental conditions with only sparse indications of retrograde degeneration. Scale bar, 50 μm.

FIGS. 23A-23B. Quantifications of axon regeneration over long-term promoted by treatment with recombinant experimental fibronectin-derived peptide or Lancl1 ORF and the enhancement of the effect by co-treatment. (FIG. 23A) Experimental timeline: 8 weeks old mice were injected intravitreally with AAV2 vectors expressing mCherry (control) or Lancl1 ORF. Optic nerve crush (ONC) was performed 2 weeks later, and immediately after ONC mice were intravitreally injected with the fibronectin-derived peptide (in emulsion) or vehicle (emulsion). Animals were sacrificed for histological analysis 6 weeks after ONC. Axonal tracer CTB was injected intravitreally 1 day prior to sacrifice. (FIG. 23B) Quantitation of CTB-labeled regenerating axons at 6 weeks after ONC (shown in FIG. 22), at increasing distances from the injury site, that received various treatments (detailed in A), as marked. Data analyzed using repeated measures ANOVA, sphericity assumed (overall F=23.7, p<0.001), with p-values of pairwise comparisons determined by postdoc LSD indicating that the differences between all the conditions (except between peptide and Lancl1 individually) were significant (p<0.05). Mean±SEM shown; n=3-5 cases per group.

FIGS. 24A-24E. Axon regeneration over long-term promoted by treatments with recombinant experimental fibronectin-derived peptide or miR-135a-1-3p mimic or miR-135b-3p mimic and the enhancement of the effect by co-treatments. (FIG. 24A) Representative images of CTB-labeled axons in the optic nerve longitudinal sections at 6 weeks after ONC from the animals pre-treated with scrambled shRNA (control), miR-135a-1-3p mimic, or miR-135b-3p mimic, with or without peptide co-treatment, as marked. The edges of the tissues were optically trimmed (i.e., cropped-out) due to artefactual autofluorescence that is common at tissue edges. Scale bar, 500 μm. (FIG. 24B) Insets: Representative images of the optic nerve regions at various distances from the injury site are magnified for better visualization of the axons or their absence. Majority of the injured axons that remained connected to RGC soma after ONC have degenerated retrogradely 6 weeks later, as indicated by the absence of CTB signal in most pre-crush areas (outlined by the dashed-line) of the control condition, compared to the pre-crush area of the experimental conditions with only sparse indications of retrograde degeneration. Scale bar, 50 μm. (FIG. 24C) Experimental timeline: 8 weeks old mice were injected intravitreally with AAV2 vectors expressing scrambled shRNA (control), miR-135a-1-3p mimic, or miR-135b-3p mimic. Optic nerve crush (ONC) was performed 2 weeks later, and immediately after ONC mice were intravitreally injected with the fibronectin-derived peptide (in emulsion) or vehicle (emulsion). Animals were sacrificed for histological analysis 6 weeks after ONC. Axonal tracer CTB was injected intravitreally 1 day prior to sacrifice. (FIGS. 24D-24E) Representative images of the optic chiasm horizontal sections with CTB-labeled axons at 6 weeks after ONC from different mice co-treated with peptide and either miR-135a-1-3p (FIG. 24D) or miR-135b-3p (FIG. 24E) mimics. Insets: Representative images of the optic chiasm regions from different co-treated animals are magnified for better visualization of the axons or their absence in the distal optic nerve regions entering the optic chiasm, medial optic chiasm, and the optic tracts. Scale bars, 500 μm (main panels), 50 μm (insets). RGD #1 and RGD #2 refer to two different cases from the same treatment condition.

FIGS. 25A-25B. Quantifications of axon regeneration over long-term promoted by treatment with miR-135a-1-3p mimic or miR-135b-3p mimic and the enhancement of the effect by co-treating with recombinant experimental fibronectin-derived peptide. (FIG. 25A) Experimental timeline: 8 weeks old mice were injected intravitreally with AAV2 vectors expressing scrambled shRNA (control), miR-135a-1-3p mimic, or miR-135b-3p mimic. Optic nerve crush (ONC) was performed 2 weeks later, and immediately after ONC mice were intravitreally injected with the fibronectin-derived peptide (in emulsion) or vehicle (emulsion). Animals were sacrificed for histological analysis 6 weeks after ONC. Axonal tracer CTB was injected intravitreally 1 day prior to sacrifice. (FIG. 25B) Quantitation of CTB-labeled regenerating axons at 6 weeks after ONC (shown in FIG. 24), at increasing distances from the injury site, that received various treatments (detailed in A), as marked. Data analyzed using repeated measures ANOVA, sphericity assumed (overall F=108.5, p<0.001), with p-values of pairwise comparisons determined by postdoc LSD indicating that the differences between the individual and combinatory treatments compared to control were significant (p<0.05) and that the differences between the individual AAV treatments compared co-treatments with peptide were also significant (p<0.05). Mean±SEM shown; n=3-5 cases per group.

FIGS. 26A-26B. RGC survival over long-term promoted by treatment with recombinant experimental vectors expressing miRNA mimics or experimental ORFs or fibronectin-derived peptide, and further enhanced by combined treatment. (FIG. 26A) Representative images of the retinal flatmounts immunostained for βIII-Tubulin (Tuj1, which labels selectively the RGCs within the retina) at 6 weeks after ONC, from the animals pre-treated with control or experimental AAV2 treatments without (left column) or with (right column) peptide co-treatment, as marked. Scale bar, 20 μm. (FIG. 26B) Quantitation of RGC survival at 6 weeks after ONC analyzed using ANOVA (overall F=14.2, p<0.001), with p-values of pairwise comparisons determined by posthoc LSD and significant differences (p<0.05) indicated by an asterisk * (See Table 8. Mean±SEM shown; n=3-5 cases per group.

FIG. 27. Example schematic showing design of a shRNA-expressing AAV2 vector. AVV2 vectors were used to express the predicted functional therapeutic nucleic acid sequences in RGC neurons. For the expression of short non-coding sequences, such as shRNAs and miRNA mimics, a cassette which is based on the percussor of miR-155 miRNA and enables co-expression of up to 4 of the same or different small non-coding RNAs, such as shRNAs and miRNA mimics was used. For knocking-down expression of target mRNAs, 4 different shRNA sequences were used to target the same mRNA in order to achieve more efficient knockdown. For knockdown of miRNA or piRNA, and for expression of miRNA mimics or other small non-coding RNA sequences, up to 4 of the same functional sequence were co-expressed from the same AAV2 vector, in order to achieve higher levels of functional RNA sequence expression in the targeted neurons. Functional therapeutic nucleic acid sequences were predicted using standard bioinformatics tools, and were synthesized and cloned into the AAV2 vector using standard nucleic acid synthesis and vector cloning methods and techniques. As an example, the schematic shows pAAV[miR155]-UBC>mRax[shRNA #1]:mRax[shRNA #2]:mRax[shRNA #3]:mRax[shRNA #4]:Myc/mCherry expressing four copies of shRNAs, in this case each shRNA with a different target sequence. Processing of the shRNA will result in four expressed functional sequences, one from each shRNA.

### DETAILED DESCRIPTION

The present inventors surprisingly and unexpected discovered that a subset of the adult RGCs survived better and regenerated axons when cultured on vitronectin, and even better when cultured on fibronectin. The present inventors further surprisingly and unexpectedly discovered that a fibronectin-based peptide (also referenced herein as “an agent,” “the recombinant peptide,” or “therapeutic peptide”) promoted axon regeneration in vivo. The inventors of the present disclosure surprisingly and unexpectedly discovered that a fibronectin-based peptide can be utilized with various signal peptides, with or without a purification tag, as a peptide therapeutic, such as a peptide therapeutic to repair neural connections or prevent damage to neural connection (e.g., a preventive treatment or co-treatment and a post-damage treatment or co-treatment). The surprising use of vitronectin, fibronectin, and fibronectin-based peptides (also referenced herein as “agents,” “recombinant peptides,” or “therapeutic peptides”) as a preventive treatment or co-treatment and a post-damage treatment or co-treatment was unexpectedly discovered by the present inventors.

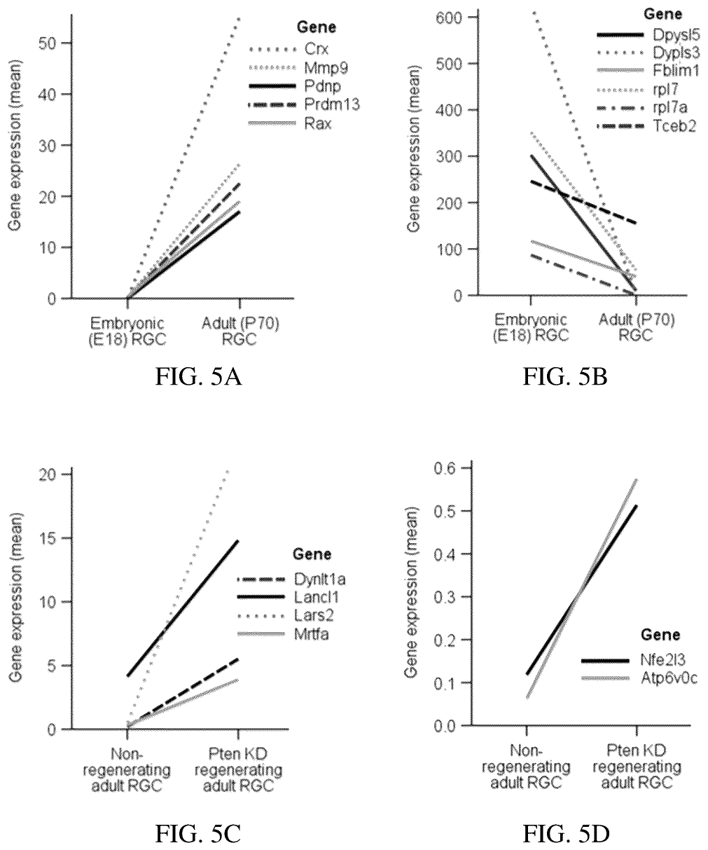

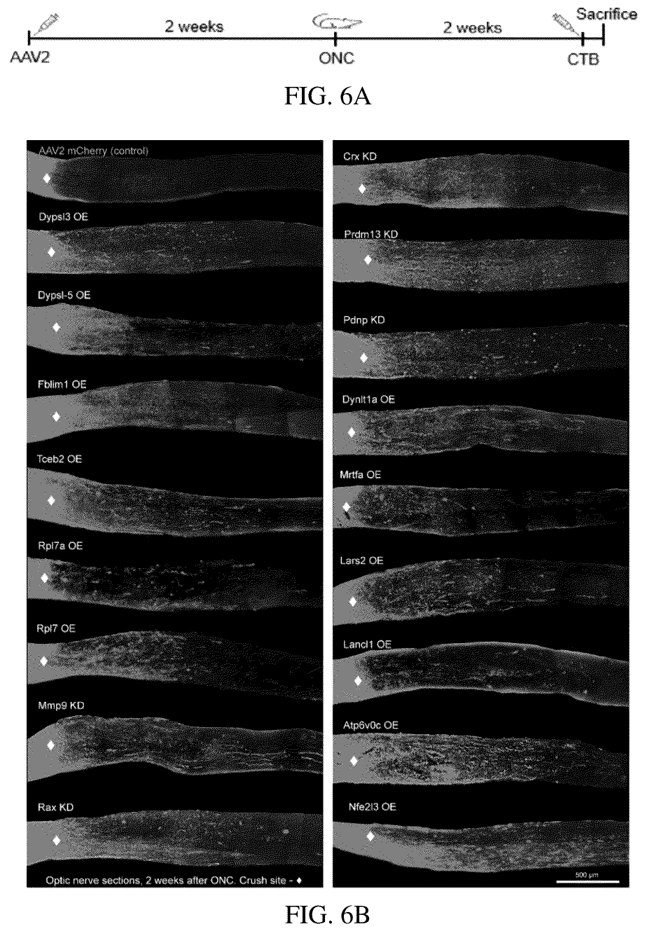

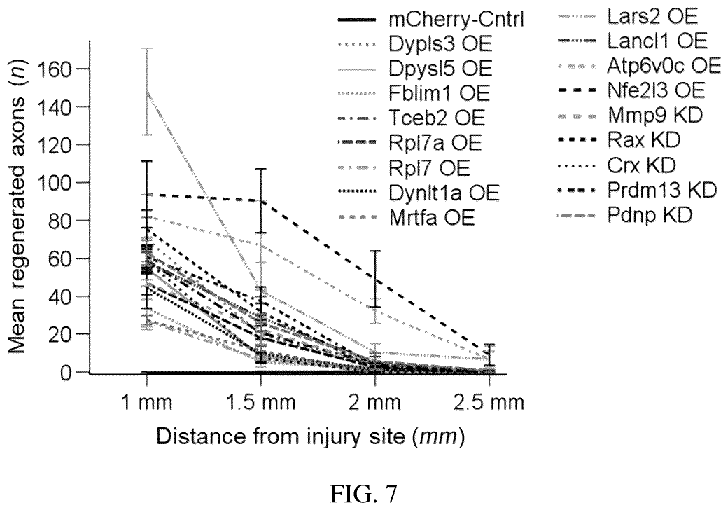

The inventors have further identified novel protein coding messenger RNAs (mRNAs) that are developmentally regulated in the retinal ganglion cell (RGC) neurons as they mature, as well as those that are differentially expressed exclusively in the small subset of RGC neurons which regenerate long-distance (at least 3 mm) axons in response to treatment with anti-Pten shRNAs. The novel targets were tested for their ability to promote axon regeneration in vivo (FIGS. 5A-5D, 6A, 6B, and 7) through overexpression of specifically designed ORFs or knocking-down expression through specifically designed shRNAs. Specifically, for overexpression: Rpl7a, Rpl7, Tceb2, Lancl1, Atp6v0c, Dynlt1a, Mrtfa, Lars2, Dpysl5, Fblim1, Dypsl3, and Nfe213; and for knock-down: Mmp9, Rax, Crx, Pdnp, Prdm13, ift20 mRNA and its lncRNA isoform ENSMUST00000128788.

RNA-seq technologies were used which can detect small-noncoding RNAs (sncRNA), in order to identify those that are developmentally regulated in the retinal ganglion cell (RGC) neurons as they mature. The inventors now have identified a number of novel microRNAs (miR) than are neuroprotective/neurogenerative targets, for overexpression, miR-210-3p, miR-5109, and miR-1247, miR-3914-1, miR-135a-3p, miR-135b-3p, and for knockdown of expression miR-129-1-3p, miR-129-5p, miR-1290, miR-6090, miR-132-3p, and miR-29a-3p. Small noncoding RNAs (sncRNA) were also identified for overexpression, VB200810-1300pah, VB200810-1013mnb, and VB200810-1256arq, sncRNA VB200810-1672kgn, and sncRNA VB200810-1670qan.

Developmentally upregulated Piwi-interacting RNAs (piRNA), which is a class of sncRNAs, whose roles in the CNS are largely unknown. Without being bound to a theory, the piRNAs, which were found to be developmentally upregulated in the maturing RGCs, are thought to be involved in the failure of mature adult RGCs to regenerate axons after optic nerve injury. Therefore, shRNAs were designed to knockdown the expression of the identified piRNAs and deliver them selectively to the RGCs in the mouse eyes using a viral vector, specifically, AAV2. Adeno-associated virus (AAV) serotype 2 (AAV2) preferentially transduces neurons, such as the retinal ganglion cells (RGCs) within the eye and is also used in clinical trials to treat neurological and eye ailments. AAV2 vectors are used to express in the targeted neurons functional therapeutic protein-coding ORFs, non-coding RNA sequences, shRNAs (to knockdown expression of endogenous mRNA, miRNAs, and various classes of non-coding RNAs), and miRNAs' (and other classes on non-coding RNAs) mimics.

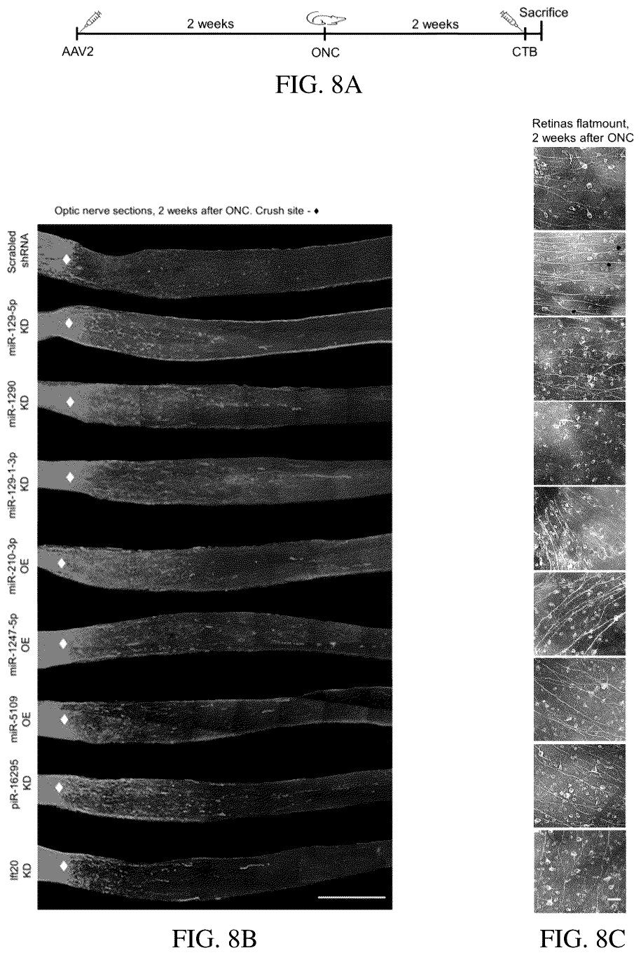

The inventors found that inhibiting piRNA (ID: piR-16295) by AAV2-delivered shRNAs promoted the survival of the RGCs and axonal regeneration, after traumatic optic nerve injury in an established mouse model of traumatic optic neuropathy. FIGS. 8A-8C demonstrates the effect of knocking-down the most potent piRNA target (ID: piR-16295), in short (2 weeks after injury) and long-term (6 weeks after injury) assays. The success in testing these novel targets provides proof-of-concept that other identified RNA targets using the same approach, are effective. Specifically, the RNAs differentially expressed by a treatment with anti-Pten shRNAs exclusively in the small subset of the RGCs that regenerated long-distance (at least 3 mm) axons as listed in Table 7.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

As used herein the term “consisting of” refers to compositions, methods, and respective components thereof as described herein, which are exclusive of any element not recited in that description of the embodiment.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically acceptable carrier” means a pharmaceutically-acceptable material, composition or vehicle, such as a liquid or solid filler, diluent, excipient, manufacturing aid or solvent encapsulating material necessary or used in formulating an active ingredient or agent for delivery to a subject. Each carrier must be “acceptable” in the sense of being compatible with the other ingredients of the formulation and not injurious to the patient.

In any aspect or embodiment described herein, the neuroprotective or regenerative ORF or hpRNAs, or vector comprising a nucleic acid sequence encoding ORFs or hpRNAs or compositions provided herein can be formulated in liposomes to promote delivery across membranes. As used herein, the term “liposome” refers to a vesicular structure having lipid-containing membranes enclosing an aqueous interior. In cell biology, a vesicular structure is a hollow, lamellar, spherical structure, and provides a small and enclosed compartment, separated from the cytosol by at least one lipid bilayer. Liposomes can have one or more lipid membranes. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand.

Liposome compositions can be prepared by a variety of methods that are known in the art. Niosomes are non-phospholipid based synthetic vesicles that have properties and function like liposomes.

As used herein, the term “nanoparticle” refers to a particle having a size between 1 and 1000 nm which can be manufactured from artificial or natural macromolecular substances. To such nanoparticles can be bound drugs or other biologically active materials by covalent, ionic or adsorptive linkage, or the latter can be incorporated into the material of the nanoparticles. Nanoparticles may or may not exhibit size-related properties that differ significantly from those observed in fine particles or bulk materials. Nanoparticles provide improved bioavailability by enhancing aqueous solubility, increasing resistance time in the body (increasing half-life for clearance/increasing specificity for its cognate receptors and targeting drug to specific location in the body (its site of action). This results in concomitant reduction in quantity of the drug required and dosage toxicity, enabling the safe delivery of toxic therapeutic drugs and protection of non-target tissues and cells from severe side effects. Non-limiting examples of nanoparticles include solid lipid nanoparticles (comprise lipids that are in solid phase at room temperature and surfactants for emulsification, the mean diameters of which range from 50 nm to 1000 nm for colloid drug delivery applications), liposomes, nanoemulsions (oil-in-water emulsions done on a nano-scale), albumin nanoparticles, and polymeric nanoparticles.

Nanoparticles can be surface coated to modulate their stability, solubility, and targeting. A coating that is multivalent or polymeric confers high stability (34). A non-limiting example includes coating with hydrophilic polymer such as polyethylene glycol or ploysorbate-80.

As used herein the term “lipophilic molecular group” refers to a lipid moiety, such as a fatty acid, glyceride or phospholipid which when coupled to a therapeutic molecule to be a targeted to the brain, increases its lipophilicity and hence movement across blood brain barrier. The lipophilic molecular group can be attached to the therapeutic molecule through an ester bond. As it relates to the present disclosure a lipophilic molecular group can enable uptake of the agents or compositions herein into the mitochondria of the neurons.

As used herein the term “carrier polypeptide” refers to a peptide which exhibits substantially no bioactivity. In some embodiments, the carrier peptide is an axon targeting peptide capable of targeting the agent to the axon. The carrier peptide can be an endogenous peptide whose receptor is present on the cerebral capillary endothelial cell, such as insulin, insulin-like growth factor (IGF), leptin and transferrin or fragments thereof. The carrier peptide can be, for example, a short cell penetrating peptide of less than 30 amino acids that are amphipathic in nature and are able to interact with lipidic membranes. Non-limiting examples of carrier peptides include SynB3, TAT (HIV-1 trans-activating transcriptor).

As used herein, the term “in combination” refers to the use of more than one prophylactic and/or therapeutic agent simultaneously or sequentially and in a manner such that their respective effects are additive or synergistic.

The term “effective amount” can be used inter-changeably with “therapeutically effective amount” as used herein, refers to an amount sufficient to affect a beneficial or desired clinical result upon treatment. Specifically, the term “effective amount” means an amount of an agent e.g., a peptide, an ORF, or a hpRNA, sufficient to measurably increase at least one of; growth in injured neurons, ii. survival of injured neurons, or iii) axon regeneration in injured neurons by at least 3 fold, at least 2.5 fold, at least 2 fold, at least 1.5 fold upon contacting with injured neurons, ex vivo or in vivo with effective amount relative to absence of contacting. The increase in at least one of the desired biological activities can result in a measurable effect in terms of neuronal repair in a treated subject against for e.g., neurodegenerative disease, brain trauma, stroke. The effective amounts may vary, as recognized by those skilled in the art, depending on the number of neurons to be contacted, the duration of contact, the specific underlying disease resulting in neuronal injury, intensity of prior therapy such as chemotherapy or radiotherapy. The effective amount of an active therapeutic agent used to practice the present disclosure for the treatment of a CNS disease or neuronal injury varies depending upon the manner of administration, the age, body weight, and general health of the subject. Ultimately, the attending clinician will decide the appropriate amount and dosage regimen. Such amount is referred to as an “effective” amount.

An effective amount can therefore result in a clinical outcome of at least one selected from; increased survival of injured neurons or increased axon regeneration in injured neurons and cause treatment, reverse, alleviate, ameliorate, inhibit, slow down or stop the progression or severity of the disease characterized by, resulting in, or due to neuronal injury.

By “promoting regeneration of axon” is meant increasing the number of axons or the distance of extension of axons relative to a control condition (e.g., in non-injured neurons). Preferably the increase is by at least 2-fold, 2.5-fold, 3-fold or more.

By “fragment” is meant a portion of a polypeptide that has at least 50% of the biological activity of the polypeptide from which it is derived. This portion contains, preferably, at least 10%, 20%, 30%, 40%, 50%, 60%, 70%, 80%, or 90% of the entire length of the reference nucleic acid molecule or polypeptide. A fragment of a polypeptide or nucleic acid molecule may contain 10, 20, 30, 40, 50, 60, 70, 80, 90, or 100, 200, 300, 400, 500, 600, 700, 800, 900, or 1000 nucleotides or amino acids.

By “neuron” is meant any nerve cell derived from the nervous system of a mammal (e.g., mature neuron of the central nervous system).

As used herein, the terms “treat,” “treatment,” “treating,” or “amelioration” refer to therapeutic treatments, wherein the object is to reverse, alleviate, ameliorate, inhibit, slow down or stop the progression or severity of a disorder or syndrome, (e.g., neuronal injury, glaucoma, stroke, head trauma, spinal injury, optic injury, ischemia, hypoxia, neurodegenerative disease, multiple sclerosis, infectious disease, cancer, and autoimmune disease) characterized by or making a patient susceptible to neuronal death and or inhibition of axon generation. The term “treating” includes reducing or alleviating at least one adverse effect or symptom of a syndrome. Treatment is generally “effective” if one or more symptoms or clinical markers are reduced. In the case of neuronal death or lack of axon generation, “effective treatment” refers to a treatment that increases the number of surviving neurons and/or increases the number of axons in the neurons) and maintains normal function of the neurons. Alternatively, or in addition, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the condition is stabilized. The term “treatment” of a disease also includes providing relief from the symptoms or side-effects of the disease (including palliative treatment).

As used herein, the term “gene expression” includes both gene transcription, whereby DNA (or RNA in the case of some RNA-containing viruses) corresponding to a gene is transcribed to generate an RNA molecule and RNA translation, whereby an RNA molecule is translated to generate a protein encoded by the gene. As used herein, the term “protein expression” is used to refer both to gene expression comprising transcription of DNA (or RNA) to form an RNA molecule and subsequent processing and translation of the RNA molecule to form protein and to gene expression comprising translation of mRNA to form protein.

The term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, a “subject”, “patient”, “individual” and like terms are used interchangeably and refers to a vertebrate, preferably a mammal, e.g., a primate, e.g., a human. Mammals include, without limitation, humans, primates, rodents, wild or domesticated animals, including feral animals, farm animals, sport animals, and pets. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. In some embodiments, the subject is a human. A subject can be male or female.

A “pharmaceutical composition” refers to a chemical or biological composition suitable for administration to a mammalian subject. Such compositions may be specifically formulated for administration via one or more of a number of routes, including but not limited to, oral, parenteral, intravenous, intraarterial, subcutaneous, intranasal, sublingual, intraspinal, intracerebroventricular, ocular and the like.

Mammals other than humans can be advantageously used as subjects that represent animal models of conditions or disorders disclosed herein. In one embodiment, the subject is a non-human primate animal in a model for neurodegeneration or nervous system (CNS or PNS) injury. Neurons derived from said subjects are also suitable for performance of the methods described herein.

A subject can be one who has been previously diagnosed with or identified as suffering from or under medical supervision for a disorder characterized by neuronal injury. A subject can be one who has undergone or will be undergoing a CNS restorative surgery or axotomy. A subject can be one who is diagnosed and currently being treated for, or seeking treatment, monitoring, adjustment or modification of an existing therapeutic treatment, or is at a risk of developing such a disorder.

As used herein, the term “administering,” refers to the placement of an agent (e.g., a fibronectin-based peptide, vitronectin, fibronectin, a hpRNA, or a vector comprising a nucleic acid sequence encoding an ORF of the invention) as disclosed herein into a subject by a method or route that results in at least partial delivery of the agent at a desired site (e.g., at or near the site of neuronal injury) such that the administering results in contact of the injured neurons with the agent.

Pharmaceutical compositions comprising the agent or cell preparation disclosed herein can be administered by any appropriate route which results in an effective treatment in the subject, e.g., intracerebroventricular (“icy”) administration, intranasal administration, intracranial administration, intracelial administration, intracelebellar administration, or intrathecal administration. Administration can be continuous or intermittent. In various aspects, a preparation or an agent can be administered therapeutically; that is, administered to treat an existing disease or condition. In further various aspects, a preparation can be administered prophylactically; that is, administered for prevention of a disease or condition (e.g., neuronal injury).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases, such as “comprising,” “including,” “carrying,” “having,” “containing,” “involving,” “holding,” “composed of,” and the like are to be understood/construed to be open-ended (i.e., to mean including but not limited to) unless otherwise noted. Only the transitional phrases “consisting of” and “consisting essentially of” shall be closed or semi-closed transitional phrases, respectively, as set forth in the United States Patent Office Manual of Patent Examining Procedures, Section 2111.03.

All methods described herein can be performed in a suitable order unless otherwise indicated herein or otherwise clearly contradicted by context. Thus, in certain methods described herein that include more than one step or act, the order of the steps or acts of the method is not necessarily limited to the order in which the steps or acts of the method are recited unless the context indicates otherwise.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The use of the terms “a” and “an” and “the” and similar referents (especially in the context of the claims) are to be construed to cover both the one or more than one (e.g., “at least one”, “plurality”, or “one or more”) of the grammatical object of the article, unless otherwise indicated herein or clearly contradicted by context. By way of example, “an element” means one element or more than one element.

The terms first, second, etc., as used herein are not meant to denote any particular ordering, but simply for convenience to denote a plurality of, for example, compositions.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The term “contacting” as used herein, refers to bringing a disclosed agent (e.g. a fibronectin-based peptide, vitronectin, fibronectin, a hpRNA or a vector comprising a nucleic acid sequence ORF) and a cell (e.g., injured neuron), a target receptor, or other biological entity together in such a manner that the agent can affect the activity of the target (e.g., neuronal cell, axon etc.), either directly; i.e., by interacting with the target itself, or indirectly; i.e., by interacting with another molecule, co-factor, factor, or protein on which the activity of the target is dependent.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof.

The term “derived” from, or “derivative” of, with respect to a reference amino acid shares a homology or identity over its entire length with a corresponding part of the reference amino acid sequence of at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In any aspect or embodiment described herein, an amino acid sequence (e.g., that of the fibronectin peptide) that is “derived” from or “corresponds” to a reference amino acid sequence is 100% homologous, or in particular 100% identical, over its entire length with a corresponding part of the reference amino acid sequence. The “homology” or “identity” of an amino acid sequence or nucleotide sequence is preferably determined according to the present disclosure over the entire length of the reference sequence or over the entire length of the corresponding part of the reference sequence that corresponds to the sequence that homology or identity is defined.

The term “fragment” is meant a portion of a polypeptide or peptide that has at least 50% of the biological activity of the polypeptide or peptide from which it is derived. This portion contains, preferably, at least 10%, at least 20%, at least 30%, at least 40%, at least 50%, at least 60%, at least 70%, at least 80%, or at least 90% of the entire length of the reference sequence (e.g., nucleic acid molecule, polypeptide, or peptide). A fragment of a polypeptide or peptide may contain at least 10, at least 20, at least 30, at least 40, at least 50, at least 60, at least 70, at least 80, at least 90, at least 100, at least 110, at least 120, at least 130, at least 140, at least 150, or at least 160 nucleotides or amino acids, such as from the reference sequence.

By “homology” is meant two or more nucleic acid or amino acid sequences is partially or completely identical. In any aspect or embodiment described here, the homologous nucleic acid or amino acid sequence has 30%, 40%, 50%, 60%, 70%, 80%, 90%, or 95% sequence similarity or identity to a nucleic acid encoding the reference nucleic acid or amino acid sequence.

The term “conservative substitution” or “conservative mutation” as used herein in the specification and claims refers to replacement of an amino acid by an amino acid of similar structure (such as size) and characteristics or chemical nature, such as where a hydrophobic amino acid is replaced by another hydrophobic amino acid (e.g., replacing a leucine with an isoleucine). In studies of sequence variations in families of naturally occurring homologous proteins, certain amino acid substitutions are more often tolerated than others, and these often show correlation with similarities in size, charge, polarity, and hydrophobicity between the original amino acid and its replacement, and such is the basis for defining “conservative substitution” or “conservative mutation”. In any aspect or embodiment described herein the term “conservative mutations” or “conservative substitutions” can refer to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

The term “neurodegeneration” refers to a physiological state caused by neuronal injury associated with neuronal loss and/or damage, or loss of axon regeneration. In specific aspects, neurodegeneration refers to neuronal injury resulting in impaired cognitive function.

The term “neuronal injury” as used herein refers to any damage or dysfunction exhibited by neurons, including but not limited to loss of myelin, dendrite retraction, dendritic spine density reduction, axonal damage, loss of axon regeneration and neuronal death.

The term “small molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

As used herein, the term “axon growth” or “axon outgrowth” includes the process by which axons or dendrites extend from a neuron. The outgrowth can result in a new neuritic projection or in the extension of a previously existing cellular process. Neurite outgrowth may include linear extension of an axonal process by five cell-diameters or more.

“Central nervous system (CNS) neurons” include the neurons of the brain, the cranial nerves and the spinal cord. The invention relates not only to CNS neurons but also to peripheral neurons that make projections (axons) in CNS, for instance dorsal root ganglion neurons.

As used herein the term “brain injury” is the destruction or degeneration of brain cells is in the brain of a living organism. Brain injuries can result from direct impacts to the head. Such injuries are for example traumatic brain injury and spinal cord injury. The present invention may also be used in treating other neuronal disorders, which include disease, disorder, or condition directly or indirectly affecting the normal functioning or anatomy of a subject's nervous system. The disorder may be a neuronal injury, which can be acute or chronic. Examples of acute injury are those that results from surgery, trauma, compression, contusion, transection or other physical injury, vascular pharmacologic or other insults including hemorrhagic or ischemic damage. Chronic neuronal injury may result from repetitive stress, inflammation/oxidative stress within a neural tissue caused by disease, neurodegenerative or other neurological diseases. The method and compositions provided herein can be beneficial in all diseases where the CSPG matrix is inhibitory for regeneration or maintenance of axons, such as TBI, SCI, multiple sclerosis (MS disease) and amyotrophic lateral sclerosis (ALS).

“Traumatic brain injury, TBI” as used herein includes the condition in which a traumatic blow to the head causes damage to the brain or connecting spinal cord, with or without penetrating the skull. It relates more specifically to the actual mechanical damage that occurs at the type of trauma, such as shearing, tearing and stretching of axons, neurons and blood vessels. Usually, the initial trauma can result in expanding hematoma, subarachnoid hemorrhage, cerebral edema, raised intracranial pressure, and cerebral hypoxia, which can, in turn, lead to severe secondary events due to low cerebral blood flow.

“A spinal cord injury, SCI” as used herein is damage to any part of the spinal cord or nerves at the end of the spinal canal. It often causes permanent changes in strength, sensation and other body functions below the site of the injury. The spinal cord injury may be a complete severing of the spinal cord, a partial severing of the spinal cord, or a crushing or compression injury of the spinal cord. Spinal cord injury SCI proceeds over minutes, hours, days and even months after the initial traumatic insult and can lead to significant expansion of the original damage. These secondary events are a consequence of delayed biochemical, metabolic and cellular changes, which are initiated by the primary injury, and includes inflammation, free radical induced cell death and glutamate excitotoxicity. Axonal sprouting, from surviving neurons, is associated with spontaneous motor and sensory recovery following TBI and SCI. Although the CNS has a limited capacity to regenerate, spontaneous pericontusional axon sprouting does take place approximately 1-2 weeks after trauma. However, this process typically fails due to an inhibitory axonal environment promoted by chon-rioting sulphate proteoglycans (CSPGs). Astrocytes, at the site of injury, produce CSPGs, beyond which the axons cannot regenerate. Inhibition of CSPG activity represents one potential approach to neuroregeneration, following either TBI or SCI. Evidence in support of this theory has been provided through the use of chondroitinase ABC (ChABC, an enzyme that degrades CSPGs) at the site of trauma in rodent models of TBI and SCI. ChABC treatment resulted in an enhanced and prolonged sprouting response with an increase in sensory, motor and autonomic function.

The terms “increased”, “increase”, “increasing”, or “enhance” are all used herein to generally mean an increase by a statically significant amount; for the avoidance of doubt, the terms “increased”, “increase”, or “enhance”, mean an increase of at least 10% as compared to a reference level, for example an increase of at least about 10%, at least about 20%), or at least about 30%, or at least about 40%, or at least about 50%, or at least about 60%), or at least about 70%, or at least about 80%, or at least about 90%, or up to and including a 100% increase or any increase between 10-100%) as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. The increase can be, for example, at least 10%, at least 20%), at least 30%, at least 40%, or more, and is preferably to a level accepted as within the range of normal for an individual without a given disease.

As used herein the term “reference level” refers to a level of expression of an agent in a “control sample”. A control sample can be one that has not been contacted with an agent of the present disclosure. In certain embodiments, a control sample is obtained prior to administration of the inhibitor. In certain embodiments, a reference standard is used as a surrogate for a control sample.

As used herein, the term “vector” is used in reference to a vehicle used to introduce a nucleic acid sequence into a cell. A viral vector is virus that has been modified to allow recombinant DNA sequences to be introduced into host cells or cell organelles. A non-viral vector delivers an amplified amount of agent or nucleic acid to a target tissue, cell or subcellular area, and comprise a lipid based or solid platform suitable for binding a number of substances (e.g., nanoparticle, liposomes etc.).

“Nucleic acid sequence”, as used herein, refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group. Nucleic acid sequences may be used herein to refer to oligonucleotides, or polynucleotides, and fragments or portions thereof, and to DNA or RNA of genomic or synthetic origin that may be single- or double-stranded and represent the sense or antisense strand. The polymer can be composed of deoxyribonucleotides, ribonucleotides, or modified nucleotides in a single- or double-stranded form and is intended to encompass nucleic acids bearing nucleotide analogs or modified backbone residues or linkages known in the art. For example, the term “nucleic acid” or “polynucleotide” includes single-, double- or multi-stranded DNA or RNA, genomic DNA, cDNA, DNA-RNA hybrids, or a polymer comprising purine or pyrimidine bases, or other natural, chemically or biochemically modified, non-natural, or derivatized nucleotide base.

The terms “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%), or at least about 80%, or at least about 90% or up to and including a 100% decrease (e.g., absent level or non-detectable level as compared to a reference level), or any decrease between 10-100%) as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “statistically significant” or “significantly” refers to statistical significance and generally means a difference of two standard deviations (2SD) or more.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: A Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, the term “stem-loop” structure refers to a structure including a double-stranded moiety (stem) formed by base-pairing through hydrogen bonding between sequences inside a single-stranded nucleic acid molecule, and an intervening single-stranded ring moiety (loop) with no intraloop hydrogen bonds. The term “stem-loop” may be used interchangeably with the term “hairpin” or “hairpin loop”. The nucleic acid molecule according to an embodiment has a stem-loop structure in an environment where a mRNA is expressed but becomes active (opens the hairpin structure) to regulate the expression of a target gene in an environment where a mRNA is present or overexpressed.

Retinal ganglion cells (RGCs) are central nervous system (CNS) cells that reside within the innermost layer of the retina, the ganglion cell layer. Their axons bundle together to form the optic nerve. Although RGCs share numerous features, they comprise over 40 discrete types in mice (although there are more RGCs in humans than in mice, it is still unknown whether they segregate into substantially more types), each with distinct morphological and physiological features. Related RGC types, called subclasses, include alpha RGCs (αRGCs), which express osteopontin (Spp1); T- and F-RGCs, defined by expression of the transcription factors Tbr1 and Foxp2, respectively; ooDSGCs, defined by physiological properties and bistratified dendrites; and intrinsically photosensitive RGCs (ipRGCs) defined by expression of melanopsin (Opn4). Single-cell RNA-seq (scRNA-seq) can be used to generate a comprehensive molecular atlas of the different types of RGCs, which are then put into clusters based on markers found in the differentially expressed genes of the cluster.

RGCs relay visual information from the eye to the brain through their axons. Optic nerve injuries induced by trauma, glaucoma or neurodegenerative diseases often result in loss of visual functions and eventually blindness. To restore vision after optic nerve injury, injured axons must regenerate the full length of the eye-to-brain pathways, a distance of more than 8 mm from the injury site. Long-distance axon regeneration, is crucial in the restoration of visual function following optic nerve injury. The majority (˜80%) of RGCs dis a few weeks after optic nerve injury, making RGC survival a major obstacle for a sufficient number of regenerating axons necessary for visual function recovery.

The present inventors surprisingly and unexpected discovered that a subset of the adult RGCs survived better and regenerated axons when cultured on vitronectin, and even better when cultured on fibronectin. The present inventors further surprisingly and unexpectedly discovered that a fibronectin-based peptide promoted axon regeneration in vivo. The inventors of the present disclosure surprisingly and unexpectedly discovered that a fibronectin-based peptide can be utilized with various signal peptides, with or without a purification tag, as a peptide therapeutic, such as a peptide therapeutic to repair neural connections or prevent damage to neural connection (e.g., a preventive treatment or co-treatment and a post-damage treatment or co-treatment). The surprising use of vitronectin, fibronectin, and fibronectin-based peptides as a preventive treatment or co-treatment and a post-damage treatment or co-treatment was unexpectedly discovered by the present inventors. The inventors of the present disclosure further discovered a novel approach for axon regeneration and RGC survival after treatment by overexpression of RNAs of specific target genes, knockdown of target RNAs using shRNAs. The inventors of the present disclosure further discovered a novel approach for axon regeneration and RGC survival by treatment or co-treatment with RNAs of specific protein-coding and/or non-coding genes, and/or knockdown of specific RNA using shRNAs, in combination with each other and/or with fibronectin or a fibronectin-based peptide.

The inventors have also identified novel target RNAs, both non-coding RNAs and mRNA expressed from target genes, the expression of which can be regulated by herewith described agents comprising novel synthetic nucleic acids for either reduced expression (knockdown or downregulate target non-coding RNA, or target mRNA or protein of a target gene), or for increased expression (overexpress or upregulate target non-coding RNA, or target mRNA or protein of a target gene), result in promotion of RGC survival, or neuroprotection, and axon regeneration, specifically long-distance axon regeneration, in vivo, in short-term (2 weeks after injury) and long-term (6 weeks after injury) studies for use alone or as a combination with a fibronectin or a therapeutic fibronectin-based peptide thereof.

An aspect of the present disclosure relates to a fibronectin-based peptide (also referenced herein as “an agent,” “the recombinant peptide,” or “therapeutic peptide”) comprising, or consisting of, a fibronectin peptide that is derived from (e.g., a fragment of), or has, the amino acid sequence

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

A further aspect of the present disclosure relates to a fibronectin-based peptide (also referenced herein as “an agent,” “the recombinant peptide,” or “therapeutic peptide”) comprising, or consisting of, a fibronectin peptide that has the amino acid sequence

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin peptide has an amino acid sequence that is at least 90% or at least 95% (e.g., at least 90%, 91%, 92%, 93%, 94%, 95%, 96%, 97%, 98%, 99%, or 99.5%) identity with (e.g., a fragment of) the amino acid sequence:

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin peptide has the amino acid sequence:

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin peptide includes one or more (e.g., 1, 2, 3, 4, 5, 6, 7, 8, 9, 10, 11, 12, 13, 14, 15, 16, 17, 18, 19, 20, or 21) conservative substitutions and/or conservative mutation relative to the reference sequence

(SEQ ID NO: 41)
DSPTGIDFSDITANSFTVHWIAPRATITGYRIRHHPEHFSGRPREDRVP
HSRNSITLTNLTPGTEYVVSIVALNGREESPLLIGQQSTVSDVPRDLEV
VAATPTSLLISWDAPAVTVRYYRITYGETGGNSPVQEFTVPGSKSTATI
SGLKPGVDYTITVYAVTGRGDSP.

In any aspect or embodiment described herein, the fibronectin-based peptide further comprises a signal peptide (e.g., a signal peptide on the N-terminus of the fibronectin peptide or C-terminus of the fibronectin peptide).

In any aspect or embodiment described herein, the signal peptide comprises or consists of the amino acid sequence MGWSCIILFLVATATGVHS (SEQ ID NO:42).

In any aspect or embodiment described herein, the fibronectin-based peptide further comprises an affinity tag (e.g., an affinity tag on the N-terminus of the fibronectin peptide or C-terminus of the fibronectin peptide).

In any aspect or embodiment described herein, the affinity tag comprises or is: biotin (e.g., binds streptavidin, avidin, or neutravidin), calmodulin-binding peptide (CBP) tag (KRRWKKNFIAVSAANRFKKISSSGAL; SEQ ID NO:43) (e.g., binds calmodulin), chitin-binding domain (CBD) tag (e.g., binds chitin), E-tag (GAPVPYPDPLEPR; SEQ ID NO:44) (e.g., binds anti-E-tag antibody), Fc-tag (e.g., binds Protein-A Sepharose), FLAG tag (DYKDDDDK; SEQ ID NO:45) (e.g., binds anti-FLAG monoclonal antibody, such as M1, M2, and M5, or a derivative thereof), glutathione S-transferase (GST) tag (e.g., binds glutathione), HA-tag (YPYDVPDYA; SEQ ID NO:46) (e.g., binds anti-hemagglutinin antibody), HaloTag (binds a reactive chloroalkane linker), Histidine tag (5-10 histidines) (e.g., binds metal ions, such as Ni²⁺, Co²⁺, Cu²⁺, Zn²⁺, Fe³⁺), Gly-His-tags (e.g. GHHHH, or GHHHHHH (SEQ ID NO:47), or GSSHHHHHH (SEQ ID NO:48)) (e.g., bind immobilized metal cations), maltose-binding protein (MBP) (binds amylose), Myc-tag (EQKLISEEDL; SEQ ID NO:49) (e.g., binds an anti-Myc antibody), NE-tag (TKENPRSNQEESYDDNES; SEQ HD NO:50) (e.g., binds anti-NE antibody), polyarginine tag (binds cation-exchange resin), polyglutamate tag (binds anion-exchange resin), SNAP-Tag® (binds, e.g., guanine or chloropyrimidine with a benzyl linker to the label)), Streptavidin-Binding Peptide (SBP)-Tag (MDEKTTGWRGGHVVEGLAGELEQLRARLEHHPQGQREP; SEQ ID NO:51) (e.g., binds streptavidin), Strep II Tag (WSHPQFEK; SEQ ID NO:52) (e.g., binds streptavidin or Strep-Tactin), S-tag (KETAAAKFERQHMDS; SEQ ID NO:53) (e.g., binds S-protein of RNase A), T7-tag (MASMTGGQQMG; SEQ ID NO:54) (e.g., binds anti-T7 antibodies), V5-tag (GKPIPNPLLGLDST; SEQ ID NO:55) (e.g., binds anti-V5 polyclonal antibody, monoclonal antibody, or nanobody), or a combination thereof.

In any aspect or embodiment described herein, the affinity tag is a histidine tag (e.g., a 6× His tag).

An additional aspect of the present disclosure relates to a composition (e.g., a composition, such as a pharmaceutical composition or therapeutic composition, for promoting neuroprotection or axon regeneration of an injured axon), comprising the fibronectin-based peptide (also referenced herein as “an agent,” “the recombinant peptide,” or “therapeutic peptide”) of the present disclosure, and optionally a pharmaceutically acceptable carrier, vehicle, additive, excipient, diluent, adjuvant, or stabilizer (e.g., the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus).

In any aspect or embodiment described herein, the second composition further comprises a vehicle, wherein the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus.

In any aspect or embodiment described herein, the composition comprising the fibronectin-based peptide further comprises (i) the nucleic acid molecule, (ii) the expression cassette, (iii) the protein product, or (iv) a combination thereof.

The inventors further used bulk and single-cell RNA-seq (scRNA-seq) technologies that can detect protein-coding messenger RNAs (mRNAs), to identify mRNAs that are developmentally regulated in the retinal ganglion cell (RGC) neurons as they mature, as well as those that are differentially expressed exclusively in the small subset of the RGC neurons, which regenerate long-distance (at least 3 mm) axons in response to pro-growth treatment with anti-Pten small hairpin RNAs (shRNAs). The inventors specifically captured the rare long-distance axon-regenerating RGCs for scRNA-seq analysis and compared their transcriptomes to embryonic RGCs. Using this approach, the inventors were able to identify novel nucleic acid open reading frames (ORF) and small hairpin RNA (shRNA) molecules that can regulate novel biological targets that promote neuroprotection by extending retinal ganglion cell survival and regeneration of damaged neurons evidenced by long-distance axon growth in the injured optic nerve model. Additionally, results from in vivo axon regeneration assays pre-treated prior to injury, or optic nerve crush (ONC), support the use of these novel RNAs for both therapeutic and preventative applications, and not just post-damage treatment.

In any aspect or embodiment described herein, the target RNA is mRNA expressed from the target gene(s). Therefore, in any aspect or embodiment described herein, the composition comprises nucleic acid molecules that target mRNA expression of the target gene. In any aspect or embodiment described herein, the composition comprises a nucleic acid molecule having one or more synthetic small hairpin RNA (shRNA) molecules comprising a region complementary to a portion of a target RNA, wherein hybridization of the complementary region of the shRNA to the target RNA blocks target RNA function and promotes axon neuroprotection and/or regeneration.

The term “small hairpin RNA (shRNA)” refers to an artificial RNA molecule with a hairpin structure that can be used for inhibiting the expression of a target gene via the phenomenon of RNA interference (RNAi). A simple stem-loop shRNA forms a stem-loop structure consisting of a 19 to 29 base pair (bp) region of double-stranded RNA (the stem) bridged by a region of predominantly single-stranded RNA (the loop). Once transcribed, shRNAs exit the nucleus, and the loop region of the shRNA is cleaved off from the shRNA precursor transcript during processing by the nuclease Dicer in the cytoplasm, and the remaining mature miRNA-like sequence, the expressed functional sequence, targets the complementary RNA to direct cleavage and subsequent degradation. Because the loop itself gets cleaved off and does not target, different loop sequences can be used from either mouse or human shRNAs. Similarly, the stem region of the stem-loop can be any region known to form a stem-loop. Design of shRNAs for expression in target cells is known in the art. Since shRNA has a relatively low decomposition rate and turnover, it can be effectively used for RNA interference (RNAi).

In addition to shRNA, in any aspect or embodiment described herein, other nucleic acids known to inhibit expression of a gene by binding to target RNA transcripts can be designed to target the expression of the target genes described herein. These include any RNA selected from the group consisting of siRNAs, microRNAs, and aptamers.

The term “small interfering RNA (siRNA)”, which is an RNAi-inducing material, refers to a short double-helix RNA strand consisting of about 20 to about 30 nucleotides. Once siRNAs are injected into a cell, they target mRNAs with a complementary nucleotide sequence thereto and thereby inhibit the expression of the corresponding genes.

The term “microRNA (miRNA)” refers to a ribonucleic acid molecule found in all eukaryotic cells, having a length of about 20 to about 25 nucleotides. The microRNAs can inhibit the expression of a particular gene by binding to a target RNA transcript with a complementary sequence thereto thereby inhibiting translation of the transcript, by histone modification, or by inducing DNA methylation to a promoter of the target gene.

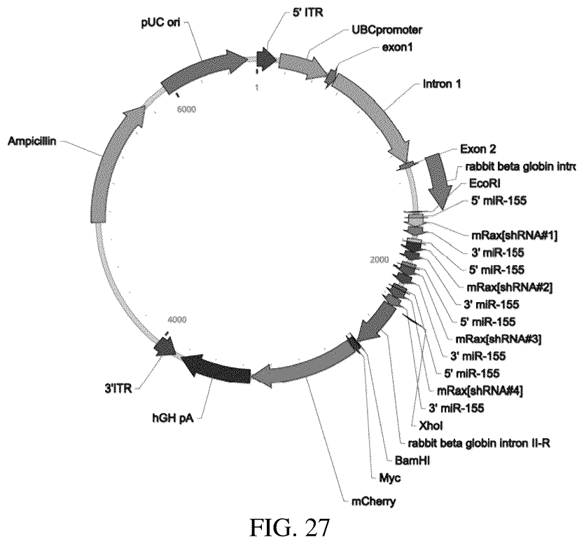

The predicted ORFs were designed with different than endogenous mRNA 3′ and 5′ untranslated region (UTR) sequences, and included a coding Myc tag at the ORFs 3′ end (shown in FIG. 27). The shRNA and other non-coding RNA sequences designed for knockdown of the known mRNA, miRNA, and piRNA targets do not exist naturally in cells. The mimics of the endogenous miRNAs were expressed from the modified precursor of miR-155, thus, the precursor sequence containing the functional miRNA-mimic for expression in cells does not exist naturally in cells. Furthermore, the design of the precursor included 4 such mimics, which enables expression of 4 functional miRNA mimics from the same RNA precursor molecule, which enables a higher level of functional miRNA-mimic expression that does not occur in cells naturally.

The target RNA expressed from the target gene, expression of which is inhibited or reduced at an mRNA level and/or a protein level by the shRNA nucleic acid molecule, or the expressed functional sequence, according to any aspect or embodiment of the present disclosure. In any aspect or embodiment described herein, the target gene expressing the target RNA may be at least one selected from the group consisting of Mmp9, Rax, Crx, Pdnp, Prdm13, or a combination thereof. As shown in the Examples below, these genes were selected through unbiased bioinformatic analysis of all genes, which identified those that were developmentally regulated based on RNA-seq data from embryonic and mature RGCs. The target genes and the therapeutic functional sequence for each are shown in Table 1. The mouse sequence shown can be used as well as the human ortholog.

TABLE 1
TARGET GENES OR mRNAS FOR KNOCKDOWN BY shRNA

| | | | Vector sequence |
|---|---|---|---|
| | | | encoding the |
| | | | sncRNA, within |
| | | | the stem-loop |
| | | Sequence of shRNA | regions of the |
| | | (SEQ ID NO) that is | shRNA, which will |
| | Murine accession no./ | complementary to the target | be expressed in |
| Target | Human ortholog | RNA. Expressed functional | cells to target an |
| gene | accession no. | sequence underlined. | mRNA target sequence |
| Mmp9 | ENSMUSG00000017737/ | #1 | pAAV[miR155]- |
| | ENSG00000100985 | TTTGAGTTTCCATAGTA | UBC>4x |
| | | AGTGGTTTTGGCCTCTG | mMmp9[shRNA] |
| | | ACTGACCACTTCTGGG | SEQ ID NO: 73 |
| | | AAACTCAAA (SEQ ID | |
| | | NO: 14, POSITION 1978- | |
| | | 2036 OF SEQ ID NO: 73) | |
| | | #2 | |
| | | TATGTCGTCTTTATTCA | |
| | | GAGGGTTTTGGCCTCTG | |
| | | ACTGACCCTCTACTAA | |
| | | GACGACATA (SEQ ID | |
| | | NO: 70, POSITION 2141- | |
| | | 2199 OF SEQ ID NO: 73) | |
| | | #3 | |
| | | TTATGATGGTCCCACTT | |
| | | GAGGCTTTTGGCCTCTG | |
| | | ACTGAGCCTCAGTGGA | |
| | | CCATCATAA (SEQ ID | |
| | | NO: 71, POSITION 1652- | |
| | | 1710 OF SEQ ID NO: 73) | |
| | | #4 | |
| | | TTTATCCTGGTCATAGT | |
| | | TGGCTTTTTGGCCTCTG | |
| | | ACTGAAGCCAATCTAC | |
| | | CAGGATAAA (SEQ ID | |
| | | NO: 72, position 1815-1873 | |
| | | of SEQ ID NO: 73) | |
| Rax | ENSMUSG00000024518/ | #1 | pAAV[miR155]- |
| | ENSG00000134438 | AGTTTGTCCCTCTGACA | UBC>mRax[shRNA#1]: |
| | | GCGAGTTTTGGCCTCTG | mRax[shRNA#2]: |
| | | ACTGACTCGCTCCTAG | mRax[shRNA#3]: |
| | | GGACAAACT (SEQ ID | mRax[shRNA#4]: |
| | | NO: 11, position 1752-1710 | Myc/mCherry |
| | | of SEQ ID NO: 74) | SEQ ID NO: 74 |
| | | #2 | |
| | | TTCGATGCTGTGCAAA | |
| | | CGCGACTTTTGGCCTCT | |
| | | GACTGAGTCGCGTGTA | |
| | | CAGCATCGAA (SEQ ID | |
| | | NO: 67, position 1815-1873 | |
| | | of SEQ ID NO: 74) | |
| | | #3 | |
| | | ATGGACGACACTTCCA | |
| | | GTTTCTTTTTGGCCTCT | |
| | | GACTGAAGAAACAGTG | |
| | | TGTCGTCCAT (SEQ ID | |
| | | NO: 68, position 1978-2036 | |
| | | of SEQ ID NO: 74) | |
| | | #4 | |
| | | ATGCCGTCTTCCTTGGT | |
| | | AAAGCTTTTGGCCTCTG | |
| | | ACTGAGCTTTAGACGA | |
| | | AGACGGCAT (SEQ ID | |
| | | NO: 69, position 2141-2199 | |
| | | of SEQ ID NO: 74) | |
| Crx | ENSMUSG00000041578/ | #1 | pAAV[miR155]- |
| | ENSG00000105392 | TTAAGAGCAACCTCCT | UBC>mCrx[shRNA#1]: |
| | | CACGTGTTTTGGCCTCT | mCrx[shRNA#2]: |
| | | GACTGACACGTGAGTG | mCrx[shRNA#3]: |
| | | TTGCTCTTAA (SEQ ID | mCrx[shRNA#4]: |
| | | NO: 10, position 1642-1710 | Myc/mCherry |
| | | of SEQ ID NO: 75) | SEQ ID NO: 75 |
| | | #2 | |
| | | TGAATTTGGAGGTCTC | |
| | | ACTTTGTTTTGGCCTCT | |
| | | GACTGACAAAGTTGCC | |
| | | TCCAAATTCA (SEQ ID | |
| | | NO: 64, position 1815-1873 | |
| | | of SEQ ID NO: 75) | |
| | | #3 | |
| | | TCTTTGTAGTCCAGAGG | |
| | | GTCCATTTTGGCCTCTG | |
| | | ACTGATGGACCTTGGA | |
| | | CTACAAAGA (SEQ ID | |
| | | NO: 65, position 1815-1873 | |
| | | of SEQ ID NO: 75) | |
| | | #4 | |
| | | ATTTCGCCCTACGATTC | |
| | | TTGAATTTTGGCCTCTG | |
| | | ACTGATTCAAGCTCTA | |
| | | GGGCGAAAT (SEQ ID | |
| | | NO: 66, position 1978-2036 | |
| | | of SEQ ID NO: 75) | |
| Pdpn | ENSMUSG00000028583/ | #1 | pAAV[miR155]- |
| | ENSG00000162493 | TATTATCCAGAAAGAT | UBC>mPdpn[shRNA#1]: |
| | | GCAGCTTTTTGGCCTCT | mPdpn[shRNA#2]: |
| | | GACTGAAGCTGCCCTT | mPdpn[shRNA#3]: |
| | | CTGGATAATA (SEQ ID | mPdpn[shRNA#4]: |
| | | NO: 8, position 1652-1710 | Myc/mCherry |
| | | of SEQ ID NO: 76) | SEQ ID NO: 76 |
| | | #2 | |
| | | TCATTAAGCCCTCCAGT | |
| | | AGCACTTTTGGCCTCTG | |
| | | ACTGAGTGCTAGTGGG | |
| | | GCTTAATGA (SEQ ID | |
| | | NO: 58, position 1815-1873 | |
| | | of SEQ ID NO: 76) | |
| | | #3 | |
| | | ATCTTTCTTATCTGTTG | |
| | | TCTGCTTTTGGCCTCTG | |
| | | ACTGAGCAGACAACAT | |
| | | AAGAAAGAT (SEQ ID | |
| | | NO: 59, | |
| | | position 1978-2036 of SEQ | |
| | | ID NO: 76) | |
| | | #4 | |
| | | TTTGACAGTGGTTGTAC | |
| | | TCTCGTTTTGGCCTCTG | |
| | | ACTGACGAGAGAACCC | |
| | | ACTGTCAAA (SEQ ID | |
| | | NO: 60, position 2141-2199 | |
| | | of SEQ ID NO: 76) | |
| Prdm13 | ENSMUSG00000040478/ | #1 | pAAV[miR155]- |
| | ENSG00000112238 | AAATAATTGCAACTTG | UBC>mPrdm13 |
| | | ACCAATTTTTGGCCTCT | [shRNA#1]:mPrdm13 |
| | | GACTGAATTGGTGCAT | [shRNA#2]:mPrdm13 |
| | | GCAATTATTT (SEQ ID | [shRNA#3]:mPrdm13 |
| | | NO: 9, position 1652-1710 | [shRNA#4]: |
| | | of SEQ ID NO: 77) | Myc/ |
| | | #2 | SEQ ID NO: 77 |
| | | GGATTAACCCTATCCA | |
| | | CTCCAGTTTTGGCCTCT | |
| | | GACTGACTGGAGCGCA | |
| | | GGGTTAATCC (SEQ ID | |
| | | NO: 61, position 1815-1873 | |
| | | of SEQ ID NO: 77) | |
| | | #3 | |
| | | TGGTAAGTCTGCAATA | |
| | | GCTTCCTTTTGGCCTCT | |
| | | GACTGAGGAAGCCGTC | |
| | | AGACTTACCA (SEQ ID | |
| | | NO: 62, position 1978-2036 | |
| | | of SEQ ID NO: 77) | |
| | | #4 | |
| | | TTTCTCATCATGCGTTG | |
| | | GAGTCTTTTGGCCTCTG | |
| | | ACTGAGACTCCGCTAT | |
| | | GATGAGAAA (SEQ ID | |
| | | NO: 63, position 2141-2199 | |
| | | of SEQ ID NO: 77) | |
| Ift20 and its | ENSMUSG00000001105 | Expression cassette: | pAAV[miR155]- |
| lncRNA | (ift20)/ | [5′ miR-155: | UBC>4x Anti- |
| isoform | ENSG00000109083 | shRNA sequence: | ift20 shRNA |
| ENSMUST | (ift20) | 3′miR-155: | VB200810- |
| 00000128788 | | 5′miR-155: | 1671efc |
| | | shRNAsequence: | SEQ ID NO: 78 |
| | | 3′miR-155: | |
| | | 5′miR-155: | |
| | | shRNA sequence: | |
| | | 3′miR-155] | |
| | | AACACTCGGAGCTTGT | |
| | | TCAGTTCATCAAATTTT | |
| | | GGCCTCTGACTGATTTG | |
| | | ATGAATAACAAGCTCC | |
| | | GAGTGTT (SEQ ID NO: | |
| | | 30, position 1652-1724, | |
| | | 1829-1901, 2006-2078, | |
| | | 2183-2255 of SEQ ID | |
| | | NO: 78) | |

In any aspect or embodiment described herein, the target RNA is a small noncoding RNA (sncRNA).

SncRNAs, such as microRNA (miRNA), small nuclear RNA, small nucleolar RNA, tRNA, derived small RNA and Piwi-interacting RNA (piRNA) are circulating RNAs that are noncoding, but in conjunction with other molecules are involved in regulation through RNAi. PiRNAs interact with PIWI-class Argonaute proteins with sequence bias for only the first 5′ nucleotide to be Uracil. PIWI proteins function at the chromatin level by guiding DNA methylation and deposition of repressive histone marks to silence transposable elements. piRNA in association with PIWI protein interacts with transposable elements to prevent insertion through methylation or transcriptional repression.

The inventors discovered that shRNAs targeted to reduce or downregulate expression of specific piRNAs, sncRNAs, or miRNAs in a neuronal cell can promote neuroprotection and/or regeneration of axons. Target piRNA are presented in Table 2 and can be any of piR-16295, piR-129-5p, or a combination thereof. Target sncRNAs are presented in Table 3, or a combination thereof. Target miRNAs are presented in Table 4, or a combination thereof. In each case, the expressed functional sequence is shown.

In any aspect or embodiment described herein, the composition comprising the (i) the nucleic acid molecule, (ii) the expression cassette, (iii) the fibronectin protein-based product, or (iv) a combination thereof.

TABLE 2
TARGET piRNA FOR KNOCKDOWN OF EXPRESSION USING DESIGNED
shRNAS

| | | Expression cassette with | |
|---|---|---|---|
| | | sequence encoding the sncRNA, | |
| | | within the stem-loop regions of | |
| | | the shRNA, which will be | |
| | | expressed in cells to target | Vector sequence encoding |
| | | piRNA target sequence including | the sncRNA, within the |
| | | the stem-loop regions of the | stem-loop regions of the |
| | Murine | shRNA in brackets. | shRNA, which will be |
| | accession no./ | The expressed functional | expressed in cells to target |
| | Human | sequence, ie. the sequence of the | piRNA target sequence |
| Target | ortholog | sncRNA that is complementary to | including the stem-loop |
| PIRNA | accession no. | the target piRNA, is underlined. | regions of the shRNA |
| piR- | DQ548183.1 | [5′ miR-155:anti(piR-16295): | pAAV[miR155]-UBC>4x |
| 16295 | (mmu) | 3′miR-155:5′miR-155:anti(piR- | anti(piR-16295) |
| | | 16295):3′miR-155:5′miR-155: | SEQ ID NO: 79 |
| | | anti(piR-16295):3′miR-155:5′miR- | |
| | | 155:anti(piR-16295:3′miR-155] | |
| | | ACACCGTCCACGGGCTGGG | |
| | | CCTCGATCATTTTGGCCTCT | |
| | | GACTGATGATCGAGCCTGCC | |
| | | CGTGGACGGTGT (SEQ ID | |
| | | NO: 29, positions 1952-1722, | |
| | | 1827-1897, 2002-2072, 2177- | |
| | | 2247 of SEQ ID NO: 79) | |

TABLE 3
TARGET sncRNA FOR OVEREXPRESSION USING DESIGNED shRNAS

| | | | Vector sequence |
|---|---|---|---|
| | | Expression cassette | encoding the sncRNA, |
| | Murine | with sequence of | within the stem-loop |
| | accession no./ | inserted shRNA in | regions of the shRNA, |
| | Human | brackets. Expressed | which will be expressed |
| Cellular target of | ortholog | functional sequence | in cells to target |
| the sncRNA | accession no. | underlined. | RNA/DNA |
| Unknown RNA or | Novel | [5′ miR-155: shRNA | pAAV[miR155]-UBC>4x |
| DNA molecule that | | sequence: 3′miR- | shRNA VB200810-1013mnb |
| contains sequences that | | 155:5′miR-155: shRNA | SEQ ID NO:80 |
| are complementary to | | sequence: 3′miR- | |
| sncRNA sequence | | 155:5′miR-155: shRNA | |
| VB200810-1013mnb | | sequence:3′miR- | |
| shRNA sequence | | 155:5′miR-155: shRNA | |
| | | sequence: 3′miR-155] | |
| | | TGAGCACCAGGCT | |
| | | GTTGAAAAATGAC | |
| | | CCATTTTGGCCTCT | |
| | | GACTGATGGGTCA | |
| | | TTTTTACAGCCTG | |
| | | GTGCTCA (SEQ ID | |
| | | NO: 31, position: | |
| | | 1652-1724, 1829- | |
| | | 1901, 2006-2078, | |
| | | 2183-2255 of SEQ ID | |
| | | NO: 80). | |
| Unknown RNA or | Novel | [5′ miR-155: shRNA | pAAV[miR155]- |
| DNA molecule that | | sequence: 3′miR- | UBC>4x shRNA |
| contains sequences that | | 155:5′miR-155: shRNA | VB200810-1300pah |
| are complementary to | | sequence: 3′miR- | SEQ ID NO: 81 |
| sncRNA sequence | | 155:5′miR-155: shRNA | |
| VB200810-1300pah | | sequence:3′miR- | |
| | | 155:5′miR-155: shRNA | |
| | | sequence: 3′miR-155] | |
| | | TGCTGTGAAGGTG | |
| | | AATCTG | |
| | | GATTTCCTTGAATT | |
| | | TTGGCC | |
| | | TCTGACTGATTCA | |
| | | AGGAAAC | |
| | | CGATTCACCTTCA | |
| | | CAGCA (SEQ ID | |
| | | NO: 33, position 1652- | |
| | | 1728, 1833-1909, 2014- | |
| | | 2090, 2195-2271 of | |
| | | SEQ ID NO: 81). | |
| Unknown RNA or | Novel | [5′ miR-155: shRNA | pAAV[miR155]- |
| DNA molecule that | | sequence: 3′miR- | UBC>4x shRNA |
| contains sequences that | | 155:5′miR-155: shRNA | VB200810-1256arq |
| are complementary to | | sequence: 3′miR- | SEQ ID NO: 82 |
| sncRNA sequence | | 155:5′miR-155: shRNA | |
| VB200810-1256arq | | sequence:3′miR- | |
| | | 155:5′miR-155: shRNA | |
| | | sequence: 3′miR-155] | |
| | | AACATCCCCACCA | |
| | | ACTCTTAGCACTG | |
| | | GATTTTGGCCTCT | |
| | | GACTGATCCAGTG | |
| | | CACGGTTGGTGGG | |
| | | GATGTT | |
| | | (SEQ ID NO: 32, | |
| | | POSITION: 1652-1722, | |
| | | 1827-1897, 2002- | |
| | | 2072, 2177-2247 OF | |
| | | SEQ ID NO: 82). | |
| Unknown RNA or | Novel | ACTCACTTGTTGG | pAAV[miR155]- |
| DNA molecule that | | GGCGCAGTCTTAA | UBC>4x shRNA |
| contains sequences that | | GCATTTTGGCCTCT | VB200810-1672kgn |
| are complementary to | | GACTGATGCTTAA | SEQ ID NO: 83 |
| sncRNA sequence | | GATATCCCCAACA | |
| sncRNA | | AGTGAGT | |
| VB200810-1672kgn | | (SEQ ID NO: 35, | |
| | | position: 1652-1724, | |
| | | 1829-1901, 2006- | |
| | | 2078, 2183-2255 of | |
| | | SEQ ID NO: 83). | |
| Unknown RNA or | Novel | TGCTCTCTGAACG | pAAV[miR155]- |
| DNA molecule that | | AGTGAGGCATCTC | UBC>4x shRNA |
| contains sequences that | | CCATTTTGGCCTCT | VB200810-1670qan |
| are complementary to | | GACTGATGGGAGA | SEQ ID NO: 84 |
| sncRNA sequence | | TGCTTCTCGTTCAG | |
| VB200810-1670qan | | AGAGCA | |
| | | (SEQ ID NO: 34, | |
| | | position: 1652-172, | |
| | | 1829-1901, 2006- | |
| | | 2078, 2183-2255 of | |
| | | SEQ ID NO: 84). | |

TABLE 4
TARGET miRNA FOR KNOCKDOWN OF EXPRESSION USING DESIGNED
shRNAS

| | | | Vector sequence encoding |
|---|---|---|---|
| | | Expression cassette with | the sncRNA, within the |
| | | miRNA sequence in | stem-loop regions of the |
| | Murine accession | brackets. Expressed | shRNA, which will be |
| Target | no./Human ortholog | functional sequence | expressed in cells to |
| miRNA | accession no. | underlined | target miRNA. |
| miR-129-1-3p | URS00004CCDA3_10090/ | [5′ miR-155:anti(mmu- | pAAV[miR155]-UBC>4x |
| | URS00004CCDA3_9606 | miR-129-1-3p:3′miR- | anti(mmu-miR-129-1-3p) |
| | | 155:5′miR-155:anti(mmu- | SEQ ID NO: 85 |
| | | miR-129-1-3p):3′miR- | |
| | | 155:5′miR-155:anti(mmu- | |
| | | miR-129-1-3p):3′miR- | |
| | | 155:5′miR0155:anti(mmu- | |
| | | miR-129-1-3p):3′miR-155] | |
| | | ATACTTTTTGGGGTAAGG | |
| | | G | |
| | | CTTTTTTGGCCTCTGACTG | |
| | | A | |
| | | AAGCCCTCTCCAAAAAGT | |
| | | AT | |
| | | (SEQ ID NO: 24, | |
| | | positions, 1652-1710, | |
| | | 1815-1873, 1978-2036, | |
| | | 2141-2199 of SEQ ID | |
| | | NO: 85). | |
| miR-129-5p | URS00004E1410_10090/ | [5′ miR-155:anti(mmu- | pAAV[miR155]-UBC>4x |
| | URS00004E1410_9606 | miR-129-5p:3′miR- | anti(mmu-miR-129-5p) SEQ |
| | | 155:5′miR-155:anti(mmu- | ID NO: 86 |
| | | miR-129-5p)3′miR- | |
| | | 155:5′miR-155:anti(mmu- | |
| | | miR-129-5p):3′miR- | |
| | | 155:5′miR0155:anti(mmu- | |
| | | miR-129-5p):3′miR-155] | |
| | | TGCAAGCCC | |
| | | AGACCGCAAA | |
| | | AAGTTTTGGC | |
| | | CTCTGACTGA | |
| | | CTTTTTCAGC | |
| | | TGGGCTTGCA | |
| | | (SEQ ID NO:23, position | |
| | | 1652-1710, 1815-1873, | |
| | | 1978-2036, 2141-2199 of | |
| | | SEQ ID NO: 86). | |
| miR-1290 | URS000043F369_9606 | [5′ miR-155:anti(hsa-mir- | pAAV[miR155]-UBC>4x |
| | (has) | 1290):3′miR-155:5′miR- | anti(hsa-mir-1290) |
| | | 155: anti(hsa-mir- | SEQ ID NO: 87 |
| | | 1290):3′miR-155:5′miR- | |
| | | 155:anti(hsa-mir- | |
| | | 1290):3′miR- | |
| | | 155:5′miR0155:anti(hsa- | |
| | | mir-1290): 3′miR-155] | |
| | | TCCCTGATCCAAAAATCC | |
| | | A | |
| | | AAATTTTGGCCTCTGACTG | |
| | | A | |
| | | TTTTGGTTTTGGATCAGGG | |
| | | A | |
| | | (SEQ ID NO: 19, positions | |
| | | 1652-1710, 1815-1873, | |
| | | 1978-2036, 2141-2199 of | |
| | | SEQ ID NO: 87) | |
| miR-6090 | URS0000759F58_9606 | [5′ miR-155:anti(has-mir- | pAAV[miR155]-UBC>4x |
| | (has) | 6090): 3′miR-155:5′miR- | anti(hsa-mir-6090) SEQ ID |
| | | 155: anti(mmu-miR-6090 | NO: 88 |
| | | 3′miR-155:5′miR-155: | |
| | | anti(mmu-miR- | |
| | | 6090:3′miR- | |
| | | 155:5′miR0155: anti(mmu- | |
| | | miR-6090: 3′miR-155] | |
| | | TGCCCCGCCCCTCGCTCCC | |
| | | CCATTTTGGCCTCTGACTG | |
| | | A | |
| | | TGGGGGGGGGGGGCGGGG | |
| | | CA | |
| | | (SEQ ID NO: 25, positions | |
| | | 1652-1710, 1815-1873, | |
| | | 1978-2036, 2141-2199 of | |
| | | SEQ ID NO: 88) | |
| miR-132-3p | URS00006054DA_10090/ | [5′ miR-155:anti(mmu- | pAAV[miR155]-UBC>4x |
| | URS00006054DA_9606 | miR-132-3p): 3′miR- | anti(mmu-miR-132-3p) |
| | | 155:5′miR-155: anti(mmu- | SEQ ID NO: 89 |
| | | miR-132-3p): 3′miR- | |
| | | 155:5′miR-155: anti(mmu- | |
| | | miR-132-3p):3′miR- | |
| | | 155:5′miR0155: anti(mmu- | |
| | | miR-132-3p): 3′miR-155] | |
| | | TCGACCATG | |
| | | GCTGTAGACT | |
| | | GTTATTTTGG | |
| | | CCTCTGACTG | |
| | | ATAACAGTTC | |
| | | TGCCATGGTC GA | |
| | | (SEQ ID NO: 20, positions | |
| | | 1652-1712, 1817-1877, | |
| | | 1982-2042, 2208-2266 of | |
| | | SEQ ID NO: 89). | |
| miR-29a-3p | URS00002F4D78_10090/ | [5′ miR-155:anti(mmu- | pAAV[miR155]-UBC>4x |
| | URS00002F4D78_9606 | miR-29a-3p): 3′miR- | anti(mmu-miR-29a-3p) |
| | | 155:5′miR-155:(mmu-miR- | SEQ ID NO: 90 |
| | | 29a-3p):3′miR-155:5′miR- | |
| | | 155:anti(mmu-miR29a- | |
| | | 3p):3′miR- | |
| | | 155:5′miR0155:anti(mmu- | |
| | | miR-29a-3p): 3′miR-155] | |
| | | TAACCGATTTCAGA | |
| | | TGGTGCTATTTTGG | |
| | | CCTCTGACTGATAG | |
| | | CACATTGAAATCG | |
| | | GTTA | |
| | | (SEQ ID NO: 18, | |
| | | position: 1652-1710, | |
| | | 1815-1873, 1978-2036, | |
| | | 2141-2199 of SEQ ID | |
| | | NO: 90). | |

In any aspect or embodiment described herein, a composition comprising nucleic acid molecules that increase the amount of target RNA of a target gene in a neuronal cell, thereby resulting in overexpression of the target gene or target RNA. In any aspect or embodiment described herein, the composition comprises one or more synthetic open reading frame (ORF) molecules designed to be sufficiently homologous to the target mRNA, thereby increasing the amount of target RNA or encoded target protein in the neuronal cell and promoting axon neuroprotection and/or regeneration. The novel identified target genes for overexpression of target RNA are presented in Table 5.

TABLE 5
TARGET GENES FOR OVEREXPRESSION USING DESIGNED ORF SEQUENCES

| Target gene | | | |
|---|---|---|---|
| mimicked by | | | |
| mRNA with | | | |
| ORF | | | Nucleic acid sequence in the vector for |
| expressed | | | expression of an functional mRNA |
| from the | Murine accession no./Human | AAV2 vector name: | containing a designed ORF sequence |
| vector | ortholog accession no. | SEQ ID NO | (SEQ ID NO) |
| Rpl7a | ENSMUSG00000062647/ | pAAV[Exp]- | Myc/Myc/mRpl7a[NM_013721.3] |
| | ENSG00000148303 | CMV > Myc/Myc/ | (SEQ ID NO: 1) |
| | | mRpl7a[NM_013721.3]: | |
| | | WPRE | |
| | | SEQ ID NO: 91 | |
| Rpl7 | ENSMUSG00000043716/ | pAAV[Exp]- | Myc/Myc/mRpl7[NM_011291.5] |
| | ENSG00000147604 | CMV > Myc/Myc/ | (SEQ ID NO: 2) |
| | | mRpl7[NM_011291.5]: | |
| | | WPRE | |
| | | SEQ ID NO: 92 | |
| Tceb2 | ENSMUSG00000055839/ | pAAV[Exp]- | Myc/Myc/mTceb2[NM_026305.2] |
| | ENSG00000103363 | CMV > Myc/Myc/ | (SEQ ID NO: 3) |
| | | mTceb2[NM_026305.2]: | |
| | | WPRE | |
| | | SEQ ID NO: 93 | |
| Lancl1 | ENSMUSG00000026000/ | pAAV[Exp]- | {2xMyc/mLancl1[NM_001190984.1]} |
| | ENSG00000115365 | CMV > {2xMyc/ | SEQ ID NO: 17 |
| | | mLancl1[NM_001190984.1]}: | |
| | | WPRE | |
| | | SEQ ID NO: 94 | |
| Atp6v0c | ENSMUSG00000024121/ | pAAV[Exp]- | {2xMyc/mAtp6v0c[NM_009729.4]} |
| | ENSG00000185883 | CMV > {2xMyc/ | SEQ ID NO: 16 |
| | | mAtp6v0c[NM_009729.4]}: | |
| | | WPRE | |
| | | SEQ ID NO: 95 | |
| Dynlt1a | ENSMUSG00000092074/ | pAAV[Exp]- | {2xMyc/mDynltla[NM_001166629.2]} |
| | N/A | CMV > {2xMyc/ | SEQ ID NO: 15 |
| | | mDynlt1a[NM_001166629.2]}: | |
| | | WPRE | |
| | | SEQ ID NO: 96 | |
| Mrtfa | ENSMUSG00000042292/ | pAAV[Exp]- | {2xMyc/mMrtfa[NM_153049.3]} |
| | ENSG00000196588 | CMV > {2xMyc/ | SEQ ID NO: 13 |
| | | mMrtfa[NM_153049.3]}: | |
| | | WPRE | |
| | | SEQ ID NO: 97 | |
| Lars2 | ENSMUSG00000035202/ | pAAV[Exp]- | {2xMyc/mLars2[NM_001348167.1]} |
| | ENSG00000011376 | CMV > {2xMyc/ | SEQ ID NO: 12 |
| | | mLars2[NM_001348167.1]}: | |
| | | WPRE | |
| | | SEQ ID NO: 98 | |
| Dpys15 | ENSMUSG00000029168/ | pAAV[Exp]- | {Myc/Myc/mDpysl5[NM_023047.3]} |
| | ENSG00000157851 | CMV > {Myc/Myc/ | SEQ ID NO: 5 |
| | | mDpys15[NM_023047.3]}: | |
| | | WPRE | |
| | | SEQ ID NO: 99 | |
| Fblim1 | ENSMUSG00000029168/ | pAAV[Exp]- | {Myc/Myc/mFblim1[NM_133754.5]} |
| | ENSG00000157851 | CMV > {Myc/Myc/ | SEQ ID NO: 6 |
| | | mFblim1[NM_133754.5]}: | |
| | | WPRE | |
| | | SEQ ID NO: 100 | |
| Dpysl3 | ENSMUSG00000024501/ | pAAV[Exp]- | {Myc/Myc/mDpysl3[NM_001136086] |
| | ENSG00000113657 | CMV > {Myc/Myc/ | SEQ ID NO: 4 |
| | | mDpysl3[NM_001136086]}: | |
| | | WPRE | |
| | | SEQ ID NO: 101 | |
| Nfe2l3 | ENSMUSG00000029832/ | pAAV[Exp]- | {Myc/Myc/mNfe213[NM_010903.2]} |
| | ENSG00000050344 | CMV > {Myc/Myc/ | SEQ ID NO: 7 |
| | | mNfe213[NM_010903.2]}: | |
| | | WPRE | |
| | | SEQ ID NO: 102 | |

In any aspect or embodiment described herein, synthetic nucleic acid molecules for overexpression of target miRNA are designed and found to promote neuroprotection and/or regeneration. Synthetic mimic miRNA nucleic acid molecules expressed in cells are presented in Table 6. Design of mimic miRNA is known in the art. Briefly, it is the same nucleic acid sequence as that of the annotated endogenous mature miRNA with the identical sequence. However, because it is processed in a cell from a viral vector precursor sequence rather than from the endogenous precursor RNA expressed from genomic DNA, it is considered a mere mimic and not an endogenous miRNA per se.

TABLE 6
TARGET miRNA FOR OVEREXPRESSION USING DESIGNED
SEQUENCES

| | | Expression cassette | |
|---|---|---|---|
| ID of the | | with designed shRNA | |
| biological miRNA | | sequence for | |
| or sncRNA, which | | expression of miRNA- | Designed nucleic |
| is mimicked by | Murine accession | mimic in cells shown | acid vector for |
| the miRNA-mimic | no./Human | in brackets. Expressed | expression of miRNA- |
| expressed from | ortholog | functional sequence | mimic in cells |
| the vector | accession no. | underlined (SEQ ID NO) | (SEQ ID NO) |
| miR-5109 and | MI0018018/ | [5′miR-155:mmu-miR- | pAAV[miR155]-UBC>4x mmu-miR- |
| miR-5109-like | miR-5109-like | 5109:3′miR-155:5′miR- | 5109 |
| sncRNA derived | sncRNA derived | 155:mmu-miR- | SEQ ID NO: 103 |
| from ribosomal | from rRNA 28S | 5109:3′miR-155:5′miR- | |
| RNA large subunit | large subunit | 155:mmu-miR- | |
| 28S | | 5109:3′miR-155:5′miR- | |
| | | 155:mmu-miR-5109:3′ | |
| | | miR-155] | |
| | | TGTTGCGGA | |
| | | CCAGGGGAAT | |
| | | CCGATTTTGG | |
| | | CCTCTGACTG | |
| | | ATCGGATTCC | |
| | | TGGTCCGCAA | |
| | | (SEQ ID NO: 28, | |
| | | positions 1652-1712, | |
| | | 1817-1877, 1982-2042, | |
| | | 2147-2207 of SEQ ID | |
| | | NO: 103) | |
| miR-1247-5p | URS000057DF36_ | [5′miR-155:mmu-miR- | pAAV[miR155]-UBC>4x mmu-miR- |
| | 10090/ | 1247-5p:3′miR- | 1247-5p |
| | URS000057DF36_ | 155:5′miR-155:mmu- | SEQ ID NO: 104 |
| | 9606 | miR-1247-5p:3′miR- | |
| | | 155:5′miR-155:mmu- | |
| | | miR-1247-5p:3′miR- | |
| | | 155:5′miR-155:mmu- | |
| | | miR-1247-5p:3′miR- | |
| | | 155] | |
| | | ACCCGTCCC | |
| | | GTTCGTCCCC | |
| | | GGATTTTGGC | |
| | | CTCTGACTGA | |
| | | TCCGGGACGA | |
| | | CGGGACGGGT | |
| | | (SEQ ID NO: 26, | |
| | | positions 1652-1710, | |
| | | 1815-1873, 1978-2036, | |
| | | 2141-2199 of SEQ ID | |
| | | NO: 104) | |
| miR-210-3p | URS000055128B_ | [5′ miR-155:(mmu- | pAAV[miR155]-UBC>4x mmu-miR- |
| | 10090/ | miR-210-3p):3′miR- | 210-3p |
| | URS000055128B_ | 155:5′miR-155: | SEQ ID NO: 105 |
| | 9606 | (mmu-miR-210-3p): | |
| | | 3′miR-155:5′miR- | |
| | | 155:(mmu-miR-210- | |
| | | 3p):3′miR- | |
| | | 155:5′miR0155:(mmu- | |
| | | miR-210-3p):3′miR- | |
| | | 155] | |
| | | TCTGTGCGTGTGACAG | |
| | | CGGCTGATTTTGGCCT | |
| | | CTGACTGATCAGCCGT | |
| | | ATACACGCACAGA | |
| | | (SEQ ID NO: 21 at | |
| | | positions 1652-1712, | |
| | | 1817-1877, 1982-2042, | |
| | | 2147-2207 of SEQ ID | |
| | | NO: 105). | |
| miR-3914-1 | URS00005547BA_ | [5′ miR-155:anti(hsa- | pAAV[miR155]-UBC>4x hsa-mir- |
| | 9606 (hsa) | mir-3914-1):3′miR- | 3914-1 |
| | | 155:5′miR-155: | SEQ ID NO: 106 |
| | | anti(hsa-mir-3914- | |
| | | 1):3′miR-155:5′miR- | |
| | | 155:anti(hsa-mir-3914- | |
| | | 1):3′miR-155:5′miR- | |
| | | 155:anti(hsa-mir-3914- | |
| | | 1):3′miR-155] | |
| | | AAGGAACCA | |
| | | GAAAATGAGA | |
| | | AGTTTTTGGC | |
| | | CTCTGACTGA | |
| | | ACTTCTATTT | |
| | | CTGGTTCCTT | |
| | | (SEQ ID NO: 27, | |
| | | positions 1652-1710, | |
| | | 1815- 873, 1978-2036, | |
| | | 2141-2199 of SEQ ID | |
| | | NO: 106) | |
| miR-135b-3p | URS0000488C83_ | [5′ miR-155:(mmu-miR- | pAAV[miR155]-UBC>4x mmu-miR- |
| | 10090/ | 135b-3p):3′miR- | 135b-3p (SEQ ID NO: 107) |
| | URS0000488C83_ | 155:5′miR-155:(mmu- | |
| | 9606 | miR-135b-3p):3′miR- | |
| | | 155:5′miR-155: | |
| | | 135b(mmu-miR-135b- | |
| | | 3p):3′miR-155:5′miR- | |
| | | 155:(mmu-miR-135b- | |
| | | 3p):3′miR-155] | |
| | | ATGTAGGGC | |
| | | TAAAAGCCAT | |
| | | GGGTTTTGGC | |
| | | CTCTGACTGA | |
| | | CCCATGCTTT | |
| | | AGCCCTACAT (SEQ | |
| | | ID NO: 22, positions | |
| | | 1652-1710, 1815-1873, | |
| | | 1978-2036, 2141-2199 | |
| | | of SEQ ID NO: 107) | |
| miR-135a-3p | URS0000169868_ | [5′ miR-155:(mmu-miR- | pAAV[miR155]-UBC>4x mmu-miR- |
| | 10090/ | 135a-3p):3′miR- | 135a-1-3p |
| | URS00005675D3_ | 155:5′miR-155:(mmu- | SEQ ID NO: 108 |
| | 9606 | miR-135a-3p):3′miR- | |
| | | 155:5′miR-155: | |
| | | 135a(mmu-miR-135a- | |
| | | 3p):3′miR-155:5′miR- | |
| | | 155:(mmu-miR-135a- | |
| | | 3p):3′miR-155] | |
| | | TATAGGGATTGG | |
| | | AGCCGTGGCGTT | |
| | | TTGGCCTCTGAC | |
| | | TGACGCCACGCT | |
| | | CAATCCCTATA | |
| | | (SEQ ID NO: 57, | |
| | | position: 1652-1710, | |
| | | 1815-1873, 1978- | |
| | | 2036, 2141-2199 of | |
| | | SEQ ID NO:108). | |

In any aspect or embodiment described herein, the nucleic acid molecules described herein can be provided in multiple copies to provide an additive or synergistic effect. For example, in any aspect or embodiment described herein and as a nonlimiting example, four copies of a shRNA, having the same or different loop sequence or stem-loop sequence, or four copies of an ORF, for the same or different target gene, can be provided simultaneously as different nucleic acid molecules or as part of the same nucleic acid molecule. In any aspect or embodiment described herein, two or more different shRNA, as part of the same or different nucleic acid molecule, or two or more different ORF as part of the same or different nucleic acid molecule, can be combined and provided. Such combinatory effects may be advantageous due to different signaling pathways downstream of these genes and sncRNAs and may result in longer distance axon regeneration. In any aspect or embodiment described herein, a fibronectin-based peptide is provided as a co-treatment with the nucleic acid molecules described herein.

Increased survival of neurons is indicated by the number of neurons surviving from a specific injury or condition upon contact with an agent disclosed herein (e.g. shRNAs and/or ORFs), as compared to the number of neurons surviving in absence of said contact, and also by the length of time the survival persists upon contact with an agent disclosed herein (e.g., shRNAs or ORFs), as compared to that in absence of contact. Survival is considered to be sustained if it persists for an extended period of time post-injury (e.g., greater than 2 weeks post-injury, greater than 3 weeks, and greater than 4 weeks postinjury). In any aspect or embodiment described herein, greater than 10% of neurons (e.g., 15%, 20%, 25%, 30%, 35%, 0%, 45%, 50%, 55%), 60%), 65%), 70%) and 75%), survive upon contact with one or more agents disclosed herein. In any aspect or embodiment described herein, greater than 20% of neurons (e.g., 25%, 30%, 35%, 0%, 45%, 50%), 55%), 60%), 65%), 70% and 75%), survive for an extended period of time post-injury.

Increased regeneration or outgrowth is indicated by the number of neurons (injured and also uninjured) and by extended length of the axonal outgrowth of the neurons upon contact with the agent disclosed herein (e.g., shRNAs and/or ORFs), as compared to that in absence of the said contact, and by the time frame post-injury that the outgrowth occurs upon contact with an agent disclosed herein (e.g. shRNAs and/or ORFs), as compared to the time frame postinjury that outgrowth occurs in absence of said contact. In any aspect or embodiment described herein, increased regeneration and axonal outgrowth occurs if greater than 10% or greater than 20% (e.g., 15%, 20%, 25%, 30%, 35%, 0%, 45%, 50%), 55%), 60%), 65%), 70% and 75%) of the neurons regenerate injured axons or generate new axons. In any aspect or embodiment described herein, the regenerated axons extend at least 0.5 mm distal to the lesion epicenter. In one embodiment, greater than 10% or greater than 20% (e.g., 15%, 20%, 25%, 30%, 35%, 0%, 45%, 50%, 55%, 60%, 65%, 70% and 75%) of neurons regenerate injured axons or generate axons over 1 millimeter distal to the lesion site. In any aspect or embodiment described herein, greater than 10% (e.g., 15%, 20%, 25%, 30%, 35%, 0%, 45%, 50%, 55%, 60%, 65%, 70% and 75%)) or greater than 20% of neurons regenerate or generate new axons that extend at least 2 mm distal from the lesion site.

In any aspect or embodiment described herein, a product of a target gene in Tables above wherein overexpression of the target RNA is shown to promote neuroprotection and/or axon regeneration, the encoded protein, or an analog or a derivative thereof can be provided. In any aspect or embodiment described herein, an agonist, a compound that enhances or stimulates the normal biological activity of the target gene described in the Tables above can be a compound that enhances or stimulates the normal biological activity of the target gene by increasing transcription or translation of the target RNA, and/or by inhibiting or blocking activity of a molecule that inhibits target gene expression or target protein activity, and/or by enhancing normal target protein biological activity (including, but not limited to enhancing the stability of the target protein or target RNA. In any aspect or embodiment described herein, the “biological activity” can be defined herein as including at least one of the activities selected from e.g., increasing neuroprotection in neurons, increasing survival of injured neurons and increasing axon regeneration of injured neurons, in vivo or in vitro. For example, in any aspect or embodiment described herein, the activity of the agonist can be at least 80%, at least 85%, at least 90%, at least 95%, at least 97%, or at least 99% of the biological activity of the target gene in Table 5.

In any aspect or embodiment described herein, an antagonist of a target gene in Tables above, wherein knockdown of expression of the target RNA is shown to promote neuroprotection and/or axon regeneration, an antagonist comprises a compound that inhibits or reduces the normal biological activity of the target gene in Tables herein can be a compound that inhibits or reduce the normal biological activity of the target gene by reducing transcription or translation of the target RNA, and/or by stimulating or enhancing activity of a molecule that inhibits target gene expression or target protein activity, and/or by reducing normal target protein biological activity (including, but not limited to reducing the stability of the target protein or target RNA. The “biological activity” can be defined herein as including at least one of the activities selected from e.g., reducing neuroprotection in neurons, reducing survival of injured neurons and reducing axon regeneration of injured neurons, in vivo or in vitro. For example, in any aspect or embodiment described herein, the activity of the antagonist can be at least 80%, at least 85%, at least 90%, at least 95%, at least 97%, or at least 99% of the biological activity of the target gene in Tables 1, 2 or 4.

In any aspect or embodiment described herein, the nucleic acid molecule encoding the shRNA or ORF described above is part of an expression cassette. In any aspect or embodiment described herein, the expression cassette can be operably linked to an expression control element. The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements. In any aspect or embodiment described herein, exemplary regulatory elements in prokaryotes include promoters, operator sequences and a ribosome binding site. In any aspect or embodiment described herein, regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

In any aspect or embodiment described herein, the expression cassette is in a vehicle for expression in a neuronal cell, in vivo or in vitro. In any aspect or embodiment described herein, the neuronal cell is an RGC. In any aspect or embodiment described herein, the vehicle is a vector for carrying and transferring a nucleic acid to a neuronal cell. In any aspect or embodiment described herein, non-limiting examples of vectors include plasmids and viral vectors (for example, AAV, lentivirus, or herpes simplex virus vectors). In on any aspect or embodiment described herein, such vectors are rAAV vectors. It will be appreciated that other cloning vectors may be used in the invention, and therefore reference to AAV herein may be taken to refer to any suitable vector.

The term “AAV” or “adeno-associated virus” refers to a Dependoparvovirus within the Parvoviridae genus of viruses. For example, in any aspect or embodiment described herein, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene.

The term “rAAV” refers to a “recombinant AAV”. In any aspect or embodiment described herein, a recombinant AAV has an AAV genome in which part or all of the rep and cap genes have been replaced with heterologous sequences.

The term “cap gene” refers to the nucleic acid sequences that encode capsid proteins that form, or contribute to the formation of, the capsid, or protein shell, of the virus. In the case of AAV, in any aspect or embodiment described herein, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

In any aspect or embodiment described herein, AAV vectors that comprise coding regions of one or more shRNA or ORF of the invention of interest are provided. In any aspect or embodiment described herein, the AAV vector can include a 5′ inverted terminal repeat (ITR) of AAV, a 3′ AAV ITR, a promoter driving expression of the shRNA or ORF nucleic acid, and a restriction site downstream of the promoter to allow insertion of a polynucleotide encoding one or more shRNA or ORF of interest, wherein the promoter and the restriction site are located downstream of the 5′ AAV ITR and upstream of the 3′ AAV ITR. In any aspect or embodiment described herein, the AAV vector includes a posttranscriptional regulatory element, for example the Woodchuck Hepatitis Virus Posttranscriptional Regulatory Element (WPRE), downstream of the restriction site and upstream of the 3′ AAV ITR. In any aspect or embodiment described herein, the AAV vectors disclosed herein can be used as AAV transfer vectors carrying a transgene encoding a protein of interest for producing recombinant AAV viruses that can express the protein of interest in a host cell.

The nucleotide sequences of AAV ITR regions are known. The ITR sequences for AAV-2 are described in the art. The skilled artisan will appreciate that AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including but not limited to, AAV-1, AAV-2, AAV-3, AAV-4, AAV-5, AAVX7, and the like. Furthermore, 5′ and 3′ ITRs which flank a selected nucleotide sequence in an AAV expression vector need not necessarily be identical or derived from the same AAV serotype or isolate, so long as the ITR's function as intended, i.e., to allow for excision and replication of the bounded nucleotide sequence of interest when AAV rep gene products are present in the cell.

In any aspect or embodiment described herein, rAAV vectors are provided herein. Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

In any aspect or embodiment described herein, the viral vectors can include additional sequences that make the vectors suitable for replication and integration in eukaryotes. In any aspect or embodiment described herein, the viral vectors disclosed herein can include a shuttle element that makes the vectors suitable for replication and integration in both prokaryotes and eukaryotes. In any aspect or embodiment described herein, the viral vectors can include additional transcription and translation initiation sequences, such as promoters and enhancers; and additional transcription and translation terminators, such as polyadenylation signals. In any aspect or embodiment described herein, the vector can also comprise regulatory control elements known to one of skill in the art to influence the expression of the RNA and/or protein products encoded by the polynucleotide within desired cells of the subject.

Expression control elements and promoters include those active in a particular tissue or cell type, referred to herein as a “tissue-specific expression control elements/promoters.” Tissue-specific expression control elements are typically active in specific cell or tissue (for example, in the brain, central nervous system, spinal cord, or retina). Expression control elements are typically active in these cells, tissues, or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. In any aspect or embodiment described herein, such elements include, but are not limited to, the cytomegalovirus (CMV) immediate early promoter/enhancer sequences, the Rous sarcoma virus (RSV) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1alpha; or synthetic elements that are not present in nature.

Alternatively, in any aspect or embodiment described herein, the regulatory sequences of the AAV vector can direct expression of the gene preferentially in a particular cell type, i.e., tissue-specific regulatory elements can be used. In any aspect or embodiment described herein, non-limiting examples of tissue-specific promoters that can be used include, central nervous system (CNS) specific promoters such as, neuron-specific promoters (e.g., the human synapsin promoter). Preferably, in any aspect or embodiment described herein, the promoter is tissue specific and is essentially not active outside the central nervous system, or the activity of the promoter is higher in the central nervous system that in other systems. For example, in any aspect or embodiment described herein, a promoter specific for the optic nerve, spinal cord, brainstem (medulla oblongata, pons, and midbrain), or combinations thereof. In any aspect or embodiment described herein, the promoter may be specific for particular cell types, such as motor neurons, sensory neurons, or interneurons in the CNS. In any aspect or embodiment described herein, the promoter is specific for RGCs.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present; the greater the amount of signal or stimuli, the greater the increase or decrease in expression.

Most AAV serotypes have the capability to transduce neurons, albeit with varying strength. For example, AAVs 1, 2, 5, 7, 8 and 9 all show strong preference for transducing neurons in vivo following brain injections. Serotype 1 (AAV2) has a natural tropism for neurons and is the most commonly used and characterized serotype. AAV tropism can also be further altered by creating recombinant versions of multiple AAV serotypes, a process known as pseudotyping. These pseudotyped viruses can have enhanced tropism for specific cell types, as well as improved transduction efficiency in neurons. Pseudotyping involves engineering new viral capsids from different serotpyes to create rAAVs with different cell-type expression (for example expression in CNS neurons).

AAV variants and rAAVs with different capabilities for targeting different cells in the nervous system, e.g., AAV-PHP.eB and AAV-PHP.S can target neurons in the CNS and PNS, respectively, when injected intravenously, bypassing the need to perform site-directed injections in the brain, are known in the art. In any aspect or embodiment described herein, the AAV variant or rAAV is AAV-PHP.eB or AAV-PHP.S.

In order to produce recombinant AAV particles, in any aspect or embodiment described herein, an AAV vector can be introduced into a suitable host cell using known techniques, such as by transfection. A number of transfection techniques are generally known in the art. Exemplary transfection methods, in any aspect or embodiment described herein, include calcium phosphate co-precipitation, direct micro-injection into cultured cells, electroporation, liposome mediated gene transfer, lipid-mediated transduction, and nucleic acid delivery using high-velocity microprojectiles.

In any aspect or embodiment described herein, exemplary host cells for producing recombinant AAV particles include, but are not limited to, microorganisms, yeast cells, insect cells, and mammalian cells, that can be, or have been, used as recipients of an exogenous nucleic acid molecule. Thus, a “host cell” as used herein generally refers to a cell which has been transfected with an exogenous nucleic acid molecule. In any aspect or embodiment described herein, the host cell includes any eukaryotic cell or cell line so long as the cell or cell line is not incompatible with the protein to be expressed, the selection system chosen, or the fermentation system employed. In any aspect or embodiment described herein, non-limiting examples include CHO dhfr− cells, 293 cells (Graham et al. (1977) J. Gen. Virol. 36: 59) or myeloma cells like SP2 or NS0.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, in any aspect or embodiment described herein, AAV helper functions include one, or both of the major AAV open reading frames, namely the rep and cap coding regions, or functional homologues thereof.

Alternatively, in any aspect or embodiment described herein, a vector of the present disclosure can be a virus other than the adeno-associated virus, or portion thereof, which allows for expression of a nucleic acid molecule introduced into the viral nucleic acid. For example, in any aspect or embodiment described herein, replication defective retroviruses, adenoviruses and/or lentivirus can be used. Protocols for producing recombinant retroviruses and for infecting cells in vitro or in vivo with such viruses can be found in standard laboratory manuals. In any aspect or embodiment described herein, examples of suitable retroviruses include pLJ, pZIP, pWE and pEM which are well known to those skilled in the art. In any aspect or embodiment described herein, examples of suitable packaging virus lines include Crip, Cre, 2 and Am. In any aspect or embodiment described herein, the genome of adenovirus can be manipulated such that it encodes and expresses the protein of interest but is inactivated in terms of its ability to replicate in a normal lytic viral life cycle. In any aspect or embodiment described herein, exemplary adenoviral vectors derived from the adenovirus strain Ad type 5 dl324 or other strains of adenovirus (e.g., Ad2, Ad3, Ad7 etc.) are well known to those skilled in the art.

Alternatively, in any aspect or embodiment described herein, the vector can be delivered using a non-viral delivery system. For example, in any aspect or embodiment described herein, the vector to the desired tissues is delivered in colloidal dispersion systems that include, for example, macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information. In any aspect or embodiment described herein, examples of lipids for liposome production include phosphatidyl compounds, such as phosphatidylglycerol, phosphatidylcholine, phosphatidylserine, phosphatidylethanolamine, sphingolipids, cerebrosides, and gangliosides. In any aspect or embodiment described herein, examples of lipids include, but are not limited to, polylysine, protamine, sulfate and 3b-[N—(N′,N′ dimethylaminoethane) carbamoyl] cholesterol.

Alternatively, in any aspect or embodiment described herein, the vector can be coupled with a carrier for delivery. In any aspect or embodiment described herein, exemplary and preferred carriers are keyhole limpet hemocyanin (KLH) and human serum albumin. In any aspect or embodiment described herein, carriers may include a variety of lymphokines and adjuvants such as INF, IL-2, IL-4, IL-8 and others. In any aspect or embodiment described herein, the vector can be conjugated to a carrier by genetic engineering techniques that are well known in the art. In any aspect or embodiment described herein, the vector is delivered in one or more combinations of the above delivery methods.

Also disclosed herein are pharmaceutical compositions comprising one or more of the rAAV viruses disclosed herein and one or more pharmaceutically acceptable carriers. In any aspect or embodiment described herein, the compositions can also comprise additional ingredients (such as diluents, stabilizers, excipients, and adjuvants). As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

In any aspect or embodiment described herein, the carriers, diluents and adjuvants can include buffers (such as phosphate, citrate, or other organic acids); antioxidants (such as ascorbic acid); low molecular weight polypeptides (e.g., less than about 10 residues); proteins (such as serum albumin, gelatin or immunoglobulins); hydrophilic polymers (such as polyvinylpyrrolidone); amino acids (such as glycine, glutamine, asparagine, arginine, or lysine); monosaccharides, disaccharides, and other carbohydrates including glucose, mannose, or dextrins; chelating agents (such as etheylenediaminetetraacetic acid (EDTA)); sugar alcohols (such as mannitol or sorbitol); salt-forming counterions (such as sodium); and/or nonionic surfactants (such as Tween™, Pluronics™ or polyethylene glycol (PEG)). In any aspect or embodiment described herein, the physiologically acceptable carrier is an aqueous pH buffered solution.

Titers of the rAAV to be administered will vary depending, for example, on the particular rAAV, the mode of administration, the treatment goal, the individual, and the cell type(s) being targeted, and can be determined by methods standard in the art.

As will be readily apparent to one skilled in the art, the useful in vivo dosage of the recombinant virus to be administered and the particular mode of administration will vary depending upon the age, weight, the severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. In any aspect or embodiment described herein, the rAAV for delivery a nucleic acid to a neuron (e.g., CNS) of a subject can be administered, for example via injection, to a subject at a dose of between 1×10⁶ genome copies (GC) and 2×10¹⁰ GC of the recombinant virus (for example, between 5×10⁷ GC and 5×10¹² GC). In any aspect or embodiment described herein, the dose of the rAAV administered to the subject is no more than 2×10¹⁰ GC. In any aspect or embodiment described herein, the dose of the rAAV administered to the subject is no more than 5×10¹² GC. In any aspect or embodiment described herein, the dose of the rAAV administered to the subject is no more than 5×10¹¹ GC.

For administration, the nucleic acids, peptides, proteins, and/or compositions of the present disclosure is contacted with the neuron using a suitable drug delivery method and treatment protocol sufficient to promote regeneration of the axon. For in vitro methods, the agent is added to the culture medium, usually at nanomolar or micromolar concentrations. For in vivo applications, the agent can be administered orally, by intravenous (i.v.) bolus, by i.v. infusion, subcutaneously, intramuscularly, ocularly (intraocularly, periocularly, retrobulbarly, intravitreally, subconjunctivally, topically, by subtenon administration, etc.), intracranially, intraperitoneally, intraventricularly, intrathecally, by epidural, etc. Depending on the intended route of delivery, the agent or compositions comprising the agent may be administered in one or more dosage form(s) (e.g., liquid, ointment, solution, suspension, emulsion, tablet, capsule, caplet, lozenge, powder, granules, cachets, douche, suppository, cream, mist, eye drops, gel, inhalant, patch, implant, injectable, infusion, etc.). The dosage forms may include a variety of other ingredients, including binders, solvents, bulking agents, plasticizers etc. In any aspect or embodiment described herein, the agent (e.g., peptide) is contacted with the neuron using an implantable device that contains the activator and that is specifically adapted for delivery to a CNS axon of neuron. Examples of devices include solid or semi-solid devices, such as controlled release biodegradable matrices, fibers, pumps, stents, adsorbable gelatin (e.g., Gelfoam), etc. The device may be loaded with premeasured, discrete and contained amounts of the activator sufficient to promote regeneration of the axon. In any aspect or embodiment described herein, the device provides continuous contact of the neuron with the activator at nanomolar or micromolar concentrations, preferably for at least 2, 5, or 10 days.

Administration is to a subject by a route that results in contacting an effective amount of the agents disclosed herein (e.g., a fibronectin-based peptide, vitronectin, fibronectin, shRNAs. And/or ORFs) to the target neuron(s). As the term is used herein, the target neuron is the neuron which is intentionally contacted by the administered agent. A target neuron can be an injured neuron or a non-injured neuron (e.g., for compensatory axonal outgrowth to a region of denervation, or used as a control in the methods herein). The target neuron may be contacted at one or more specific target sites of the neuron. As the term is used herein, the target site of the neuron is the region of the neuron to which the agent is intentionally contacted. Regions of the neuron include the dendrites, cell body, and the axon. In some embodiments, the target site of the neuron is the mitochondria of the neuron. Methods for targeting the compositions to mitochondria of a cell are known in the art. Non-limiting exemplary methods for targeting the compositions disclosed herein to the mitochondria of a neuron can include for e.g., conjugation of an agent with a lipohilic molecular group, encapsulation of agents in a liposome or nanoparticle.

Since regeneration and axonal generation in the treatment of a neuronal injury includes compensatory promotion of axonal outgrowth of uninjured neurons, benefit is expected from mere delivery of the nucleic acids, peptides, proteins, and/or compositions to an injury site. As such, suitable target neurons are actual damaged neurons, and also neurons that are in the immediate area of an injury site or an area of denervation. The specific location and extent of an injury site can be determined by the skilled practitioner. Examples of injury sites are the site of physical damage or disruption of neuronal activity. The immediate area of an injury site will vary with respect to the specific injury, the nature of the injury, and the nature of the injured neurons (e.g., axonal length, specific function, etc.) and can be determined by the skilled practitioner. Typically, a lesion is in the axon of the injured neuron.

In any aspect or embodiment described herein, the immediate area of the injury site is within about 1-10 mm of identified damaged neurons (e.g., 1, 2, 3, 4, 5, 6, 7, 8, 9, or 10 mm). In one embodiment, the administration is localized so as to be highly targeted to a specific site. In one embodiment, the administration is systemic, and results in delivery of the appropriate concentration to the specific site.

Depending on the intended route of delivery, the nucleic acids, peptides, proteins, and/or compositions may be administered in one or more dosage form(s) (e.g., liquid, ointment, solution, suspension, emulsion, tablet, capsule, caplet, lozenge, powder, granules, cachets, douche, suppository, cream, mist, eye drops, gel, inhalant, patch, implant, injectable, infusion, etc.). The dosage forms may include a variety of other ingredients, including binders, solvents, bulking agents, plasticizers etc.

In any aspect or embodiment described herein, the nucleic acids peptides, proteins, and/or compositions are contacted with the neuron using an implantable device that contains the compositions and that is specifically adapted for delivery to a neuron. Examples of devices include solid or semi-solid devices such as controlled release biodegradable matrices, fibers, pumps, stents, adsorbable gelatin (e.g., Gelfoam), etc. The device may be loaded with premeasured, discrete and contained amounts of the compositions sufficient to promote sustained regeneration or sustained survival of the neuron. In one embodiment, the device provides continuous contact of the neuron with the compositions at nanomolar or micromolar concentrations, (e.g., for at least 2, 5, or 10 days, or for at least 2, 3, or 4 weeks, or for greater than 4 weeks, e.g., 5, 6, 7, or 8 weeks).

In any aspect or embodiment described herein, administration of a nucleic acid peptide and/or composition disclosed herein to a subject (e.g., in a single or in different pharmaceutical compositions, with or without other agents described herein) results in the compositions directly contacting an injured neuron in need of regeneration. In any aspect or embodiment described herein, administration results in contacting neurons proximal to a site of neuronal injury. Neurons can be contacted at any point along their length (e.g., at the axon, dendrite and/or the cell body).

Administration to the subject can be by any one or combination of a variety of methods (e.g., intravenously, intracortically, intracerebrally, intrathecally, intranasally, ocularly, parenterally, enterally and/or topically or locally at the injured neuron.). The appropriate method(s) will depend upon the circumstances of the individual (e.g., the location of the target neuron(s), the condition of the individual, the desired duration of the contact, whether local or systemic treatment is desired). The administration can be by any methods described herein that will result in contact of sufficient agent(s) to the target neuron to promote survival of neuron, axon regeneration, or a combination of both. For instance, parenteral, enteral, and topical administration can be used. The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, intracortical, intracerebral, intranasal, ocular, sub capsular, subarachnoid, intraspinal, intracerebro spinal, and intrasternal injection and infusion. Enteral administration involves the esophagus, stomach, and small and large intestines (i.e., the gastrointestinal tract). The phrases “systemic administration,” “administered systemically”, “peripheral administration” and “administered peripherally” as used herein mean the administration of a compound other than directly into the central nervous system, such that it enters the animal's system and, thus, is subject to metabolism and other like processes, for example, subcutaneous administration. Administration may be topical (including ophthalmic, vaginal, rectal, intranasal, epidermal, and transdermal), oral or pulmonary administration, e.g., by inhalation or insufflation, or intracranial, e.g., intrathecal or intraventricular, administration, topically to the eye, or by intraocular injection.

Specific routes of administration and the dosage regimen will be determined by skilled clinicians based on factors such as the exact nature of the condition being treated, the severity of the condition, and the age and general physical condition of the patient.

Provided herein are methods for promoting survival of neuron, axon regeneration, or a combination of both in an injured neuron of central nervous system following an injury. The method involves administering to a subject vitronectin, fibronectin, and/or the fibronectin-based peptide (also referenced herein as “an agent,” “the recombinant peptide,” or “therapeutic peptide”) of the present disclosure, or a composition of the present disclosure comprising the fibronectin-based peptide to thereby contact the site of injury, or a composition comprising fibronectin and nucleic acids as described herein. Another aspect of the present disclosure relates to a method for promoting axon regeneration (e.g., long-distance axon regeneration) or neuroprotection of axons (e.g., long-distance axons) in a subject, the method comprising administering to the subject vitronectin, fibronectin, and/or the fibronectin-based peptide (also referenced herein as “an agent,” “the recombinant peptide,” or “therapeutic peptide”) of the present disclosure, or the composition of the present disclosure comprising the fibronectin-based peptide of the present disclosure.

In any aspect or embodiment described herein, administration occurs following neuronal injury in the subject, not prior to or at the time of neuronal injury. In any aspect or embodiment described herein, the agent(s) (e.g., a fibronectin-based peptide, vitronectin, fibronectin, and/or nucleic acids described herein) and/or composition is administered into a subject intrathecally. As used herein, the term “intrathecal administration” is intended to include delivering a formulation directly into the cerebrospinal fluid of a subject, by techniques including lateral cerebroventricular injection through a burrhole or cisternal or lumbar puncture or the like. The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord.

Administration of an agent(s) (e.g., a fibronectin-based peptide, vitronectin, fibronectin, and/or nucleic acids described herein) or composition to any of the above-mentioned sites can be achieved by direct injection of the agent(s) formulation or by the use of infusion pumps. For injection, the agent(s) or formulation/compositions comprising the same can be formulated in liquid solutions, preferably in physiologically compatible buffers such as Hank's solution or Ringer's solution. In addition, the inhibitor(s) formulation may be formulated in solid form and re-dissolved or suspended immediately prior to use. Lyophilized forms are also included. The injection can be, for example, in the form of a bolus injection or continuous infusion (e.g., using infusion pumps) of the agent(s) formulation.

In any aspect or embodiment described herein, said agent(s) or formulation/composition comprising the same is administered by lateral cerebroventricular injection into the brain of a subject in the inclusive period from the time of the injury to a time determined by the skilled practitioner (e.g., 100 hours). The injection can be made, for example, through a burr hole made in the subject's skull. In another embodiment, said encapsulated therapeutic agent is administered through a surgically inserted shunt into the cerebral ventricle of a subject in the inclusive period from the time of the injury to a time determined by the skilled practitioner (e.g., 100 hours thereafter). For example, the injection can be made into the lateral ventricles, which are larger, even though injection into the third and fourth smaller ventricles can also be made.

In any aspect or embodiment described herein, said agent(s) or formulation/compositions comprising the same is administered by injection into the cisterna magna, or lumbar area of a subject in the inclusive period from the time of the injury to a time determined by the skilled practitioner (e.g., 100 hours thereafter). Administration can be continuous or can be by repeated doses. In any aspect or embodiment described herein, the repeated doses are formulated so that an effective amount of the agent(s) is continually present at the injury site.

Viral and non-viral-based gene transfer methods can be used to introduce nucleic acids to cells or target tissues of the subject. Such methods can be used to administer the nucleic acids compositions or molecules to cells in vitro. Alternatively, or in addition, such polynucleotides can be administered for in vivo or ex vivo gene therapy uses. Non-viral vector delivery systems include DNA plasmids, naked nucleic acid, and nucleic acid complexed with, for example, a liposome or other delivery vehicle. Viral vector delivery systems include both DNA and RNA viruses and can have either episomal or integrated genomes after delivery to the cell. For therapy (e.g., of neurological disorders which may be ameliorated by a specific gene product) a therapeutically effective amount or dose of the vector expressing the therapeutic shRNAs or ORFs described herein, or a combination thereof, and/or the fibronectin peptide described herein is administered to a subject in need of such treatment. The use of the vector disclosed herein in the manufacture of a medicament for providing therapy to a subject is within the scope of the present application.

Methods of non-viral delivery of nucleic acids of the invention include lipofection, microinjection, biolistics, virosomes, liposomes, immunoliposomes, polycation or lipid:nucleic acid conjugates, naked DNA, artificial virions, and agent-enhanced uptake of DNA. Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art.

RNA or DNA viral based systems can be used to target the delivery of polynucleotides carried by the virus to specific cells in the body and deliver the polynucleotides to the nucleus. Viral vectors can be administered directly to patients (in vivo) or they can be used to transfect cells in vitro. In some cases, the transfected cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene, and high transduction efficiencies.

In any aspect or embodiment described herein, other agents that might be useful in promoting neuroprotection and/or regeneration can be administered or combined with the agents described herein. Examples of other agents include an activator of protein translation (e.g., a mTOR pathway activator; a PTEN inhibitor; a TSCl/2 inhibitor; an Akt activator; a Ras/MEK pathway activator; or a PRAS40 inhibitor can promote the survival of, or axon regeneration in the neuron. PTEN inhibitors can be useful in the methods and compositions herein, in combination with the agents disclosed herein (e.g., shRNAs and/or ORFs). Inhibition of SOCS3 was reported to promote neuron regeneration (US20110124706 Al, the contents of which are incorporated by reference herein in its entirety). Other agents include nerve growth factor, trophic factor, or hormone that promotes neural cell survival, growth, and/or differentiation, such as brain-derived neurotrophic factor (BDNF), ciliary neurotrophic factor (CNTF), inosine, oncomodulin, NT-3, etc. Accordingly, in any aspect or embodiment described herein, the method further comprises contacting or administering to neurons (e.g., injured neurons) with other agents that can promote at least one of neuroprotection, survival of injured axons, or axon regeneration in injured neurons in combination with the agents disclosed herein (e.g., a fibronectin-based peptide, vitronectin, fibronectin, shRNAs, and/or ORFs).

In any aspect or embodiment described herein, the agents are administered in combination with one or more factors that facilitate neuronal synapse formation. Examples of such factors include, without limitation, cAMP modulator and/or an asogenic factor, such as AF-1, AF-2, a purine such as inosine, mannose, glucosese, or glucose-6-phosphate, TGF β, and oncomodulin, SOC3 inhibitors, polypeptide growth factors such as BDNF, NGF, NT-3, CNTR, LIF, and GDNF. activators of Rab3A, NMDA-I, synapsin-1, tetanus toxin receptor, BDNF-receptor and a GAB A receptor. Such factors are described in U.S. Patent Application Publication 2008/0214458. Neuronal synapse formation can be modulated, for example, by modulating the activity of the transcriptional factor myocyte enhancer factor 2 (MEF2) (e.g., MEF2A), MEF2C, MEF2D, dMEF2, CeMEF2, Activating transcription factor 6 beta (ATF6), Estrogen related receptor alpha (ERR1), Estrogen related receptor beta (ERR2), Estrogen related receptor gamma (ERR3), Erythroblastosis virus E26 oncogene homolog 1 (ETS1), Forkhead box protein C2 (FOXC2), Gata binding factor 1 (GATA-1), Heat shock factor 1 (HSF1), HSF4, MLL3, Myeloblastosis oncogene homolog (MYB), Nuclear receptor coactivator 2 (NCOA2), Nuclear receptor corepressor 1 (NCOR1), Peroxisome proliferative activated receptor gamma (PPARg), SMAD nuclear interacting protein 1 (SNIP1), SRY-box containing protein 3 (SOX3), SOX8, SOX9, Sterol regulatory element-binding transcription factor 2 (SREBP2), or Thyroid hormone receptor beta-1 (THRB1) (described in U.S. Patent Application Publication 20100112600).

The other agent(s) can be administered to the same site or to a different site as the agents of the present disclosure (e.g., a fibronectin-based peptide, vitronectin, fibronectin, shRNAs, and/or ORFs). The other agent may be contacted to the same site of the neuron or to a different site of the neuron. In any aspect or embodiment described herein, the agents of the present disclosure (e.g., a fibronectin-based peptide, vitronectin, fibronectin, shRNAs, and/or ORFs) is contacted to the neuron(s) at the neuron's region of origin in the brain (e.g., by administration to cortical neurons at the cerebral ventricle) and the other agent is contacted to the neuron at the site of injury (e.g., the lesioned axon such as a cortical spinal tract axon). Other combinations of site of contact and routes of administration discussed herein are also envisioned.

The contacting with one or more of the other agents (e.g., activator of protein translation, PTEN inhibitor, inhibitor or SOCS3, BDNF, CNTF, inosine, oncomodulin, NT-3) can occur prior to, with or after the contacting with the agents disclosed herein (e.g., a fibronectin-based peptide, vitronectin, fibronectin, shRNAs, and/or ORFs).

The respective other agents can be administered by the same route of administration or through different routes of administration e.g., orally, by intravenous (i.v.) bolus, by i.v. infusion, subcutaneously, intramuscularly, ocularly (e.g., intraocularly, periocularly, retrobulbarly, intravitreally, and/or subconjunctivally), topically, by subtenon administration, etc., intracranially, intraperitoneally, intraventricularly, intrathecally, by epidural, etc. The administration of the respective other agents can be for differing prolonged periods, or for the same length of period such that their activities on the contacted neurons completely or substantially overlap. The respective other agents can be administered in a formulation which contains one or more of other agents (a pharmaceutical composition, as described herein), or they can be in separate formulations (separate pharmaceutical compositions) for separate administration.

A “therapeutically effective amount” refers to an amount effective, at dosages and for periods of time necessary, to achieve the desired therapeutic result. A therapeutically effective amount of the shRNA and/or ORFs, or vectors thereof, may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In instances where human dosages for the vector have been established for at least some condition, those same dosages, or dosages that are between about 0.1% and 500%, more preferably between about 25% and 250% of the established human dosage can be used. Where no human dosage is established, as will be the case for newly discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

A therapeutically effective amount of the vector can be administered to a subject at various points of time. For example, the vector can be administered to the subject prior to, during, or after the subject has developed a disease or disorder. The vector can also be administered to the subject prior to, during, or after the occurrence of a disease or disorder.

The dosing frequency of the vector can vary. For example, in any aspect or embodiment described herein, the vector can be administered to the subject once in a lifetime. In any aspect or embodiment described herein, the dosing can be about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the vector is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

The agents disclosed herein (e.g., a fibronectin-based peptide, recombinant peptide, vitronectin, fibronectin, shRNAs, and/or ORFs) can be administered to a subject (e.g., a human) in a method of treating a neurological disease, for neuroprotection prior to injury, or for promoting regeneration of long-distance axons after injury. Thus, another aspect of the present disclosure relates to a method for promoting axon regeneration (e.g., long-distance axon regeneration) or neuroprotection of axons (e.g., long-distance axons) in a subject, the method comprising administering to the subject vitronectin, fibronectin, and/or the fibronectin-based peptide (also referenced herein as “an agent,” “the recombinant peptide,” or “therapeutic peptide”) of the present disclosure, or the composition of the present disclosure comprising the fibronectin-based peptide of the present disclosure. A further aspect of the present disclosure relates to a method of repairing neural connections or preventing damage to neural connections in white matter of the central nervous system (e.g., brain, spinal cord, visual system, etc.), the method comprising administering to or contacting neuronal cells vitronectin, fibronectin, and/or the fibronectin-based peptide (also referenced herein as “an agent,” “the recombinant peptide,” or “therapeutic peptide”) of the present disclosure, or the composition of the present disclosure comprising the fibronectin-based peptide of the present disclosure. An additional aspect of the present disclosure relates to a method of treating or preventing a disease or disorder associated with the disassociation of or damage to the neuronal connections in the white matter of the central nervous system (e.g., brain, spinal cord, visual system, etc.), the method comprising administering/contacting a neuron(s) and/or central nervous system (e.g., a subject) with vitronectin, fibronectin, and/or the fibronectin-based peptide (also referenced herein as “an agent,” “the recombinant peptide,” or “therapeutic peptide”) of the present disclosure, or the composition of the present disclosure comprising the fibronectin-based peptide of the present disclosure.

In any aspect or embodiment described herein, the subject has neuronal injury or a brain injury. In any aspect or embodiment described herein, the subject is at risk for neuron injury or a brain injury. In any aspect or embodiment described herein, administering the agent(s) (e.g., a fibronectin-based peptide, vitronectin, fibronectin, shRNAs, and/or ORFs) and/or composition comprising the agent(s) ameliorates the neurological disease, lengthens survival of neurons, and promotes long-distance axon regeneration (e.g., for at least 6 weeks). In any aspect or embodiment described herein, the subject has (i) a disease or disorder of the optic nerve, (ii) a disease or disorder of the spinal cord, or (iii) a combination thereof.

The methods, peptides, and compositions described herein are suited for the promotion of survival of, and/or axon regeneration in and sustained axonal outgrowth of CNS (central nervous system) neurons. It is contemplated herein that the methods, peptides, and compositions of the present disclosure are not limited to neurons of the CNS but can also be adapted for PNS (peripheral nervous system) neurons. CNS neurons include, without limitation, a cerebellar granule neuron, or an ocular neuron. In any aspect or embodiment described herein, the neuron is the optic nerve. In one embodiment, the neuron is a sensory neuron (e.g., dorsal root ganglion (DRG) sensory neuron). As used herein, the term “PNS neurons” is intended to include the neurons commonly understood as categorized in the peripheral nervous system, including sensory neurons and motor neurons. The present invention provides methods, peptides, and compositions for preventing and/or treating peripheral nerve damage (peripheral neuropathy) in a subject. Peripheral nerves, such as dorsal root ganglia, otherwise known as spinal ganglia, are known to extend down the spinal column. These nerves can be injured as a result of spinal injury. Such peripheral nerve damage associated with spinal cord injury can also benefit from neuron axonal outgrowth produced by the methods described herein. In any aspect or embodiment described herein, the neurons are in the spinal cord. In any aspect or embodiment described herein, the neurons are in the optic nerve. In any aspect or embodiment described herein, the neurons are axotomized neurons (for example during surgery). In any aspect or embodiment described herein, the neuron is human. In any aspect or embodiment described herein, the neuron is a terminally differentiated neuron. In any aspect or embodiment described herein, the neuron is an adult neuron (e.g., in a subject that has reached maturity, such as in humans older than 18 years). In any aspect or embodiment described herein, the neuron is non-embryonic. In any aspect or embodiment described herein, the neuron is in an immature organism (e.g., embryo, infant, child). All mammals are suitable subjects for performance of the methods described herein. In any aspect or embodiment described herein, the mammal is a human, non-human primate, companion animal (e.g., dog, cat), livestock animal (e.g., horse, cow, pig, sheep), or rodent (mouse, rat, rabbit). In any aspect or embodiment described herein, the subject is a non-human primate animal in a model for neurodegeneration or nervous system (CNS or PNS) injury. Neurons derived from said subjects are also suitable for performance of the methods described herein. In any aspect or embodiment described herein, the neurons are injured neurons.

Neuronal injury. As used in the art, the term injury refers to damage (e.g., to a system or a cell). Damage to a system is evidenced by aberrant function, reduction of function, loss of function of the system, or loss of essential components (e.g., specialized cells such as neurons).

Damage or injury to a specific neuron is also evidenced by aberrant function, loss of function, reduced function, and/or cell death. Some forms of injury to a neuron can be directly detected (e.g., by visualization as with a severed or crushed neuronal axon). Accordingly, in any aspect or embodiment described herein, the methods disclosed herein comprises an antecedent step of determining that the neuron is injured and/or has axotomy-induced stress. Neuronal injury can result from a variety of insults, including, but not limited to physical injury (e.g., severing, crushing), toxic effects, atrophy (e.g., due to lack of trophic factors), etc.

The term “neuronal injury” as used herein refers to any damage or dysfunction exhibited by neurons, including but not limited to loss of myelin, dendrite retraction, dendritic spine density reduction, axonal damage, loss of axon regeneration and neuronal death. Neuronal injury may be complete loss of a neuron, or loss of a part of the neuron (e.g., an axon). Such damage may result from acute or traumatic injury to the neuron (e.g., crush, severing) such as the result of external physical trauma to the subject (e.g., contusion, laceration, acute spinal cord injury, traumatic brain injury, cortical impact, etc.). Acute or traumatic injury to a neuron can also result from an acute condition, such as stroke, that results in acute ischemia to the neuron resulting in acute injury. The specific location of neuronal injury will vary with the specific cause of the injury, and the specific individual. In one embodiment, the injured CNS neuron is located in CNS white matter, particularly white matter that has been subjected to traumatic injury. The specific location of a lesion to a specific neuron will vary with respect to the injury. In one embodiment, the injury is in the axon or dendrite of a neuron. In on embodiment, the injured neuron is in the spinal cord. In one embodiment, the injured neuron is in the optic nerve.

Injury to a neuron may also be incurred from a chronic injury (e.g., repetitive stress injury) or condition (e.g., chronic inflammation or disease). Chronic injury leads to neurodegeneration such as caused by neurotoxicity or a neurological disease or disorder (e.g., Huntington's disease, Parkinson's disease, Alzheimer's disease, multiple system atrophy (MSA), etc.).

In any aspect or embodiment described herein, injured neurons results from an ocular injury or disorder (e.g. toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, eye damage from free radicals, etc.), or an optic neuropathy (e.g. ischemic optic neuropathies, toxic optic neuropathies, ocular ischemic syndrome, optic nerve inflammation, infection of the optic nerve, optic neuritis, optic neuropathy, papilledema, papillitis, retrobulbar neuritis, commotio retinae, glaucoma, macular degeneration, retinitis pigmentosa, retinal detachment, retinal tears or holes, diabetic retinopathy, iatrogenic retinopathy, optic nerve drusen, etc.). Injury to a neuron can be detected by the skilled practitioner through a variety of assays known in the art. Loss of function assays can be used to determine neuronal injury. Physical damage to the neuron (e.g., axonal crushing or severing) can sometimes be observed diagnostically through routine methods. One way to detect a lesion is through detection of axotomy-induced stress.

The methods, peptides, nucleic acids, and compositions disclosed herein are useful for the treatment of neuronal injury, which may be caused by a disease or disorder. In any aspect or embodiment described herein, the method prevents damage to neural connection or repairs neural connections (e.g., neural connections in white matter of the central version system (e.g., brain, spinal cord, visual system, etc.). The methods, nucleic acids, peptides, and compositions of the present disclosure are useful for treatment of diseases or disorders resulting from or leading to the neuronal injury described herein. In any aspect or embodiment described herein, the composition, the second composition, or a combination thereof is delivered with other drugs or agents used for neuroprotection or axon regeneration.

In any aspect or embodiment described herein, the method further comprises administering (e.g., simultaneously or co-administering with the vitronetin, fibronectin, fibronectin-based peptide, or a composition comprising one or more thereof): (i) a nucleic acid molecule comprising one or more synthetic small hairpin RNA (shRNA) molecules with one or more shRNA stem-loop regions, wherein the shRNA comprises one or more regions complementary to a portion of the target RNA, wherein hybridization of the complementary region of the shRNA to the target RNA of a target gene blocks target RNA function and promotes axon neuroprotection and/or regeneration; (ii) an expression cassette, the expression cassette comprising a synthetic nucleic acid open reading frame (ORF) molecule of a gene involved in axon regeneration, wherein the gene comprises Rpl7, Rpl7a, Tceb2, Lancl1, Atp6v0c, Dynlt1a, Mrtfa, Lars2, Dpysl5, Fblim1, Dypsl3, or Nfe213, wherein expression of the ORF promotes axon neuroprotection and/or regeneration; (iii) a protein product expressed from one or more of the open reading frames in (ii), wherein the protein product promotes axon neuroprotection and/or regeneration; or (iv) a combination thereof. In any aspect or embodiment described herein, a second composition comprises (i) the nucleic acid molecule, (ii) the expression cassette, (iii) the protein product, or (iv) the combination thereof, wherein the second composition is administered. In any aspect or embodiment described herein, the second composition further comprises a vehicle, wherein the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus.

In any aspect or embodiment described herein, the target RNA in is an mRNA or a small noncoding RNA (sncRNA). In any aspect or embodiment described herein, the target RNA in is an mRNA of target gene Mmp9, Rax, Crx, Pdnp, Prdm13, or ift20 mRNA and its lncRNA isoform ENSMUST00000128788.

In any aspect or embodiment described herein, the nucleic acid comprises at least 2 shRNA stem-loop regions, or at least 3 shRNA stem-loop regions, or at least 4 shRNA stem-loop regions.

In any aspect or embodiment described herein, the synthetic ORF for target gene Rpl7 comprises the Rpl7 sequence identified in SEQ ID NO: 2, the synthetic ORF for target gene Rpl7a comprises the Rpl7a sequence identified in SEQ ID NO: 1, ORF for target gene Tceb2 comprises the Tceb2 sequence identified in SEQ ID NO: 3, the synthetic ORF for target gene Lancl1 comprises the Lancl1 sequence identified in SEQ ID NO:17, the synthetic ORF for target gene Atp6v0c comprises the Atp6v0c sequence identified in SEQ ID NO: 16, the synthetic ORF for target gene Dynlt1a comprises the Dynlt1a sequence identified in SEQ ID NO: 15, the synthetic ORF for target gene Mrtfa comprises the Mrtfa sequence identified in SEQ ID NO:13, the synthetic ORF for target gene Lars2 comprises the Lars2 sequence identified in SEQ ID NO: 12, the synthetic ORF of target gene Dpys15 comprises the Dpysl5 sequence identified in SEQ ID NO: 5, the synthetic ORF for target gene Fblim1 comprises the Fblim1 sequence identified in SEQ ID NO: 6, ORF for target gene Dypsl3 comprises the Dypsl3 sequence identified in SEQ ID NO: 4, ORF of target gene Nfe213 comprises the Nfe213 sequence identified in SEQ ID NO:7. In any aspect or embodiment described herein, the target gene is Mmp9 and the shRNA sequence is identified in SEQ ID NO: 14, SEQ ID NO: 70, SEQ ID NO: 71, and SEQ ID NO: 72, the target gene is Rax and shRNA sequence is identified in SEQ ID NO: 11, SEQ ID NO: 67, SEQ ID NO: 68, and SEQ ID NO: 69, the target gene is Crx and the shRNA sequence is identified in SEQ ID NO:10, SEQ ID NO:64, SEQ ID NO: 65, and SEQ ID NO: 66, the target gene is Pdnp and the shRNA sequence is identified in SEQ ID NO: 8, SEQ ID NO: 58, SEQ ID NO: 59, and SEQ ID NO: 60, or the target gene is Prdm13 and the shRNA sequence is identified in SEQ ID NO: 9, SEQ ID NO: 61, SEQ ID NO: 62, and SEQ ID NO: 63.

In any aspect or embodiment described herein, the sncRNA is Piwi-interacting RNA (piRNA). For example, in any aspect or embodiment described herein, the piRNA is piR-16295. By way of further example, in any aspect or embodiment described herein, the piRNA is piR-16295 and the shRNA sequence is SEQ ID NO: 29. In any aspect or embodiment described herein, the shRNA molecule is expressed from a viral vector selected from the group consisting of retrovirus, lentivirus, adenovirus, herpesvirus, poxvirus, alpha virus, vaccinia virus, and adeno-associated (AAV) virus (e.g., the ORF is expressed from a viral vector selected from the group consisting of retrovirus, lentivirus, adenovirus, herpesvirus, poxvirus, alpha virus, vaccinia virus, and adeno-associated (AAV) virus).

In any aspect or embodiment described herein, the disorder is selected from the group consisting of glaucoma, stroke, head trauma, spinal injury, optic injury, ischemia, hypoxia, neurodegenerative disease, multiple sclerosis, infectious disease, cancer, and autoimmune disease. In any aspect or embodiment described herein, the subject has a disease or disorder comprising a neurodegenerative diseases, optic neuropathy, traumatic optic neurapathy, stroke or optic nerve damage from a stroke, spinal cord injury or traumatic spinal cord injury, glaucoma, head injury or head trauma, or a combination thereof. In any aspect or embodiment described herein, the subject has a neurodegenerative disease selected from the group consisting of sporadic Parkinson's disease, autosomal recessive early-onset Parkinson's disease, Alzheimer's disease, Friedreich ataxia, Lewy body disease, Spinal muscular atrophy, stroke, amyotrophic lateral sclerosis, Binswanger's disease, Huntington's Disease, Huntington's chorea, multiple sclerosis, myasthenia gravis, and Pick's disease, Alpers' disease, Batten disease, cerebro-oculo-facio-skeletal syndrome, corticobasal degeneration, Gerstmann-Straussler-Scheinker disease, kuru, Leigh Syndrome, Monomelic amyotrophy, Multiple system atrophy, neurodegeneration with brain iron accumulation, opsoclonus myoclonus, striatonigral degeneration, transmissible spongiform encephalopathies, neuromyelitis optica, glaucoma, and optic nerve diseases.

In some embodiments, the methods, peptides, and compositions described herein can be used specifically to treat damage associated with peripheral neuropathies including, but not limited to, the following: diabetic neuropathies, virus-associated neuropathies, including acquired immunodeficiency syndrome (AIDS) related neuropathy, infectious mononucleosis with polyneuritis, viral hepatitis with polyneuritis; Guillian-Barre syndrome; botulism-related neuropathy; toxic polyneuropathies including lead and alcohol-related neuropathies; nutritional neuropathies including subacute combined degeneration; angiopathic neuropathies including neuropathies associated with systemic lupus erythematosis; sarcoid-associated neuropathy; carcinomatous neuropathy; compression neuropathy (e.g. carpal tunnel syndrome) and hereditary neuropathies, such as Charcot-Marie-Tooth disease, peripheral nerve damage associated with spinal cord injury can also be treated with the present method. The subject is treated in accordance with the present method for CNS or peripheral nerve damage as the result injury, including those listed above. Subjects at risk for developing such CNS or damage are also so treated.

As used herein, the term “treatment” refers to an intervention made in response to a disease, disorder, or physiological condition manifested by a patient or subject, particularly a patient or subject suffering from one or more disease or disorders that causes neuronal injury, such as damaging neuronal connections in the central nervous system (e.g., brain, spinal cord, visual system, etc.) or peripheral nervous system. For example, in any aspect or embodiment described herein, a disease or disorder associated with the disassociation of or damage to the neuronal connections-such as, disassociation of or damage to the neuronal connection in the white matter of the central nervous system. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In any aspect or embodiment described herein, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. This can take place at primary, secondary and/or tertiary prevention levels, wherein: (a) primary prevention avoids the development of symptoms/disorder/condition; (b) secondary prevention activities are aimed at early stages of the condition/disorder/symptom treatment, thereby increasing opportunities for interventions to prevent progression of the condition/disorder/symptom and emergence of symptoms; and (c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The invention is further illustrated by the following non-limiting examples.

### EXAMPLES

### Animal Use, Surgeries, Cell Labeling and Isolation.

All animal studies were performed at the University of Connecticut Health Center with approval of the Institutional Animal Care and Use Committee and of the Institutional Biosafety Committee and performed in accordance with the ARVO Statement for the Use of Animals in Ophthalmic and Visual Research. Mice were housed in the animal facility with a 12-h light/12-h dark cycle (lights on from 7:00 AM to 7:00 PM) and a maximum of five adult mice per cage. The study used wild-type 129S1/SvImJ mice (The Jackson Laboratory). Optic nerve surgeries and injections, and intravitreal injections, were carried out on mice of both sexes 8-12 weeks of age (average body weight 20-26 g) under general anesthesia, as described previously. The viruses included AAV2 vectors expressing anti-Pten shRNA or scrambled shRNA control (both co-expressing mCherry reporter and utilizing published sequences), as well as Dynlt1a (ORF of ENSMUST00000169415.2), Lars2 (ORF of ENSMUST00000038863.8), and mCherry (titers ˜1×10¹² GC/mL; VectorBuilder, Inc.). Viruses (2 ml per eye) were injected intravitreally, avoiding injury to the lens, in 8-week-old mice, which were randomly assigned to experimental or control conditions, 2 weeks prior to ONC surgery. This lead time allowed for sufficient transduction and expression of the shRNAs and transgenes in RGCs at the time of ONC. Transduction efficiency was approximately 30%, which is similar to prior reports that also used AAV2 to target the RGCs.

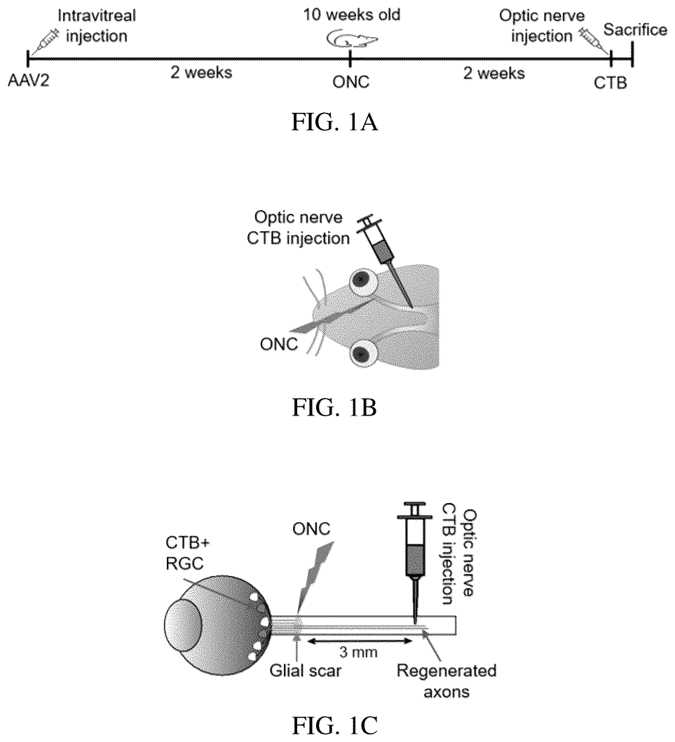

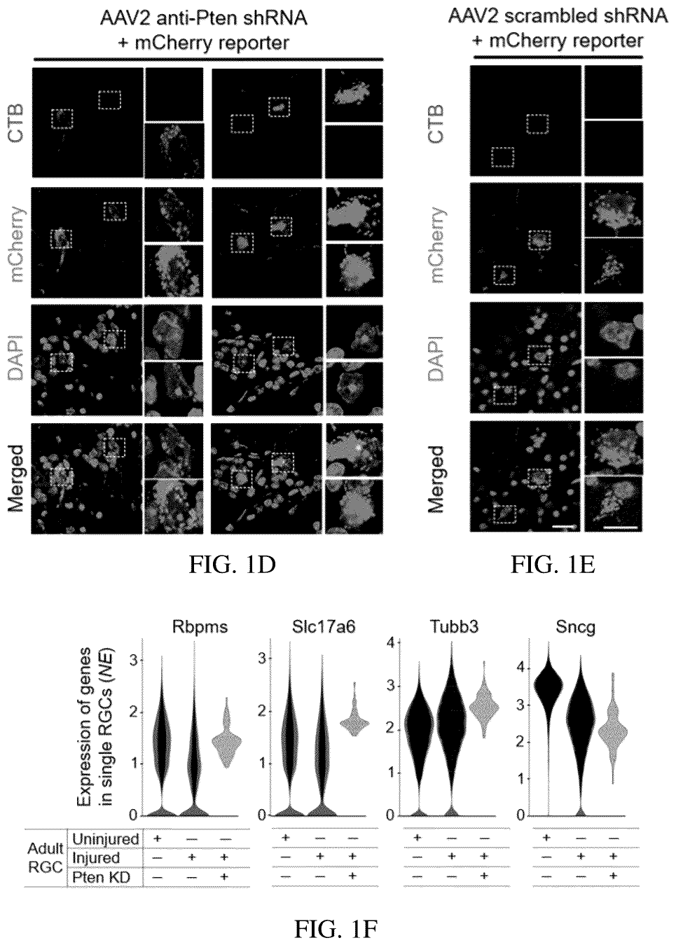

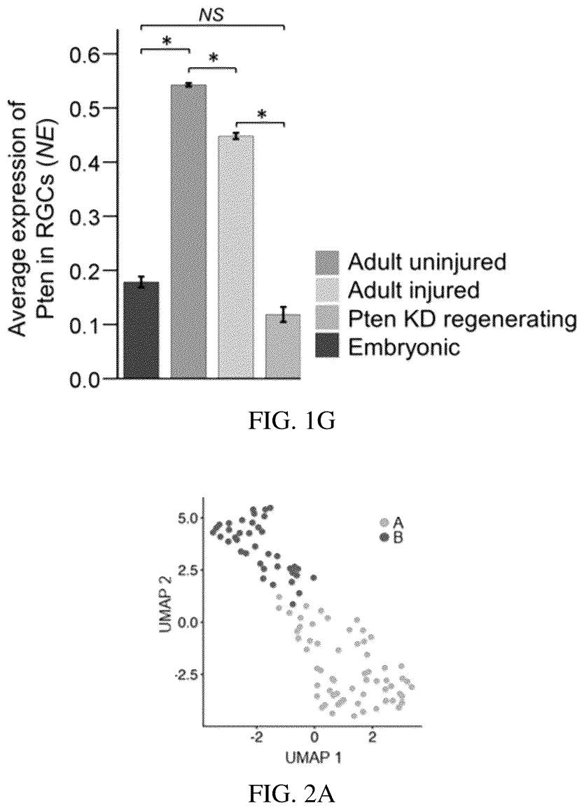

To label the RGCs which regenerated axons in response to treatment with anti-Pten shRNA, axonal tracer cholera toxin subunit B (CTB) conjugated to Alexa Fluor™ 488 dye (C34775, ThermoFisher Scientific) was injected (1% CTB in 1 ml PBS) into the optic nerve, approximately 3 mm from the ONC injury site, 12 hours prior to the enrichment of the RGCs from retinal single cell suspension by immunopanning (as we described previously) for FACS (using BD Biosciences FACSAria™ cell sorter with FACSDiva™ v8.0 software) at 2 weeks following ONC. No optic nerve-injected CTB was detected in the injured RGCs treated with scrambled shRNA control (FIG. 1). To inject CTB into the distal region of the optic nerve, the anesthetized mouse skull was exposed, drilled on the Bregma, and cut along the coronal suture line. Then, ˜5 mm width of the bone was removed laterally up to the cortical area, under which the optic nerve enters the optic chiasm. Parts of frontal cortex, striatum, thalamic nuclei, and nucleus of the stria terminalis of amygdala were removed with a surgical spatula to expose the optic nerves along the anterior cranial fossa (taking care to minimize damage to the blood vessels) just enough to enable visualization of the optic nerve segment that enters the optic chiasm. To inject CTB, using stereotaxic apparatus, a 50 mm dimeter glass needle (pulled from borosilicate glass capillaries Cat #BF150-86-10 using Flaming/Brown P-97 micropipette puller) was guided and inserted into the optic nerve segment adjacent to the optic chiasm. After visually-confirmed injection, coagulant was added to stop bleeding and the skin was sutured.

Standard stereotaxic surgery analgesic regimen was administered. Mice were kept warm using a heating-pad, food and water placed in a petri dish, and mice were checked regularly through sacrifice 12 hours later.

### Tissue Processing

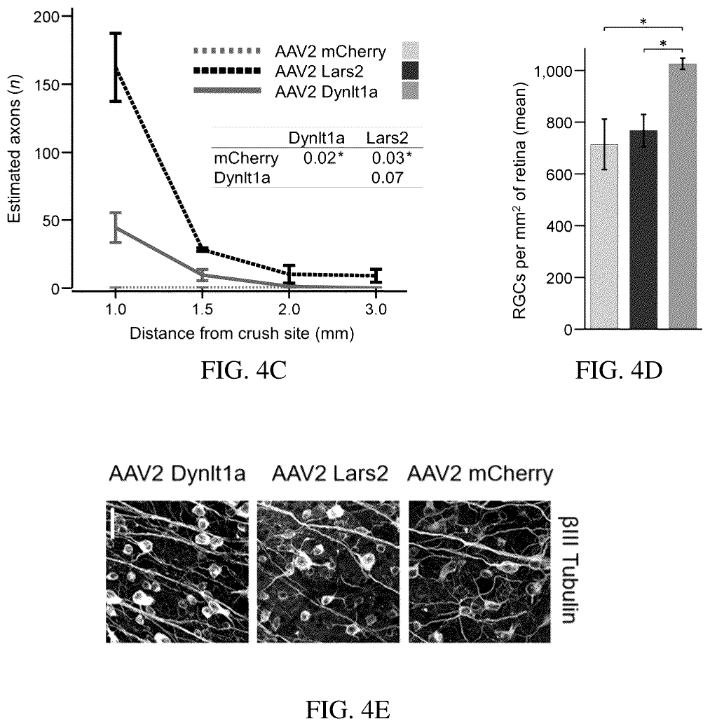

Standard histological procedures were used, as described previously. Briefly, anesthetized mice were transcardially perfused with isotonic saline followed by 4% paraformaldehyde (PFA) at 2 weeks after ONC, the eyes and optic nerves were dissected, postfixed 2 hours, the retinas were dissected out, and optic nerves were transferred to 30% sucrose overnight at 4° C. The optic nerves were then embedded in OCT Tissue Tek Medium (Sakura© Finetek), frozen, cryostat-sectioned longitudinally at 14 μm, and then mounted for imaging on coated glass slides. Free-floating retinas were immunostained in 24-well plate wells and, after making 4 symmetrical slits, flat-mounted on coated glass slides for imaging. For immunostaining, free-floating retinas were blocked with the appropriate sera, incubated overnight at 4° C. with primary anti-bIII-Tubulin (1:500, rabbit polyclonal; Abcam, Ab18207) antibody, then washed three times, incubated with fluorescent secondary antibody (1:500; Alexa Fluor™, ThermoFisher Scientific) 4 hours at room temperature, washed three times again, and mounted for imaging. Images of the regenerating axons in the optic nerve and surviving RGCs in FIG. 4 were acquired using fluorescent microscope (Zeiss, AxioObserver.Z1); retinal flatmount images of RGCs positive for CTB and/or mCherry in FIG. 1 were acquired using confocal microscope (Zeiss, Confocal LSM800).

### Quantification of Regenerated Axons and RGC Survival.

To visualize the regenerating axons or their absence after treating with the viral vectors expressing Dynlt1a, Lars2, or mCherry control, axonal tracer (Alexa Fluor™ 488-conjugated CTB 1% in 3 μl PBS) was intravitreally injected 1 day before animals were euthanized 2 weeks following ONC. Longitudinal sections of the optic nerve were examined for possible axon sparing. No spared axons were found in control, and no evidence of axon sparing was found in experimental conditions (i.e., at 2 weeks after injury, no axons were found at distal from the injury region of the optic nerve). Regenerated axons (defined as continuous fibers, which are absent in controls and are discernible from background puncta and artefactual structures) were counted manually using a fluorescent microscope (Zeiss, AxioObserver.Z1) in at least 4 longitudinal sections per optic nerve at 0.5 mm, 1 mm, 1.5 mm, 2 mm, and 3 mm distances from the injury site (identified by the abrupt disruption of the densely packed axons near the optic nerve head, as marked by a rhombus in FIG. 4B), and these values were used to estimate the total number of regenerating axons per nerve, as described. RGC survival was quantified in retinal flatmounts as previously described, by immunostaining with an antibody to anti-βIII-Tubulin (neuronal marker Tuj1) and counterstained with DAPI to label the nuclei, taking advantage of the selective expression of βIII-tubulin in RGCs. ImageJ software was used to count βIII-Tubulin positive cells from images taken at 1-2 mm from the optic nerve head in four directions, then averaged to estimate overall RGC survival per mm² of the retina. Investigators performing the surgeries and quantifications were masked to the group identity by another researcher until the end of the experiment.

Plate-Based Droplet scRNA-Seq of Pten KD Long-Distance Axon-Regenerating RGCS.

Custom designed Drop-Seq barcodes from Integrated DNA Technologies (IDT) were delivered into wells of two 384-well plates. All primers in one well shared the same unique cell barcode and billions of different unique molecular identifiers (UMIs). An Echo® 525 liquid handler was used to sequentially dispense lysis buffer, primers (custom designed Drop-Seq barcodes from Integrated DNA Technologies), and reaction reagents, totaling 1 ml into each well in the plate for the cell lysis and cDNA synthesis using a modified Drop-Seq/SmartSeq2 protocol. Following cDNA synthesis, the contents of each well were collected and pooled into one tube using a Caliper SciClone Liquid Handler. After treatment with exonuclease to remove unextended primers, the cDNA was PCR amplified for 13 cycles and the cDNA was fragmented and amplified for sequencing using a Nextera® XT DNA sample prep kit (Illumina®) using the following custom primers that enabled the specific amplification of only the 3′ ends:

| | [25 nt universal anchor (or PCR handle)--12 nt barcode--8 nt UMI--30 nt |
|---|---|
| Oligo dT for | poly-dT] |
| RT (ex. Well | AAGCAGTGGTATCAACGCAGAGTACaacaagtcgctgNNNNNNNNTTT |
| A1) | TTTTTTTTTTTTTTTTTTTTTTTTTTTVN TSO_RT 5′- (SEQ ID NO: 36) |
| TSO_RT | 5′-AAGCAGTGGTATCAACGCAGAGTGAATrGrG+G-3′ (SEQ ID |
| | NO: 37) |
| TSO_ISPCR_ | 5′-AAGCAGTGGTATCAACGCAGAGT-3′ (SEQ ID NO: 38) |
| cDNA | |
| P5-TSO- | AATGATACGGCGACCACCGAGATCTACACGCCTGTCCGCGGAAG |
| Hybrid_LibP | CAGTGGTATCAACGCAGAGT*A*C-3′ (SEQ ID NO: 39) |
| Custom | 5′-CGGAAGCAGTGGTATCAACGCAGAGTAC-3′ (SEQ ID NO: 40) |
| Read 1 | |
| Primer | |

P5 is the name of a standard sequence, which is adaptor that allows the library to bind and generate clusters on the flow cell for sequencing. Asterix denotes a phosphorothioate base modification in the primer.

Paired-end FASTQs were generated using BCL2FASTQ v2.18.0.12 (Illumina®). Digital expression matrix was constructed for each pair of FASTQs using Drop-seq tools v1.13 (http://mccarrolllab.com/dropseq) as follows: Bam creation with Picard (v2.9.3) FastqToSam; cell and UMI tagging, filtering, trimming with Drop-seq tools TagBamWithReadSequenceExtended, FilterBAM, TrimStartingSequence, PolyATrimmer; alignment with STAR (v2.5.4a) to the mm10-1.2.0_genome and transcriptome from CellRanger (for comparisons to 10× datasets); sorting with Picard (v2.9.3) SortSam; merging and tagging with Picard (v2.9.3) MergeBamAlignment and Dropseq_tools (v1.13) TagReadWithGeneExon; and gene-cell expression matrix of raw 3? end counts (in CSV format) was produced with Drop-seq_tools (v1.13) DigitalExpression. Cells not expressing RGC genes such as Rbpms and P3III-Tubulin, as well as poor quality cells or doublets, were excluded.

### Data Availability

The raw and processed datasets we generated for this study are available through the NCBI GEO accession GE210137 and Table 7.

Normalized expression (NE) levels of the differentially expressed genes (DEGs), which are differentially enriched or unenriched in the anti-Pten shRNA-treated (i.e., Pten KD) long-distance axon-regenerating regenerating RGCs compared to injured control RGCs (upregulated: log 2 FC≥1.5 and Pten KD RGC expression ≥0.5 NE; downregulated: log 2 FC≤−1.5 and ONC RGC expression ≥0.5 NE; p≤0.05; Mann-Whitney U test). Expression of these genes is also shown for embryonic and adult RGCs, as marked.

TABLE 7
GENES DIFFERENTIALLY ENRICHED OR UNENRICHED IN THE LONG-
DISTANCE AXON-REGENERATING RGCS AFTER TREATMENT WITH
ANTI-PTEN shRNAS.

| Gene ID | Gene | Embryonic | Atlas_C33_C40 | ONC_C30_C40 | Pten KD |
|---|---|---|---|---|---|
| ENSMUSG00000086026 | Gm12374 | 0.00 | 0.00 | 0.00 | 3.65 |
| ENSMUSG00000037738 | Nek5 | 0.00 | 0.00 | 0.00 | 1.43 |
| ENSMUSG00000033826 | Dnah8 | 0.00 | 0.01 | 0.00 | 1.26 |
| ENSMUSG00000001506 | Col1a1 | 0.00 | 0.00 | 0.00 | 0.97 |
| ENSMUSG00000029188 | Slc34a2 | 0.00 | 0.00 | 0.00 | 0.93 |
| ENSMUSG00000020713 | Gh | 0.00 | 0.00 | 0.00 | 0.89 |
| ENSMUSG00000024778 | Fas | 0.00 | 0.00 | 0.00 | 0.63 |
| ENSMUSG00000026712 | Mrc 1 | 0.00 | 0.00 | 0.00 | 0.56 |
| ENSMUSG00000105376 | Gm42487 | 0.00 | 0.00 | 0.00 | 0.51 |
| ENSMUSG00000029661 | Col1a2 | 0.00 | 0.00 | 0.00 | 1.19 |
| ENSMUSG00000075014 | Gm10800 | 0.00 | 0.00 | 0.00 | 1.40 |
| ENSMUSG00000032643 | Fhl3 | 0.00 | 0.00 | 0.00 | 0.89 |
| ENSMUSG00000038599 | Capn8 | 0.00 | 0.00 | 0.00 | 1.24 |
| ENSMUSG00000033060 | Lmo7 | 0.00 | 0.00 | 0.00 | 1.51 |
| ENSMUSG00000020122 | Egfr | 0.00 | 0.00 | 0.00 | 1.10 |
| ENSMUSG00000020286 | 1700093K21Rik | 0.00 | 0.01 | 0.01 | 1.97 |
| ENSMUSG00000067149 | Jchain | 0.00 | 0.00 | 0.00 | 0.63 |
| ENSMUSG00000094685 | Gm5900 | 0.00 | 0.00 | 0.00 | 0.51 |
| ENSMUSG00000020143 | Dock2 | 0.00 | 0.00 | 0.00 | 0.60 |
| ENSMUSG00000086324 | Gm15564 | 0.00 | 0.00 | 0.01 | 1.21 |
| ENSMUSG00000091732 | Gm17541 | 0.00 | 0.00 | 0.00 | 0.53 |
| ENSMUSG00000097242 | Gm16907 | 0.00 | 0.00 | 0.00 | 0.66 |
| ENSMUSG00000067158 | Col4a4 | 0.00 | 0.00 | 0.01 | 1.38 |
| ENSMUSG00000002769 | Gnmt | 0.00 | 0.00 | 0.01 | 0.80 |
| ENSMUSG00000050122 | Vwa3b | 0.00 | 0.00 | 0.01 | 0.79 |
| ENSMUSG00000042010 | Acacb | 0.01 | 0.01 | 0.01 | 0.89 |
| ENSMUSG00000068706 | Gm10250 | 0.0 | 0.12 | 0.01 | 0.89 |
| ENSMUSG00000097312 | Gm26870 | 0.01 | 0.00 | 0.01 | 0.52 |
| ENSMUSG00000084849 | Gm16105 | 0.12 | 0.01 | 0.01 | 1.00 |
| ENSMUSG00000039883 | Lrrc17 | 0.00 | 0.01 | 0.01 | 0.65 |
| ENSMUSG00000020325 | Fstl3 | 0.01 | 0.02 | 0.01 | 0.88 |
| ENSMUSG00000000632 | Sez6 | 0.01 | 0.08 | 0.01 | 1.01 |
| ENSMUSG00000078612 | 1700024P16Rik | 0.00 | 0.00 | 0.01 | 1.11 |
| ENSMUSG00000029563 | Foxp2 | 0.01 | 0.05 | 0.01 | 0.53 |
| ENSMUSG00000043091 | Tuba1c | 0.01 | 0.01 | 0.01 | 0.56 |
| ENSMUSG00000031491 | Chrna6 | 0.00 | 0.31 | 0.01 | 0.57 |
| ENSMUSG00000059040 | Eno1b | 0.05 | 0.04 | 0.02 | 1.04 |
| ENSMUSG00000031492 | Chrnb3 | 0.04 | 0.17 | 0.01 | 0.52 |
| ENSMUSG00000084947 | Gm15594 | 0.01 | 0.01 | 0.01 | 0.53 |
| ENSMUSG00000091449 | Gm10269 | 0.02 | 0.02 | 0.01 | 0.53 |
| ENSMUSG00000020472 | Zkscan17 | 0.02 | 0.01 | 0.01 | 0.51 |
| ENSMUSG00000035202 | Lars2 | 0.04 | 0.05 | 0.05 | 2.24 |
| ENSMUSG00000097201 | Gm26700 | 0.00 | 0.00 | 0.02 | 0.84 |
| ENSMUSG00000078765 | U2af1l4 | 0.05 | 0.00 | 0.02 | 0.64 |
| ENSMUSG00000024687 | Osbp | 0.03 | 0.05 | 0.02 | 0.61 |
| ENSMUSG00000033653 | Vps8 | 0.09 | 0.08 | 0.09 | 2.49 |
| ENSMUSG00000050761 | Gp1bb | 0.01 | 0.04 | 0.03 | 0.79 |
| ENSMUSG00000022994 | Adcy6 | 0.02 | 0.03 | 0.02 | 0.53 |
| ENSMUSG00000028211 | Trp53inp1 | 0.09 | 0.03 | 0.03 | 0.66 |
| ENSMUSG00000022952 | Runx1 | 0.01 | 0.00 | 0.02 | 0.62 |
| ENSMUSG00000092074 | Dynlt1a | 0.40 | 0.02 | 0.02 | 0.55 |
| ENSMUSG00000028582 | Cc2d1b | 0.03 | 0.05 | 0.0 | 0.72 |
| ENSMUSG00000031523 | Dlc1 | 0.05 | 0.06 | 0.02 | 0.52 |
| ENSMUSG00000050359 | Sprr1a | 0.00 | 0.00 | 0.10 | 2.14 |
| ENSMUSG00000027364 | Usp50 | 0.21 | 0.04 | 0.03 | 0.59 |
| ENSMUSG00000099839 | Gm29374 | 0.00 | 0.00 | 0.05 | 0.95 |
| ENSMUSG00000039405 | Prss23 | 0.01 | 0.00 | 0.03 | 0.59 |
| ENSMUSG00000004110 | Cacna1e | 0.02 | 0.06 | 0.03 | 0.65 |
| ENSMUSG00000025821 | Zfp282 | 0.06 | 0.03 | 0.03 | 0.56 |
| ENSMUSG00000036907 | C1ql2 | 0.00 | 0.06 | 0.05 | 0.91 |
| ENSMUSG00000050737 | Ptges | 0.00 | 0.01 | 0.03 | 0.51 |
| ENSMUSG00000031558 | Slit2 | 0.24 | 0.06 | 0.04 | 0.66 |
| ENSMUSG00000032010 | Usp2 | 0.01 | 0.09 | 0.04 | 0.63 |
| ENSMUSG00000021704 | Mtx3 | 0.02 | 0.06 | 0.04 | 0.63 |
| ENSMUSG00000004885 | Crabp2 | 0.47 | 0.01 | 0.04 | 0.60 |
| ENSMUSG00000031503 | Col4a2 | 0.03 | 0.01 | 0.03 | 0.55 |
| ENSMUSG00000097234 | Gm26518 | 0.18 | 0.01 | 0.03 | 0.55 |
| ENSMUSG00000060019 | Gm10073 | 0.03 | 0.13 | 0.08 | 1.28 |
| ENSMUSG00000037989 | Wnk2 | 0.13 | 0.13 | 0.04 | 0.64 |
| ENSMUSG00000059775 | Rps26-ps1 | 0.05 | 0.06 | 0.04 | 0.57 |
| ENSMUSG00000000560 | Gabra2 | 0.09 | 0.13 | 0.06 | 0.94 |
| ENSMUSG00000045062 | Pcdhb7 | 0.06 | 0.06 | 0.04 | 0.56 |
| ENSMUSG00000062456 | Rp19-ps6 | 0.03 | 0.13 | 0.06 | 0.95 |
| ENSMUSG00000037593 | BC030499 | 0.00 | 0.15 | 0.04 | 0.52 |
| ENSMUSG00000105361 | AY036118 | 0.19 | 0.07 | 0.35 | 4.80 |
| ENSMUSG00000062070 | Pgk1 | 0.42 | 0.22 | 0.12 | 1.59 |
| ENSMUSG00000008226 | Scrn3 | 0.02 | 0.08 | 0.05 | 0.68 |
| ENSMUSG00000018593 | Sparc | 0.03 | 0.03 | 0.07 | 0.89 |
| ENSMUSG00000038368 | Focad | 0.01 | 0.03 | 0.04 | 0.51 |
| ENSMUSG00000103034 | Gm8797 | 0.11 | 0.08 | 0.08 | 1.03 |
| ENSMUSG00000054115 | Skp2 | 0.08 | 0.03 | 0.04 | 0.54 |
| ENSMUSG00000054889 | Dsp | 0.00 | 0.00 | 0.05 | 0.69 |
| ENSMUSG00000067369 | Trmt2b | 0.02 | 0.08 | 0.04 | 0.52 |
| ENSMUSG00000068699 | Flnc | 0.01 | 0.00 | 0.07 | 0.88 |
| ENSMUSG00000020790 | Ankfy1 | 0.11 | 0.06 | 0.08 | 0.94 |
| ENSMUSG00000045690 | Wdr89 | 0.10 | 0.08 | 0.10 | 1.26 |
| ENSMUSG00000022816 | Fstl1 | 0.20 | 0.10 | 0.05 | 0.59 |
| ENSMUSG00000042834 | Nrep | 1.02 | 0.17 | 0.09 | 1.01 |
| ENSMUSG00000056055 | Sag | 0.14 | 0.02 | 0.05 | 0.58 |
| ENSMUSG00000035967 | Ddx26b | 0.05 | 0.06 | 0.08 | 0.88 |
| ENSMUSG00000012609 | Ttll5 | 0.04 | 0.10 | 0.08 | 0.91 |
| ENSMUSG00000024867 | Pip5k1b | 0.01 | 0.04 | 0.06 | 0.65 |
| ENSMUSG00000005089 | Slc1a2 | 0.06 | 0.06 | 0.05 | 0.61 |
| ENSMUSG00000074264 | Amy1 | 0.01 | 0.04 | 0.04 | 0.50 |
| ENSMUSG00000022749 | Tbc1d23 | 0.08 | 0.07 | 0.06 | 0.63 |
| ENSMUSG00000024513 | Mbd2 | 0.06 | 0.12 | 0.05 | 0.58 |
| ENSMUSG00000042581 | Thsd7b | 0.32 | 0.22 | 0.24 | 2.61 |
| ENSMUSG00000017210 | Med24 | 0.15 | 0.08 | 0.06 | 0.61 |
| ENSMUSG00000040560 | Wdr7 | 0.10 | 0.21 | 0.11 | 1.18 |
| ENSMUSG00000020256 | Aldh1l2 | 0.03 | 0.01 | 0.05 | 0.58 |
| ENSMUSG00000004031 | Brinp2 | 0.18 | 0.05 | 0.07 | 0.69 |
| ENSMUSG00000094910 | D430019H16Rik | 0.14 | 0.04 | 0.07 | 0.69 |
| ENSMUSG00000028456 | Unc13b | 0.15 | 0.08 | 0.05 | 0.53 |
| ENSMUSG00000026575 | Nme7 | 0.11 | 0.06 | 0.08 | 0.84 |
| ENSMUSG00000026249 | Serpine2 | 0.09 | 0.13 | 0.06 | 0.57 |
| ENSMUSG00000055745 | Ldoc1l | 0.08 | 0.13 | 0.07 | 0.69 |
| ENSMUSG00000063873 | Slc24a3 | 0.08 | 0.18 | 0.10 | 1.05 |
| ENSMUSG00000005268 | Prlr | 0.00 | 0.16 | 0.05 | 0.50 |
| ENSMUSG00000042272 | Sestd1 | 0.14 | 0.07 | 0.08 | 0.81 |
| ENSMUSG00000051159 | Cited1 | 0.01 | 0.02 | 0.05 | 0.53 |
| ENSMUSG00000005087 | Cd44 | 0.01 | 0.00 | 0.05 | 0.53 |
| ENSMUSG00000034908 | Sidt2 | 0.03 | 0.05 | 0.07 | 0.64 |
| ENSMUSG00000078193 | Gm2000 | 0.28 | 0.06 | 0.13 | 1.27 |
| ENSMUSG00000046139 | Patl1 | 0.08 | 0.04 | 0.05 | 0.51 |
| ENSMUSG00000036155 | Mgat5 | 0.10 | 0.14 | 0.08 | 0.80 |
| ENSMUSG00000020883 | Fbx120 | 0.05 | 0.09 | 0.07 | 0.65 |
| ENSMUSG00000037999 | Arap2 | 0.00 | 0.09 | 0.06 | 0.52 |
| ENSMUSG00000048486 | Fitm2 | 0.02 | 0.08 | 0.06 | 0.58 |
| ENSMUSG00000063696 | Gm8730 | 0.11 | 0.06 | 0.07 | 0.66 |
| ENSMUSG00000050299 | Gm9843 | 0.10 | 0.34 | 0.14 | 1.33 |
| ENSMUSG00000033350 | Chst2 | 0.06 | 0.06 | 0.08 | 0.69 |
| ENSMUSG00000047496 | Rnf152 | 0.01 | 0.10 | 0.06 | 0.57 |
| ENSMUSG00000018417 | Myo1b | 0.36 | 0.28 | 0.09 | 0.82 |
| ENSMUSG00000061589 | Dot1l | 0.07 | 0.09 | 0.11 | 1.04 |
| ENSMUSG00000039714 | Cplx3 | 0.01 | 0.15 | 0.06 | 0.53 |
| ENSMUSG00000058006 | Mdn1 | 0.11 | 0.09 | 0.08 | 0.68 |
| ENSMUSG00000056268 | Dennd1b | 0.06 | 0.07 | 0.06 | 0.54 |
| ENSMUSG00000024908 | Ppp6r3 | 0.09 | 0.10 | 0.09 | 0.78 |
| ENSMUSG00000038375 | Trp53inp2 | 0.12 | 0.10 | 0.06 | 0.53 |
| ENSMUSG00000054843 | Atrnl1 | 0.12 | 0.14 | 0.07 | 0.62 |
| ENSMUSG00000023961 | Enpp4 | 0.03 | 0.05 | 0.07 | 0.66 |
| ENSMUSG00000049313 | Sorl1 | 0.00 | 0.30 | 0.11 | 0.94 |
| ENSMUSG00000028223 | Decr1 | 0.12 | 0.1 | 0.08 | 0.67 |
| ENSMUSG00000030110 | Ret | 0.13 | 0.07 | 0.10 | 0.86 |
| ENSMUSG00000032060 | Cryab | 0.00 | 0.00 | 0.07 | 0.64 |
| ENSMUSG00000040111 | Gramd1b | 0.04 | 0.17 | 0.12 | 1.04 |
| ENSMUSG00000021536 | Adcy2 | 0.01 | 0.20 | 0.14 | 1.20 |
| ENSMUSG00000042524 | Sun2 | 0.06 | 0.03 | 0.07 | 0.60 |
| ENSMUSG00000017376 | Nlk | 0.27 | 0.16 | 0.10 | 0.86 |
| ENSMUSG00000020448 | Rnf185 | 0.11 | 0.09 | 0.13 | 1.09 |
| ENSMUSG00000026659 | Dusp12 | 0.04 | 0.11 | 0.09 | 0.78 |
| ENSMUSG00000021745 | Ptprg | 0.22 | 0.12 | 0.07 | 0.53 |
| ENSMUSG00000068663 | Clec16a | 0.03 | 0.07 | 0.07 | 0.57 |
| ENSMUSG00000033781 | Asb13 | 0.15 | 0.13 | 0.12 | 0.95 |
| ENSMUSG00000071064 | Zfp827 | 0.06 | 0.09 | 0.08 | 0.62 |
| ENSMUSG00000023927 | Satb1 | 0.19 | 0.19 | 0.13 | 1.06 |
| ENSMUSG00000033147 | Slc22a15 | 0.08 | 0.12 | 0.10 | 0.83 |
| ENSMUSG00000044072 | Eml6 | 0.03 | 0.03 | 0.07 | 0.59 |
| ENSMUSG00000034295 | Fhod3 | 0.01 | 0.16 | 0.10 | 0.83 |
| ENSMUSG00000037851 | Iars | 0.18 | 0.14 | 0.12 | 0.90 |
| ENSMUSG00000062590 | Armc9 | 0.09 | 0.10 | 0.10 | 0.78 |
| ENSMUSG00000064210 | Ano6 | 0.13 | 0.04 | 0.07 | 0.54 |
| ENSMUSG00000050335 | Lgals3 | 0.00 | 0.00 | 0.15 | 1.18 |
| ENSMUSG00000023971 | Rrp36 | 0.09 | 0.15 | 0.10 | 0.74 |
| ENSMUSG00000037426 | Depdc5 | 0.05 | 0.09 | 0.07 | 0.52 |
| ENSMUSG00000031441 | Atp11a | 0.02 | 0.11 | 0.12 | 0.90 |
| ENSMUSG00000024952 | Rps6ka4 | 0.00 | 0.15 | 0.09 | 0.68 |
| ENSMUSG00000031993 | Snx19 | 0.05 | 0.12 | 0.09 | 0.65 |
| ENSMUSG00000029484 | Anxa3 | 0.02 | 0.02 | 0.14 | 1.07 |
| ENSMUSG00000029212 | Gabrb1 | 0.01 | 0.16 | 0.07 | 0.55 |
| ENSMUSG00000020220 | Vps13d | 0.06 | 0.12 | 0.10 | 0.75 |
| ENSMUSG00000020431 | Adcy1 | 0.09 | 0.26 | 0.10 | 0.76 |
| ENSMUSG00000057101 | Zfp180 | 0.06 | 0.07 | 0.09 | 0.71 |
| ENSMUSG00000029513 | Prkab1 | 0.05 | 0.08 | 0.07 | 0.52 |
| ENSMUSG00000023951 | Vegfa | 0.02 | 0.06 | 0.08 | 0.64 |
| ENSMUSG00000033365 | Ipo13 | 0.09 | 0.09 | 0.07 | 0.55 |
| ENSMUSG00000026944 | Abca2 | 0.03 | 0.20 | 0.10 | 0.78 |
| ENSMUSG00000025153 | Fasn | 0.34 | 0.32 | 0.14 | 1.01 |
| ENSMUSG00000038205 | Prkab2 | 0.05 | 0.11 | 0.09 | 0.69 |
| ENSMUSG00000034761 | Map4k5 | 0.05 | 0.11 | 0.10 | 0.77 |
| ENSMUSG00000025198 | Erlin1 | 0.03 | 0.05 | 0.09 | 0.63 |
| ENSMUSG00000052105 | Mtcl1 | 0.11 | 0.06 | 0.09 | 0.66 |
| ENSMUSG00000000915 | Hip1r | 0.06 | 0.07 | 0.07 | 0.53 |
| ENSMUSG00000033066 | Gas7 | 0.03 | 0.04 | 0.07 | 0.52 |
| ENSMUSG00000045573 | Penk | 0.01 | 0.09 | 0.07 | 0.52 |
| ENSMUSG00000022964 | Tmem50b | 0.11 | 0.30 | 0.18 | 1.29 |
| ENSMUSG00000061762 | Tac1 | 0.17 | 0.16 | 0.24 | 1.70 |
| ENSMUSG00000022797 | Tfrc | 0.16 | 0.12 | 0.09 | 0.63 |
| ENSMUSG00000040896 | Kcnd3 | 0.09 | 0.18 | 0.09 | 0.66 |
| ENSMUSG00000020628 | Trappc12 | 0.08 | 0.14 | 0.12 | 0.84 |
| ENSMUSG00000006494 | Pdk1 | 0.03 | 0.14 | 0.09 | 0.62 |
| ENSMUSG00000026276 | 2-Sep | 0.42 | 0.10 | 0.09 | 0.66 |
| ENSMUSG00000062234 | Gak | 0.08 | 0.12 | 0.14 | 0.98 |
| ENSMUSG00000021730 | Hcn1 | 0.01 | 0.12 | 0.12 | 0.84 |
| ENSMUSG00000059669 | Taf1b | 0.12 | 0.10 | 0.11 | 0.77 |
| ENSMUSG00000033075 | Senp1 | 0.15 | 0.12 | 0.08 | 0.52 |
| ENSMUSG00000021824 | Ap3m1 | 0.26 | 0.14 | 0.09 | 0.63 |
| ENSMUSG00000044465 | Fam160a2 | 0.05 | 0.14 | 0.14 | 0.92 |
| ENSMUSG00000029312 | Klhl8 | 0.04 | 0.08 | 0.08 | 0.52 |
| ENSMUSG00000033054 | Npat | 0.09 | 0.06 | 0.08 | 0.53 |
| ENSMUSG00000026618 | Iars2 | 0.03 | 0.13 | 0.10 | 0.67 |
| ENSMUSG00000061374 | Fiz1 | 0.23 | 0.11 | 0.10 | 0.69 |
| ENSMUSG00000045665 | Mfsd5 | 0.05 | 0.07 | 0.09 | 0.61 |
| ENSMUSG00000032594 | Ip6k1 | 0.08 | 0.22 | 0.22 | 1.45 |
| ENSMUSG00000038602 | Slc35f1 | 0.08 | 0.22 | 0.11 | 0.70 |
| ENSMUSG00000042211 | Fbxo38 | 0.10 | 0.08 | 0.08 | 0.54 |
| ENSMUSG00000043531 | Sorcs1 | 0.01 | 0.09 | 0.08 | 0.52 |
| ENSMUSG00000033174 | Mgll | 0.02 | 0.63 | 0.08 | 0.51 |
| ENSMUSG00000020740 | Gga3 | 0.04 | 0.10 | 0.09 | 0.58 |
| ENSMUSG00000027977 | Ndst3 | 0.07 | 0.09 | 0.11 | 0.68 |
| ENSMUSG00000109336 | Samd4b | 0.13 | 0.13 | 0.12 | 0.74 |
| ENSMUSG00000019790 | Stxbp5 | 0.03 | 0.15 | 0.15 | 0.94 |
| ENSMUSG00000062949 | Atp11c | 0.12 | 0.02 | 0.08 | 0.53 |
| ENSMUSG00000021819 | Zswim8 | 0.09 | 0.13 | 0.08 | 0.52 |
| ENSMUSG00000025558 | Dock9 | 0.01 | 0.11 | 0.10 | 0.66 |
| ENSMUSG00000035007 | Rundc1 | 0.01 | 0.06 | 0.10 | 0.62 |
| ENSMUSG00000041879 | Ipo9 | 0.13 | 0.16 | 0.12 | 0.77 |
| ENSMUSG00000053693 | Mast1 | 0.10 | 0.17 | 0.10 | 0.62 |
| ENSMUSG00000050910 | Cdr2l | 0.06 | 0.05 | 0.10 | 0.62 |
| ENSMUSG00000040797 | Iqsec3 | 0.03 | 0.34 | 0.25 | 1.58 |
| ENSMUSG00000049100 | Pcdh10 | 0.07 | 0.22 | 0.19 | 1.15 |
| ENSMUSG00000042429 | Adora 1 | 0.00 | 0.34 | 0.15 | 0.92 |
| ENSMUSG00000027397 | Slc20a1 | 0.18 | 0.20 | 0.17 | 1.02 |
| ENSMUSG00000018796 | Acsl1 | 0.03 | 0.08 | 0.10 | 0.58 |
| ENSMUSG00000025085 | Ablim1 | 0.45 | 0.34 | 0.21 | 1.29 |
| ENSMUSG00000040463 | Mybbp1a | 0.19 | 0.05 | 0.08 | 0.50 |
| ENSMUSG00000038489 | Polr21 | 0.52 | 0.16 | 0.19 | 1.16 |
| ENSMUSG00000010277 | 2610507B11Rik | 0.07 | 0.21 | 0.20 | 1.22 |
| ENSMUSG00000025060 | Slk | 0.06 | 0.08 | 0.10 | 0.58 |
| ENSMUSG00000034850 | Tmem127 | 0.06 | 0.17 | 0.09 | 0.54 |
| ENSMUSG00000021893 | Capn7 | 0.06 | 0.09 | 0.10 | 0.59 |
| ENSMUSG00000038023 | Atp6v0a2 | 0.04 | 0.10 | 0.09 | 0.56 |
| ENSMUSG00000036882 | Arhgap33 | 0.24 | 0.07 | 0.09 | 0.57 |
| ENSMUSG00000029821 | Dfna5 | 0.24 | 0.15 | 0.23 | 1.39 |
| ENSMUSG00000029313 | Aff1 | 0.03 | 0.05 | 0.09 | 0.51 |
| ENSMUSG00000029178 | Klf3 | 0.08 | 0.10 | 0.11 | 0.67 |
| ENSMUSG00000009647 | Mcu | 0.05 | 0.10 | 0.11 | 0.63 |
| ENSMUSG00000053581 | Zfand2a | 0.06 | 0.14 | 0.14 | 0.82 |
| ENSMUSG00000005505 | Kbtbd4 | 0.18 | 0.13 | 0.09 | 0.51 |
| ENSMUSG00000025817 | Nudt5 | 0.32 | 0.13 | 0.10 | 0.58 |
| ENSMUSG00000027313 | Chac1 | 0.01 | 0.09 | 0.21 | 1.26 |
| ENSMUSG00000064145 | Arih2 | 0.04 | 0.08 | 0.14 | 0.81 |
| ENSMUSG00000025475 | Adgra1 | 0.06 | 0.34 | 0.18 | 1.05 |
| ENSMUSG00000040724 | Kcna2 | 0.05 | 0.38 | 0.18 | 1.05 |
| ENSMUSG00000027016 | Zfp385b | 0.01 | 0.04 | 0.09 | 0.50 |
| ENSMUSG00000023032 | Slc4a8 | 0.08 | 0.13 | 0.18 | 1.05 |
| ENSMUSG00000042744 | Gm15800 | 0.27 | 0.31 | 0.21 | 1.22 |
| ENSMUSG00000029016 | Clcn6 | 0.11 | 0.26 | 0.16 | 0.91 |
| ENSMUSG00000022390 | Zc3h7b | 0.17 | 0.15 | 0.12 | 0.66 |
| ENSMUSG00000025531 | Chm | 0.15 | 0.10 | 0.10 | 0.59 |
| ENSMUSG00000032350 | Gclc | 0.05 | 0.12 | 0.12 | 0.65 |
| ENSMUSG00000073557 | Ppp1r12b | 0.05 | 0.13 | 0.15 | 0.84 |
| ENSMUSG00000032329 | Hmg20a | 0.13 | 0.13 | 0.12 | 0.65 |
| ENSMUSG00000035314 | Gdpd5 | 0.09 | 0.04 | 0.11 | 0.59 |
| ENSMUSG00000048349 | Pou4f1 | 1.05 | 0.39 | 0.19 | 1.04 |
| ENSMUSG00000032131 | Abcg4 | 0.02 | 0.10 | 0.09 | 0.51 |
| ENSMUSG00000028780 | Sema3c | 0.00 | 0.01 | 0.15 | 0.84 |
| ENSMUSG00000073643 | Wdfy1 | 0.06 | 0.10 | 0.15 | 0.83 |
| ENSMUSG00000036585 | Fgf1 | 0.00 | 0.27 | 0.16 | 0.85 |
| ENSMUSG00000023912 | Slc25a27 | 0.09 | 0.12 | 0.09 | 0.52 |
| ENSMUSG00000069372 | Ctxn3 | 0.02 | 0.45 | 0.09 | 0.51 |
| ENSMUSG00000024271 | Elp2 | 0.43 | 0.16 | 0.14 | 0.75 |
| ENSMUSG00000021495 | Fam193b | 0.06 | 0.08 | 0.10 | 0.56 |
| ENSMUSG00000049106 | Dcaf5 | 0.09 | 0.18 | 0.13 | 0.69 |
| ENSMUSG00000007411 | Mark3 | 0.19 | 0.15 | 0.17 | 0.92 |
| ENSMUSG00000006526 | Tmem110 | 0.01 | 0.08 | 0.11 | 0.6 |
| ENSMUSG00000039178 | Tbc1d19 | 0.26 | 0.16 | 0.10 | 0.56 |
| ENSMUSG00000021591 | Glrx | 0.33 | 0.20 | 0.19 | 1.03 |
| ENSMUSG00000067995 | Gtf2f2 | 0.32 | 0.12 | 0.13 | 0.72 |
| ENSMUSG00000021650 | Ptcd2 | 0.21 | 0.20 | 0.15 | 0.79 |
| ENSMUSG00000040125 | Gpr26 | 0.01 | 0.07 | 0.11 | 0.61 |
| ENSMUSG00000031386 | Hcfc1 | 0.28 | 0.19 | 0.22 | 1.16 |
| ENSMUSG00000029207 | Apbb2 | 1.25 | 0.47 | 0.25 | 1.30 |
| ENSMUSG00000020042 | Btbd11 | 0.01 | 0.15 | 0.14 | 0.73 |
| ENSMUSG00000036529 | Sbf1 | 0.05 | 0.13 | 0.11 | 0.60 |
| ENSMUSG00000004567 | Mcoln1 | 0.04 | 0.10 | 0.11 | 0.57 |
| ENSMUSG00000022594 | Lynx1 | 0.00 | 0.40 | 0.23 | 1.22 |
| ENSMUSG00000032387 | Rbpms2 | 0.59 | 0.54 | 0.13 | 0.66 |
| ENSMUSG00000029426 | Scarb2 | 0.14 | 0.32 | 0.28 | 1.44 |
| ENSMUSG00000013076 | Amotl1 | 0.08 | 0.13 | 0.12 | 0.63 |
| ENSMUSG00000005683 | Cs | 0.39 | 0.30 | 0.26 | 1.34 |
| ENSMUSG00000033392 | Clasp2 | 0.46 | 0.18 | 0.24 | 1.22 |
| ENSMUSG00000029088 | Kcnip4 | 0.03 | 0.59 | 0.16 | 0.84 |
| ENSMUSG00000047228 | BC048546 | 0.00 | 0.34 | 0.15 | 0.77 |
| ENSMUSG00000014355 | Anapc1 | 0.16 | 0.17 | 0.12 | 0.63 |
| ENSMUSG00000026456 | Cyb5r1 | 0.05 | 0.04 | 0.27 | 1.42 |
| ENSMUSG00000026484 | Rnf2 | 0.43 | 0.09 | 0.13 | 0.68 |
| ENSMUSG00000019852 | Arfgef3 | 0.04 | 0.15 | 0.19 | 0.98 |
| ENSMUSG00000021285 | Ppp1r13b | 0.04 | 0.09 | 0.10 | 0.50 |
| ENSMUSG00000024486 | Hbegf | 0.03 | 0.08 | 0.18 | 0.90 |
| ENSMUSG00000050751 | Pgbd5 | 0.04 | 0.18 | 0.10 | 0.50 |
| ENSMUSG00000037257 | Aagab | 0.13 | 0.10 | 0.11 | 0.55 |
| ENSMUSG00000020876 | Snx11 | 0.10 | 0.13 | 0.13 | 0.64 |
| ENSMUSG00000033530 | Ttc7b | 0.04 | 0.18 | 0.10 | 0.53 |
| ENSMUSG00000017390 | Aldoc | 0.01 | 1.07 | 0.21 | 1.08 |
| ENSMUSG00000024068 | Spast | 0.13 | 0.08 | 0.12 | 0.60 |
| ENSMUSG00000031985 | Gnpat | 0.14 | 0.17 | 0.10 | 0.52 |
| ENSMUSG00000034613 | Ppm1h | 0.04 | 0.11 | 0.12 | 0.59 |
| ENSMUSG00000022265 | Ank | 0.03 | 0.14 | 0.13 | 0.67 |
| ENSMUSG00000028414 | Fktn | 0.14 | 0.14 | 0.15 | 0.76 |
| ENSMUSG00000053519 | Kcnip1 | 0.02 | 0.23 | 0.14 | 0.70 |
| ENSMUSG00000016346 | Kcnq2 | 0.13 | 0.34 | 0.18 | 0.90 |
| ENSMUSG00000027865 | Gdap2 | 0.14 | 0.08 | 0.12 | 0.59 |
| ENSMUSG00000030753 | Prkrir | 0.05 | 0.10 | 0.12 | 0.60 |
| ENSMUSG00000032625 | Thsd7a | 0.44 | 0.19 | 0.15 | 0.77 |
| ENSMUSG00000035183 | Slc24a5 | 0.81 | 0.17 | 0.22 | 1.09 |
| ENSMUSG00000063919 | Srrm4 | 0.79 | 0.12 | 0.10 | 0.50 |
| ENSMUSG00000025400 | Tac2 | 0.00 | 0.07 | 0.10 | 0.52 |
| ENSMUSG00000026663 | Atf6 | 0.09 | 0.12 | 0.14 | 0.71 |
| ENSMUSG00000001419 | Mef2d | 0.04 | 0.21 | 0.14 | 0.7 |
| ENSMUSG00000020454 | Eif4enif1 | 0.12 | 0.10 | 0.11 | 0.53 |
| ENSMUSG00000003660 | Snrnp200 | 0.27 | 0.11 | 0.20 | 0.99 |
| ENSMUSG00000058230 | Arhgap35 | 0.21 | 0.19 | 0.18 | 0.90 |
| ENSMUSG00000064329 | Scn1a | 0.05 | 0.45 | 0.24 | 1.15 |
| ENSMUSG00000053046 | Brsk2 | 0.08 | 0.14 | 0.12 | 0.60 |
| ENSMUSG00000061232 | H2-K1 | 0.01 | 0.13 | 0.20 | 0.99 |
| ENSMUSG00000017639 | Rab11fip4 | 0.13 | 0.34 | 0.19 | 0.94 |
| ENSMUSG00000003949 | Hlf | 0.03 | 0.54 | 0.16 | 0.79 |
| ENSMUSG00000020850 | Prpf8 | 0.21 | 0.21 | 0.18 | 0.89 |
| ENSMUSG00000019873 | Reep3 | 0.18 | 0.09 | 0.12 | 0.59 |
| ENSMUSG00000027674 | Pex5l | 0.02 | 0.14 | 0.21 | 1.01 |
| ENSMUSG00000037339 | Fam53a | 0.01 | 0.10 | 0.13 | 0.65 |
| ENSMUSG00000021061 | Sptb | 0.00 | 0.34 | 0.17 | 0.84 |
| ENSMUSG00000020271 | Fbxw11 | 0.09 | 0.12 | 0.13 | 0.60 |
| ENSMUSG00000025375 | Aatk | 0.02 | 0.30 | 0.14 | 0.68 |
| ENSMUSG00000023079 | Gtf2ird1 | 0.13 | 0.11 | 0.13 | 0.61 |
| ENSMUSG00000025764 | Jade1 | 0.06 | 0.08 | 0.11 | 0.50 |
| ENSMUSG00000027342 | Pcna | 0.33 | 0.06 | 0.15 | 0.72 |
| ENSMUSG00000020307 | Cdc34 | 0.10 | 0.11 | 0.11 | 0.52 |
| ENSMUSG00000022257 | Laptm4b | 0.13 | 0.30 | 0.21 | 0.98 |
| ENSMUSG00000018909 | Arrb1 | 0.06 | 0.30 | 0.22 | 1.05 |
| ENSMUSG00000037872 | Ackr1 | 0.05 | 0.15 | 0.11 | 0.53 |
| ENSMUSG00000090112 | Shprh | 0.21 | 0.11 | 0.17 | 0.80 |
| ENSMUSG00000042050 | Wdr60 | 0.15 | 0.08 | 0.11 | 0.51 |
| ENSMUSG00000041417 | Pik3r1 | 0.27 | 0.15 | 0.16 | 0.76 |
| ENSMUSG00000020929 | Eftud2 | 0.35 | 0.20 | 0.17 | 0.78 |
| ENSMUSG00000060803 | Gstp1 | 0.24 | 0.35 | 0.22 | 1.01 |
| ENSMUSG00000031969 | Acad8 | 0.09 | 0.15 | 0.12 | 0.57 |
| ENSMUSG00000019878 | Hsf2 | 0.28 | 0.08 | 0.13 | 0.58 |
| ENSMUSG00000031749 | St3gal2 | 0.05 | 0.09 | 0.12 | 0.55 |
| ENSMUSG00000022407 | Adsl | 0.12 | 0.13 | 0.14 | 0.64 |
| ENSMUSG00000063801 | Ap3s2 | 0.44 | 0.32 | 0.22 | 0.99 |
| ENSMUSG00000029551 | Psmg3 | 0.20 | 0.24 | 0.14 | 0.64 |
| ENSMUSG00000024065 | Ehd3 | 0.12 | 0.30 | 0.27 | 1.25 |
| ENSMUSG00000064127 | Med14 | 0.05 | 0.13 | 0.11 | 0.51 |
| ENSMUSG00000031012 | Cask | 0.45 | 0.29 | 0.23 | 1.05 |
| ENSMUSG00000041303 | Gtf3c3 | 0.20 | 0.08 | 0.12 | 0.55 |
| ENSMUSG00000098178 | Gm42418 | 0.37 | 0.66 | 1.28 | 5.84 |
| ENSMUSG00000027457 | Snph | 0.01 | 0.24 | 0.22 | 1.00 |
| ENSMUSG00000025810 | Nrp1 | 0.60 | 0.14 | 0.12 | 0.55 |
| ENSMUSG00000004788 | Eif2b2 | 0.28 | 0.26 | 0.22 | 0.99 |
| ENSMUSG00000029636 | Wasf3 | 0.13 | 0.34 | 0.22 | 1.00 |
| ENSMUSG00000037119 | D15Ertd621e | 0.05 | 0.18 | 0.12 | 0.56 |
| ENSMUSG00000014867 | Surf4 | 0.16 | 0.14 | 0.13 | 0.59 |
| ENSMUSG00000033159 | Cnppd1 | 0.33 | 0.10 | 0.19 | 0.84 |
| ENSMUSG00000028483 | Snapc3 | 0.38 | 0.21 | 0.16 | 0.73 |
| ENSMUSG00000045095 | Magi1 | 0.24 | 0.28 | 0.23 | 1.03 |
| ENSMUSG00000014601 | Strip1 | 0.15 | 0.21 | 0.15 | 0.67 |
| ENSMUSG00000029819 | Npy | 0.00 | 0.00 | 0.13 | 0.59 |
| ENSMUSG00000046442 | Ppm1e | 0.06 | 0.19 | 0.13 | 0.60 |
| ENSMUSG00000027346 | Gpcpd1 | 0.13 | 0.18 | 0.12 | 0.53 |
| ENSMUSG00000031543 | Ank1 | 0.00 | 0.11 | 0.12 | 0.53 |
| ENSMUSG00000020142 | Slc1a4 | 0.04 | 0.16 | 0.14 | 0.64 |
| ENSMUSG00000034820 | Cpsf7 | 0.17 | 0.15 | 0.17 | 0.76 |
| ENSMUSG00000026883 | Dab2ip | 0.09 | 0.18 | 0.18 | 0.79 |
| ENSMUSG00000026082 | Rev1 | 0.08 | 0.17 | 0.17 | 0.73 |
| ENSMUSG00000066306 | Numa1 | 0.20 | 0.12 | 0.13 | 0.57 |
| ENSMUSG00000030605 | Mfge8 | 0.05 | 0.00 | 0.13 | 0.59 |
| ENSMUSG00000037331 | Larp1 | 0.10 | 0.17 | 0.13 | 0.58 |
| ENSMUSG00000055296 | Tmem245 | 0.07 | 0.17 | 0.16 | 0.71 |
| ENSMUSG00000029878 | Dbpht2 | 0.04 | 0.09 | 0.15 | 0.67 |
| ENSMUSG00000095041 | PISD | 0.72 | 0.44 | 0.34 | 1.50 |
| ENSMUSG00000021171 | Esyt2 | 0.04 | 0.05 | 0.13 | 0.58 |
| ENSMUSG00000019810 | Fuca2 | 0.10 | 0.24 | 0.19 | 0.83 |
| ENSMUSG00000002103 | Acp2 | 0.14 | 0.17 | 0.13 | 0.56 |
| ENSMUSG00000021373 | Cap2 | 0.01 | 0.22 | 0.17 | 0.76 |
| ENSMUSG00000040209 | Zfp704 | 0.14 | 0.21 | 0.27 | 1.20 |
| ENSMUSG00000026687 | Aldh9a1 | 0.09 | 0.12 | 0.12 | 0.50 |
| ENSMUSG00000021357 | Exoc2 | 0.15 | 0.09 | 0.12 | 0.52 |
| ENSMUSG00000041245 | Wnk3 | 0.33 | 0.10 | 0.12 | 0.52 |
| ENSMUSG00000034157 | Cipc | 0.15 | 0.21 | 0.17 | 0.72 |
| ENSMUSG00000027951 | Adar | 0.05 | 0.38 | 0.30 | 1.28 |
| ENSMUSG00000021222 | Dcaf4 | 0.12 | 0.13 | 0.12 | 0.51 |
| ENSMUSG00000029775 | Klhdc10 | 0.10 | 0.18 | 0.16 | 0.71 |
| ENSMUSG00000040945 | Rcc2 | 0.21 | 0.17 | 0.15 | 0.67 |
| ENSMUSG00000002222 | Rmnd5a | 0.10 | 0.19 | 0.17 | 0.72 |
| ENSMUSG00000016382 | Pls3 | 0.09 | 0.31 | 0.27 | 1.18 |
| ENSMUSG00000052299 | Ltn1 | 0.12 | 0.09 | 0.12 | 0.54 |
| ENSMUSG00000024258 | Polr2d | 0.65 | 0.17 | 0.13 | 0.56 |
| ENSMUSG00000024743 | Syt7 | 0.13 | 0.59 | 0.29 | 1.25 |
| ENSMUSG00000036550 | Cnot1 | 0.25 | 0.12 | 0.16 | 0.69 |
| ENSMUSG00000035969 | Rusc2 | 0.12 | 0.25 | 0.21 | 0.90 |
| ENSMUSG00000037646 | Vps13b | 0.06 | 0.17 | 0.15 | 0.63 |
| ENSMUSG00000051344 | Plekhm3 | 0.06 | 0.17 | 0.14 | 0.61 |
| ENSMUSG00000020115 | Tbk1 | 0.10 | 0.10 | 0.12 | 0.50 |
| ENSMUSG00000057229 | Atp5sl | 0.12 | 0.19 | 0.15 | 0.64 |
| ENSMUSG00000032869 | Psmf1 | 0.21 | 0.25 | 0.21 | 0.88 |
| ENSMUSG00000020430 | Pes1 | 0.29 | 0.18 | 0.17 | 0.74 |
| ENSMUSG00000017307 | Acot8 | 0.10 | 0.22 | 0.17 | 0.70 |
| ENSMUSG00000054408 | Spcs3 | 0.14 | 0.16 | 0.16 | 0.69 |
| ENSMUSG00000020027 | Socs2 | 0.12 | 0.26 | 0.18 | 0.75 |
| ENSMUSG00000032020 | Ubash3b | 0.22 | 0.22 | 0.20 | 0.86 |
| ENSMUSG00000031729 | Ist1 | 0.38 | 0.16 | 0.20 | 0.83 |
| ENSMUSG00000053774 | Ubxn7 | 0.22 | 0.08 | 0.14 | 0.58 |
| ENSMUSG00000072582 | Ptrh2 | 0.19 | 0.14 | 0.16 | 0.66 |
| ENSMUSG00000075376 | Rc3h2 | 0.29 | 0.23 | 0.21 | 0.90 |
| ENSMUSG00000068099 | 1500009C09Rik | 0.01 | 0.74 | 0.17 | 0.72 |
| ENSMUSG00000069227 | Gprin1 | 0.33 | 0.11 | 0.14 | 0.59 |
| ENSMUSG00000024617 | Camk2a | 0.01 | 0.25 | 0.15 | 0.61 |
| ENSMUSG00000097545 | Mir124a-1hg | 0.30 | 0.09 | 0.14 | 0.61 |
| ENSMUSG00000007836 | Hnrnpa0 | 0.43 | 0.26 | 0.19 | 0.81 |
| ENSMUSG00000038733 | Wdr26 | 0.36 | 0.27 | 0.30 | 1.24 |
| ENSMUSG00000022472 | Desi1 | 0.05 | 0.20 | 0.18 | 0.76 |
| ENSMUSG00000063904 | Dpp3 | 0.54 | 0.18 | 0.16 | 0.69 |
| ENSMUSG00000054814 | Usp46 | 0.19 | 0.26 | 0.23 | 0.96 |
| ENSMUSG00000024376 | Epb41l4a | 0.25 | 0.08 | 0.13 | 0.54 |
| ENSMUSG00000024241 | Sos1 | 0.11 | 0.15 | 0.13 | 0.56 |
| ENSMUSG00000059263 | Usp47 | 0.25 | 0.18 | 0.24 | 1.00 |
| ENSMUSG00000004364 | Cul3 | 0.25 | 0.28 | 0.26 | 1.06 |
| ENSMUSG00000020964 | Sel11 | 0.18 | 0.23 | 0.28 | 1.14 |
| ENSMUSG00000031540 | Kat6a | 0.26 | 0.09 | 0.18 | 0.73 |
| ENSMUSG00000036073 | Galt | 0.09 | 0.15 | 0.15 | 0.63 |
| ENSMUSG00000008730 | Hipk1 | 0.05 | 0.18 | 0.19 | 0.80 |
| ENSMUSG00000031309 | Rps6ka3 | 0.11 | 0.09 | 0.13 | 0.52 |
| ENSMUSG00000045038 | Prkce | 0.17 | 0.37 | 0.21 | 0.85 |
| ENSMUSG00000020262 | Adarb1 | 0.03 | 0.48 | 0.40 | 1.65 |
| ENSMUSG00000037652 | Phc3 | 0.11 | 0.19 | 0.15 | 0.63 |
| ENSMUSG00000058351 | Smim4 | 0.34 | 0.11 | 0.13 | 0.55 |
| ENSMUSG00000033557 | Fam20b | 0.09 | 0.20 | 0.18 | 0.72 |
| ENSMUSG00000010066 | Cacna2d2 | 0.06 | 0.32 | 0.16 | 0.65 |
| ENSMUSG00000033209 | Ttc28 | 0.74 | 0.36 | 0.16 | 0.65 |
| ENSMUSG00000031688 | Pou4f2 | 0.49 | 0.23 | 0.16 | 0.64 |
| ENSMUSG00000028059 | Arhgef2 | 0.19 | 0.05 | 0.20 | 0.81 |
| ENSMUSG00000020486 | 4-Sep | 0.10 | 0.38 | 0.24 | 0.99 |
| ENSMUSG00000044068 | Zrsr1 | 0.08 | 0.14 | 0.15 | 0.62 |
| ENSMUSG00000000131 | Xpo6 | 0.23 | 0.18 | 0.18 | 0.73 |
| ENSMUSG00000057716 | Tmem178b | 0.10 | 0.12 | 0.13 | 0.51 |
| ENSMUSG00000026577 | Blzf1 | 0.12 | 0.16 | 0.14 | 0.56 |
| ENSMUSG00000057963 | Itpk1 | 0.02 | 0.14 | 0.13 | 0.54 |
| ENSMUSG00000014243 | Zswim7 | 0.03 | 0.17 | 0.15 | 0.61 |
| ENSMUSG00000028957 | Per3 | 0.02 | 0.29 | 0.14 | 0.55 |
| ENSMUSG00000040687 | Madd | 0.09 | 0.29 | 0.29 | 1.15 |
| ENSMUSG00000072772 | Grcc10 | 0.84 | 0.30 | 0.35 | 1.42 |
| ENSMUSG00000027130 | Slc12a6 | 0.05 | 0.13 | 0.19 | 0.75 |
| ENSMUSG00000029552 | Tes | 0.49 | 0.04 | 0.18 | 0.70 |
| ENSMUSG00000029446 | Psph | 0.15 | 0.12 | 0.22 | 0.88 |
| ENSMUSG00000032261 | Sh3bgrl2 | 0.14 | 0.38 | 0.20 | 0.80 |
| ENSMUSG00000027665 | Pik3ca | 0.35 | 0.35 | 0.25 | 1.00 |
| ENSMUSG00000019971 | Cep290 | 0.24 | 0.23 | 0.19 | 0.77 |
| ENSMUSG00000024924 | Vldlr | 0.24 | 0.22 | 0.19 | 0.76 |
| ENSMUSG00000028572 | Hook1 | 0.10 | 0.27 | 0.23 | 0.91 |
| ENSMUSG00000044071 | Fam19a2 | 0.03 | 0.07 | 0.14 | 0.56 |
| ENSMUSG00000027465 | Tbc1d20 | 0.03 | 0.15 | 0.33 | 1.32 |
| ENSMUSG00000020436 | Gabrg2 | 0.33 | 0.55 | 0.30 | 1.19 |
| ENSMUSG00000086429 | Gt(ROSA)26Sor | 0.64 | 0.24 | 0.14 | 0.56 |
| ENSMUSG00000005886 | Ncoa2 | 0.10 | 0.19 | 0.13 | 0.51 |
| ENSMUSG00000033389 | Arhgap44 | 0.04 | 0.18 | 0.18 | 0.72 |
| ENSMUSG00000048170 | Mcmbp | 0.36 | 0.08 | 0.14 | 0.54 |
| ENSMUSG00000032637 | Atxn21 | 0.12 | 0.19 | 0.23 | 0.92 |
| ENSMUSG00000050812 | AI314180 | 0.15 | 0.35 | 0.27 | 1.05 |
| ENSMUSG00000024640 | Psat1 | 0.22 | 0.06 | 0.20 | 0.78 |
| ENSMUSG00000058454 | Dhcr7 | 0.60 | 0.25 | 0.24 | 0.93 |
| ENSMUSG00000026904 | Slc4a10 | 0.01 | 0.23 | 0.15 | 0.58 |
| ENSMUSG00000052981 | Ube2ql1 | 0.07 | 0.35 | 0.22 | 0.87 |
| ENSMUSG00000046822 | Slc39a3 | 0.04 | 0.20 | 0.14 | 0.54 |
| ENSMUSG00000025212 | Sfxn3 | 0.30 | 0.21 | 0.18 | 0.72 |
| ENSMUSG00000021484 | Lman2 | 0.14 | 0.15 | 0.16 | 0.62 |
| ENSMUSG00000048920 | Fkrp | 0.03 | 0.23 | 0.22 | 0.85 |
| ENSMUSG00000027381 | Bcl2l11 | 0.02 | 0.04 | 0.26 | 1.02 |
| ENSMUSG00000028708 | Mknk1 | 0.03 | 0.08 | 0.14 | 0.54 |
| ENSMUSG00000059995 | Atxn7l3 | 0.07 | 0.18 | 0.15 | 0.58 |
| ENSMUSG00000059439 | Bcas3 | 0.23 | 0.21 | 0.15 | 0.59 |
| ENSMUSG00000007880 | Arid1a | 0.68 | 0.27 | 0.20 | 0.76 |
| ENSMUSG00000031765 | Mt1 | 0.01 | 0.23 | 0.40 | 1.54 |
| ENSMUSG00000041997 | Tlk1 | 0.17 | 0.14 | 0.15 | 0.59 |
| ENSMUSG00000029571 | Tmem106b | 0.07 | 0.25 | 0.25 | 0.96 |
| ENSMUSG00000054728 | Phactr1 | 0.26 | 0.27 | 0.20 | 0.74 |
| ENSMUSG00000079157 | Fam155a | 0.06 | 0.15 | 0.15 | 0.57 |
| ENSMUSG00000039716 | Dock3 | 0.04 | 0.26 | 0.22 | 0.82 |
| ENSMUSG00000022191 | Drosha | 0.19 | 0.15 | 0.15 | 0.58 |
| ENSMUSG00000035325 | Sec31a | 0.21 | 0.22 | 0.22 | 0.85 |
| ENSMUSG00000023033 | Scn8a | 0.05 | 0.37 | 0.16 | 0.59 |
| ENSMUSG00000028184 | Adgrl2 | 0.20 | 0.14 | 0.14 | 0.52 |
| ENSMUSG00000029168 | Dpys15 | 0.64 | 0.25 | 0.17 | 0.62 |
| ENSMUSG00000004789 | Dlst | 0.14 | 0.14 | 0.20 | 0.76 |
| ENSMUSG00000041921 | Metap1d | 0.16 | 0.18 | 0.16 | 0.60 |
| ENSMUSG00000036099 | Vezt | 0.32 | 0.22 | 0.19 | 0.72 |
| ENSMUSG00000048078 | Tenm4 | 0.39 | 0.64 | 0.48 | 1.81 |
| ENSMUSG00000032386 | Trip4 | 0.16 | 0.18 | 0.18 | 0.68 |
| ENSMUSG00000035681 | Kcnc2 | 0.03 | 0.20 | 0.14 | 0.54 |
| ENSMUSG00000019647 | Sema6a | 0.35 | 0.22 | 0.23 | 0.87 |
| ENSMUSG00000017707 | Serinc3 | 0.17 | 0.29 | 0.29 | 1.07 |
| ENSMUSG00000019951 | Uhrf1bp1l | 0.06 | 0.16 | 0.25 | 0.93 |
| ENSMUSG00000019146 | Cacng2 | 0.13 | 0.23 | 0.17 | 0.62 |
| ENSMUSG00000022521 | Crebbp | 0.43 | 0.28 | 0.28 | 1.05 |
| ENSMUSG00000018707 | Dync1h1 | 0.55 | 0.45 | 0.54 | 1.99 |
| ENSMUSG00000037060 | Prkcdbp | 0.00 | 0.16 | 0.19 | 0.68 |
| ENSMUSG00000027575 | Arfgap1 | 0.18 | 0.3 | 0.27 | 0.99 |
| ENSMUSG00000034708 | Grn | 0.10 | 0.16 | 0.17 | 0.61 |
| ENSMUSG00000030714 | Sgf29 | 0.16 | 0.16 | 0.14 | 0.51 |
| ENSMUSG00000047766 | Lrrc49 | 0.52 | 0.32 | 0.29 | 1.08 |
| ENSMUSG00000036591 | Arhgap21 | 0.21 | 0.19 | 0.30 | 1.11 |
| ENSMUSG00000041216 | Clvs1 | 0.26 | 0.18 | 0.16 | 0.58 |
| ENSMUSG00000026247 | Ecel1 | 0.05 | 0.00 | 0.79 | 2.88 |
| ENSMUSG00000015002 | Efr3a | 0.06 | 0.37 | 0.28 | 1.01 |
| ENSMUSG00000015889 | Lta4h | 0.14 | 0.14 | 0.14 | 0.51 |
| ENSMUSG00000015290 | Ubl4a | 0.25 | 0.27 | 0.24 | 0.87 |
| ENSMUSG00000042508 | Dmtf1 | 0.33 | 0.18 | 0.18 | 0.67 |
| ENSMUSG00000029033 | Acap3 | 0.08 | 0.10 | 0.18 | 0.67 |
| ENSMUSG00000045752 | Tssc4 | 0.27 | 0.33 | 0.33 | 1.20 |
| ENSMUSG00000061244 | Exoc5 | 0.20 | 0.21 | 0.21 | 0.77 |
| ENSMUSG00000024740 | Ddb1 | 0.44 | 0.29 | 0.27 | 0.95 |
| ENSMUSG00000033499 | Larp4b | 0.35 | 0.39 | 0.29 | 1.05 |
| ENSMUSG00000064264 | Zfp428 | 0.21 | 0.50 | 0.39 | 1.39 |
| ENSMUSG00000034780 | B3galt1 | 0.06 | 0.11 | 0.16 | 0.59 |
| ENSMUSG00000070565 | Rasal2 | 0.18 | 0.18 | 0.14 | 0.50 |
| ENSMUSG00000040990 | Sh3kbp1 | 0.12 | 0.17 | 0.14 | 0.51 |
| ENSMUSG00000029094 | Afap1 | 0.71 | 0.07 | 0.15 | 0.52 |
| ENSMUSG00000069237 | Fam8a1 | 0.10 | 0.25 | 0.23 | 0.83 |
| ENSMUSG00000056158 | Car10 | 0.16 | 0.20 | 0.22 | 0.76 |
| ENSMUSG00000044308 | Ubr3 | 0.17 | 0.27 | 0.18 | 0.65 |
| ENSMUSG00000034926 | Dhcr24 | 0.17 | 0.49 | 0.20 | 0.71 |
| ENSMUSG00000029603 | Dtx1 | 0.09 | 0.23 | 0.14 | 0.51 |
| ENSMUSG00000020184 | Mdm2 | 0.17 | 0.13 | 0.23 | 0.82 |
| ENSMUSG00000025571 | Tnrc6c | 0.22 | 0.29 | 0.21 | 0.76 |
| ENSMUSG00000059890 | Ube4a | 0.18 | 0.18 | 0.16 | 0.55 |
| ENSMUSG00000031400 | G6pdx | 0.16 | 0.21 | 0.18 | 0.62 |
| ENSMUSG00000038740 | Mvb12b | 0.09 | 0.19 | 0.16 | 0.56 |
| ENSMUSG00000039474 | Wfs1 | 0.02 | 0.30 | 0.20 | 0.69 |
| ENSMUSG00000039361 | Picalm | 0.29 | 0.22 | 0.30 | 1.04 |
| ENSMUSG00000040855 | Reps2 | 0.01 | 0.31 | 0.18 | 0.64 |
| ENSMUSG00000053580 | Tanc2 | 0.17 | 0.46 | 0.28 | 0.97 |
| ENSMUSG00000039988 | Ankrd13c | 0.06 | 0.23 | 0.21 | 0.74 |
| ENSMUSG00000003657 | Calb2 | 0.49 | 1.90 | 0.76 | 2.66 |
| ENSMUSG00000044792 | Isca1 | 0.11 | 0.15 | 0.18 | 0.63 |
| ENSMUSG00000001334 | Fndc5 | 0.04 | 0.22 | 0.16 | 0.54 |
| ENSMUSG00000037235 | Mxd4 | 0.16 | 0.28 | 0.17 | 0.60 |
| ENSMUSG00000092417 | Gpank1 | 0.13 | 0.11 | 0.17 | 0.61 |
| ENSMUSG00000074607 | Tox2 | 0.23 | 0.40 | 0.32 | 1.12 |
| ENSMUSG00000037503 | Fam168b | 0.16 | 0.28 | 0.28 | 0.97 |
| ENSMUSG00000052889 | Prkcb | 0.14 | 0.38 | 0.23 | 0.81 |
| ENSMUSG00000066232 | Ipo7 | 0.22 | 0.21 | 0.24 | 0.84 |
| ENSMUSG00000038828 | Tmem214 | 0.09 | 0.14 | 0.19 | 0.64 |
| ENSMUSG00000027808 | Serp1 | 0.09 | 0.15 | 0.17 | 0.58 |
| ENSMUSG00000041459 | Tardbp | 0.73 | 0.33 | 0.32 | 1.10 |
| ENSMUSG00000005198 | Polr2a | 0.19 | 0.18 | 0.25 | 0.87 |
| ENSMUSG00000024404 | Riok3 | 0.20 | 0.25 | 0.25 | 0.86 |
| ENSMUSG00000028792 | Ak2 | 0.46 | 0.18 | 0.19 | 0.66 |
| ENSMUSG00000040430 | Pitpnc1 | 0.45 | 0.30 | 0.16 | 0.55 |
| ENSMUSG00000019558 | Slc6a8 | 0.05 | 0.10 | 0.18 | 0.61 |
| ENSMUSG00000026442 | Nfasc | 0.44 | 0.39 | 0.28 | 0.97 |
| ENSMUSG00000042605 | Atxn2 | 0.23 | 0.29 | 0.18 | 0.62 |
| ENSMUSG00000025867 | Cplx2 | 0.50 | 0.39 | 0.29 | 1.00 |
| ENSMUSG00000017843 | Ppp2r5c | 0.19 | 0.17 | 0.16 | 0.55 |
| ENSMUSG00000002608 | Ccdc97 | 0.09 | 0.12 | 0.16 | 0.56 |
| ENSMUSG00000074797 | Itpa | 0.63 | 0.55 | 0.38 | 1.29 |
| ENSMUSG00000020882 | Cacnb1 | 0.04 | 0.16 | 0.19 | 0.66 |
| ENSMUSG00000036902 | Neto2 | 0.07 | 0.16 | 0.17 | 0.56 |
| ENSMUSG00000022307 | Oxr1 | 0.23 | 0.53 | 0.44 | 1.47 |
| ENSMUSG00000017776 | Crk | 0.19 | 0.39 | 0.32 | 1.07 |
| ENSMUSG00000019254 | Ppp1r12c | 0.02 | 0.19 | 0.25 | 0.83 |
| ENSMUSG00000046607 | Hrk | 0.03 | 0.01 | 0.37 | 1.23 |
| ENSMUSG00000029767 | Calu | 0.29 | 0.13 | 0.19 | 0.63 |
| ENSMUSG00000028657 | Ppt1 | 0.34 | 0.50 | 0.48 | 1.62 |
| ENSMUSG00000056367 | Actr3b | 0.02 | 0.27 | 0.15 | 0.51 |
| ENSMUSG00000030302 | Atp2b2 | 0.10 | 0.50 | 0.39 | 1.32 |
| ENSMUSG00000036435 | Exoc1 | 0.24 | 0.14 | 0.16 | 0.52 |
| ENSMUSG00000035215 | Lsm7 | 0.77 | 0.13 | 0.31 | 1.02 |
| ENSMUSG00000027698 | Nceh1 | 0.03 | 0.28 | 0.20 | 0.67 |
| ENSMUSG00000022721 | Trmt2a | 0.15 | 0.15 | 0.20 | 0.65 |
| ENSMUSG00000066621 | Tecpr1 | 0.15 | 0.42 | 0.26 | 0.88 |
| ENSMUSG00000024644 | Cndp2 | 0.25 | 0.31 | 0.20 | 0.66 |
| ENSMUSG00000042453 | Reln | 0.36 | 0.29 | 0.26 | 0.86 |
| ENSMUSG00000022972 | 1110004E09Rik | 0.10 | 0.29 | 0.29 | 0.95 |
| ENSMUSG00000062901 | Klh124 | 0.45 | 0.15 | 0.23 | 0.77 |
| ENSMUSG00000028960 | Ube4b | 0.28 | 0.28 | 0.27 | 0.88 |
| ENSMUSG00000029265 | Dr1 | 0.05 | 0.13 | 0.25 | 0.81 |
| ENSMUSG00000029578 | Wipi2 | 0.08 | 0.24 | 0.25 | 0.82 |
| ENSMUSG00000063802 | Hspbp1 | 0.43 | 0.38 | 0.33 | 1.07 |
| ENSMUSG00000009090 | Ap1b1 | 0.16 | 0.22 | 0.22 | 0.72 |
| ENSMUSG00000016257 | Slmo2 | 0.42 | 0.55 | 0.32 | 1.06 |
| ENSMUSG00000031565 | Fgfr1 | 0.04 | 0.07 | 0.25 | 0.82 |
| ENSMUSG00000047963 | Stbd1 | 0.04 | 0.07 | 0.25 | 0.81 |
| ENSMUSG00000048814 | Lonrf2 | 0.08 | 0.34 | 0.23 | 0.75 |
| ENSMUSG00000032563 | Mrpl3 | 0.05 | 0.25 | 0.19 | 0.62 |
| ENSMUSG00000032231 | Anxa2 | 0.00 | 0.08 | 0.17 | 0.56 |
| ENSMUSG00000020668 | Kif3c | 0.20 | 0.22 | 0.27 | 0.89 |
| ENSMUSG00000036473 | Tbc1d24 | 0.07 | 0.18 | 0.21 | 0.68 |
| ENSMUSG00000033615 | Cplx1 | 0.22 | 0.42 | 0.29 | 0.95 |
| ENSMUSG00000024240 | Epc1 | 0.22 | 0.15 | 0.22 | 0.72 |
| ENSMUSG00000022489 | Pde1b | 0.19 | 0.09 | 0.29 | 0.94 |
| ENSMUSG00000030002 | Dusp11 | 0.38 | 0.21 | 0.26 | 0.83 |
| ENSMUSG00000030298 | Sec13 | 0.51 | 0.30 | 0.25 | 0.80 |
| ENSMUSG00000028803 | Nipal3 | 0.03 | 0.27 | 0.24 | 0.77 |
| ENSMUSG00000023050 | Map3k12 | 0.17 | 0.17 | 0.22 | 0.72 |
| ENSMUSG00000031728 | Zfp821 | 0.13 | 0.14 | 0.22 | 0.72 |
| ENSMUSG00000017631 | Abr | 0.08 | 0.52 | 0.27 | 0.87 |
| ENSMUSG00000036817 | Sun1 | 0.14 | 0.19 | 0.16 | 0.52 |
| ENSMUSG00000025576 | Rbfox3 | 0.17 | 0.44 | 0.32 | 1.02 |
| ENSMUSG00000032468 | Armc8 | 0.28 | 0.34 | 0.28 | 0.91 |
| ENSMUSG00000032024 | Clmp | 0.16 | 0.15 | 0.28 | 0.92 |
| ENSMUSG00000063632 | Sox11 | 2.86 | 0.13 | 0.49 | 1.57 |
| ENSMUSG00000022463 | Srebf2 | 0.32 | 0.28 | 0.27 | 0.87 |
| ENSMUSG00000019818 | Cd164 | 0.09 | 0.15 | 0.16 | 0.51 |
| ENSMUSG00000048915 | Efna5 | 0.42 | 0.15 | 0.17 | 0.56 |
| ENSMUSG00000046032 | Snx12 | 0.31 | 0.20 | 0.19 | 0.62 |
| ENSMUSG00000038174 | Fam126b | 0.15 | 0.33 | 0.28 | 0.90 |
| ENSMUSG00000021244 | Ylpm1 | 0.34 | 0.27 | 0.32 | 1.03 |
| ENSMUSG00000005442 | Cic | 0.09 | 0.17 | 0.25 | 0.81 |
| ENSMUSG00000022240 | Ctnnd2 | 0.42 | 0.28 | 0.17 | 0.53 |
| ENSMUSG00000038738 | Shank1 | 0.17 | 0.27 | 0.37 | 1.17 |
| ENSMUSG00000031026 | Trim66 | 0.00 | 0.10 | 0.18 | 0.56 |
| ENSMUSG00000034757 | Tmub2 | 0.28 | 0.25 | 0.23 | 0.73 |
| ENSMUSG00000042502 | Cd2bp2 | 0.21 | 0.16 | 0.22 | 0.69 |
| ENSMUSG00000021700 | Rab3c | 0.43 | 0.59 | 0.42 | 1.32 |
| ENSMUSG00000021458 | 2010111I01Rik | 0.03 | 0.19 | 0.30 | 0.94 |
| ENSMUSG00000024302 | Dtna | 0.05 | 0.13 | 0.26 | 0.83 |
| ENSMUSG00000032244 | Fem1b | 0.23 | 0.28 | 0.30 | 0.93 |
| ENSMUSG00000039953 | Clstn1 | 0.28 | 1.06 | 0.68 | 2.15 |
| ENSMUSG00000030772 | Dkk3 | 0.53 | 0.53 | 0.26 | 0.81 |
| ENSMUSG00000062661 | Ncs1 | 0.68 | 0.40 | 0.27 | 0.85 |
| ENSMUSG00000020257 | Wdr82 | 0.19 | 0.16 | 0.18 | 0.56 |
| ENSMUSG00000022957 | Itsn1 | 0.18 | 0.24 | 0.22 | 0.68 |
| ENSMUSG00000033124 | Atg9a | 0.09 | 0.20 | 0.16 | 0.51 |
| ENSMUSG00000015597 | Zfp318 | 0.24 | 0.16 | 0.18 | 0.56 |
| ENSMUSG00000025745 | Hadha | 0.23 | 0.19 | 0.18 | 0.56 |
| ENSMUSG00000018412 | Kansl1 | 0.19 | 0.18 | 0.23 | 0.73 |
| ENSMUSG00000032418 | Me1 | 0.03 | 0.33 | 0.30 | 0.94 |
| ENSMUSG00000029434 | Vps33a | 0.11 | 0.11 | 0.16 | 0.51 |
| ENSMUSG00000034158 | Lrrc58 | 0.29 | 0.26 | 0.30 | 0.94 |
| ENSMUSG00000002413 | Braf | 0.22 | 0.35 | 0.30 | 0.93 |
| ENSMUSG00000036469 | 1-Mar | 0.16 | 0.19 | 0.26 | 0.81 |
| ENSMUSG00000044024 | Rell2 | 0.03 | 0.37 | 0.22 | 0.68 |
| ENSMUSG00000071660 | Ttc9c | 0.21 | 0.14 | 0.17 | 0.54 |
| ENSMUSG00000029544 | Cabp1 | 0.01 | 0.18 | 0.23 | 0.72 |
| ENSMUSG00000024516 | Sec11c | 0.21 | 0.69 | 0.49 | 1.51 |
| ENSMUSG00000020523 | Fam114a2 | 0.52 | 0.33 | 0.27 | 0.84 |
| ENSMUSG00000029007 | Agtrap | 0.01 | 0.07 | 0.26 | 0.81 |
| ENSMUSG00000031442 | Mcf2l | 0.20 | 0.32 | 0.18 | 0.55 |
| ENSMUSG00000019797 | 1700021F05Rik | 0.26 | 0.28 | 0.25 | 0.78 |
| ENSMUSG00000028647 | Mycbp | 0.21 | 0.21 | 0.18 | 0.54 |
| ENSMUSG00000027828 | Ssr3 | 0.50 | 0.28 | 0.40 | 1.22 |
| ENSMUSG00000030500 | Slc17a6 | 0.31 | 0.70 | 0.59 | 1.81 |
| ENSMUSG00000030759 | Far1 | 0.39 | 0.38 | 0.25 | 0.77 |
| ENSMUSG00000046329 | Slc25a23 | 0.10 | 0.20 | 0.17 | 0.53 |
| ENSMUSG00000028080 | Lrba | 0.14 | 0.29 | 0.16 | 0.50 |
| ENSMUSG00000018750 | Zbtb4 | 0.02 | 0.45 | 0.29 | 0.90 |
| ENSMUSG00000025283 | Sat1 | 0.25 | 0.24 | 0.32 | 0.98 |
| ENSMUSG00000041168 | Lonp1 | 0.05 | 0.17 | 0.37 | 1.13 |
| ENSMUSG00000045636 | Mtus1 | 0.23 | 0.11 | 0.19 | 0.58 |
| ENSMUSG00000038991 | Txndc5 | 0.04 | 0.12 | 0.18 | 0.55 |
| ENSMUSG00000041164 | Zmiz2 | 0.23 | 0.23 | 0.17 | 0.5 |
| ENSMUSG00000020300 | Cpeb4 | 0.11 | 0.20 | 0.22 | 0.68 |
| ENSMUSG00000052572 | Dlg2 | 0.15 | 0.67 | 0.47 | 1.42 |
| ENSMUSG00000061360 | Phf5a | 0.65 | 0.43 | 0.38 | 1.15 |
| ENSMUSG00000001729 | Akt1 | 0.22 | 0.23 | 0.19 | 0.57 |
| ENSMUSG00000068240 | Gm11808 | 0.49 | 0.12 | 0.19 | 0.56 |
| ENSMUSG00000047193 | Dync2h1 | 0.15 | 0.18 | 0.20 | 0.60 |
| ENSMUSG00000035189 | Ano4 | 0.13 | 0.06 | 0.19 | 0.58 |
| ENSMUSG00000097767 | Miat | 0.76 | 0.35 | 0.26 | 0.77 |
| ENSMUSG00000037486 | Asxl2 | 0.15 | 0.19 | 0.17 | 0.51 |
| ENSMUSG00000006010 | BC003331 | 0.24 | 0.41 | 0.36 | 1.06 |
| ENSMUSG00000040537 | Adam22 | 0.06 | 0.36 | 0.30 | 0.88 |
| ENSMUSG00000020114 | Cand1 | 0.17 | 0.37 | 0.27 | 0.81 |
| ENSMUSG00000037965 | Zc3h7a | 0.30 | 0.19 | 0.17 | 0.52 |
| ENSMUSG00000021945 | Zmym2 | 0.36 | 0.14 | 0.19 | 0.56 |
| ENSMUSG00000084957 | Bbip1 | 0.84 | 0.31 | 0.29 | 0.86 |
| ENSMUSG00000018199 | Trove2 | 0.51 | 0.32 | 0.28 | 0.84 |
| ENSMUSG00000026718 | Stam | 0.20 | 0.24 | 0.24 | 0.71 |
| ENSMUSG00000026761 | Orc4 | 0.29 | 0.20 | 0.23 | 0.68 |
| ENSMUSG00000032187 | Smarca4 | 1.17 | 0.53 | 0.36 | 1.08 |
| ENSMUSG00000022247 | Brix1 | 0.48 | 0.26 | 0.25 | 0.73 |
| ENSMUSG00000058064 | Gm10036 | 0.02 | 0.17 | 0.42 | 1.24 |
| ENSMUSG00000097868 | A330069E16Rik | 0.09 | 0.10 | 0.25 | 0.73 |
| ENSMUSG00000028266 | Lmo4 | 0.15 | 0.18 | 0.23 | 0.67 |
| ENSMUSG00000035776 | Cd99l2 | 0.25 | 0.33 | 0.35 | 1.04 |
| ENSMUSG00000001576 | Ergic1 | 0.14 | 0.26 | 0.32 | 0.95 |
| ENSMUSG00000040549 | Ckap5 | 0.84 | 0.35 | 0.33 | 0.96 |
| ENSMUSG00000023150 | Ivns1abp | 0.38 | 0.48 | 0.43 | 1.26 |
| ENSMUSG00000035566 | Pcdh17 | 0.18 | 0.13 | 0.19 | 0.57 |
| ENSMUSG00000033998 | Kcnk1 | 0.02 | 0.23 | 0.29 | 0.85 |
| ENSMUSG00000030615 | Tmem126a | 0.44 | 0.43 | 0.32 | 0.95 |
| ENSMUSG00000030688 | Stard10 | 0.03 | 0.17 | 0.23 | 0.68 |
| ENSMUSG00000101892 | 9130401M01Rik | 0.11 | 0.33 | 0.28 | 0.83 |
| ENSMUSG00000028958 | Tmub1 | 0.27 | 0.31 | 0.23 | 0.66 |
| ENSMUSG00000052516 | Robo2 | 0.84 | 0.39 | 0.32 | 0.93 |
| ENSMUSG00000026787 | Gad2 | 0.20 | 0.36 | 0.21 | 0.62 |
| ENSMUSG00000000439 | Mkrn2 | 0.18 | 0.16 | 0.21 | 0.60 |
| ENSMUSG00000048495 | Tyw5 | 0.07 | 0.11 | 0.18 | 0.51 |
| ENSMUSG00000062252 | Lhfpl5 | 0.05 | 0.40 | 0.21 | 0.62 |
| ENSMUSG00000024963 | Dnajc4 | 0.07 | 0.22 | 0.19 | 0.55 |
| ENSMUSG00000026000 | Lancl1 | 0.22 | 0.53 | 0.51 | 1.48 |
| ENSMUSG00000030655 | Smg1 | 0.16 | 0.20 | 0.20 | 0.58 |
| ENSMUSG00000028528 | Dnajc6 | 0.10 | 0.33 | 0.25 | 0.72 |
| ENSMUSG00000004113 | Cacna1b | 0.23 | 0.32 | 0.30 | 0.86 |
| ENSMUSG00000033065 | Pfkm | 0.19 | 0.56 | 0.36 | 1.03 |
| ENSMUSG00000047368 | Abhd17b | 0.08 | 0.14 | 0.20 | 0.57 |
| ENSMUSG00000071648 | Rom1 | 0.04 | 0.37 | 0.25 | 0.71 |
| ENSMUSG00000020166 | Cnot2 | 0.41 | 0.24 | 0.26 | 0.75 |
| ENSMUSG00000026384 | Ptpn4 | 0.05 | 0.23 | 0.21 | 0.61 |
| ENSMUSG00000028975 | Pex14 | 0.15 | 0.32 | 0.26 | 0.74 |
| ENSMUSG00000030844 | Rgs10 | 0.16 | 0.35 | 0.19 | 0.55 |
| ENSMUSG00000013921 | Clip3 | 0.41 | 0.75 | 0.67 | 1.92 |
| ENSMUSG00000029095 | Ablim2 | 0.00 | 0.31 | 0.25 | 0.72 |
| ENSMUSG00000034951 | Cog7 | 0.07 | 0.26 | 0.24 | 0.68 |
| ENSMUSG00000027210 | Meis2 | 0.18 | 0.30 | 0.27 | 0.79 |
| ENSMUSG00000042726 | Trafd1 | 0.18 | 0.14 | 0.18 | 0.50 |
| ENSMUSG00000041720 | Pi4ka | 0.12 | 0.27 | 0.20 | 0.56 |
| ENSMUSG00000029920 | Smarcad1 | 0.33 | 0.12 | 0.19 | 0.53 |
| ENSMUSG00000042506 | Usp22 | 0.56 | 0.51 | 0.52 | 1.50 |
| ENSMUSG00000040618 | Pck2 | 0.03 | 0.13 | 0.19 | 0.55 |
| ENSMUSG00000027433 | Xrn2 | 0.89 | 0.19 | 0.24 | 0.68 |
| ENSMUSG00000027244 | Atg13 | 0.10 | 0.19 | 0.18 | 0.50 |
| ENSMUSG00000005125 | Ndrg1 | 0.10 | 0.27 | 0.23 | 0.66 |
| ENSMUSG00000024969 | Mark2 | 0.12 | 0.32 | 0.30 | 0.85 |
| ENSMUSG00000012535 | Tnpo3 | 0.54 | 0.35 | 0.31 | 0.89 |
| ENSMUSG00000058355 | Abce1 | 0.42 | 0.18 | 0.21 | 0.60 |
| ENSMUSG00000000561 | Wdr77 | 0.25 | 0.26 | 0.21 | 0.60 |
| ENSMUSG00000019132 | BC005537 | 0.40 | 0.53 | 0.45 | 1.29 |
| ENSMUSG00000021557 | Agtpbp1 | 0.19 | 0.31 | 0.26 | 0.73 |
| ENSMUSG00000019158 | Tmem160 | 0.08 | 0.83 | 0.64 | 1.82 |
| ENSMUSG00000020456 | Ogdh | 0.20 | 0.47 | 0.36 | 1.03 |
| ENSMUSG00000032565 | Nudt16 | 0.05 | 0.22 | 0.20 | 0.55 |
| ENSMUSG00000027347 | Brinp1 | 0.00 | 0.04 | 0.05 | 0.16 |
| ENSMUSG00000034981 | Bex4 | 0.03 | 0.13 | 0.06 | 0.17 |
| ENSMUSG00000075334 | Csnk1a1 | 0.34 | 0.63 | 0.18 | 0.36 |
| ENSMUSG00000021799 | Pdcd10 | 0.02 | 0.08 | 0.18 | 0.30 |
| ENSMUSG00000046167 | Hdgfrp3 | 0.00 | 0.01 | 0.06 | 0.10 |
| ENSMUSG00000030638 | Dnajb9 | 0.02 | 0.10 | 0.11 | 0.18 |
| ENSMUSG00000021948 | Flot1 | 0.11 | 0.07 | 0.12 | 0.15 |
| ENSMUSG00000032446 | Ubtf | 0.20 | 0.10 | 0.13 | 0.16 |
| ENSMUSG00000029875 | St3gal5 | 0.15 | 0.56 | 0.19 | 0.20 |
| ENSMUSG00000028351 | Aamp | 0.04 | 0.31 | 0.32 | 0.32 |
| ENSMUSG00000045777 | Mrps36 | 0.00 | 0.04 | 0.17 | 0.15 |
| ENSMUSG00000002012 | Zfp467 | 0.13 | 0.37 | 0.27 | 0.23 |
| ENSMUSG00000030554 | Ube2i | 0.29 | 0.12 | 0.09 | 0.08 |
| ENSMUSG00000042761 | Gpx4 | 0.00 | 0.04 | 0.09 | 0.07 |
| ENSMUSG00000003411 | Acp1 | 0.27 | 0.08 | 0.22 | 0.18 |
| ENSMUSG00000032878 | Sptssa | 0.12 | 0.30 | 0.10 | 0.08 |
| ENSMUSG00000024256 | Rhoq | 0.16 | 0.12 | 0.28 | 0.20 |
| ENSMUSG00000019986 | Pnck | 0.14 | 0.59 | 0.60 | 0.43 |
| ENSMUSG00000047844 | Ttc33 | 0.51 | 0.30 | 0.38 | 0.25 |
| ENSMUSG00000044408 | Parm1 | 0.72 | 0.26 | 0.27 | 0.17 |
| ENSMUSG00000068551 | Samd14 | 0.04 | 0.64 | 0.40 | 0.22 |
| ENSMUSG00000070304 | Psmc4 | 0.05 | 0.77 | 0.31 | 0.17 |
| ENSMUSG00000029608 | Scn2b | 0.54 | 1.42 | 0.55 | 0.30 |
| ENSMUSG00000003273 | Eml2 | 0.03 | 0.70 | 0.41 | 0.21 |
| ENSMUSG00000056091 | Pfn1 | 0.09 | 0.26 | 0.35 | 0.18 |
| ENSMUSG00000047139 | Tia1 | 2.73 | 0.03 | 0.18 | 0.08 |
| ENSMUSG00000061474 | Ccdc184 | 0.67 | 0.81 | 0.43 | 0.20 |
| ENSMUSG00000059714 | Spg21 | 0.20 | 0.78 | 0.56 | 0.25 |
| ENSMUSG00000024143 | Rab3b | 0.03 | 0.11 | 0.43 | 0.19 |
| ENSMUSG00000036764 | Sh3gl3 | 0.29 | 0.38 | 0.36 | 0.15 |
| ENSMUSG00000020923 | Cdh2 | 0.22 | 0.36 | 0.50 | 0.21 |
| ENSMUSG00000006731 | Morf4l1 | 0.05 | 0.41 | 0.36 | 0.15 |
| ENSMUSG00000006299 | Rprm | 0.72 | 0.74 | 0.74 | 0.29 |
| ENSMUSG00000015120 | Dnajc12 | 0.90 | 0.34 | 0.49 | 0.18 |
| ENSMUSG00000027835 | Rwdd1 | 0.67 | 0.65 | 0.61 | 0.22 |
| ENSMUSG00000022199 | B4galnt1 | 0.14 | 1.22 | 0.70 | 0.25 |
| ENSMUSG00000041096 | Jund | 0.07 | 0.29 | 0.32 | 0.12 |
| ENSMUSG00000025104 | Ypel4 | 0.64 | 0.79 | 0.75 | 0.27 |
| ENSMUSG00000071076 | Actg1 | 0.23 | 0.89 | 1.97 | 0.70 |
| ENSMUSG00000075706 | Ubxn4 | 1.64 | 2.30 | 2.00 | 0.69 |
| ENSMUSG00000024304 | Smarca5 | 0.60 | 0.38 | 0.47 | 0.16 |
| ENSMUSG00000024480 | Dazap1 | 0.36 | 0.85 | 0.49 | 0.17 |
| ENSMUSG00000014905 | Sf3b2 | 0.09 | 0.37 | 0.55 | 0.19 |
| ENSMUSG00000046434 | Car11 | 2.44 | 1.09 | 0.93 | 0.32 |
| ENSMUSG00000024245 | Wac | 0.02 | 0.20 | 0.35 | 0.12 |
| ENSMUSG00000024576 | Btf3l4 | 0.59 | 1.23 | 1.20 | 0.41 |
| ENSMUSG00000040811 | Hnrnpa1 | 0.03 | 0.62 | 0.62 | 0.21 |
| ENSMUSG00000047181 | Rasgrp1 | 0.17 | 0.69 | 0.52 | 0.17 |
| ENSMUSG00000006930 | Ahi1 | 0.01 | 0.10 | 0.22 | 0.07 |
| ENSMUSG00000044573 | Rph3a | 1.18 | 1.20 | 1.06 | 0.34 |
| ENSMUSG00000109006 | Rpl29 | 0.00 | 0.18 | 0.35 | 0.11 |
| ENSMUSG00000031715 | RP23-407N2.2 | 0.63 | 0.36 | 0.60 | 0.19 |
| ENSMUSG00000022151 | Cbx3 | 0.24 | 0.46 | 0.59 | 0.19 |
| ENSMUSG00000018293 | Pdia3 | 0.78 | 1.18 | 1.37 | 0.42 |
| ENSMUSG00000032297 | Ap3s1 | 0.05 | 0.17 | 0.17 | 0.05 |
| ENSMUSG00000034059 | Tmed2 | 0.07 | 0.42 | 0.54 | 0.16 |
| ENSMUSG00000031534 | Pten | 0.03 | 0.19 | 0.44 | 0.13 |
| ENSMUSG00000032388 | Slc22a17 | 0.27 | 0.47 | 0.52 | 0.16 |
| ENSMUSG00000071337 | Smim19 | 1.03 | 0.53 | 0.54 | 0.16 |
| ENSMUSG00000041084 | Set | 1.06 | 0.56 | 0.35 | 0.10 |
| ENSMUSG00000062270 | Fbxo2 | 2.80 | 2.66 | 2.76 | 0.78 |
| ENSMUSG00000052837 | Gldn | 0.02 | 0.33 | 0.47 | 0.13 |
| ENSMUSG00000019782 | Prkcd | 0.40 | 0.83 | 0.79 | 0.21 |
| ENSMUSG00000032562 | Flywch1 | 0.45 | 0.62 | 0.50 | 0.13 |
| ENSMUSG00000044783 | Pdia6 | 0.24 | 0.30 | 0.25 | 0.07 |
| ENSMUSG00000013663 | Ostc | 0.18 | 0.54 | 0.45 | 0.12 |
| ENSMUSG00000041556 | Wbp11 | 0.00 | 1.13 | 0.62 | 0.16 |
| ENSMUSG00000030603 | Phf3 | 1.09 | 1.07 | 0.90 | 0.23 |
| ENSMUSG00000024853 | Ifitm10 | 1.58 | 1.34 | 1.39 | 0.36 |
| ENSMUSG00000062825 | Sec61g | 3.98 | 2.50 | 2.58 | 0.67 |
| ENSMUSG00000024283 | Bri3 | 0.39 | 0.44 | 0.53 | 0.13 |
| ENSMUSG00000026353 | Snhg12 | 0.59 | 0.91 | 0.86 | 0.22 |
| ENSMUSG00000078974 | Ralbp1 | 2.14 | 0.75 | 1.06 | 0.26 |
| ENSMUSG00000020571 | Tmem178 | 1.35 | 0.55 | 0.54 | 0.13 |
| ENSMUSG00000023328 | Tmem222 | 0.07 | 0.68 | 0.66 | 0.15 |
| ENSMUSG00000021250 | Erlec1 | 0.32 | 0.56 | 0.96 | 0.22 |
| ENSMUSG00000040097 | Tspyl2 | 0.23 | 1.26 | 0.91 | 0.21 |
| ENSMUSG00000024096 | Ube2v2 | 0.19 | 0.89 | 0.72 | 0.16 |
| ENSMUSG00000069565 | Adcyap1 | 0.24 | 0.49 | 0.66 | 0.15 |
| ENSMUSG00000022577 | Thrap3 | 0.24 | 1.44 | 0.31 | 0.07 |
| ENSMUSG00000028857 | Opn4 | 0.32 | 0.70 | 0.60 | 0.13 |
| ENSMUSG00000048758 | Fam220a | 2.23 | 1.03 | 1.67 | 0.37 |
| ENSMUSG00000027248 | Mapre3 | 0.57 | 0.48 | 0.77 | 0.17 |
| ENSMUSG00000030216 | Srp54b | 0.84 | 0.46 | 0.59 | 0.12 |
| ENSMUSG00000048874 | Ccm2 | 0.30 | 0.35 | 0.56 | 0.12 |
| ENSMUSG00000029166 | BC005624 | 0.08 | 1.27 | 0.80 | 0.16 |
| ENSMUSG00000054766 | Synm | 1.38 | 0.93 | 1.21 | 0.24 |
| ENSMUSG00000028568 | Eomes | 1.05 | 0.66 | 0.82 | 0.16 |
| ENSMUSG00000029836 | Ccdc85a | 1.73 | 0.49 | 0.74 | 0.14 |
| ENSMUSG00000020311 | Mrap2 | 0.33 | 0.58 | 0.57 | 0.10 |
| ENSMUSG00000072966 | Gnai2 | 0.29 | 0.58 | 0.40 | 0.07 |
| ENSMUSG00000086290 | Hap1 | 0.09 | 0.31 | 0.55 | 0.09 |
| ENSMUSG00000022674 | Fos | 1.19 | 1.49 | 0.94 | 0.16 |
| ENSMUSG00000083012 | Rpp21 | 0.10 | 0.42 | 0.50 | 0.09 |
| ENSMUSG00000079108 | Hjurp | 1.03 | 0.93 | 0.92 | 0.15 |
| ENSMUSG00000025791 | Ndufs5 | 0.25 | 0.50 | 0.32 | 0.05 |
| ENSMUSG00000047843 | Ache | 0.19 | 0.92 | 1.15 | 0.18 |
| ENSMUSG00000043962 | Celf6 | 1.00 | 0.52 | 0.66 | 0.10 |
| ENSMUSG00000026851 | Pgm2 | 0.63 | 0.67 | 0.69 | 0.10 |
| ENSMUSG00000086503 | Junb | 0.99 | 1.27 | 1.49 | 0.20 |
| ENSMUSG00000000378 | Cd24a | 0.10 | 0.53 | 0.66 | 0.08 |
| ENSMUSG00000024446 | Slbp | 0.49 | 0.41 | 0.48 | 0.06 |
| ENSMUSG00000028648 | Gprasp2 | 1.44 | 1.78 | 1.52 | 0.16 |
| ENSMUSG00000004642 | Arf5 | 0.62 | 0.43 | 0.39 | 0.04 |
| ENSMUSG00000037624 | Rnps1 | 0.02 | 0.07 | 0.27 | 0.02 |
| ENSMUSG00000020440 | Ly6h | 0.19 | 1.35 | 1.30 | 0.06 |
| ENSMUSG00000034681 | Sumo2 | 1.02 | 0.64 | 0.86 | 0.03 |
| ENSMUSG00000020738 | mt-Co3 | 2.74 | 1.95 | 1.43 | 0.05 |
| ENSMUSG00000064358 | Kcnk2 | 4.04 | 4.42 | 3.53 | 0.08 |
| ENSMUSG00000064357 | mt-Atp6 | 4.16 | 4.62 | 3.78 | 0.05 |
| ENSMUSG00000064354 | mt-Co2 | 3.20 | 3.86 | 3.03 | 0.03 |
| ENSMUSG00000029019 | Polr2k | 0.00 | 0.02 | 0.06 | 0.00 |
| ENSMUSG00000045996 | Nppb | 1.14 | 0.65 | 0.64 | 0.00 |
| ENSMUSG00000060860 | mt-Nd3 | 0.11 | 0.72 | 1.06 | 0.00 |
| ENSMUSG00000064360 | Ube2s | 0.96 | 1.20 | 1.09 | 0.00 |

Embryonic, Adult, and Injured RGC scRNA-Seq Datasets Procurement and Initialization.

BAM files, raw counts, normalized matrices, and cell metadata (e.g., type assignment) were obtained for the mouse embryonic RGCs from the Gene Expression Omnibus (GEO) accession number GSE12246635, and for the mouse adult RGC atlas and the injured RGCs from the GEO accession number GSE13740033. BAM files were converted to FASTQ files using CellRanger's bamtofastq software. FASTQ files were aggregated where appropriate using CellRanger and then mapped to the CellRanger mm10-1.2.0 transcriptome. Batch correction was performed for separate batches using the FindIntegrationAnchors and IntegrateData functions from Seurat v. 4.0.368,69. The same cells that passed the original quality checks, and the same cell-to-type assignments from the original analyses, were used in the present study.

### Bioinformatic Analyses of Long- and Short-Distance Axon-Regenerating RGCS.

The plate-based-generated SmartSeq2 scRNA-seq dataset (Pten KD long-distance axon-regenerating RGCs) was merged with the 10× single cell platform-generated scRNA-seq datasets (embryonic, adult atlas, and adult injured RGCs) using SAVER70, with default parameters for the plate-based-generated scRNA-seq, for the downstream comparative analyses between the datasets. All datasets were normalized using Seurat's NormalizeData function with default parameters. The sex-specific genes, Xist, Eif2s3y, and Ddx3y, were excluded for dimensionality reduction but retained for downstream analyses. Embryonic and adult RGCs were aligned using the mutual nearest neighbor's algorithm from Batchelor as part of the Monocle v.372, which was used to generate the merged embryonic and adult atlas RGC UMAP. Monocle was also used to determine the pseudo-timeline structure of the merged (embryonic/adult RGC) UMAP. The UMAP cell embeddings, generated by Monocle, were transferred to a Seurat object containing the same datasets, and the hyperparameters (umap.n.neighbors=10; umap.metric=“euclidean”; umap.min.dist=0.1; n_epochs=200; learning_rate=1; repulsion_strength 1; negative_sample_rate=5; approx._pow=0; spread=1) were used to generate a Seurat UMAP model.

CellTools' algorithm was used to map the Pten KD regenerating RGCs to their cell type origins, and the injured and Pten KD long-distance axon-regenerating RGCs were individually assigned the same pseudo-timeline score as their nearest reference neighbor. The counts-matrix for the plate-based-generated SmartSeq2 scRNA-seq dataset of the 120 short-distance axon-regenerating RGCs was obtained from the GEO accession number GSE20215526 and merged as above with the 10× single cell platform-generated scRNA-seq datasets using SAVER70, with default parameters for the plate-based-generated scRNA-seq, for the downstream comparative analyses between the datasets. Normalization, mapping to the reference UMAP, and assignment of the pseudotimeline scores for the short-distance axon-regenerating RGCs, also was performed as above. Comparative analysis of scRNA-seq datasets was performed using the R package Seurat v4.3.0.

### Heatmaps and Violin Plots.

Heatmaps were generated using Superheat, with each gene's average expression per group scaled using Z-scores, as we previously published. The genes were ordered by the log 2 fold-change between average expression in the Pten KD long-distance axon-regenerating RGCs and average expression in the injured control RGCs from clusters C33 and C40. Violin plots were generated using ggplot2 and Seurat's VlnPlot function. Expression data for specific genes was extracted using Seurat's VlnPlot function. Violin plots were generated using ggplot2's geom_violin function, and overlayed categorical scatter (violin point) plots were generated using ggbeeswarm.

### Gene-Concept Network Plot and Functional Enrichment Analysis.

Functional enrichment analysis was performed using the R package gprofiler2 on genes upregulated (determined using Seurat's FindMarkers function) in Pten KD long-distance axon-regenerating RGCs relative to injured untreated RGCs (excluding C33 and C40), with all genes expressed in injured RGCs set as background. False discovery rate (FDR) was used for multiple testing correction. A subset of GO:BP terms containing the genes Dynlt1a and Lars2 were plotted in a Gene-Concept Network Plot using the clusterProfiler and enrichplot R packages.

### Statistical Analyses.

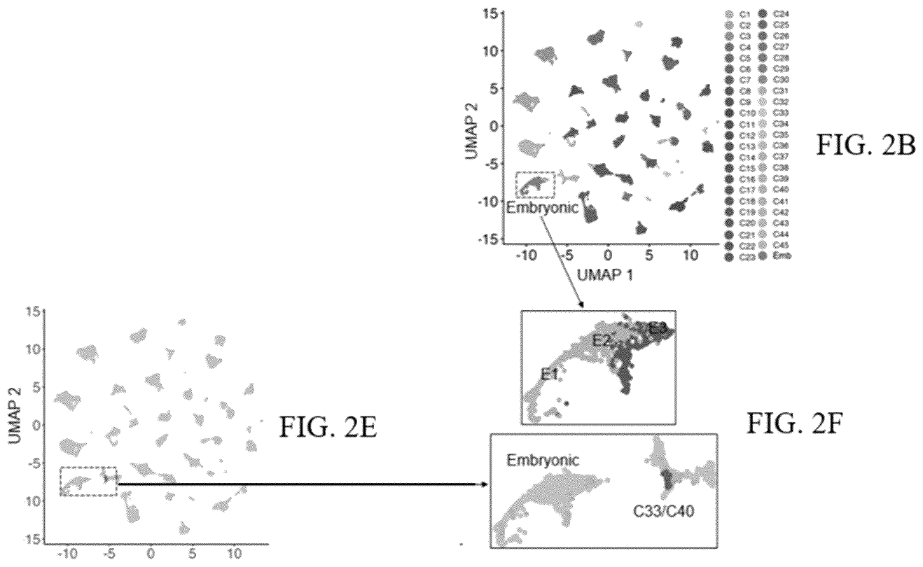

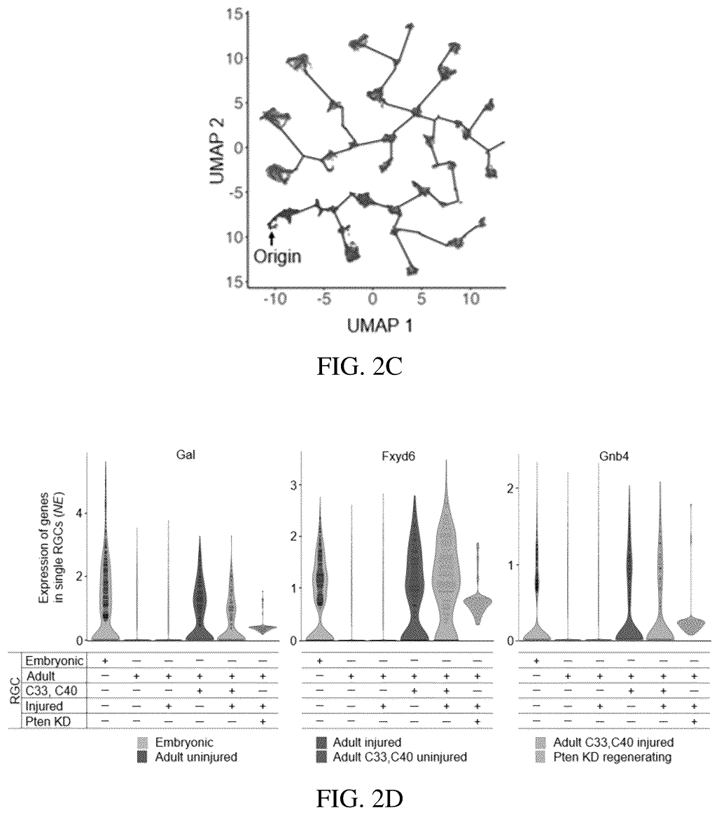

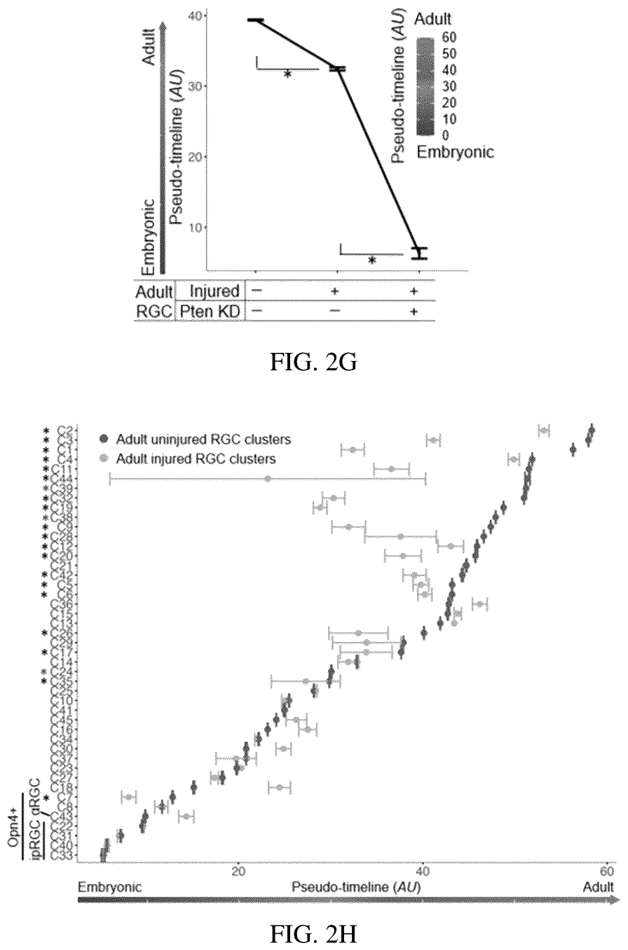

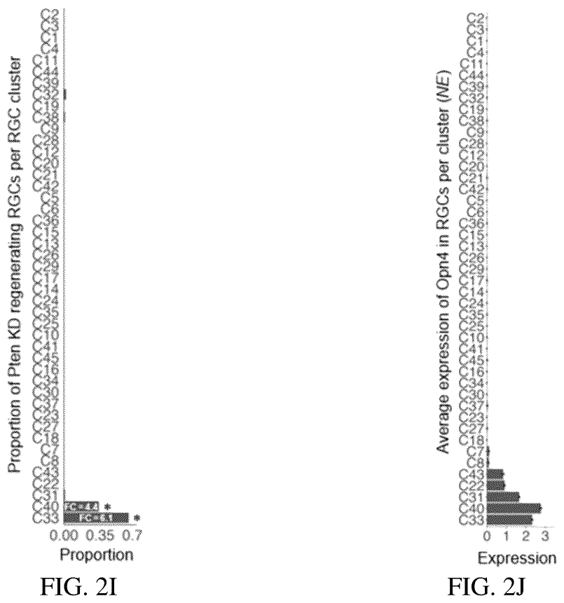

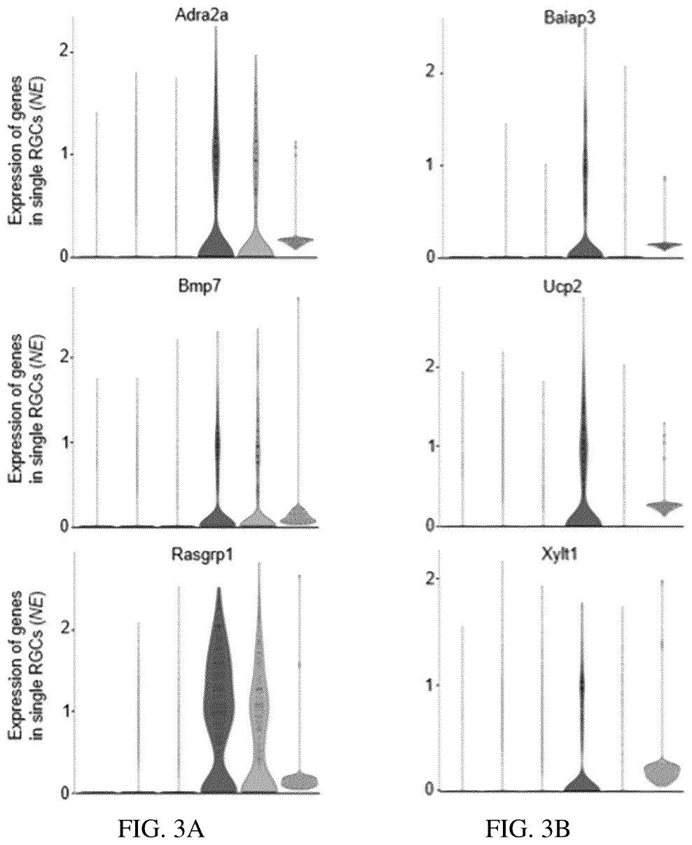

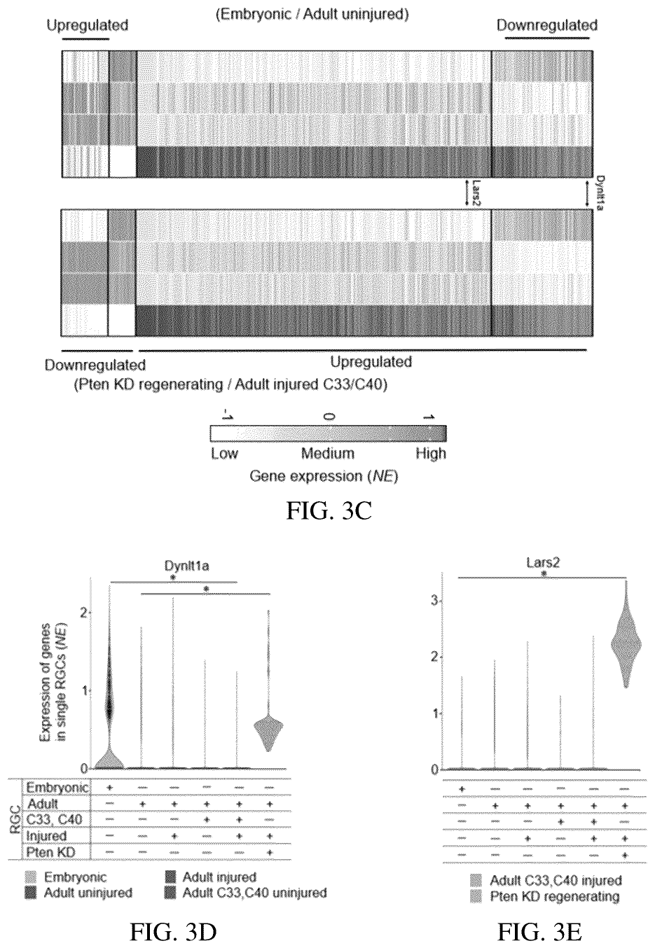

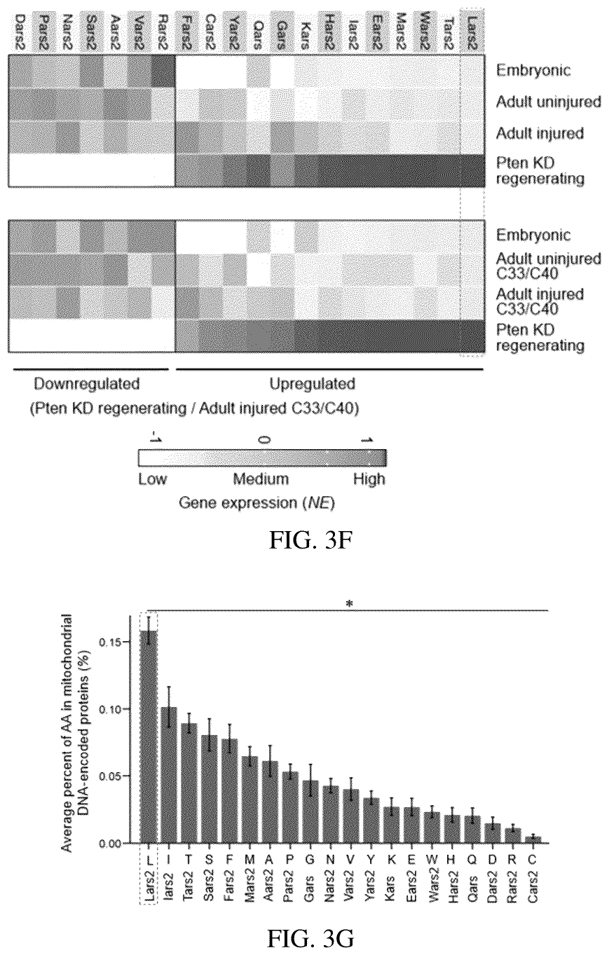

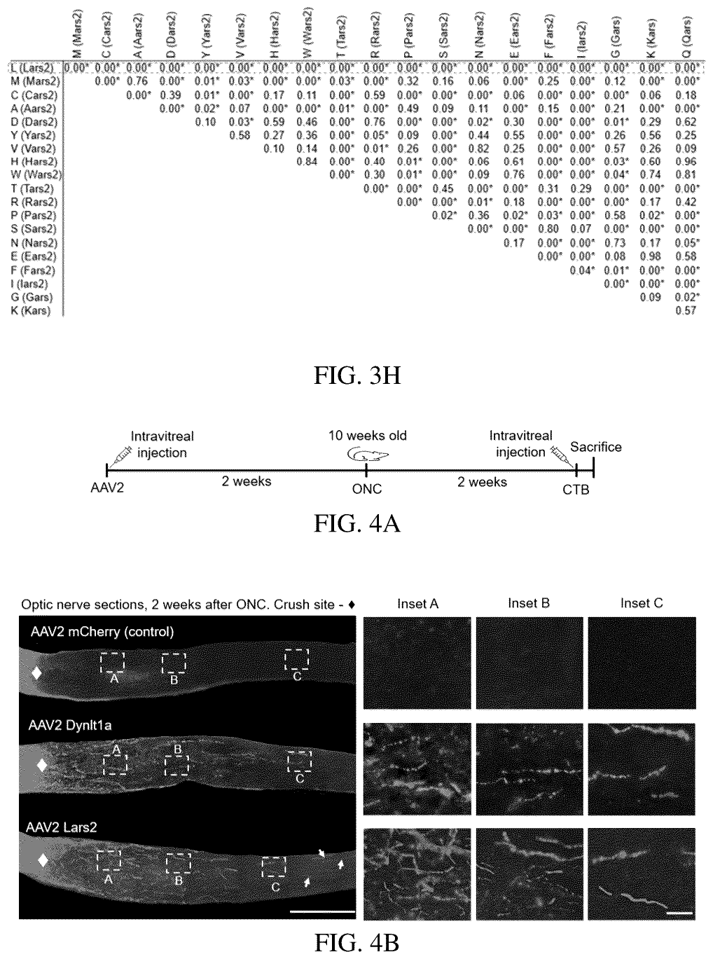

All tissue processing, quantification, and data analysis were done masked throughout the study. Sample sizes were based on accepted standards in the literature and our prior experiences. Sample size (n) represents total number of biological replicates in each condition. All experiments included appropriate controls. No cases were excluded in our data analysis, although a few animals that developed a cataract in the injured eye were excluded from the study, and their tissues were not processed. The data are presented as Means±SEM and was analyzed (as specified in the applicable Figure legends) by ANOVA with or without Repeated Measures and a posthoc LSD test (SPSS). Significance of enrichment fold-change in FIG. 2I was determined using the EdgeR algorithm77. Significance for DEGs in the heatmaps in FIG. 3 was determined by independent samples Mann-Whitney U test, using R software as we previously published. All differences were considered significant at p<0.05.

### Example 1: Transcriptomic Profiling of RGCS that Regenerated Long-Distance Axons in Response to Pten KD

Using scRNA-seq, we analyzed the transcriptomes of RGCs which responded to Pten KD by regenerating long-distance axons (i.e., 3 mm or longer beyond the injury site). Pten was KD in adult RGCs using an intravitreally injected adeno-associated virus serotype 2 (AAV2) vector, which preferentially transduces RGCs and expresses anti-Pten shRNAs known to promote axon regeneration after optic nerve crush (ONC) injury. Two weeks after ONC, long-distance axon-regenerating RGCs were isolated by Fluorescence-Activated Cell Sorting (FACS) from a single cell retinal suspension. At 12 hours prior to sacrifice, Alexa Fluor 488 conjugated Cholera toxin subunit B (CTB) axonal tracer was injected into the optic nerve 3 mm distally from the ONC site. CTB was retrogradely transported to the RGC soma in the retina via long-distance regenerated axons. mCherry reporter (identifying the AAV2-transduced cells) and Alexa Fluor 488 (identifying long-distance regenerating RGCs) double-positive RGCs were isolated by FACS (FIGS. 1A-1C).

CTB was injected distally from the injury site (as opposed to proximally, at 1.5 mm, as was done in another study), in order to identify the RGCs which regenerated long-distance axons (3 mm or longer). Multiple experimental approaches stimulate short-distance (i.e., up to 1.5 mm past the injury site) axon regeneration, but few stimulate long-distance regeneration. Moreover, even in the approaches which lead to long-distance regeneration, only a rare subset of RGCs regenerate axons 3 mm or longer, whereas the majority of the responding RGCs regenerate axons only a short-distance and stall growth. Because long-distance regeneration is more relevant for providing clues to developing approaches for full-length axon regeneration, we developed a surgical technique (that allows visual confirmation of appropriate targeting) for injecting CTB into the optic nerve 3 mm distally from the injury site (see Methods). This method prioritized the identification of this rare subset of long-distance axon-regenerating RGCs over capturing many RGCs but mostly those that regenerate only short-distance. Accordingly, only a small subset of the Pten KD-treated RGCs (identified by the expression of mCherry reporter) were CTB+(FIG. 1D), which is consistent with reports that only a rare subset of RGCs regenerate long-distance axons in response to Pten KD. No CTB+ RGCs were found in the retinas treated with the control vector expressing scrambled shRNAs and an mCherry reporter (FIG. 1E), as there were no regenerated axons in the control to uptake the CTB that was injected distally from the injury site. Because FACS'ing millions of retinal cells (from 10 retinas) would adversely affect primary neurons such as RGCs, which comprise only <1% of all the retinal cell types (e.g., recovery of RGCs from whole retinas for scRNA-seq was limited to only 432 RGCs from multiple retinas), RGCs were first enriched by immunopanning for Thy1 from the retinal cell suspension (as described previously). Cells were then FACS'ed for mCherry+/CTB+ cells, and immediately processed by plate-based droplet scRNA-seq. The transcriptomes of 101 long-distance axon regenerating RGCs passed quality control (QC; see Methods), which is a statistically appropriate representative sample of the target population comprised of only ˜90 RGCs per retina (representing 0.02% of the total retinal RGCs) that respond to Pten KD by regenerating long-distance axons at 2 weeks after ONC.

We began by comparing the transcriptomes of RGCs, which regenerated long-distance axons in response to Pten KD, to adult uninjured and injured RGC scRNA-seq transcriptomes and found that these long-distance axon-regenerating RGCs expressed canonical pan-RGC markers (Rbpms, Slc17a6, Tubb3, and Scng) in a similar range as uninjured or injured (non-treated) adult RGCs (FIG. 1F). We then analyzed developmental regulation, and KD efficiency, of Pten gene expression, by comparing RGC scRNA-seq transcriptomes of adult Pten KD-treated and untreated to embryonic. We found that the expression of Pten gene was substantially upregulated during RGC maturation from embryonic to adult age, and that KD of Pten by AAV2 expressing anti-Pten shRNAs substantially reduced Pten expression in the Pten KD long-distance axon-regenerating RGCs to even below embryonic level (FIG. 1G). We also found that the transcriptomes of the Pten KD long-distance axon-regenerating RGCs segregated into 2 clusters (FIG. 2A).

### Example 2: RGC Subtypes Retaining Features of an Embryonic State Exist in Adult Retina

As RGCs are born in waves, early and late developmental stage RGCs are present in the retina at the same time. Thus, it is possible for various stage embryonic RGCs to be isolated from the same-age retinas. Together with the adult RGC scRNA-seq atlas, embryonic RGC scRNA-seq dataset enabled generating a developmental pseudo-timeline spanning the progression from embryonic into adult cell states (FIGS. 2B-C). In analyzing the relative transcriptomic changes progressing from immature embryonic into a mature adult RGC state (along the developmental pseudo-timeline), we unexpectedly found that within the adult RGC subtypes themselves, some remain more similar to the embryonic state whereas others change more during developmental maturation (FIG. 2C). We then identified marker genes, Gal, Fxyd6, and Gnb4, that are expressed in embryonic RGCs and in adult RGC subtypes that are the most similar to embryonic, C33 and C40, which further support the existence of adult neuronal subtypes that retained features of embryonic cell state (FIG. 2D).

### Example 3: Pten KD Regenerates Long-Distance Axons Through Dedifferentiating RGCS Towards an Embryonic State

We then bioinformatically mapped (using an integration algorithm, see Methods) the Pten KD long-distance regenerating RGCs to the UMAP of embryonic and adult atlas RGCs, and found that almost all Pten KD long-distance regenerating RGCs were assigned to embryonic RGCs and to adult ipRGC clusters C33/C40 that are the closest to embryonic RGCs (FIG. 2E). Specifically, Pten KD long-distance regenerating RGC cluster A mapped to the ipRGC cluster C40 and embryonic RGCs, whereas Pten KD long-distance regenerating RGC cluster B mapped to the ipRGC cluster C33 (FIG. 2F). When the Pten KD long-distance axon-regenerating RGCs are bioinformatically mapped (see Methods) only to the adult atlas RGC UMAP (without embryonic RGCs), almost all (96%) are assigned only to the non-α ipRGC clusters C33 and C40. By contrast, using the same method to bioinformatically map the Pten KO mostly short-distance axon-regenerating RGCs to the atlas RGC UMAP, the cells are assigned primarily to αRGCs, consistent with the original report from which the scRNA-seq dataset on the Pten KO short-distance axon-regenerating RGCs was obtained (data not shown). Modest upregulation of only one αRGC marker Spp1 in the C33/C40 Pten KD long-distance axon-regenerating is consistent with prior reports of that Pten KO leads to Spp1 upregulation (data not shown).

We also found that transcriptome of even untreated adult RGCs overall, and particularly transcriptomes of 19 clusters, that survived 2 weeks after injury (FIG. 2G-H), partially reverted towards embryonic state, but not as close as the Pten KD long-distance regenerating RGC transcriptomes (which are the closest to the embryonic state; FIG. 2G). Moreover, the subtypes to which the Pten KD long-distance axon-regenerating RGCs mapped (C33 and C40) are the closest to embryonic state even in an uninjured state (FIG. 2H), and the proportion of Pten KD long-distance regenerating RGCs that mapped to these clusters is substantially enriched compared to the proportion of C33 and C40 in the RGC atlas (FIG. 2I).

### Example 4: RGCS that Regenerate Long-Distance Axons Originate from the Adult Embryonic-Like Subtypes

The existence of adult neuronal subtypes that retained features of embryonic cell state raises the possibility that they might be the RGCs which regenerated long-distance axons in response to Pten KD, particularly considering that the long-distance regenerating RGCs transcriptomically mapped almost exclusively to these embryonic-like adult RGC subtypes, C33 and C40. C33 and C40 are ipRGC subtypes within a subset of Opn4+ clusters, C7, C8, C43, C22, C31, C40, and C33, of which the last four are ipRGCs (see Table S2 in Tran et al. Neuron 12, 2019, 104:1039-1055). Although all Opn4+ clusters (FIG. 2J) are closer to embryonic state on the pseudo-timeline compared to all other clusters (FIG. 2H), the RGCs which regenerated long-distance axons in response to Pten KD mapped only to the two closest to embryonic state ipRGC clusters (C33 and C40) (FIG. 2H-I). Pten KD long-distance axon-regenerating RGCs mapping to C33 and C40 is partially consistent with the hypothesis that the Opn4+α and ip RGC types that are more resilient to injury are more responsive to Pten inhibition for regenerating axons. Therefore, RGCs which regenerated long-distance axons in response to Pten KD may have originated from the non-α ipRGC subtypes C33 and C40. It is also possible, however, that other RGC subtypes responded to Pten KD and regenerated long-distance axons but dedifferentiated towards an embryonic state (which facilitated long-distance axon regeneration) and thus transcriptomically mapped to embryonic-like RGC subtypes, C33 and C40.

To resolve between these possibilities, we analyzed whether Pten KD long-distance axon-regenerating RGCs are enriched only for markers of C33 and C40 that are also enriched in embryonic RGCs (which could be a consequence of any subtype upregulating embryonic RGC genes during dedifferentiation towards embryonic state), or are they also enriched for markers of C33 and C40 that are unique to a mature cell state and not expressed in embryonic RGCs (which would suggest origination of the long-distance axon-regenerating RGCs from subtypes C33 and C40). We found that a substantial portion of the differentially expressed genes (DEGs) in the Pten KD long-distance regenerating RGCs reverted their levels of expression towards an embryonic RGC state (FIG. 3C), while retaining the expression of embryonic RGC, and embryonic-like adult RGC subtypes, markers, Gal, Fxyd6, and Gnb4 (FIG. 2D). We also found that markers of C33 and C40 subtypes that are not expressed in embryonic RGCs, are also enriched in the Pten KD long-distance axon-regenerating RGCs, namely, Adra2a, Bmp7, and Rasgrp1, as well as Baiap3, Ucp2, and Xylt1, whose injury-induced downregulation was rescued by Pten KD (FIG. 3A-B). These data suggest that the long-distance axon-regenerating RGCs' transcriptomic similarity to embryonic-like subtypes C33 and C40 is a consequence of origination from these subtypes, rather than being a consequence of dedifferentiation from other subtypes to an embryonic-like state.

### Example 5: Developmentally-Regulated and Non-Regulated Genes are Differentially Expressed in Long-Distance Axon-Regenerating RGCs

Next, we identified groups of up- and down-regulated genes, which reverted their levels of expression towards an embryonic RGC state, as well as those that are not developmentally-regulated in RGCs (FIG. 3C and data not shown). The hypothesis that axonal regeneration after lesion requires recapitulation of the molecular mechanisms involved in developmental embryonic axon growth has received substantial support. However, the adult CNS environment is quite different from embryonic, and tissue-remodeling after lesion alters this environment even further. For example, the glial scar, reactive astrocytes, and immune cells (that responded to lesion) were not present along the axonal path during developmental axon growth of embryonic RGCs, and the hypothesis that axonal regeneration after lesion requires involvement of the molecular mechanisms that were not involved in developmental axon growth of embryonic RGCs has also received substantial support. DEG analysis of Pten KD long-distance axon-regenerating RGCs revealed patterns of gene expression that are consistent with both hypotheses (FIG. 3C). Therefore, we selected representative DEG candidates consistent with each hypothesis for testing in in vivo axon regeneration assay.

### Example 6: Mitochondria-Associated Dynlt1A and Lars2 are Enriched in Long-Distance Axon-Regenerating RGCs

We selected Dynlt1a, which reverted level of its expression towards an embryonic RGC state (FIG. 3D), and the non-developmentally-regulated Lars2, which was highly upregulated in long-distance axon-regenerating RGCs but otherwise expressed at a relatively low basal level across conditions (FIG. 3E). We focused on these genes, because they could regulate mitochondrial dynamics involved in axonal regeneration. Dynlt1a is involved in bi-directional cargo (such as mitochondria) transport along microtubules and regulates developmental neurite growth of hippocampal neurons. Lars2 is a nuclear DNA (nucDNA)-encoded aminoacyl-tRNA synthetase (aaRS), required for translation of mitochondrial DNA (mtDNA)-encoded proteins, by aminoacylation with Leucine (L) the mtDNA-encoded tRNA-L. MtDNA encodes the full set of tRNAs required for translation of mtDNA-encoded genes (which cannot be compensated for by the nucDNA-encoded tRNAs), and nucDNA encodes the full set of aaRSs that aminoacylate mtDNA-encoded tRNAs with their cognate amino acids (which cannot be compensated for by the aaRSs that aminoacylate the nucDNA-encoded tRNAs). We found that all aaRSs specific to the mt-tRNAs are differentially expressed in the Pten KD long-distance regenerating RGCs, with Lars2 being the most highly upregulated (FIG. 3F). Moreover, Lars2-aminoacylated L is significantly more enriched than any other amino acid in all 13 mtDNA-encoded proteins (FIG. 3G-H).

Therefore, we hypothesized that involvement of Lars2 and Dynlt1a in regulation of axonal mitochondrial dynamics may render them plausible downstream effectors of Pten KD for promoting long-distance axon regeneration.

### Example 7: Dynlt1A and Lars2 Promote Long-Distance Axon Regeneration after Optic Nerve Injury

We used a well-established 2-weeks after ONC axon regeneration assay, to test weather Dynlt1a and Lars2 are sufficient to promote long-distance axon regeneration. AAV2 vectors expressing Dynlt1a, Lars2, or mCherry control, were injected intravitreally in adult mice, and 2 weeks later ONC was performed. To visualize the regenerating axons or their absence, CTB was injected 1 day prior to sacrifice at 2 weeks after ONC. The number of regenerating axons was quantified in longitudinal sections of the optic nerve, and RGC survival was quantified in retinal flatmounts (see Methods for details; experimental timeline in FIG. 4A). We found that Dynlt1a and Lars2 promoted long-distance axon regeneration at least 2 mm and 3 mm past the injury site, respectively, compared to only minor axonal sprouting (as expected) in control animals (FIGS. 4B-4C). No spared axons were detected in either group. Dynlt1a (but not Lars2) also promoted RGC survival compared to the control group (FIGS. 4D-4E).

### Example 8: Gene Network Involving Lars2 and Dynlt1a Shows Association of Mitochondrial and Axonal Growth Biological Processes

To gain further insight into the mechanisms through which Lars2 and Dynlt1a promote axon growth, we analyzed the gene network upregulated in the Pten KD long-distance axon-regenerating RGCs. We found that the biological processes most co-enriched in the gene network (involving Lars2 and Dynlt1a) upregulated in the Pten KD long-distance axon-regenerating RGCs were related to mitochondria, axonal growth, and neurodevelopment (data not shown). Furthermore, our finding that Lars2 promotes axon regeneration, which belongs to the gene-ontology biological processes (GO:BP) “positive regulation of neuronal axonal projection”, has linked this biological process to the GO:BP “mitochondrial translation” (under which Lars2 was previously annotated). Thus, a cross-talk between mitochondrial translation and axonal growth processes, and involvement of a subset of co-upregulated gene network, may underlie our finding that overexpression of Lars2 or Dynlt1a can promote axon regeneration independently from each other.

### Example 9: Novel Target RNAs Play A Role in Axon Regeneration

These studies led to the identification of a number of developmentally regulated mRNAs, and mRNAs differentially expressed by a treatment with anti-Pten shRNAs exclusively in the small subset of the RGCs that regenerated long-distance (at least 3 mm) axons (see Table 7).

Some of the revealed mRNAs were already known to play a role in axon regeneration, however, many novel targets were found, and some novel targets were tested in an axon regeneration assay in vivo. We identified 17 novel mRNAs, which expressing (through specifically designed ORFs) or knocking-down (through specifically designed shRNAs) promoted axon regeneration in vivo (FIGS. 5-7).

Specifically designed mRNAs for overexpression were: Rpl7a, Rpl7, Tceb2, Lancl1, Atp6v0c, Dynlt1a, Mrtfa, Lars2, Dpysl5, Fblim1, Dypsl3, and Nfe213.

Specifically designed shRNAs for knock-down were: Mmp9, Rax, Crx, Pdnp, Prdm13, and ift20 mRNA and its lncRNA isoform ENSMUST00000128788.

A number of the identified novel targets, which we have not tested yet in axon regeneration assay, are also candidates for regulating axon regeneration and neuroprotection. Because many of those which we tested in fact regulated axon regeneration, this provides a proof-of-concept that others, which we identified using the same approach but not tested yet, are also promising targets, specifically, the RNAs differentially expressed by a treatment with anti-Pten shRNAs exclusively in the small subset of the RGCs that regenerated long-distance (at least 3 mm) axons, as listed in Table 7.

In the in vivo axon regeneration assay, which is an established mouse model of traumatic optic neuropathy, the AAV2 for expressing or knocking down expression of target RNAs was injected 2 weeks prior to injury, in order to allow sufficient time for ORF and shRNA expression. These pre-treatment studies are important for supporting the therapeutic use of these specifically designed RNAs as preventive compounds, and not only for post damage treatment.

### Example 10: Knockdown of Expression in RGCs of Upregulated piRNAS by Specifically Designed ShRNAs Promotes Axonal Regeneration Short Term and Long Term

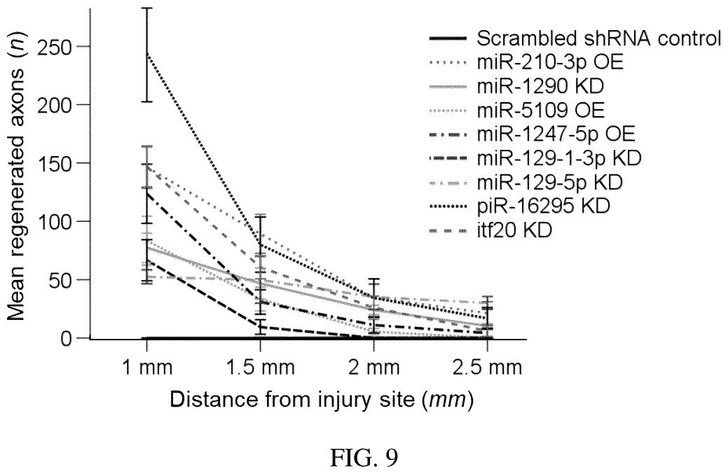

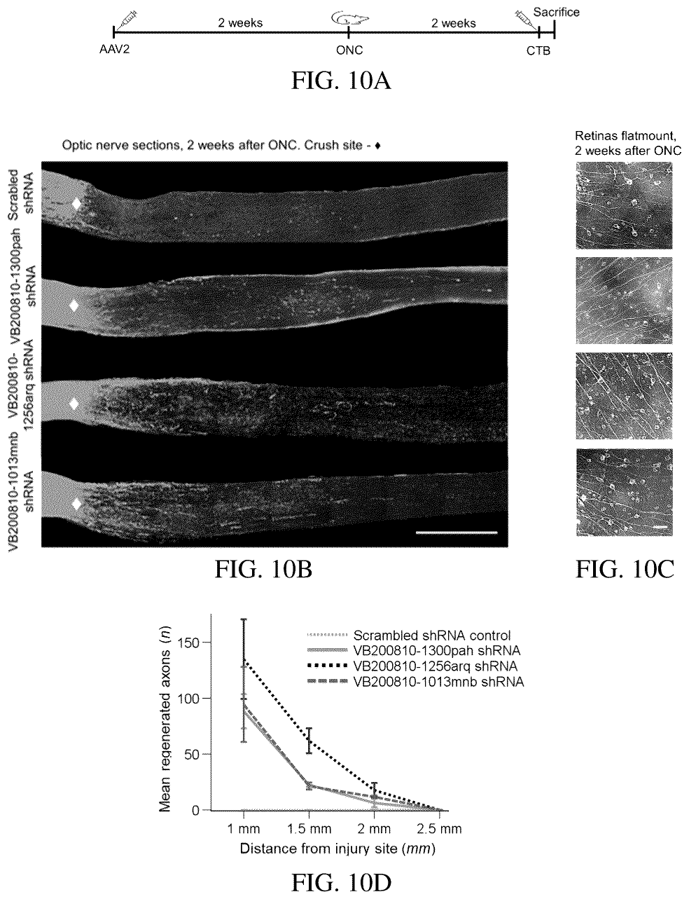

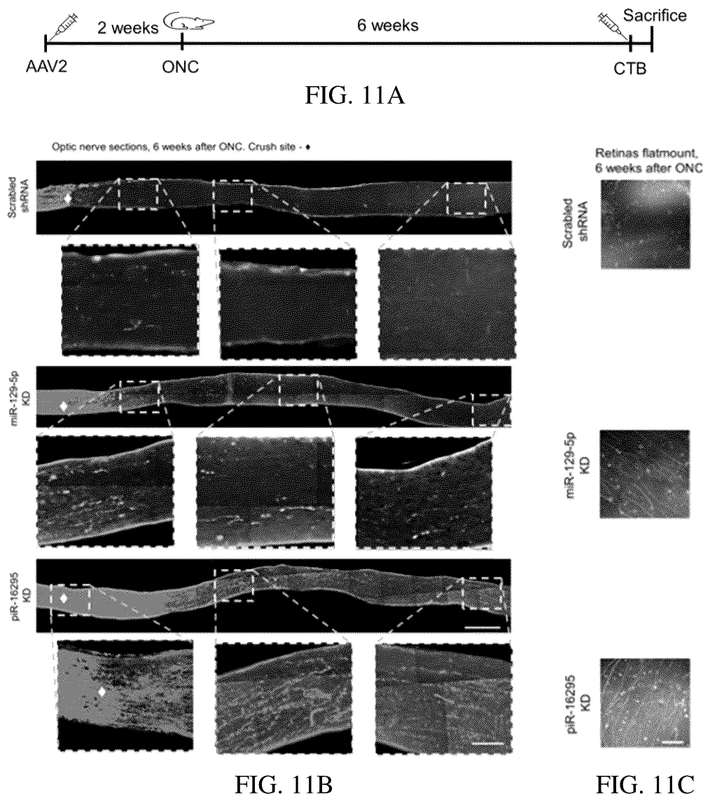

RNA-seq technology which can detect small-noncoding RNAs (sncRNA), was used to identify snRNAs that are developmentally regulated in the retinal ganglion cell (RGC) neurons as they mature. We hypothesized that the sncRNAs, which we found to be developmentally upregulated in the maturing RGCs, are involved in the failure of mature adult RGCs to regenerate axons after optic nerve injury. Therefore, we designed the shRNAs to knockdown or upregulate the expression of the identified sncRNAs and delivered them selectively to the RGCs in the mouse eyes using AAV2. These studies led to the identification of a number of developmentally upregulated miRNAs and Piwi-interacting RNAs (piRNA), which is a class of sncRNAs, whose roles in the CNS are largely unknown, expressing (through shRNAs we designed and engineered for expression from viral vectors) or knocking-down (also through shRNAs we designed and engineered for expression from viral vectors) which promoted axon regeneration after injury in vivo. Specifically, for overexpression miR-210-3p, miR-5109, and miR-1247-5p; for KD: miR-129-1-3p, miR-129-5p, miR-1290, piR-16295, and ift20 mRNA and its lncRNA isoform ENSMUST00000128788 (FIGS. 8-9; the full sequence and AAV2 vector design IDs shown in the sequence listing). We also designed shRNA sncRNAs that may act on several putative targets (VB200810-1300pah, VB200810-1013mnb, and VB200810-1256arq), expressing which promoted axon regeneration after injury in vivo (FIGS. 10A-10D). We also identified several less potent but still promising novel neuroprotective/neuroregenerative sncRNA targets for OE (miR-3914-1, miR-135b-3p, miR-135a-3p, sncRNA VB200810-1672kgn, and sncRNA VB200810-1670qan), and for KD (miR-6090, miR-132-3p, and miR-29a-3p), which elicited marginally less axon regeneration than the sncRNAs in FIGS. 8-10. Furthermore, knocking-down either piR-16295 or miR-129-5p promotes RGC survival and axon regeneration not only in short-term (2 weeks after injury) but even in long-term (6 weeks after injury) assays (FIGS. 11A-11C). Two-way (or more ways) combinations with other disclosed herein novel nucleic acid molecules, or their targets, are expected to promote neuronal survival and axon regeneration synergistically or additively more effectively over longer-term than each alone.

In these assays, the AAV2 was injected 2 weeks prior to injury, in order to allow sufficient time for shRNA expression. These pre-treatment studies provide support for the use of the shRNAs for a preventative therapeutic, and not only for post-damage treatment.

### Example 11: Adult Retinal Ganglion Cells Survive and Regenerate Axons when Cultured on Vitronectin or Fibronectin

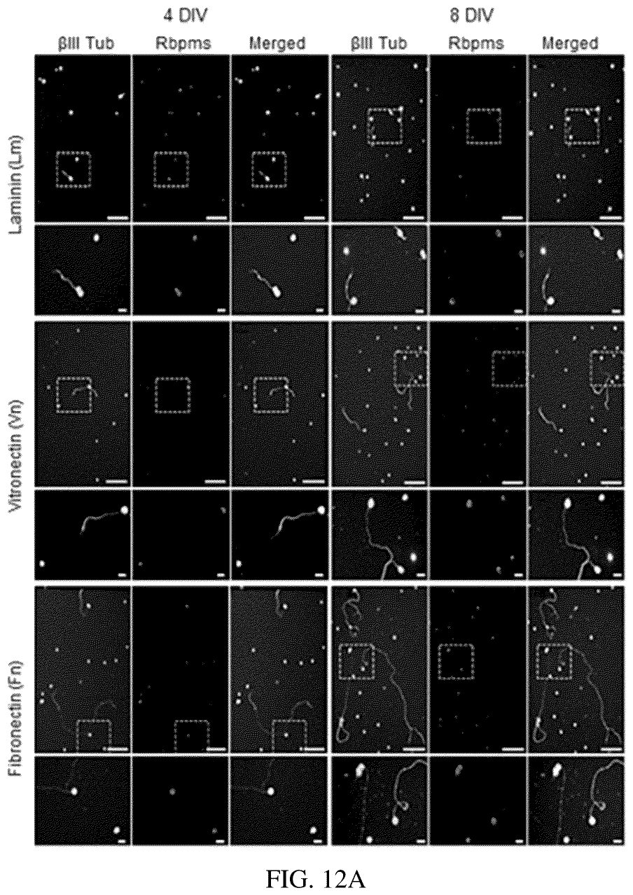

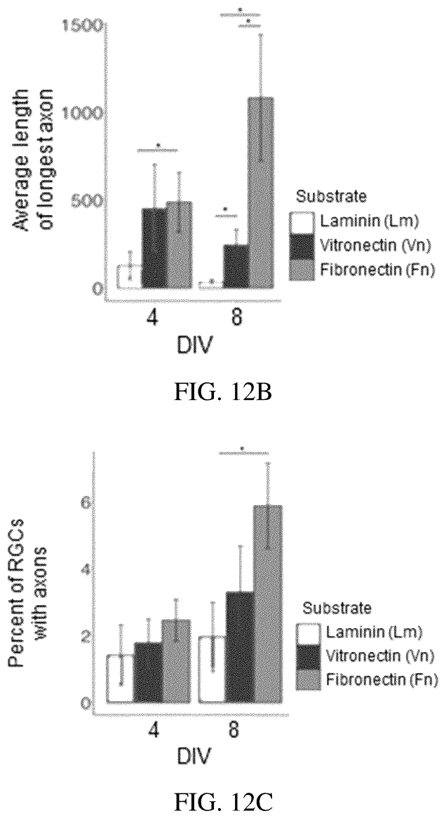

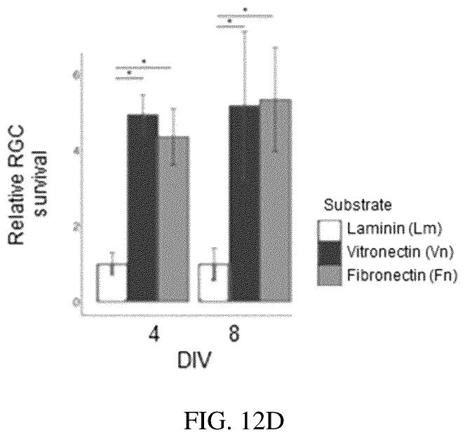

Previous research showed that the CNS projection neurons lose the capacity for regenerating axons in vivo as they mature. Mature CNS projection neurons, such as the retinal ganglion cells (RGCs), also do not grow axons in culture on laminin-coated surface. However, there is robust axon growth when immature RGCs, and other CNS neurons, are grown on laminin. The present inventors surprisingly and unexpected discovered that a subset of the adult RGCs survived better and regenerated axons when cultured on vitronectin, and even better when cultured on fibronectin (FIGS. 12A-12D).

FIG. 12A shows representative images of 10-week-old adult RGCs cultured on either laminin, vitronectin, or fibronectin for 4 days in vitro (DIV) or 8 DIV. Quantification showed that the fibronectin condition had the longest regenerating axons (FIG. 12B) and the most regenerating axons (FIG. 12C), specifically at 8 DIV. As shown in FIG. 12D, quantifications of RGC survival showed that by 4 DIV and 8 DIV both fibronectin and vitronectin treatment/treatment had significantly higher RGC survival as compared to laminin condition/treatment. N=3; n≥45; * p<0.05 by ANOVA; Error bars, SEM. Scale bars, main panels=50 μm; insets=10 μm. Lm: Laminin; Vn: Vitronectin; Fn: Fibronectin.

### Example 12: Axon Regeneration Promoted, or Enhanced in a Combination with Another Treatment, by Experimental Treatment with a Fibronectin-Derived Peptide

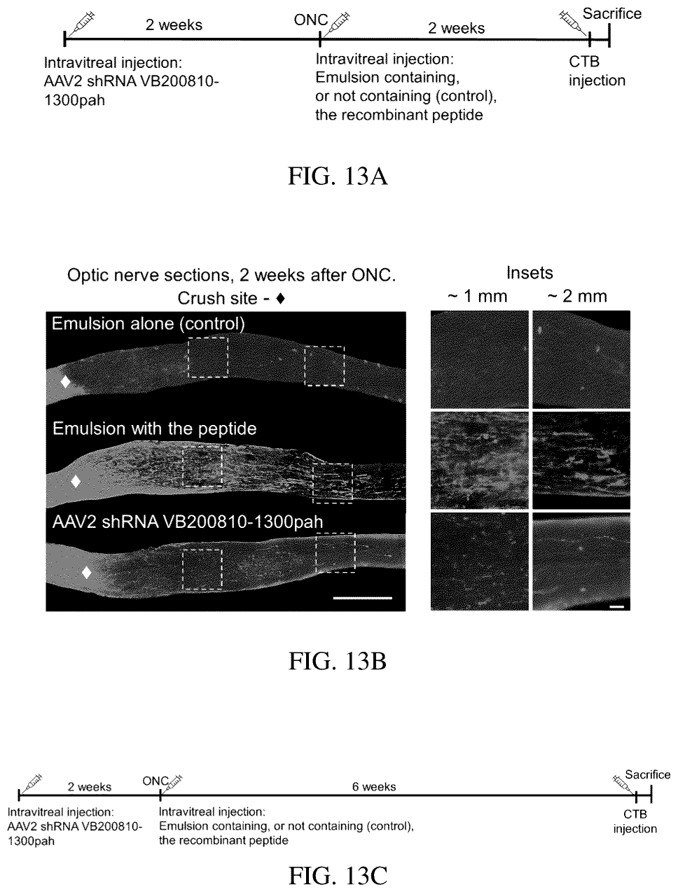

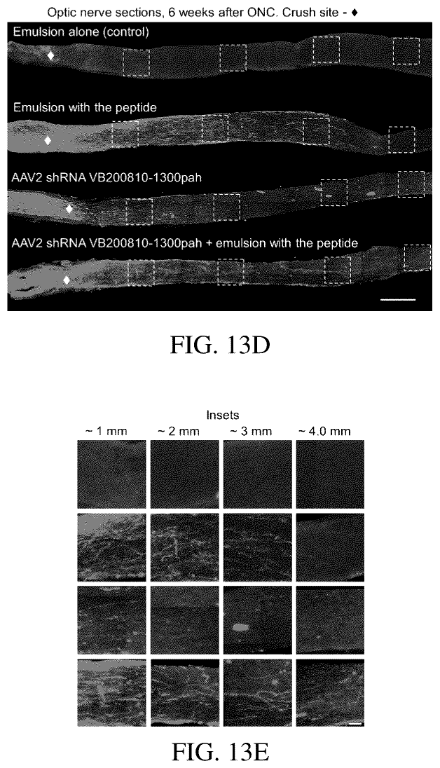

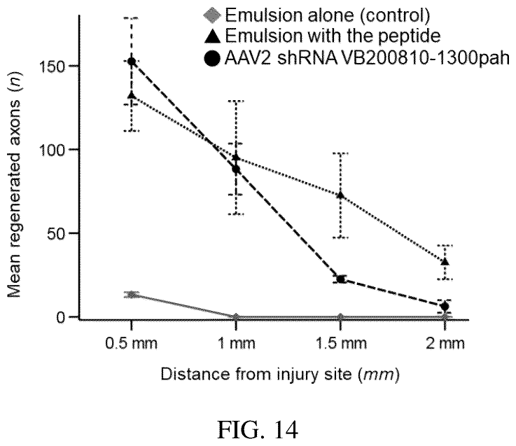

Fibronectin was previously shown to inhibit differentiation and myelination, and the present inventors recently discovered that post-injury born oligodendrocytes integrate into the glial scar and inhibit axon regeneration in the optic nerve. Therefore, fibronectin could promote axon regeneration by acting on injured neurons directly, and also by preventing post-injury born and surviving oligodendrocyte lineage cells from inhibiting axon regeneration. However, fibronectin is a large protein, thereby making the delivery of fibronectin into a site of injury not clinically feasible. Therefore, based on fibronectin, the present inventors designed and examined the ability of various small recombinant peptides to promote axon regeneration. The present inventors surprisingly and unexpectedly discovered that a recombinant peptide comprising fibronectin amino acids 1359-1528 promoted axon regeneration in vivo (FIGS. 13A-13E and FIG. 14).

The recombinant peptide design was based on the amino acids 1359-1528 of fibronectin variant GenBank ID AAI17177.1, which contains the Arg-Gly-Asp (RGD)-motif within the fibronectin type III domain. This amino acid sequence is found at different locations depending on the fibronectin isoform. For example, in fibronectin GenBank ID KAI2526833.1 its amino acids 166-335, in fibronectin GenBank ID EAW70547.1 its amino acids 434-603, and in fibronectin GenBank ID KAI2526830.1 its amino acids 1450-1619. The 195 amino acid recombinant peptide is shown below and further includes a signal peptide added to the N-terminus of the fibronectin-based sequence (underlined), and a His tag peptide added to the C-terminus of the fibronectin-based sequence (bold and italicized).

(SEQ ID NO: 56)
MGWSCIILFLVATATGVHSDSPTGIDFSDITANSFTVHWIAPRATITGYR
IRHHPEHFSGRPREDRVPHSRNSITLTNLTPGTEYVVSIVALNGREESPL
LIGQQSTVSDVPRDLEVVAATPTSLLISWDAPAVTVRYYRITYGETGG
NSPVQEFTVPGSKSTATISGLKPGVDYTITVYAVTGRGDSPHHHHHH

The affinity tag (His tag) of the recombinant peptide enables isolation and purification of the peptide, which was then concentrated to about 0.2 mg/ml in phosphate buffered saline (PBS, pH 7.2), sterilized via a 0.22 μm filter, and aseptically aliquoted for long-term storage at −80° C. The recombinant peptide is stable and retains its therapeutic efficacy for at least a year when stored properly.

The present data demonstrates that the fibronectin-based peptide can be utilized with various signal peptides, with or without any appropriate purification tag, as a peptide therapeutic, such as a peptide therapeutic to repair neural connections or prevent damage to neural connection.

The in vivo axon regeneration assay is an established mouse model of traumatic optic neuropathy, wherein an emulsion with the peptide was injected intravitreally into the eye at the time of injury. A water-in-oil (w/o) emulsion of the recombinant peptide in PBS and silicon oil was used, because the emulsion distributes slowly in the eye, enabling the continuous, slow-release of the recombinant peptide over time after injury. In addition, silicon oil injected into the eye is known to propagate into the optic nerve, where the recombinant peptide can act on the injured axonal stamps (which become growth cones) and on the oligodendrocyte lineage cells. As such, injection of the recombinant peptide in PBS alone leads to a less robust axon regeneration. The emulsion was made as follows to prepare the recombinant peptide for targeted injection. To make approximately 300 μl of the recombinant peptide emulsion, 232 μl of silicon oil (purified polydimethylsiloxane with viscosity of 1000 cs; Silikon 1000, catalog #8065601187, Alcon) was added to a 0.6 ml tube. Next, 100 μl of 0.2 mg/ml of the recombinant peptide in PBS was added, followed by 1 μl of surfactant (7.5% Triton 100× diluted in water). Next, a homogenizer (Dremel® model 400, 120V) with a steel rotary rod (part #191, Dremel®) was used at 12500 rotations per minute (RPM) to create an emulsion of oil-based phase and water-based phase, by mixing them through immersing in and out of the mix (not too fast nor too slow) without touching walls or bottom of the tube until an intermediate cloudy foam-like phase (emulsion, approximately 40 μl) appeared, which takes about 2 minutes. Mixing longer can make the phase too dense. The tube was stored at 4° C. overnight to allow the phases to separate better, and the emulsion phase (the intermediate cloudy phase) was used for injections within a week. On the day of injection, 5 μl was pulled from the middle of the intermediate cloudy phase and dispensed into a tube cap. Three μl from the tube cap was then immediately injected intravitreally with a needle right after optic nerve crush (ONC) injury.

In order to test whether the recombinant peptide could enhance neuroregenerative effects of other treatments, the RGCs in mice were pre-treated by an intravitreal injection of an AAV2 vector expressing shRNA with the expressed functional sequence GCTGTGAAGGTGAATCTGGATTTCCTTGAA (SEQ ID NO: 33), which as discussed herein promotes neuroprotection and axon regeneration (the full sequence and AAV2 vector design ID VB200810-1300pah was discussed above). Two weeks after pre-treatment with the shRNA AAV2 (vector design ID VB200810-1300pah), optic nerve crush (ONC) was preformed and mice were split into two groups, in one group mice were co-treated with the intravitreally injected recombinant peptide immediately after injury (as described above), and another group were sham treated for control. It was surprisingly and unexpectedly discovered that the combinatory treatment yielded the longest distance axon regeneration in the long-term (i.e., 6 weeks after injury end point) studies, with the axons regenerating through the entire length of the optic nerve, and some axons regenerating even further through the optic chiasm and into the optic tract (FIGS. 13A-13E, FIG. 14 and FIG. 15).

FIG. 13A illustrates the experimental timeline for the short-term study: 8 weeks old mice were pre-treated with experimental AAV2 vector VB200810-1300pah expressing shRNA. Optic nerve crush (ONC) injury was performed 2 weeks later, followed by treatment with the (1) experimental emulsion containing the recombinant peptide in PBS or (2) control emulsion containing PBS without the peptide. Animals were sacrificed for histological analysis 2 weeks later. Axonal tracer CTB was injected intravitreally 1 day prior to sacrifice. Representative images of the optic nerve longitudinal sections with axonal tracer Cholera toxin subunit B (CTB)-labeled axons through the optic nerve at 2 weeks after ONC from the animals treated with experimental emulsion containing the (1) recombinant peptide in PBS, (2) control emulsion containing the PBS without the peptide, or (3) pre-treated with AAV2 shRNA VB200810-1300pah, are shown in FIG. 13B, as marked. No axons regenerating past the injury site were present in the control, but axons regenerating substantial lengths were present in the experimental conditions. Optic nerve regions approximately 1 mm proximal and approximately 2.0 mm distal to the injury site are magnified and shown in the insets of FIG. 13B for better visualization of the regenerating axons or their absence in control condition. Scale bars: 500 μm (main panel), 50 μm (insets). FIG. 13C illustrates the experimental timeline for the long-term study: 8 weeks old mice were pre-treated with experimental AAV2 vector VB200810-1300pah expressing shRNA. ONC injury was performed 2 weeks later, followed by treatment with (1) experimental emulsion containing the recombinant peptide in PBS or (2) control emulsion containing the PBS without the peptide. Animals were sacrificed for histological analysis 6 weeks later. Axonal tracer CTB was injected intravitreally 1 day prior to sacrifice. Representative images of the optic nerve longitudinal sections with axonal tracer CTB-labeled axons through the optic nerve at 6 weeks after ONC, from the animals treated with (1) experimental emulsion containing the recombinant peptide in PBS, (2) control emulsion containing the PBS without the peptide, (3) AAV2 shRNA VB200810-1300pah, or (4) co-treated with emulsion containing the recombinant peptide and AAV2 shRNA VB200810-1300pah, are shown in FIG. 13D, as marked. No axons regenerating past the injury site were present in the control, but axons regenerating substantial lengths were present in the experimental conditions, with the longest (through the end of the optic nerve) regenerating axons detected in the co-treatment condition. Optic nerve regions approximately 1 mm proximal, approximately 2 mm middle, approximately 3 mm distal, and as well as at the end of the optic nerve (approximately 4 mm) to the injury site were magnified and shown in the insets of FIG. 13D (FIG. 13E) for better visualization of the regenerating axons or their absence in control conditions. Scale bars: 500 μm (main panel), 50 μm (insets).

The axon regeneration data from FIGS. 13A-13E and shown in FIG. 14. In particular, FIG. 14 shows the quantitation of CTB-labeled regenerating axons at 2 weeks after ONC at increasing distances from the injury site, as marked (mean±S.E.M shown; n=3 cases per group). The data was analyzed using ANOVA with repeated measures, sphericity assumed, overall F=10.4, p<0.05 by posthoc LSD pairwise comparisons for the main effect of the treatment condition compared to control.

### Example 13: Axon Regeneration after ONC in the Optic Chiasm and Tract in Response to Co-Treatment with the Emulsion Containing Recombinant Peptide and AAV2 shRNA VB200810-1300pah

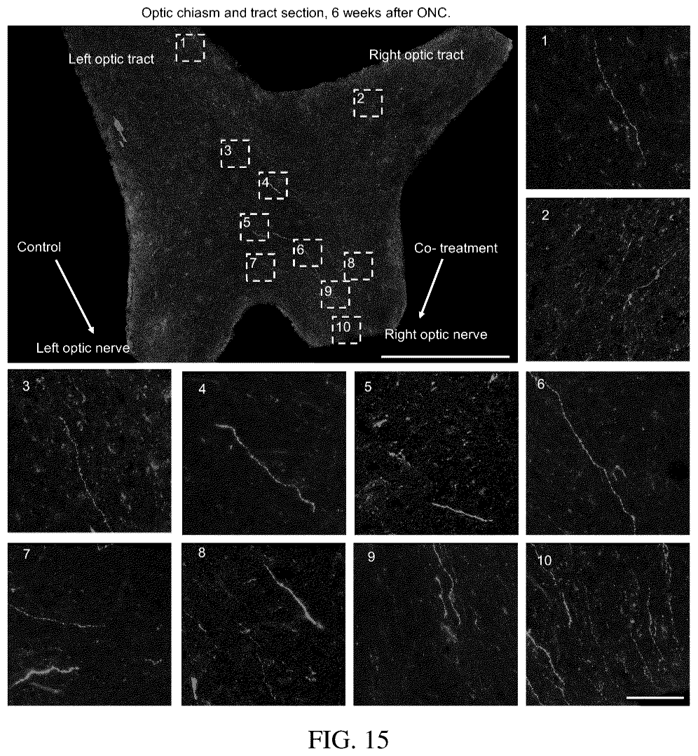

FIG. 15 shows axon regeneration over 6 weeks after ONC in the optic chiasm, tract in response to co-treatment with emulsion containing the recombinant peptide and AAV2 shRNA VB200810-1300pah. Top left is a representative image of the optic chiasm horizontal section, which includes partial optic nerves and tracts sections, with axonal tracer CTB-labeled RGC axons at 6 weeks after ONC from the animal that received co-treatment with emulsion containing the recombinant peptide and AAV2 shRNA VB200810-1300pah in the right eye, and control treatment into the left eye (indicated by arrows). Insets 1 and 2 shows the axons regenerating through the optic tracks. Insets 3 and 7 show the axons regenerating through the middle of optic chiasm. Insets 8 and 10 shows the regenerating axons from the right optic nerve entering the optic chiasm. Scale bars: 500 μm (main image, top left), 50 μm (insets).

### Discussion for Examples 1-10

The failure of CNS projection neurons to spontaneously regenerate long-distance axons after injury or in neurodegenerative disease presents a major medical problem, as even in the approaches targeting clinically-concerning tumorigenic factors only a rare subset of axons regenerated the full-length in animal models. To tackle this problem, studies showed that intrinsic axon growth capacity, which declines during maturation in mammalian CNS, can be reactivated to some extent by targeting neuronal developmentally-regulated genes (e.g., Klf4, Klf9), whereas axonal injury itself tilts the transcriptome of adult CNS neurons towards an embryonic state (although failing to elicit axon regeneration). On the other hand, targeting the tumor suppressor Pten promotes various extents of axon regeneration, mostly short-distance from αRGC and other RGC types, but also long-distance regeneration from a rare subset of α and/or ip RGC subtypes.

Here, the inventors demonstrate that although bulk-RNA-seq previously left unresolved whether injury itself tilts the transcriptome of all or only some adult CNS neuronal subtypes towards an embryonic state, scRNA-seq-enabled cluster-specific analysis revealed that only a subset of RGC subtypes revert their transcriptomes towards an embryonic state. However, marginal dedifferentiation by injury alone fails to elicit spontaneous axon regeneration (as although neurons attempt, they fail to regenerate axons after injury). Unexpectedly, it was also found that the existence of adult RGC subtypes that retained features of an embryonic cell state, and identified Gal, Fxyd6, and Gnb4, as markers which are expressed in embryonic RGCs and then downregulated during maturation in all RGC subtypes except a subset of Opn4+ RGCs, primarily subtypes C33 and C40 (that are transcriptomically more similar to embryonic RGCs than any other RGC subtype). The inventors then showed that Pten inhibition promotes long-distance axon regeneration through partially dedifferentiating towards an embryonic state the M1 ipRGCs from subtypes C33 and C40, which are able to respond because during maturation they retained features of an embryonic cell state (in contrast to other RGC subtypes).

The inventors also identified novel mitochondrial factors involved in axon regeneration. Dynlt1a is involved in bi-directional axonal transport of mitochondria along microtubules, which may be needed to supply mitochondria-generated energy (and ‘building materials’) for assembling the regenerating axonal segments. Lars2-aminoacylated L (to mt-tRNA-L) was found to be the most prevalent amino acid in (and therefore Lars2 is a liming factor for production of) all mtDNA-encoded proteins, and Lars2 is the most highly upregulated mt-tRNA-specific aaRS in the Pten regenerating RGCs. Thus, Lars2 may enable synthesis of axonal mitochondria that is needed to supply energy for assembling the regenerating axonal segments. The inventors also provided further insight into the mechanisms through which Lars2 and Dynlt1a promote axon growth, by showing that the biological processes most co-enriched in the gene network (which involved Lars2 and Dynlt1a) upregulated in the Pten KD long-distance axon-regenerating RGCs were related to mitochondria, axonal growth, and neurodevelopment. Furthermore, this finding that Lars2 promotes axon regeneration, which belongs to the gene-ontology biological processes (GO:BP) “positive regulation of neuronal axonal projection”, has linked this biological process to the GO:BP “mitochondrial translation” (under which Lars2 was previously annotated). Thus, a cross-talk between mitochondrial translation and axonal growth processes, and involvement of a subset of co-upregulated gene network, may underlie Lars2 or Dynlt1a ability to promote axon regeneration independently from each other.

The inventors' experimental approach prioritized identification of the rare subset of RGCs that regenerate long-distance axons (3 mm and longer), over capturing many RGCs but mostly those that regenerate axons only over short-distance (up to 1.5 mm). In order to reliably inject axonal tracer into the optic nerve 3 mm beyond the injury site, the inventors developed a novel surgical technique for appropriate targeting with visual confirmation (see Methods). This approach yielded insights into long-distance axon regeneration that are more relevant for providing clues to developing full-length axon regeneration treatments, and the non-tumorigenic Lars2 and Dynlt1a (identified through this approach) are viable candidates for the development of axon regeneration therapies. Experimental approach in another study by Jacobi et al. (2022, Neuron 110:2625-2645) injected retrograde tracer proximally to the injury site (1.5 mm away), which enabled the capture of many RGCs that regenerate short-distance axons, and also did not utilize comparative analysis to embryonic RGCs as provided here. Therefore, that study revealed different factors and insights than what is reported here. For example, it showed that primarily (82%) αRGCs (and 18% of multiple other RGC subtypes of which non-α ipRGCs represented only ˜1.8%; see FIG. 2G in that study) respond to Pten knockout by regenerating axons short-distance, whereas the present study revealed that primarily non-α M1 ipRGC subtypes C33 and C40 respond to Pten KD by regenerating long-distance axons (comparative analysis between short-distance axon-regenerating RGCs from that study and long-distance axon-regenerating RGCs identified herein, data not shown, but this analysis did not include cells from another similar study by Li et al. (2022, Neuron 110: 2646-2663) that also found short-distance axon-regenerating genes, because retrospect analysis found those cells to be microglia/macrophage). On the other hand, targeting the tumor suppressor Pten promotes various extents of axon regeneration, mostly short-distance from αRGC and other RGC types, but also long-distance regeneration from a rare subset of α and/or ip RGC subtypes. Also, Gal, which was previously implicated in PNS axon regeneration, was one of few identified RGC axon regeneration-promoting genes in the Jacobi study, whereas the present inventors identified Gal as one of the markers of adult RGC subtypes C33/C40 that retained features of an embryonic cell state, which enabled them to regenerate long-distance axons in response to Pten KD. Gal is enriched in RGC subtypes C33/C40 nearly 3-fold relative to αRGCs and even more relative to other RGC subtypes in the uninjured RGC atlas. However, after ONC and Pten KO Gal is also upregulated in αRGCs, although these cells did not dedifferentiate towards embryonic cell state.

The present study suggests that neuron's proximity to embryonic transcriptomic state is a predictor of its responsiveness to axon regeneration treatments, and that the adult neuronal subtypes that retain embryonic cell state features are able to regenerate long-distance axons in response to treatment. Considering that Pten KD downstream factors, Dynlt1a and more so Lars2, achieved comparable to Pten KD extent of long-distance (despite lesser extent of short-distance) axon regeneration, and considering that targeting other factors in the Pten pathway also promotes axon regeneration (even without complementary co-treatments), it is possible that several effectors may promote long-distance axon regeneration through tapping into subtype-specific and non-subtype-specific pathways, which could tilt the transcriptome towards an embryonic transcriptomic state. For example, the inventors found that expression of the axon regeneration-facilitating Tet1 demethylase (which is upregulated by a dedifferentiation treatment co-expressing Oct4/Sox2/Klf4/Myc) is substantially downregulated in the injured RGCs, but its expression is preserved (and even modestly upregulated) in the long-distance axon-regenerating RGCs that responded to Pten KD. Moreover, Sox2 component of that treatment, which is not expressed in injured or uninjured RGCs, is upregulated in the long-distance axon-regenerating RGCs that responded to Pten KD. Furthermore, several other known axon regeneration-promoting genes were also co-upregulated in the long-distance axon-regenerating RGCs by Pten KD, including Braf, Akt3, Igf1R, and Sprr1a.

Taken together, these findings reveal the existence of adult neuronal subtypes that retained features of embryonic cell state, demonstrate that the RGCs which regenerate long-distance axons in response to Pten KD originate from these embryonic-like adult subtypes and dedifferentiate towards an embryonic cell state, and identify novel long-distance axon regeneration-promoting genes, which suggest that mitochondrial protein synthesis may be rate-limiting in axon regeneration.

Furthermore, the inventors identified novel biological targets, and designed novel nucleic acid molecules, shRNAs and ORFs, that were tested for their ability to regulate both RNA expression and production of protein from the RNAs. These novel nucleic acid molecules were found to promote long-distance axon neuroprotection prior to injury and regeneration post-injury, thereby providing a therapeutic that can be used alone or in combination with other therapeutics for protection from neuropathies, prevention of progression of neuropathies, and treatment of neuropathies.

### Discussion for Examples 11-13

The present inventors surprisingly discovered that a subset of the adult RGCs survived better and regenerated axons when cultured on vitronectin, and even better when cultured on fibronectin. The present inventors surprisingly discovered that a fibronectin-peptide promoted axon regeneration in vivo.

The recombinant peptide data of Examples 11-13 demonstrates the use of the vitronectin, fibronectin, and fibronectin-based peptides as a preventive treatment or co-treatment and a post-damage treatment or co-treatment. The data demonstrates that a fibronectin-based peptide can be utilized with various signal peptides, with or without any appropriate purification tag, as a peptide therapeutic, such as a peptide therapeutic to repair neural connections or prevent damage to neural connection, such as a preventive treatment or co-treatment and a post-damage treatment or co-treatment.

### Example 14

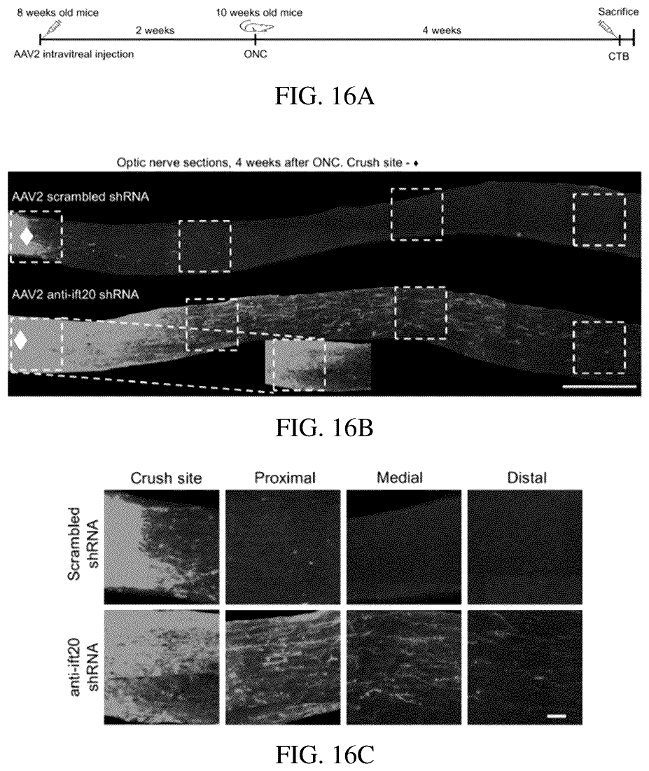

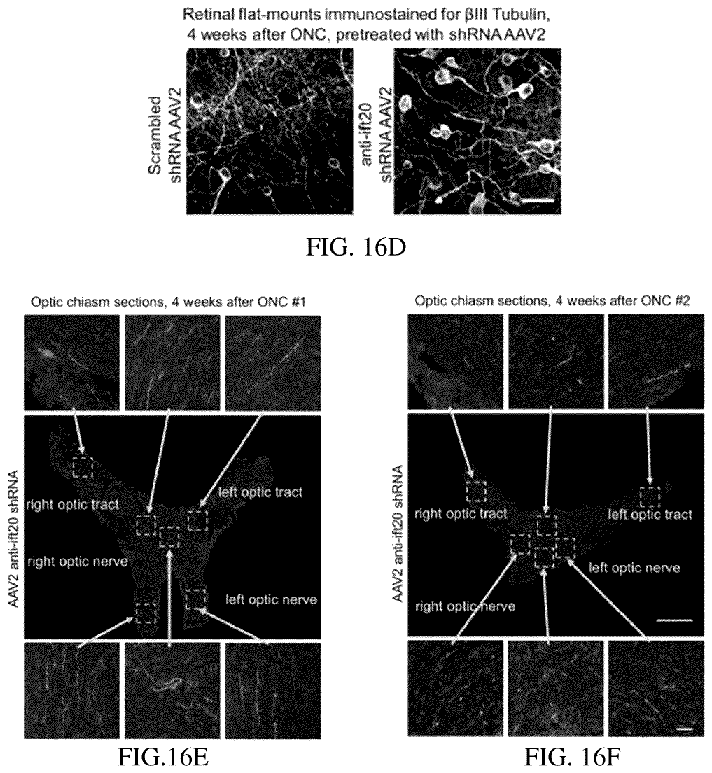

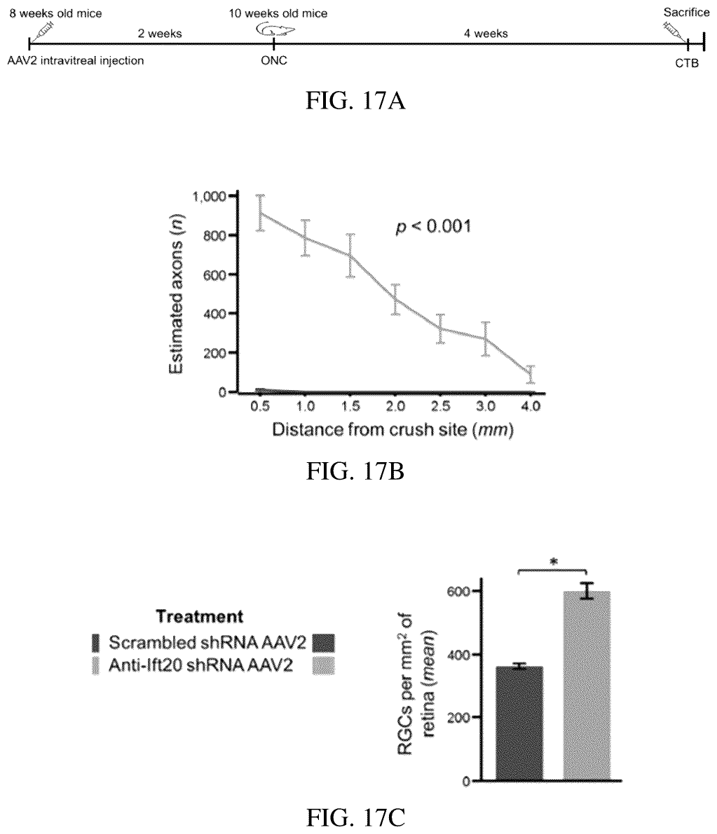

For a proof-of-concept regarding long-term effects, the long-term therapeutic potential of several molecules initially discovered to promote neuroprotection and axon regeneration at least through 2 weeks after optic nerve crush (ONC) injury was tested. Using the methods, controls, and experimental design described above for testing neuroprotection and axon regeneration at 2 weeks after ONC injury, the potential for neuroprotection and axon regeneration elicited by AAV2 vector expressing anti-ift20 was tested confirming shRNA persists by 4 weeks after ONC (experimental timeline is shown in FIG. 16A). Data show that, relative to absence of regeneration (as expected) in control condition, in the experimentally-treated condition axons regenerated through the full-length of the optic nerve (FIGS. 16B-16C, FIG. 17B), with some axons regenerating through the optic chiasm and into the optic tracts (FIGS. 16 E-16F). RGC survival also was significantly promoted by the anti-itf20 shRNA AAV2 at 4 weeks post-injury, compared to the control-treated animals (FIGS. 16D, 17C).

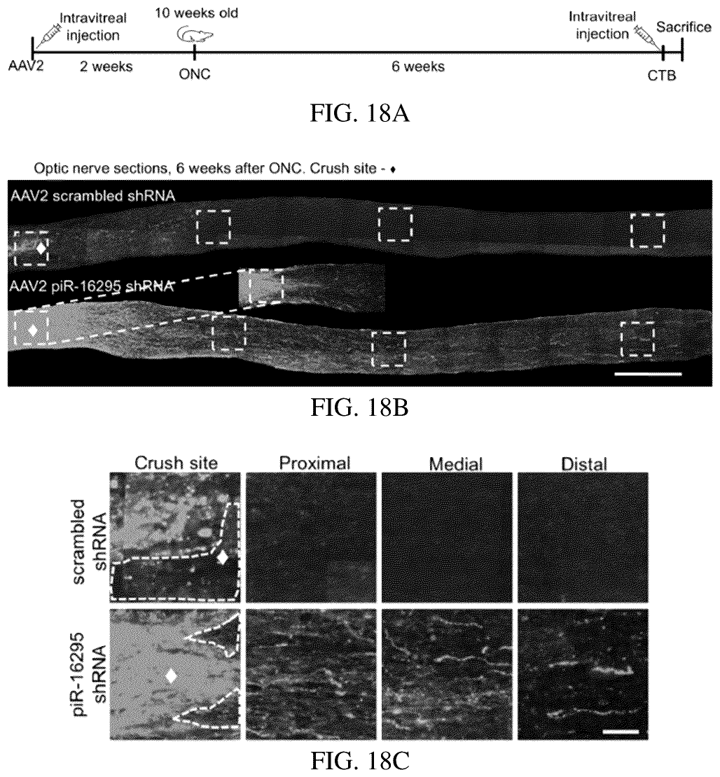

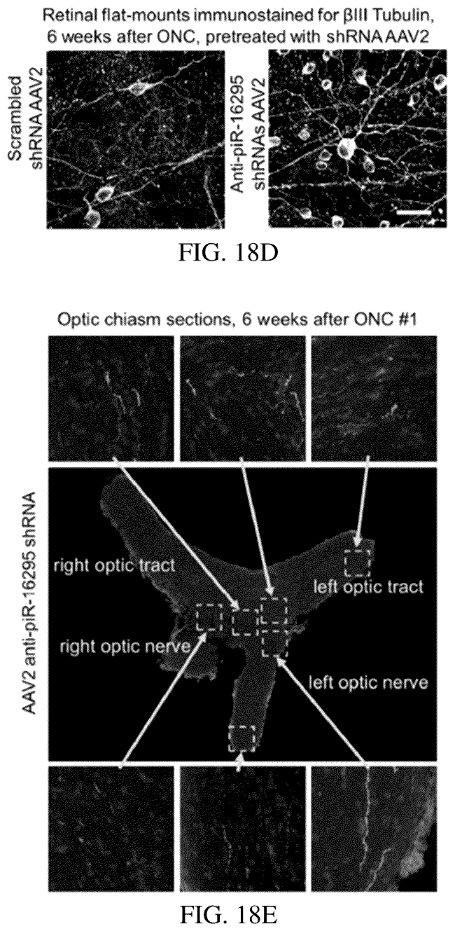

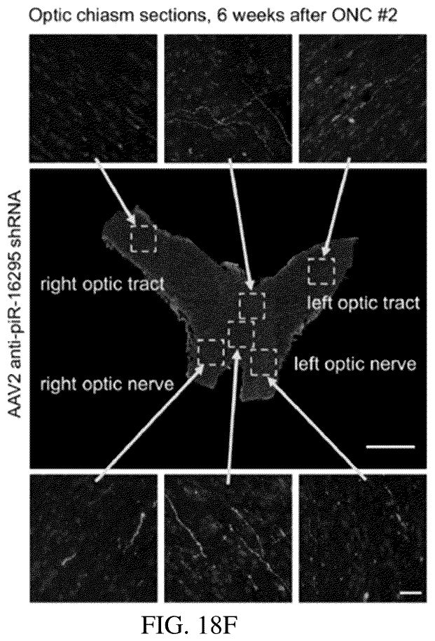

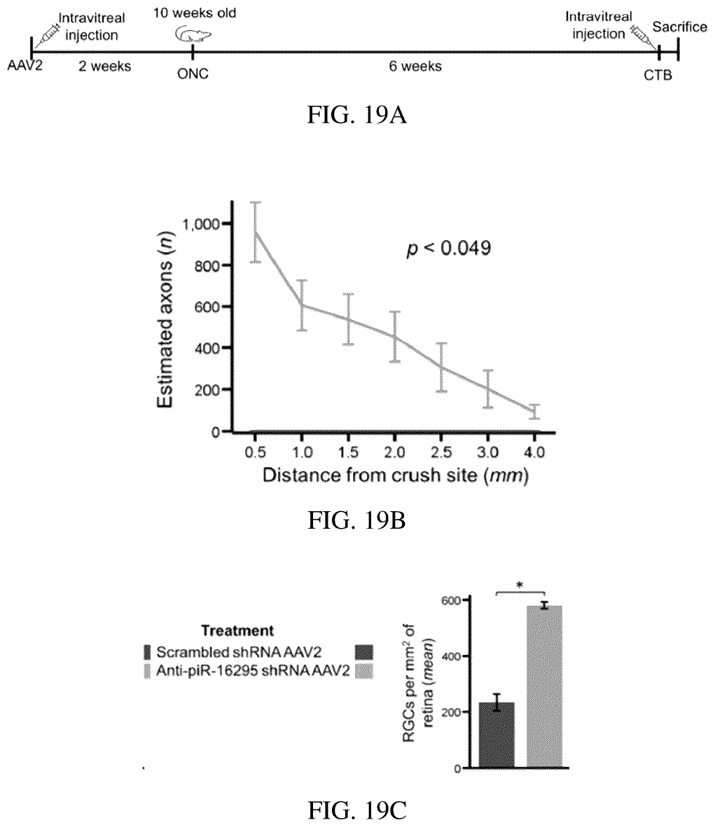

In a similar manner, the AAV2 vector expressing anti-piR16295 shRNA was then tested, except the effects were assessed at an even longer post-injury timepoint, 6 weeks after ONC (experimental timeline is shown in FIG. 18A). Data show that, relative to signs of retrograde degeneration (as expected) in control animals, in the experimentally-treated condition axons regenerated through the full-length of the optic nerve (FIGS. 18B-18C, FIG. 19B), with some axons regenerating through the optic chiasm and into the optic tracts (FIG. 18E-F). RGC survival also was significantly promoted by the anti-piR16295 shRNA AAV2 at 6 weeks post-injury, compared to the control-treated animals (FIG. 18D, FIG. 19C).

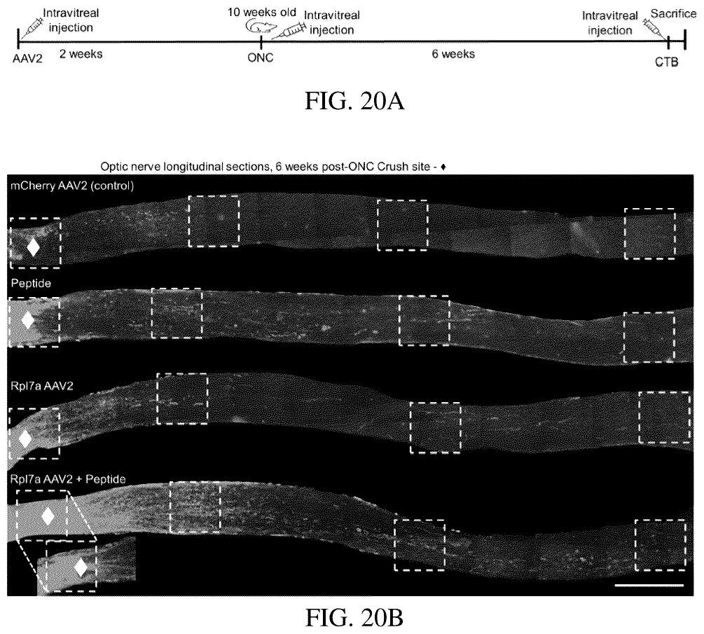

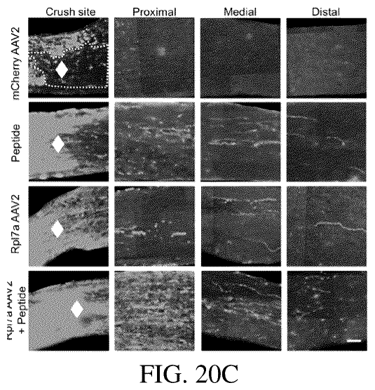

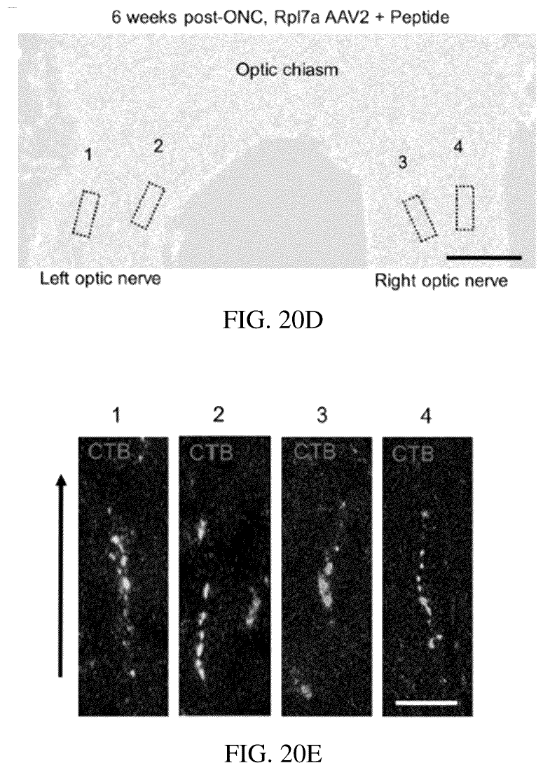

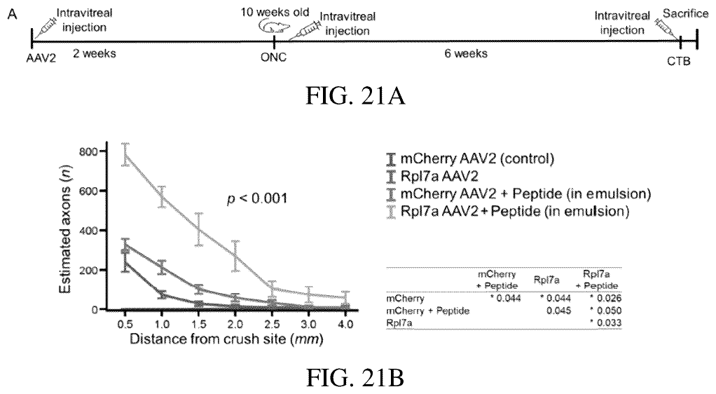

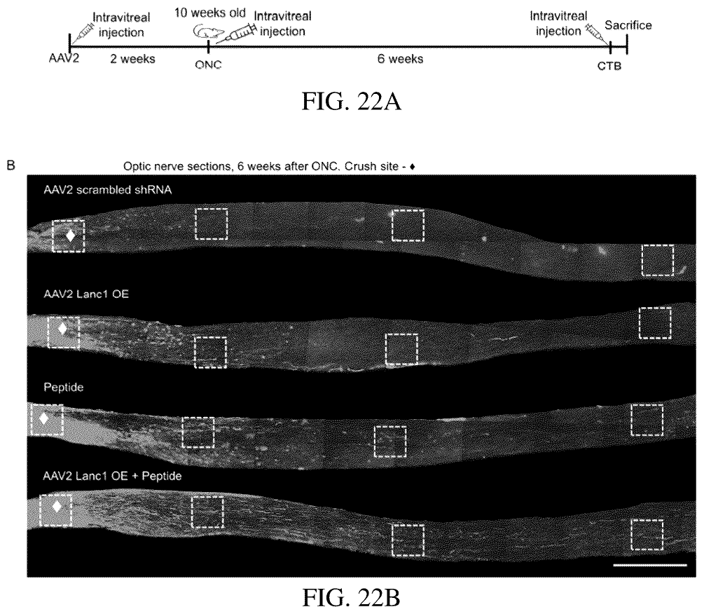

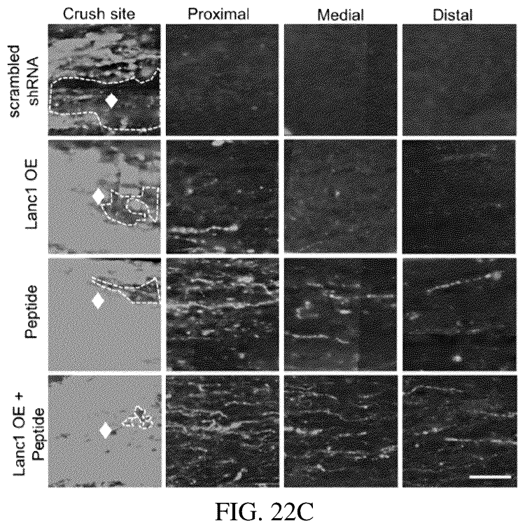

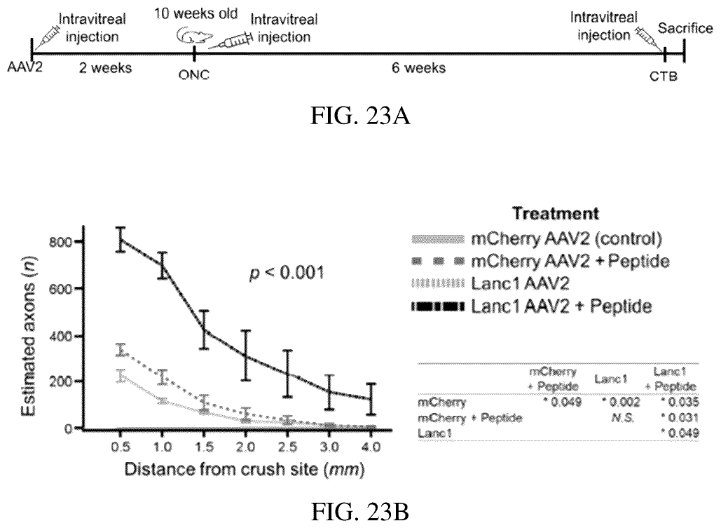

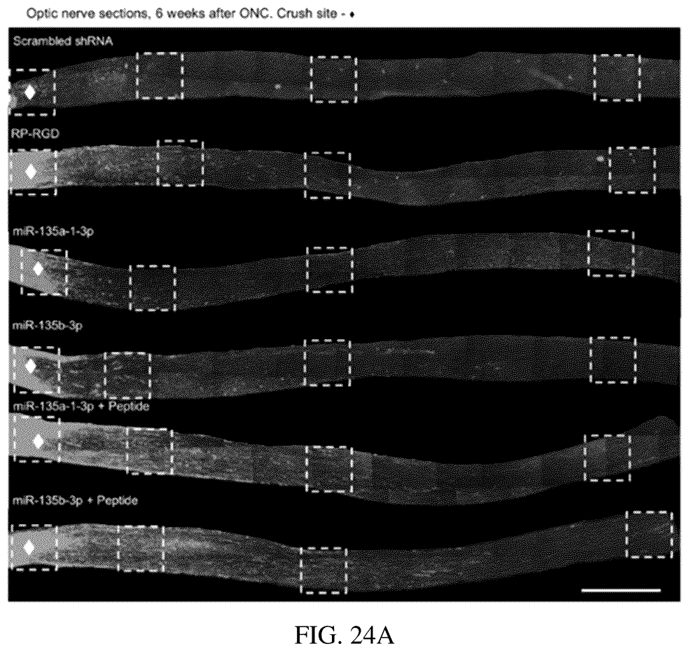

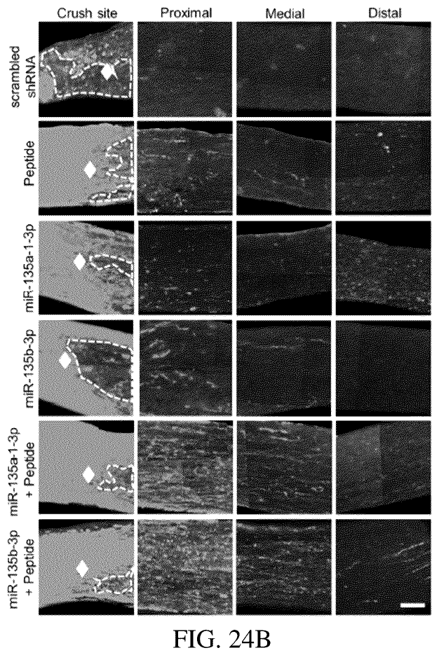

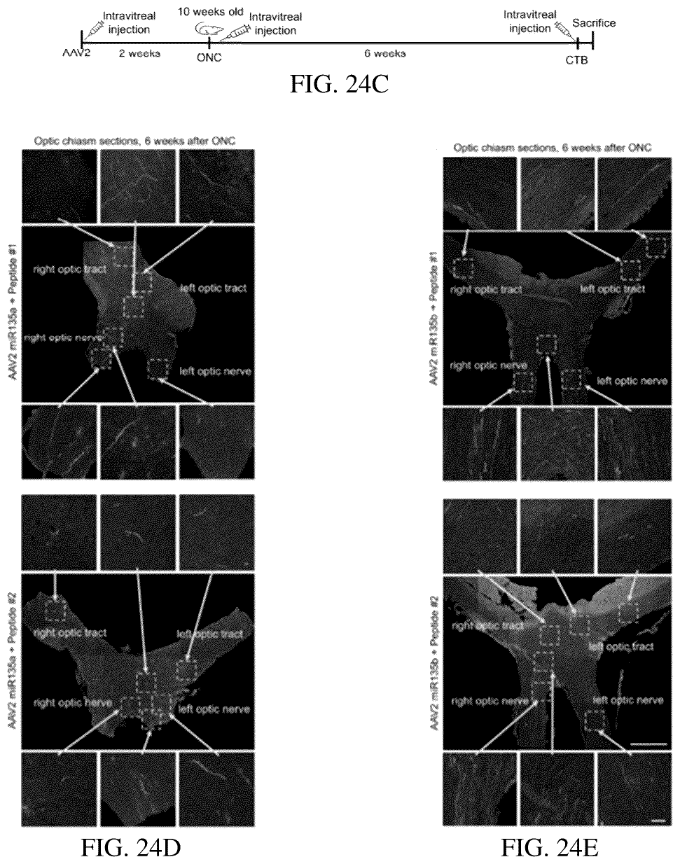

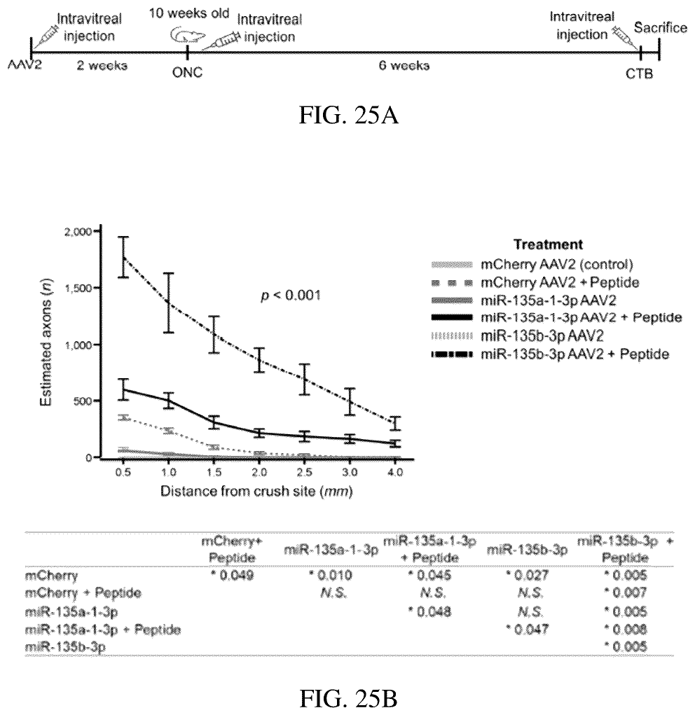

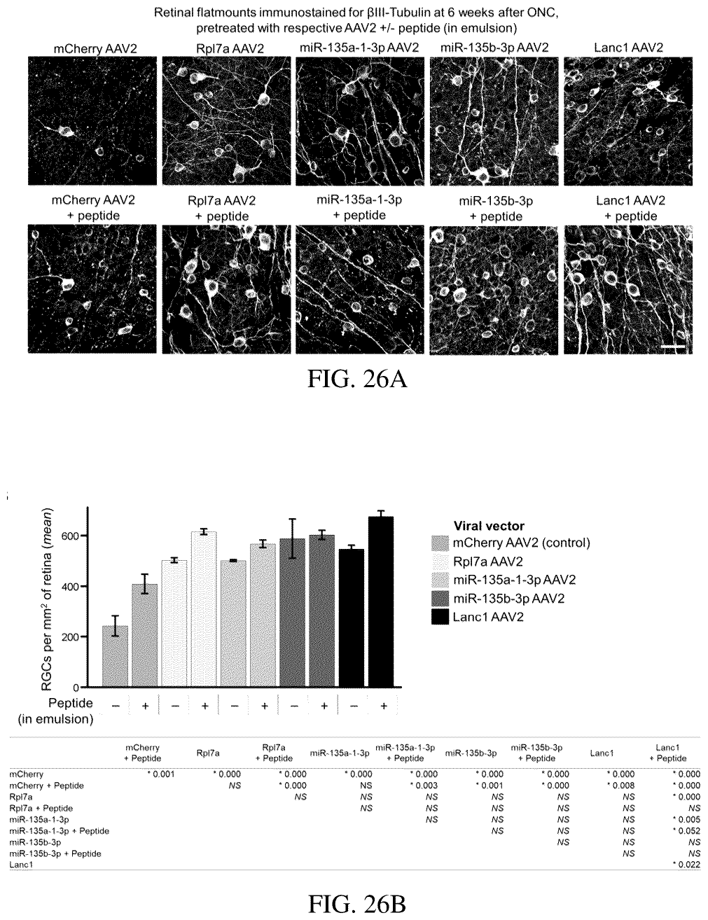

For a proof-of-concept regarding long-term effects of combinatory treatments, the long-term therapeutic potential of co-treating with the fibronectin-based recombinant peptide and Rpl7a or Lancl1 or miR-135a-1-3p or miR-135b-3p gene therapies, which were initially discovered promote neuroprotection and axon regeneration at least through 2 weeks after ONC injury, were tested each on its own. Using the methods, controls, and experimental design described above, the extent of neuroprotection and axon regeneration elicited by the co-treatments were tested (experimental timelines are shown in FIGS. 20A, 22A, 24C). For the co-treatments of fibronectin-based recombinant peptide and Rpl7a or Lancl1, data show that by 6 weeks after ONC, relative to signs of retrograde degeneration (as expected) in control animals, in the experimentally-co-treated conditions axons regenerated through the full-length of the optic nerve, whereas the individual experimental treatments, although significant, were less effective (FIGS. 20B-20E, 21B, 22B-22C, 23B). For the co-treatments of fibronectin-based recombinant peptide and miR-135a-1-3p-mimic or miR-135b-3p-mimic, data show that by 6 weeks after ONC, relative to signs of retrograde degeneration (as expected) in control animals, in the experimentally-co-treated conditions axons regenerated through the full-length of the optic nerve, whereas the individual experimental treatments, although significant, were less effective (FIGS. 24A-24B, 25B). Moreover, in the co-treatment conditions, some axons regenerating through the optic chiasm and into the optic tracts (FIGS. 24C-24E). RGC survival also was significantly promoted by each of the individual treatments, and further enhanced, albeit marginally, by the co-treatments, at 6 weeks post-injury, compared to the control-treated animals (FIG. 26A and Table 8 below showing quantitation of RGC survival at 6 weeks after ONC analyzed using ANOVA (overall F=14.2, p<0.001), with p-values of pairwise comparisons determined by posthoc LSD and significant differences (p<0.05) indicated by an asterisk *. Mean±SEM shown; n=3-5 cases per group an asterisk *.

TABLE 8

| | | | | | miR- | | | | |
|---|---|---|---|---|---|---|---|---|---|
| | mCherry + | | Rp17a + | miR- | 135a-1-3p + | | miR135b-3p + | | Lanc1 + |
| | Peptide | Rp17a | Peptide | 135a-1-3p | Peptide | miR135b-3p | Peptide | Lanc1 | Peptide |
| mCherry | * 0.001 | * 0.000 | * 0.000 | * 0.000 | * 0.000 | * 0.000 | * 0.000 | * 0.000 | * 0.000 |
| mCherry + Peptide | | NS | * 0.000 | NS | * 0.003 | * 0.001 | * 0.000 | * 0.008 | * 0.000 |
| Rp17a | | | NS | NS | NS | NS | NS | NS | * 0.000 |
| Rp17a + Peptide | | | | NS | NS | NS | NS | NS | NS |
| miR-135a-1-3p | | | | | NS | NS | NS | NS | * 0.005 |
| miR-135a-1-3p + | | | | | | NS | NS | NS | * 0.052 |
| Peptide | | | | | | | | | |
| miR135b-3p | | | | | | | NS | NS | NS |
| miR135b-3p + | | | | | | | | NS | NS |
| Peptide | | | | | | | | | |
| Lanc1 | | | | | | | | | * 0.022 |

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A fibronectin-based peptide comprising a fibronectin peptide that is SEQ ID NO:41 or that has at least 90%, 91%, 92%, 93%, 94%, 95%, 96%, 97%, 98%, 99%, or 99.5% sequence identity with the amino acid sequence:
SEQ ID NO:41.

2. The fibronectin-based peptide of claim 1, further comprising a signal peptide or an affinity tag.

3. The fibronectin-based peptide of claim 2, wherein the signal peptide comprises the amino acid sequence MGWSCIILFLVATATGVHS (SEQ ID NO:42) and the affinity tag is a Histidine tag.

4. A composition for promoting neuroprotection or axon regeneration of an injured axon, comprising the fibronectin-based peptide of claim 1, and optionally a pharmaceutically acceptable carrier, vehicle, additive, excipient, diluent, adjuvant, or stabilizer.

5. A composition for promoting neuroprotection or axon regeneration of an injured axon, comprising one or more of:
(i) a nucleic acid molecule comprising one or more synthetic small hairpin RNA (shRNA) molecules with one or more shRNA stem-loop regions, wherein the shRNA comprises one or more regions complementary to a portion of the target RNA, wherein hybridization of the complementary region of the shRNA to the target RNA of a target gene blocks target RNA function and promotes axon neuroprotection and/or regeneration,
(ii) an expression cassette, the expression cassette comprising a synthetic nucleic acid open reading frame (ORF) molecule of a gene involved in axon regeneration, wherein the gene is Rpl7, Rpl7a, Tceb2, Lancl1, Atp6v0c, Dynlt1a, Mrtfa, Lars2, Dpysl5, Fblim1, Dypsl3, or Nfe213, wherein expression of the ORF promotes axon neuroprotection and/or regeneration,
(iii) a nucleic acid molecule comprising an miRNA sequence or an miRNA mimic of said miRNA involved in axon regeneration, wherein the miRNA is miR-5109, miR-1247-5p, miR-210-3p, miR-3914-1, miR-135b-3p, miR-135a-3p, wherein expression of the miRNA promotes axon neuroprotection and/or regeneration;
(iv) a protein product expressed from one or more of the open reading frames in (ii), wherein the protein product promotes axon neuroprotection and/or regeneration.

6. The composition of claim 5 wherein
(a) the target RNA in (i) is an mRNA or a small noncoding RNA (sncRNA);
(b) the target RNA in (i) is an mRNA of target gene Mmp9, Rax, Crx, Pdnp, Prdm13, or ift20 mRNA and its lncRNA isoform ENSMUST00000128788;
(c) the nucleic acid in (i) comprises at least 2 shRNA stem-loop regions, or at least 3 shRNA stem-loop regions, or at least 4 shRNA stem-loop regions;
(d) the synthetic ORF in (ii) for target gene Rpl7 comprises the Rpl7 sequence identified in SEQ ID NO: 2, the synthetic ORF for target gene Rpl7a comprises the Rpl7a sequence identified in SEQ ID NO: 1, ORF for target gene Tceb2 comprises the Tceb2 sequence identified in SEQ ID NO: 3, the synthetic ORF for target gene Lancl1 comprises the Lancl1 sequence identified in SEQ ID NO:17, the synthetic ORF for target gene Atp6v0c comprises the Atp6v0c sequence identified in SEQ ID NO: 16, the synthetic ORF for target gene Dynlt1a comprises the Dynlt1a sequence identified in SEQ ID NO: 15, the synthetic ORF for target gene Mrtfa comprises the Mrtfa sequence identified in SEQ ID NO:13, the synthetic ORF for target gene Lars2 comprises the Lars2 sequence identified in SEQ ID NO: 12, the synthetic ORF of target gene Dpys15 comprises the Dpysl5 sequence identified in SEQ ID NO: 5, the synthetic ORF for target gene Fblim1 comprises the Fblim1 sequence identified in SEQ ID NO: 6, ORF for target gene Dypsl3 comprises the Dypsl3 sequence identified in SEQ ID NO: 4, ORF of target gene Nfe213 comprises the Nfe213 sequence identified in SEQ ID NO:7;
(e) the target RNA in (i) is sncRNA and is Piwi-interacting RNA (piRNA).

7. The composition of claim 6, wherein the piRNA is piR-16295, wherein the piRNA is piR-16295 and the shRNA sequence is identified in SEQ ID NO:29.

8. The composition of claim 5, wherein the target RNA in (i) is Mmp9 and the shRNA sequence is identified in SEQ ID NO: 14, the target gene is Rax and shRNA sequence is identified in SEQ ID NO: 11, the target gene is Crx and the shRNA sequence is identified in SEQ ID NO:10, the target gene is Pdnp and the shRNA sequence is identified in SEQ ID NO: 8, or the target gene is Prdm13 and the shRNA sequence is identified in SEQ ID NO: 9.

9. The composition of claim 5, wherein the shRNA molecule or ORF is expressed from a viral vector selected from the group consisting of retrovirus, lentivirus, adenovirus, herpesvirus, poxvirus, alpha virus, vaccinia virus, and adeno-associated (AAV) virus.

10. The composition of claim 5, wherein the composition further comprises a vehicle, wherein the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus.

11. A method for promoting axon regeneration or neuroprotection of axons in a subject, the method comprising administering to the subject the composition of claim 1.

12. The method of claim 11, wherein the method further comprises co-administering the composition of claim 5 to said subject.

13. The method of claim 12, wherein the nucleic acid in (i) is one or more of the following:
(a) the target RNA in (i) is an mRNA or a small noncoding RNA (sncRNA);
(b) the target RNA in (i) is an mRNA of target gene Mmp9, Rax, Crx, Pdnp, Prdm13, or ift20 mRNA and its lncRNA isoform ENSMUST00000128788;
(c) the nucleic acid in (i) comprises at least 2 shRNA stem-loop regions, or at least 3 shRNA stem-loop regions, or at least 4 shRNA stem-loop regions;
(d) the target RNA in (i) is sncRNA and is Piwi-interacting RNA (piRNA).

14. The method of claim 12, wherein the synthetic ORF in (ii) is one or more of the following:
for target gene Rpl7 comprises the Rpl7 sequence identified in SEQ ID NO: 2, the synthetic ORF for target gene Rpl7a comprises the Rpl7a sequence identified in SEQ ID NO: 1, ORF for target gene Tceb2 comprises the Tceb2 sequence identified in SEQ ID NO: 3, the synthetic ORF for target gene Lancl1 comprises the Lancl1 sequence identified in SEQ ID NO:17, the synthetic ORF for target gene Atp6v0c comprises the Atp6v0c sequence identified in SEQ ID NO: 16, the synthetic ORF for target gene Dynlt1a comprises the Dynlt1a sequence identified in SEQ ID NO: 15, the synthetic ORF for target gene Mrtfa comprises the Mrtfa sequence identified in SEQ ID NO:13, the synthetic ORF for target gene Lars2 comprises the Lars2 sequence identified in SEQ ID NO: 12, the synthetic ORF of target gene Dpys15 comprises the Dpys15 sequence identified in SEQ ID NO: 5, the synthetic ORF for target gene Fblim1 comprises the Fblim1 sequence identified in SEQ ID NO: 6, ORF for target gene Dypsl3 comprises the Dypsl3 sequence identified in SEQ ID NO: 4, ORF of target gene Nfe213 comprises the Nfe213 sequence identified in SEQ ID NO:7.

15. The method according to claim 13, wherein:
the target gene of (b) is Mmp9 and the shRNA sequence is identified in SEQ ID NO: 14, the target gene is Rax and shRNA sequence is identified in SEQ ID NO: 11, the target gene is Crx and the shRNA sequence is identified in SEQ ID NO:10, the target gene is Pdnp and the shRNA sequence is identified in SEQ ID NO: 8, or the target gene is Prdm13 and the shRNA sequence is identified in SEQ ID NO: 9.

16. The method of claim 13, wherein:
the piRNA is piR-16295; or
the piRNA is piR-16295 and the shRNA sequence is SEQ ID NO: 29.

17. The method of claim 13, wherein the shRNA molecule or the ORF is expressed from a viral vector selected from the group consisting of retrovirus, lentivirus, adenovirus, herpesvirus, poxvirus, alpha virus, vaccinia virus, and adeno-associated (AAV) virus.

18. The method of claim 12, wherein the composition further comprises a vehicle, wherein the vehicle comprises a lipid molecule, a liposome, a micelle, a cationic lipid, a protein particle, an inorganic nanoparticle, a nanoparticle, a cationic lipid, a cationic polymer, a nanorod, microbubbles, a liposphere, or a virus.

19. The method of claim 12, wherein administration is prior to injury or post injury, intravitreally, intravenously, intracortically, intracerebrally, intrathecally, intranasally, ocularly, or locally at the injured neuron.

20. The method of claim 12 wherein the subject has a disease or disorder comprising a neurodegenerative diseases, optic neuropathy, traumatic optic neuropathy, stroke or optic nerve damage from a stroke, spinal cord injury or traumatic spinal cord injury, glaucoma, head injury or head trauma, or a combination thereof, or wherein the subject has a neurodegenerative disease selected from the group consisting of sporadic Parkinson's disease, autosomal recessive early-onset Parkinson's disease, Alzheimer's disease, Friedreich ataxia, Lewy body disease, Spinal muscular atrophy, stroke, amyotrophic lateral sclerosis, Binswanger's disease, Huntington's Disease, Huntington's chorea, multiple sclerosis, myasthenia gravis, and Pick's disease, Alpers' disease, Batten disease, cerebro-oculo-facio-skeletal syndrome, corticobasal degeneration, Gerstmann-Straussler-Scheinker disease, kuru, Leigh Syndrome, Monomelic amyotrophy, Multiple system atrophy, neurodegeneration with brain iron accumulation, opsoclonus myoclonus, striatonigral degeneration, transmissible spongiform encephalopathies, neuromyelitis optica, glaucoma, and optic nerve diseases.
